(19) United States
(12) Patent Application Publication
(10) Pub. No.: US 2022/0031676 A1
(43) Pub. Date: Feb. 3, 2022

(54) METHODS OF TREATING DISEASE WITH MAGL INHIBITORS

(71) Applicant: H. Lundbeck A/S, Valby (DK)

(72) Inventors: Channing Rodney BEALS, San Diego, CA (US); Dallas JONES, San Diego, CA (US); Jason Robert CLAPPER, San Diego, CA (US); Gary Paul O'NEILL, San Diego, CA (US); Iain Peter FRASER, San Diego, CA (US); Jacqueline Lorayne BLANKMAN, San Diego, CA (US); John J.M. WIENER, San Diego, CA (US); Cheryl A. GRICE, San Diego, CA (US)

(21) Appl. No.: 17/296,489

(22) PCT Filed: Nov. 26, 2019
(86) PCT No.: PCT/US19/63451

§ 371 Date: May 24, 2021

Related U.S. Application Data
(60) Provisional application No. 62/772,554, filed on Nov. 28, 2018.

(51) Int. Cl. A61K 31/438; A61K 31/496; A61K 31/5386; A61K 31/506

(52) U.S. Cl. CPC A61K 31/438; A61K 31/506; A61K 31/5386; A61K 31/496

(57) ABSTRACT

Provided herein are methods for the treatment of disease with monoacylglycerol lipase (MAGL) inhibitors.

### CROSS-REFERENCE

This application claims benefit of U.S. Provisional Application No. 62/772,554, filed on Nov. 28, 2018, which is herein incorporated by reference in its entirety.

### BACKGROUND

Monoacylglycerol lipase (MAGL) is an enzyme responsible for hydrolyzing endocannabinoids such as 2-AG (2-arachidonoylglycerol), an arachidonate based lipid, in the nervous system.

### BRIEF SUMMARY OF THE INVENTION

This disclosure provides, for example, methods for treating disease with compounds and pharmaceutical compositions which are modulators of MAGL. The disclosure also provides for the use of disclosed compounds as medicaments and/or in the manufacture of medicaments for the inhibition of MAGL activity in warm-blooded animals such as humans.

In some embodiments is a method for treating a disease or a condition in a patient wherein the disease or condition is selected from atopic dermatitis, bladder dysfunction associated with multiple sclerosis, cardiovascular disease, contact dermatitis, cystic fibrosis, dermatomyositis, eczema, endometriosis, enteritis, fibromyalgia, Tourette syndrome, inflammatory bowel disease, interstitial cystitis, irritable bowel syndrome, ischemia, labor, abdominal pain, abdominal pain associated with irritable bowel syndrome, acute pain, back pain, cancer pain, chest pain, functional chest pain, joint pain, menstrual pain, metabolic disorders, musculoskeletal diseases, neuropathy, osteoarthritis, pancreatitis, pharyngitis, post mastectomy pain syndrome, post trigeminal neuralgia, post operative pain, renal ischemia, rheumatoid arthritis, skeletal muscle contusion, skin diseases, sunburn, systemic lupus erythematosus, toothache, vasoocclusive painful crises in sickle cell disease, and visceral pain, comprising administering to the patient in need thereof a therapeutically effective amount of a compound of Formula (I) having the structure:

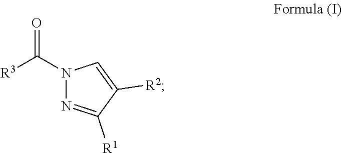

wherein:
R¹ is —C(O)OR¹⁵ or —C(O)NR¹⁰R¹¹;
R² is H, halogen, C₁₋₆alkyl, or C₁₋₆haloalkyl;
R³ is

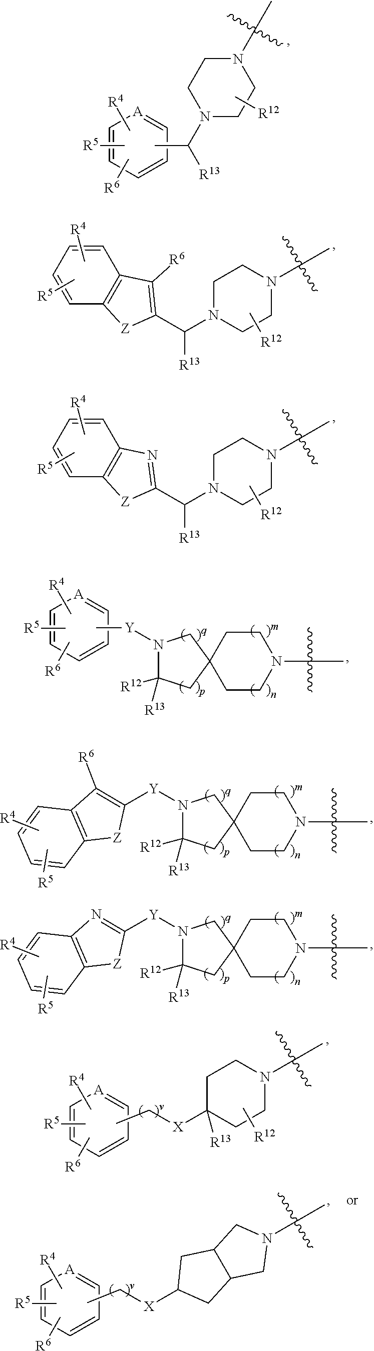

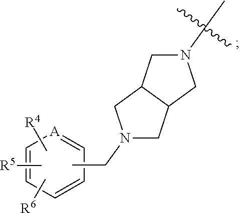

A is N or C(H);
X is —O—, —N(R¹⁶)—, or —CH₂N(R¹⁶)CH₂—;
Y is —CH₂— or —C(O)—;
Z is —S—, —O—, or —N(R¹⁸)—;
R⁴ is H, halogen, —OR⁷, C₁₋₆alkyl, C₂₋₆alkenyl, C₂₋₆alkynyl, C₁₋₆haloalkyl, —C(O)NR⁸R⁹, C₃₋₈cycloalkyl, C₂₋₉heterocycloalkyl, —C₁₋₆alkyl-C₂₋₉heterocycloalkyl, C₆₋₁₀aryl, or C₁₋₉heteroaryl, wherein C₃₋₈cycloalkyl, C₂₋₉heterocycloalkyl, —C₁₋₆alkyl-C₂₋₉ heterocycloalkyl, C₆₋₁₀aryl, or C₁₋₉heteroaryl are optionally substituted with 1 or 2 R¹⁴;
R⁵ is H, halogen, C₁₋₆alkyl, C₁₋₆haloalkyl, C₁₋₆haloalkoxy, or phenyl;
R⁶ is H, halogen or C₁₋₆alkyl;
R⁷ is H, C₁₋₆alkyl, C₁₋₆haloalkyl, —C₁₋₆alkyl-OH, C₂₋₉heterocycloalkyl, C₆₋₁₀aryl, or C₁₋₉heteroaryl, wherein C₂₋₉heterocycloalkyl, C₆₋₁₀aryl, or C₁₋₉heteroaryl are optionally substituted with 1 or 2 R¹⁴;
each R⁸ and each R⁹ are independently selected from H and C₁₋₆alkyl; or R⁸ and R⁹ together with the nitrogen to which they are attached are combined to form a heterocycloalkyl ring;
R¹⁰ and R¹¹ are each independently H or C₁₋₆alkyl;
R¹² is H, halogen, or C₁₋₆alkyl;
R¹³ is H or C₁₋₆alkyl;
each R¹⁴ is independently selected from halogen, —OH, C₁₋₆alkyl, C₁₋₆haloalkyl, C₁₋₆alkoxy, —C₁₋₆alkyl-OH, C₃₋₈cycloalkyl, —C(O)OH, —C(O)NR⁸R⁹, —SO₂—C₁₋₆alkyl, and —N(R¹⁷)C(O)—C₁₋₆alkyl;
R¹⁵ is H or C₁₋₆alkyl;
R¹⁶ is H, C₁₋₆alkyl, —C(O)—C₁₋₆alkyl, —C₁₋₆alkyl-OH, or —CH₂CO₂H;
R¹⁷ is H or C₁₋₆alkyl;
R¹⁸ is H or C₁₋₆alkyl;
v is 0 or 1;
n is 0 or 1;
m is 0 or 1;
p is 0, 1, or 2; and
q is 0, 1 or 2;
   or a solvate, hydrate, tautomer, N-oxide, stereoisomer, or pharmaceutically acceptable salt thereof.

In some embodiments is a method for treating a disease or a condition in a patient wherein the disease or condition is selected from atopic dermatitis, bladder dysfunction associated with multiple sclerosis, cardiovascular disease, contact dermatitis, cystic fibrosis, dermatomyositis, eczema, endometriosis, enteritis, fibromyalgia, Tourette syndrome, inflammatory bowel disease, interstitial cystitis, irritable bowel syndrome, ischemia, labor, abdominal pain, abdominal pain associated with irritable bowel syndrome, acute pain, back pain, cancer pain, chest pain, functional chest pain, joint pain, menstrual pain, metabolic disorders, musculoskeletal diseases, neuropathy, osteoarthritis, pancreatitis, pharyngitis, post mastectomy pain syndrome, post trigeminal neuralgia, post operative pain, renal ischemia, rheumatoid arthritis, skeletal muscle contusion, skin diseases, sunburn, systemic lupus erythematosus, toothache, vasoocclusive painful crises in sickle cell disease, and visceral pain, comprising administering to the patient in need thereof a therapeutically effective amount of a compound of Formula (I′) having the structure:

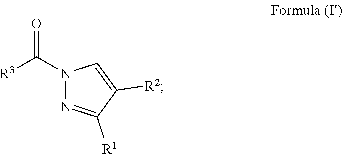

wherein:
R¹ is —C(O)OR¹⁵ or —C(O)NR¹⁰R¹¹;
R² is H, halogen, C₁₋₆alkyl, or C₁₋₆haloalkyl;
R³ is

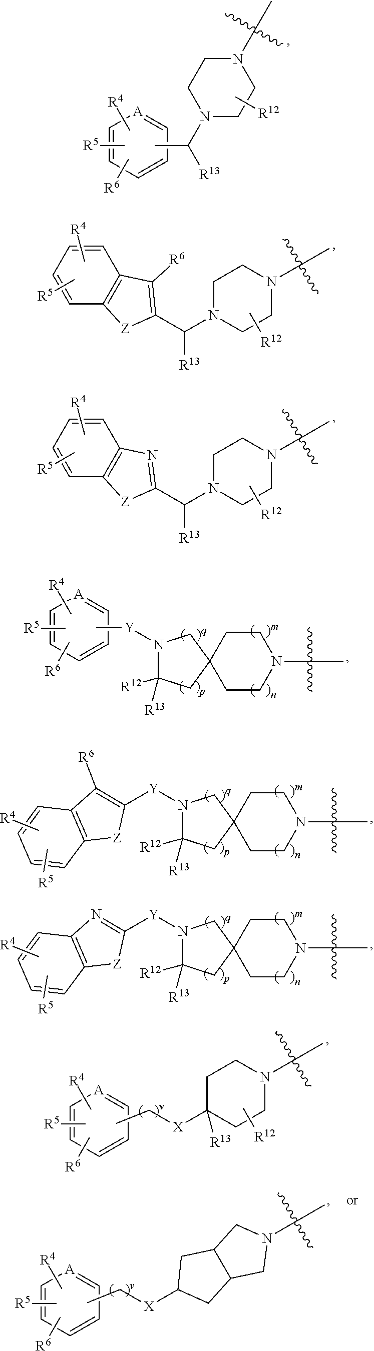

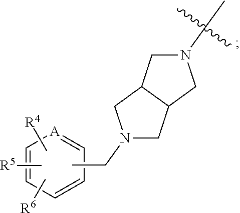

A is N or C(H);
X is —O—, —N(R¹⁶)—, or —CH₂N(R¹⁶)CH₂—;
Y is —CH₂— or —C(O)—;
Z is —S—, —O—, or —N(R¹⁸)—;
R⁴ is H, halogen, —OR⁷, C₁₋₆alkyl, C₂₋₆alkenyl, C₂₋₆alkynyl, C₁₋₆haloalkyl, —C(O)NR⁸R⁹, C₂₋₉heterocycloalkyl, —C₁₋₆alkyl-C₂₋₉heterocycloalkyl, C₆₋₁₀aryl, or C₁₋₉heteroaryl, wherein C₂₋₉heterocycloalkyl, —C₁₋₆alkyl-C₂₋₉heterocycloalkyl, C₆₋₁₀aryl, or C₁₋₉heteroaryl are optionally substituted with 1 or 2 R¹⁴;
R⁵ is H, halogen, C₁₋₆alkyl, C₁₋₆haloalkyl, C₁₋₆haloalkoxy, or phenyl;
R⁶ is H, halogen or C₁₋₆alkyl;
R⁷ is H, C₁₋₆alkyl, C₁₋₆haloalkyl, C₂₋₉heterocycloalkyl, C₆₋₁₀aryl, or C₁₋₉heteroaryl, wherein C₂₋₉heterocycloalkyl, C₆₋₁₀aryl, or C₁₋₉heteroaryl are optionally substituted with 1 or 2 R¹⁴;
each R⁸ and each R⁹ are independently selected from H and C₁₋₆alkyl; or R⁸ and R⁹ together with the nitrogen to which they are attached are combined to form a heterocycloalkyl ring;
R¹⁰ and R¹¹ are each independently H or C₁₋₆alkyl;
R¹² is H, halogen, or C₁₋₆alkyl;
R¹³ is H or C₁₋₆alkyl;
each R¹⁴ is independently selected from halogen, —OH, C₁₋₆alkyl, C₁₋₆haloalkyl, C₁₋₆alkoxy, —C₁₋₆alkyl-OH, C₃₋₈cycloalkyl, —C(O)NR⁸R⁹, —SO₂—C₁₋₆alkyl, and —N(R¹⁷)C(O)—C₁₋₆alkyl;
R¹⁵ is H or C₁₋₆alkyl;
R¹⁶ is H, C₁₋₆alkyl, —C(O)—C₁₋₆alkyl, or —CH₂CO₂H;
R¹⁷ is H or C₁₋₆alkyl;
R¹⁸ is H or C₁₋₆alkyl;
v is 0 or 1;
n is 0 or 1;
m is 0 or 1;
p is 0, 1, or 2; and
q is 0, 1 or 2;
or a solvate, hydrate, tautomer, N-oxide, stereoisomer, or pharmaceutically acceptable salt thereof.

In some embodiments of the methods for treating a disease or a condition with a compound of Formula (I) or (I′), or a solvate, hydrate, tautomer, N-oxide, stereoisomer, or pharmaceutically acceptable salt thereof, R³ is

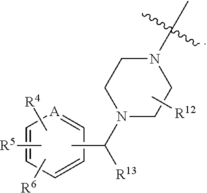

In some embodiments of the methods for treating a disease or a condition with a compound of Formula (I) or (I′), or a solvate, hydrate, tautomer, N-oxide, stereoisomer, or pharmaceutically acceptable salt thereof, A is C(H). In some embodiments of the methods for treating a disease or a condition with a compound of Formula (I) or (I′), or a solvate, hydrate, tautomer, N-oxide, stereoisomer, or pharmaceutically acceptable salt thereof, A is N. In some embodiments of the methods for treating a disease or a condition with a compound of Formula (I) or (I′), or a solvate, hydrate, tautomer, N-oxide, stereoisomer, or pharmaceutically acceptable salt thereof, R⁶ is H. In some embodiments of the methods for treating a disease or a condition with a compound of Formula (I) or (I′), or a solvate, hydrate, tautomer, N-oxide, stereoisomer, or pharmaceutically acceptable salt thereof, R³ is

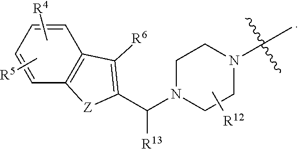

In some embodiments of the methods for treating a disease or a condition with a compound of Formula (I) or (I′), or a solvate, hydrate, tautomer, N-oxide, stereoisomer, or pharmaceutically acceptable salt thereof, R³ is

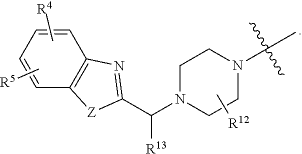

In some embodiments of the methods for treating a disease or a condition with a compound of Formula (I) or (I′), or a solvate, hydrate, tautomer, N-oxide, stereoisomer, or pharmaceutically acceptable salt thereof, Z is —S—. In some embodiments of the methods for treating a disease or a condition with a compound of Formula (I) or (I′), or a solvate, hydrate, tautomer, N-oxide, stereoisomer, or pharmaceutically acceptable salt thereof, Z is —N(H)—. In some embodiments of the methods for treating a disease or a condition with a compound of Formula (I) or (I′), or a solvate, hydrate, tautomer, N-oxide, stereoisomer, or pharmaceutically acceptable salt thereof, R³ is

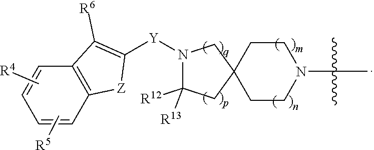

In some embodiments of the methods for treating a disease or a condition with a compound of Formula (I) or (I′), or a solvate, hydrate, tautomer, N-oxide, stereoisomer, or pharmaceutically acceptable salt thereof, Z is —S—. In some embodiments of the methods for treating a disease or a condition with a compound of Formula (I) or (I′), or a solvate, hydrate, tautomer, N-oxide, stereoisomer, or pharmaceutically acceptable salt thereof, R³ is

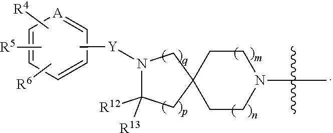

In some embodiments of the methods for treating a disease or a condition with a compound of Formula (I) or (I′), or a solvate, hydrate, tautomer, N-oxide, stereoisomer, or pharmaceutically acceptable salt thereof, m is 1, n is 1, q is 1, and p is 1. In some embodiments of the methods for treating a disease or a condition with a compound of Formula (I) or (I′), or a solvate, hydrate, tautomer, N-oxide, stereoisomer, or pharmaceutically acceptable salt thereof, m is 0, n is 1, q is 2, and p is 1. In some embodiments of the methods for treating a disease or a condition with a compound of Formula (I) or (I′), or a solvate, hydrate, tautomer, N-oxide, stereoisomer, or pharmaceutically acceptable salt thereof, m is 1, n is 1, q is 0, and p is 2. In some embodiments of the methods for treating a disease or a condition with a compound of Formula (I) or (I′), or a solvate, hydrate, tautomer, N-oxide, stereoisomer, or pharmaceutically acceptable salt thereof, m is 1, n is 1, q is 1, and p is 0. In some embodiments of the methods for treating a disease or a condition with a compound of Formula (I) or (I′), or a solvate, hydrate, tautomer, N-oxide, stereoisomer, or pharmaceutically acceptable salt thereof, Y is —CH₂—. In some embodiments of the methods for treating a disease or a condition with a compound of Formula (I) or (I′), or a solvate, hydrate, tautomer, N-oxide, stereoisomer, or pharmaceutically acceptable salt thereof, Y is —C(O)—. In some embodiments of the methods for treating a disease or a condition with a compound of Formula (I) or (I′), or a solvate, hydrate, tautomer, N-oxide, stereoisomer, or pharmaceutically acceptable salt thereof, R¹³ is H. In some embodiments of the methods for treating a disease or a condition with a compound of Formula (I) or (I′), or a solvate, hydrate, tautomer, N-oxide, stereoisomer, or pharmaceutically acceptable salt thereof, R¹² is H. In some embodiments of the methods for treating a disease or a condition with a compound of Formula (I) or (I′), or a solvate, hydrate, tautomer, N-oxide, stereoisomer, or pharmaceutically acceptable salt thereof, R³ is

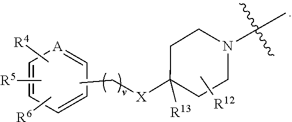

In some embodiments of the methods for treating a disease or a condition with a compound of Formula (I) or (I′), or a solvate, hydrate, tautomer, N-oxide, stereoisomer, or pharmaceutically acceptable salt thereof, R¹³ is H. In some embodiments of the methods for treating a disease or a condition with a compound of Formula (I) or (I′), or a solvate, hydrate, tautomer, N-oxide, stereoisomer, or pharmaceutically acceptable salt thereof, R¹³ is —CH₃. In some embodiments of the methods for treating a disease or a condition with a compound of Formula (I) or (I′), or a solvate, hydrate, tautomer, N-oxide, stereoisomer, or pharmaceutically acceptable salt thereof, R¹² is H. In some embodiments of the methods for treating a disease or a condition with a compound of Formula (I) or (I′), or a solvate, hydrate, tautomer, N-oxide, stereoisomer, or pharmaceutically acceptable salt thereof, R³ is

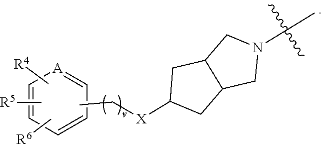

In some embodiments of the methods for treating a disease or a condition with a compound of Formula (I) or (I′), or a solvate, hydrate, tautomer, N-oxide, stereoisomer, or pharmaceutically acceptable salt thereof, v is 0. In some embodiments of the methods for treating a disease or a condition with a compound of Formula (I) or (I′), or a solvate, hydrate, tautomer, N-oxide, stereoisomer, or pharmaceutically acceptable salt thereof, v is 1. In some embodiments of the methods for treating a disease or a condition with a compound of Formula (I) or (I′), or a solvate, hydrate, tautomer, N-oxide, stereoisomer, or pharmaceutically acceptable salt thereof, X is —O—. In some embodiments of the methods for treating a disease or a condition with a compound of Formula (I) or (I′), or a solvate, hydrate, tautomer, N-oxide, stereoisomer, or pharmaceutically acceptable salt thereof, X is —N(CH₃)—. In some embodiments of the methods for treating a disease or a condition with a compound of Formula (I) or (I′), or a solvate, hydrate, tautomer, N-oxide, stereoisomer, or pharmaceutically acceptable salt thereof, X is —CH₂N(CH₃)CH₂—. In some embodiments of the methods for treating a disease or a condition with a compound of Formula (I) or (I′), or a solvate, hydrate, tautomer, N-oxide, stereoisomer, or pharmaceutically acceptable salt thereof, R³ is

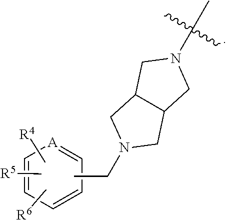

In some embodiments of the methods for treating a disease or a condition with a compound of Formula (I) or (I′), or a solvate, hydrate, tautomer, N-oxide, stereoisomer, or pharmaceutically acceptable salt thereof, A is C(H). In some embodiments of the methods for treating a disease or a condition with a compound of Formula (I) or (I′), or a solvate, hydrate, tautomer, N-oxide, stereoisomer, or pharmaceutically acceptable salt thereof, A is N. In some embodiments of the methods for treating a disease or a condition with a compound of Formula (I) or (I′), or a solvate, hydrate, tautomer, N-oxide, stereoisomer, or pharmaceutically acceptable salt thereof, R⁶ is H. In some embodiments of the methods for treating a disease or a condition with a compound of Formula (I) or (I′), or a solvate, hydrate, tautomer, N-oxide, stereoisomer, or pharmaceutically acceptable salt thereof, R⁴ is halogen, —OR⁷, C₁₋₆alkyl, C₂₋₆alkynyl, C₁₋₆haloalkyl, —C(O)NR⁸R⁹, C₂₋₉heterocycloalkyl, C₆₋₁₀aryl, or C₁₋₉heteroaryl, wherein C₂₋₉heterocycloalkyl, —C₁₋₆alkyl-C₂₋₉heterocycloalkyl, C₆₋₁₀aryl, or C₁₋₉heteroaryl are optionally substituted with 1 or 2 R¹⁴. In some embodiments of the methods for treating a disease or a condition with a compound of Formula (I) or (I′), or a solvate, hydrate, tautomer, N-oxide, stereoisomer, or pharmaceutically acceptable salt thereof, R⁴ is halogen, —OR⁷, C₁₋₆haloalkyl, C₂₋₉heterocycloalkyl, C₆₋₁₀aryl, or C₁₋₉heteroaryl, wherein C₂₋₉heterocycloalkyl, C₆₋₁₀aryl, or C₁₋₉heteroaryl are optionally substituted with 1 or 2 R¹⁴. In some embodiments of the methods for treating a disease or a condition with a compound of Formula (I) or (I′), or a solvate, hydrate, tautomer, N-oxide, stereoisomer, or pharmaceutically acceptable salt thereof, R⁴ is halogen. In some embodiments of the methods for treating a disease or a condition with a compound of Formula (I) or (I′), or a solvate, hydrate, tautomer, N-oxide, stereoisomer, or pharmaceutically acceptable salt thereof, R⁴ is —Cl. In some embodiments of the methods for treating a disease or a condition with a compound of Formula (I) or (I′), or a solvate, hydrate, tautomer, N-oxide, stereoisomer, or pharmaceutically acceptable salt thereof, R⁴ is C₁₋₆haloalkyl. In some embodiments of the methods for treating a disease or a condition with a compound of Formula (I) or (I′), or a solvate, hydrate, tautomer, N-oxide, stereoisomer, or pharmaceutically acceptable salt thereof, R⁴ is —CF₃. In some embodiments of the methods for treating a disease or a condition with a compound of Formula (I) or (I′), or a solvate, hydrate, tautomer, N-oxide, stereoisomer, or pharmaceutically acceptable salt thereof, R⁴ is —OR⁷. In some embodiments of the methods for treating a disease or a condition with a compound of Formula (I) or (I′), or a solvate, hydrate, tautomer, N-oxide, stereoisomer, or pharmaceutically acceptable salt thereof, R⁷ is C₁₋₆haloalkyl, C₆₋₁₀ aryl, or C₁₋₉heteroaryl, wherein C₆₋₁₀aryl or C₁₋₉heteroaryl are optionally substituted with 1 or 2 R¹⁴. In some embodiments of the methods for treating a disease or a condition with a compound of Formula (I) or (I′), or a solvate, hydrate, tautomer, N-oxide, stereoisomer, or pharmaceutically acceptable salt thereof, R⁷ is phenyl, pyridyl, pyrimidinyl, pyridizinyl, or pyrazinyl, wherein phenyl, pyridyl, pyrimidinyl, pyridizinyl, or pyrazinyl are optionally substituted with halogen or C₁₋₆haloalkyl. In some embodiments of the methods for treating a disease or a condition with a compound of Formula (I) or (I′), or a solvate, hydrate, tautomer, N-oxide, stereoisomer, or pharmaceutically acceptable salt thereof, R⁴ is C₂₋₉heterocycloalkyl optionally substituted with 1 or 2 R¹⁴. In some embodiments of the methods for treating a disease or a condition with a compound of Formula (I) or (I′), or a solvate, hydrate, tautomer, N-oxide, stereoisomer, or pharmaceutically acceptable salt thereof, R⁴ is an unsubstituted C₂₋₉heterocycloalkyl. In some embodiments of the methods for treating a disease or a condition with a compound of Formula (I) or (I′), or a solvate, hydrate, tautomer, N-oxide, stereoisomer, or pharmaceutically acceptable salt thereof, R⁴ is C₂₋₉heterocycloalkyl substituted with 1 or 2 R¹⁴. In some embodiments of the methods for treating a disease or a condition with a compound of Formula (I) or (I′), or a solvate, hydrate, tautomer, N-oxide, stereoisomer, or pharmaceutically acceptable salt thereof, R⁴ is

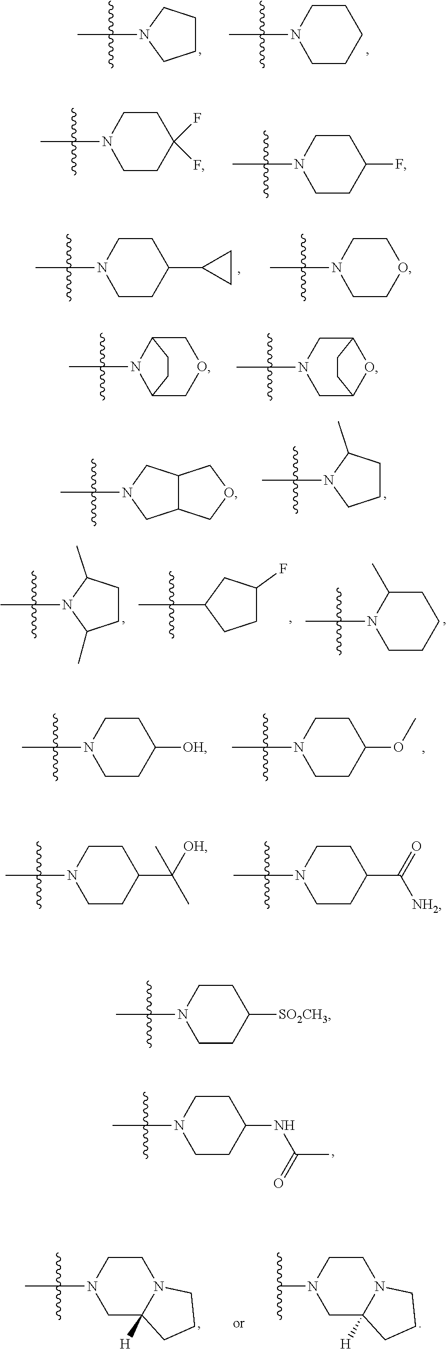

In some embodiments of the methods for treating a disease or a condition with a compound of Formula (I) or (I′), or a solvate, hydrate, tautomer, N-oxide, stereoisomer, or pharmaceutically acceptable salt thereof, R⁴ is C₁₋₉heteroaryl optionally substituted with 1 or 2 R¹⁴. In some embodiments of the methods for treating a disease or a condition with a compound of Formula (I) or (F), or a solvate, hydrate, tautomer, N-oxide, stereoisomer, or pharmaceutically acceptable salt thereof, R⁴ is an unsubstituted C₁₋₉heteroaryl. In some embodiments of the methods for treating a disease or a condition with a compound of Formula (I) or (I′), or a solvate, hydrate, tautomer, N-oxide, stereoisomer, or pharmaceutically acceptable salt thereof, R⁴ is C₂₋₉heteroaryl substituted with 1 or 2 R¹⁴. In some embodiments of the methods for treating a disease or a condition with a compound of Formula (I) or (I′), or a solvate, hydrate, tautomer, N-oxide, stereoisomer, or pharmaceutically acceptable salt thereof, R⁴ is

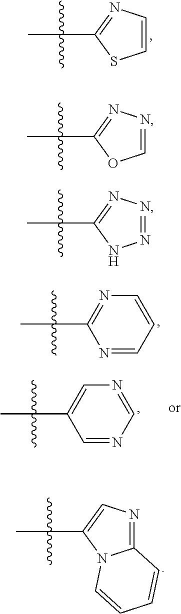

In some embodiments of the methods for treating a disease or a condition with a compound of Formula (I) or (I′), or a solvate, hydrate, tautomer, N-oxide, stereoisomer, or pharmaceutically acceptable salt thereof, R⁵ is H, halogen, C₁₋₆alkyl, C₁₋₆haloalkyl, or C₁₋₆haloalkoxy. In some embodiments of the methods for treating a disease or a condition with a compound of Formula (I) or (I′), or a solvate, hydrate, tautomer, N-oxide, stereoisomer, or pharmaceutically acceptable salt thereof, R⁵ is H. In some embodiments of the methods for treating a disease or a condition with a compound of Formula (I) or (F), or a solvate, hydrate, tautomer, N-oxide, stereoisomer, or pharmaceutically acceptable salt thereof, R⁵ is halogen. In some embodiments of the methods for treating a disease or a condition with a compound of Formula (I) or (I′), or a solvate, hydrate, tautomer, N-oxide, stereoisomer, or pharmaceutically acceptable salt thereof, R⁵ is —Cl. In some embodiments of the methods for treating a disease or a condition with a compound of Formula (I) or (I′), or a solvate, hydrate, tautomer, N-oxide, stereoisomer, or pharmaceutically acceptable salt thereof, R⁵ is C₁₋₆haloalkyl. In some embodiments of the methods for treating a disease or a condition with a compound of Formula (I) or (I′), or a solvate, hydrate, tautomer, N-oxide, stereoisomer, or pharmaceutically acceptable salt thereof, R⁵ is —CF₃. In some embodiments of the methods for treating a disease or a condition with a compound of Formula (I) or (I′), or a solvate, hydrate, tautomer, N-oxide, stereoisomer, or pharmaceutically acceptable salt thereof, R² is H. In some embodiments of the methods for treating a disease or a condition with a compound of Formula (I) or (I′), or a solvate, hydrate, tautomer, N-oxide, stereoisomer, or pharmaceutically acceptable salt thereof, R² is halogen. In some embodiments of the methods for treating a disease or a condition with a compound of Formula (I) or (I′), or a solvate, hydrate, tautomer, N-oxide, stereoisomer, or pharmaceutically acceptable salt thereof, R² is C₁₋₆alkyl. In some embodiments of the methods for treating a disease or a condition with a compound of Formula (I) or (I′), or a solvate, hydrate, tautomer, N-oxide, stereoisomer, or pharmaceutically acceptable salt thereof, R¹ is —C(O)OR¹⁵. In some embodiments of the methods for treating a disease or a condition with a compound of Formula (I) or (I′), or a solvate, hydrate, tautomer, N-oxide, stereoisomer, or pharmaceutically acceptable salt thereof, R¹⁵ is H. In some embodiments of the methods for treating a disease or a condition with a compound of Formula (I) or (I′), or a solvate, hydrate, tautomer, N-oxide, stereoisomer, or pharmaceutically acceptable salt thereof, R¹ is —C(O)NR¹⁰R¹¹. In some embodiments of the methods for treating a disease or a condition with a compound of Formula (I) or (I′), or a solvate, hydrate, tautomer, N-oxide, stereoisomer, or pharmaceutically acceptable salt thereof, R¹⁰ and R¹¹ are each H.

In some embodiments is method for treating a disease or a condition in a patient wherein the disease is selected from atopic dermatitis, bladder dysfunction associated with multiple sclerosis, cardiovascular disease, contact dermatitis, cystic fibrosis, dermatomyositis, eczema, endometriosis, enteritis, fibromyalgia, Tourette syndrome, inflammatory bowel disease, interstitial cystitis, irritable bowel syndrome, ischemia, labor, abdominal pain, abdominal pain associated with irritable bowel syndrome, acute pain, back pain, cancer pain, chest pain, functional chest pain, joint pain, menstrual pain, metabolic disorders, musculoskeletal diseases, neuropathy, osteoarthritis, pancreatitis, pharyngitis, post mastectomy pain syndrome, post trigeminal neuralgia, post operative pain, renal ischemia, rheumatoid arthritis, skeletal muscle contusion, skin diseases, sunburn, systemic lupus erythematosus, toothache, vasoocclusive painful crises in sickle cell disease, and visceral pain, comprising administering to the patient in need thereof a therapeutically effective amount of a compound having the structure:

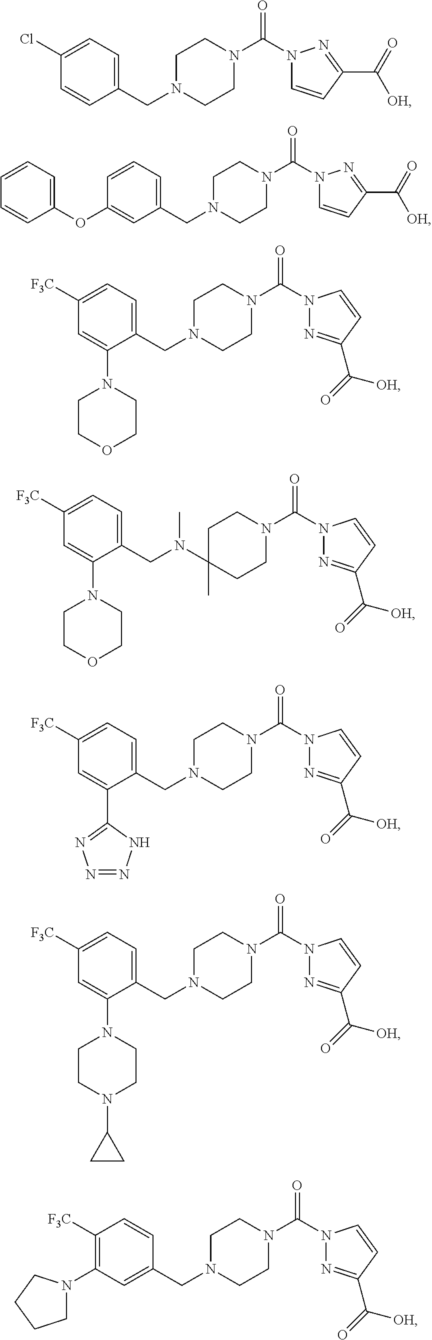

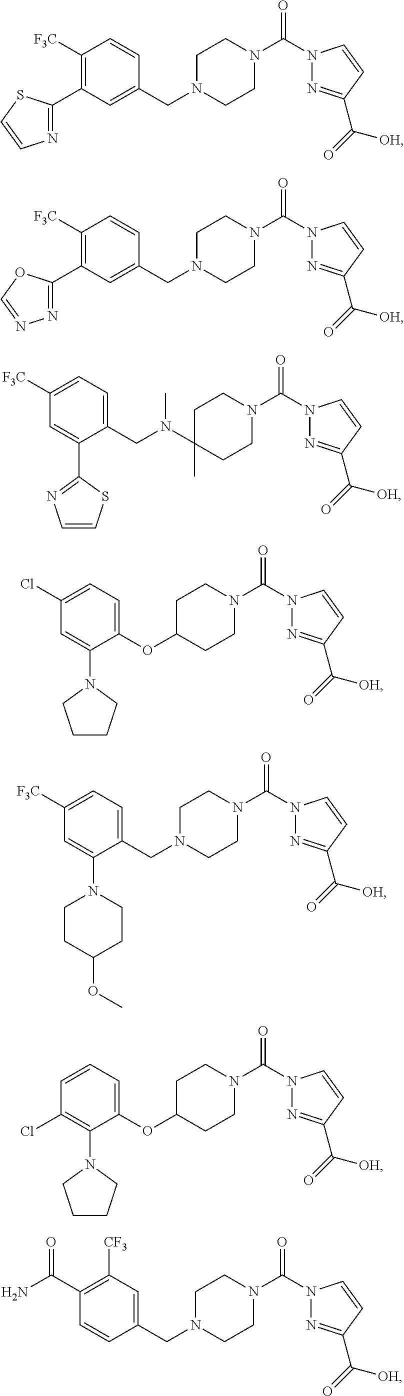

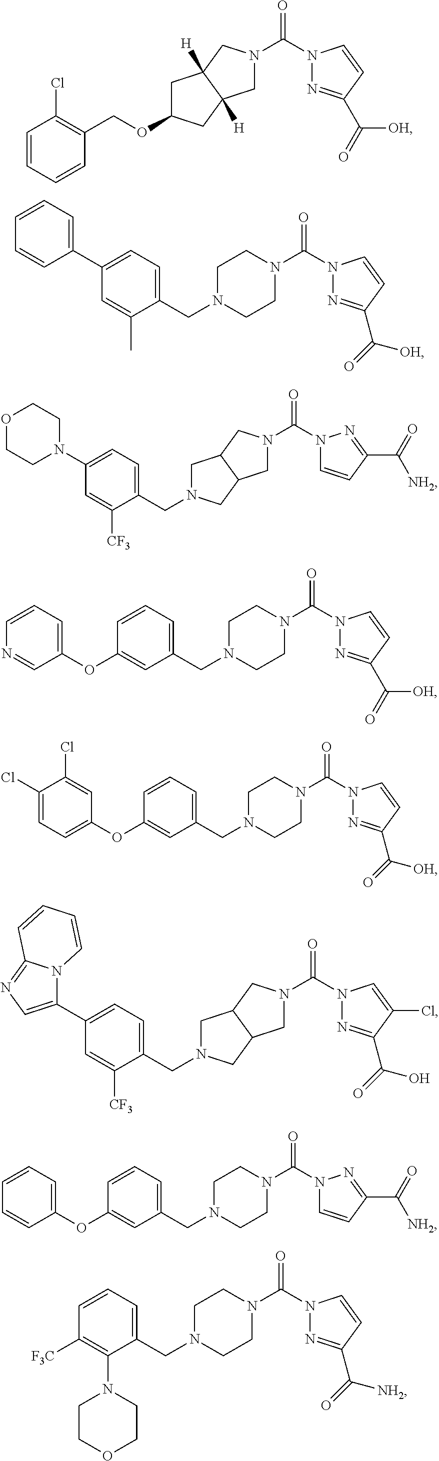

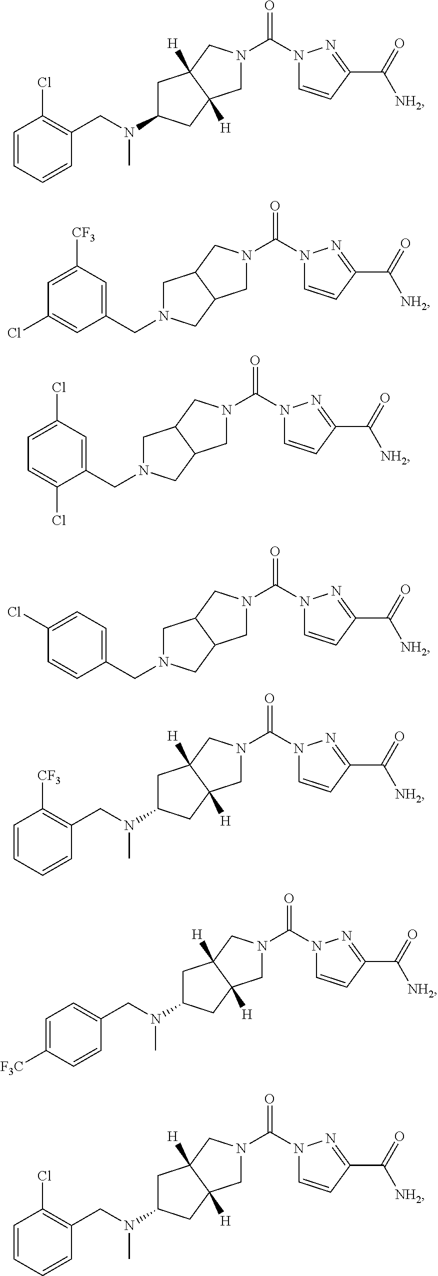

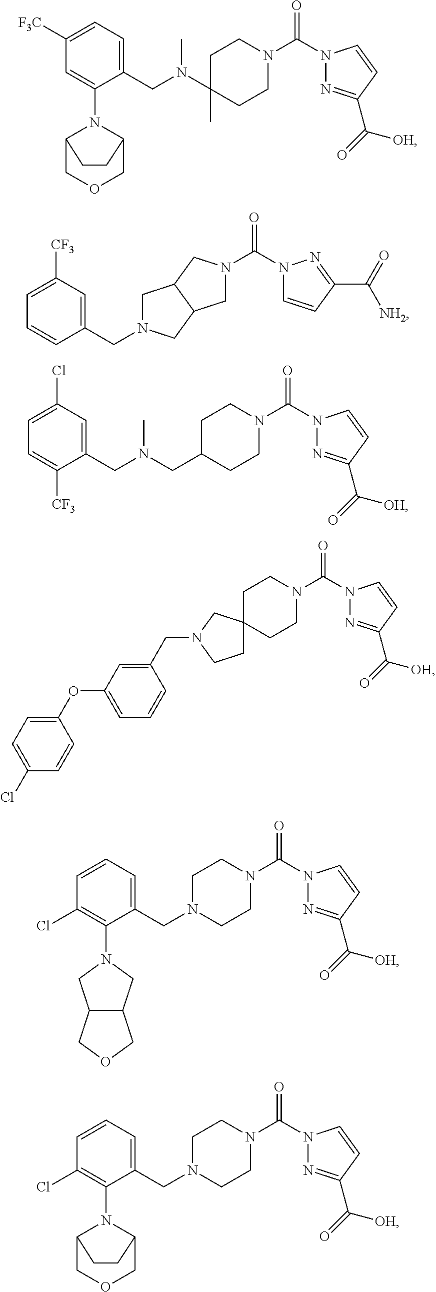

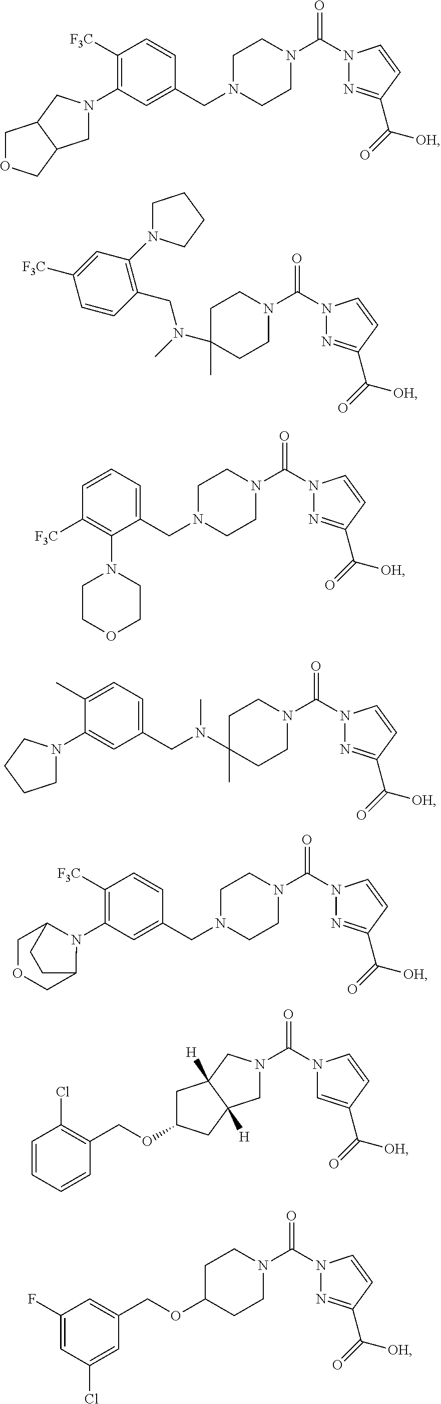

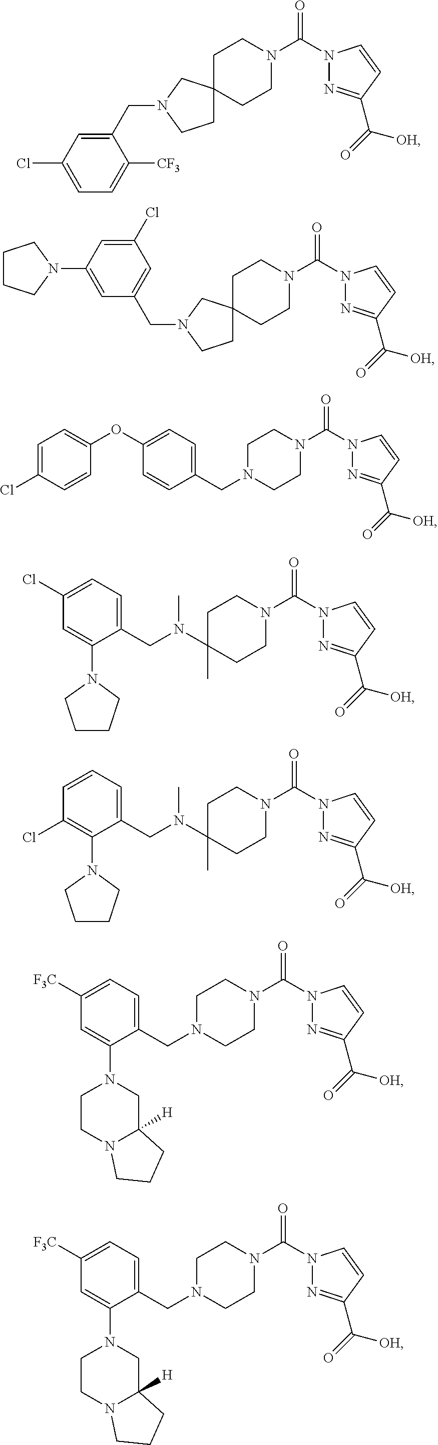

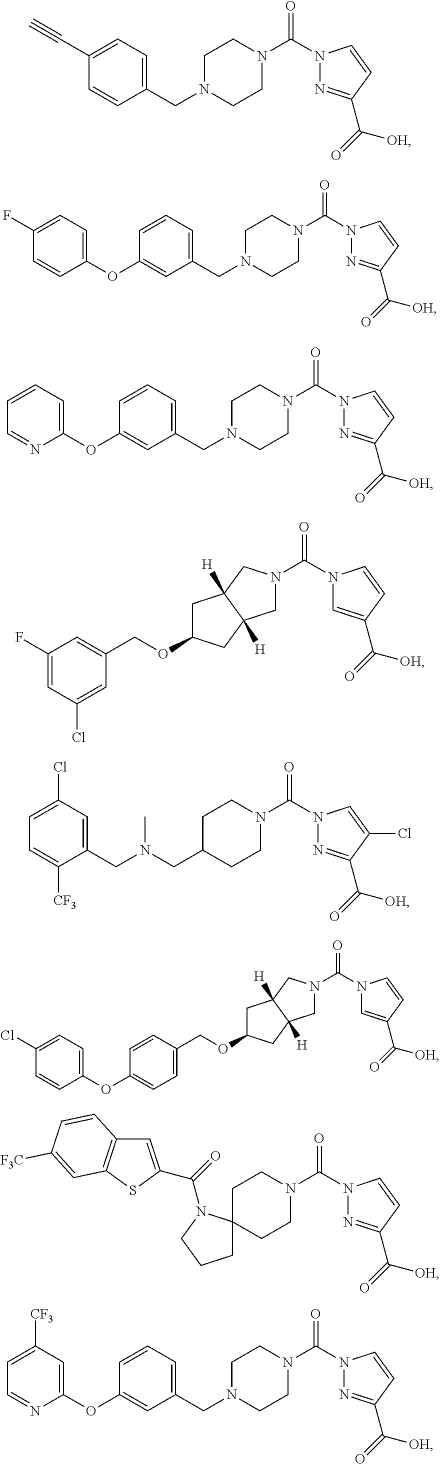

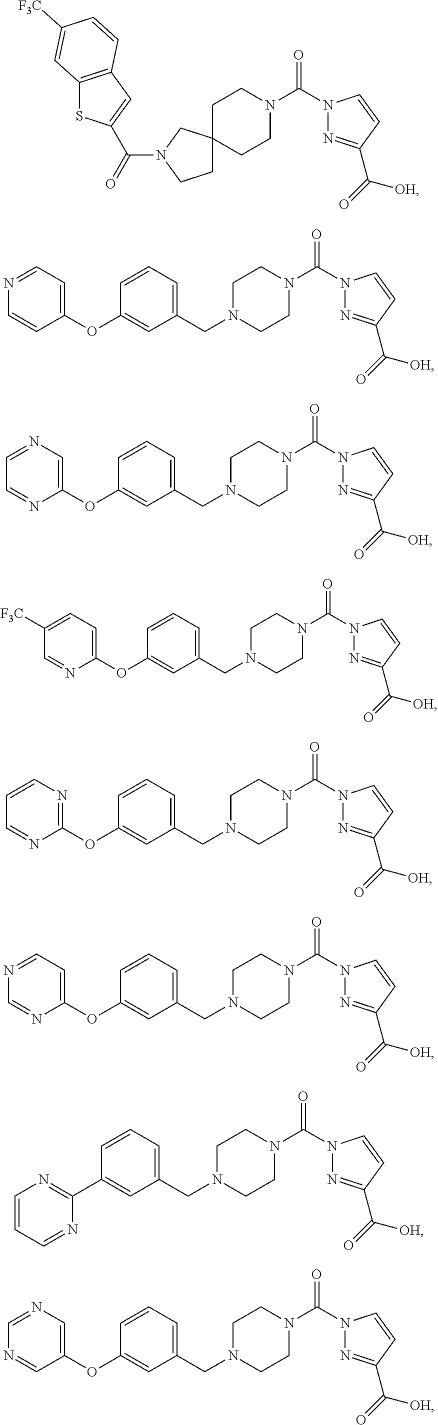

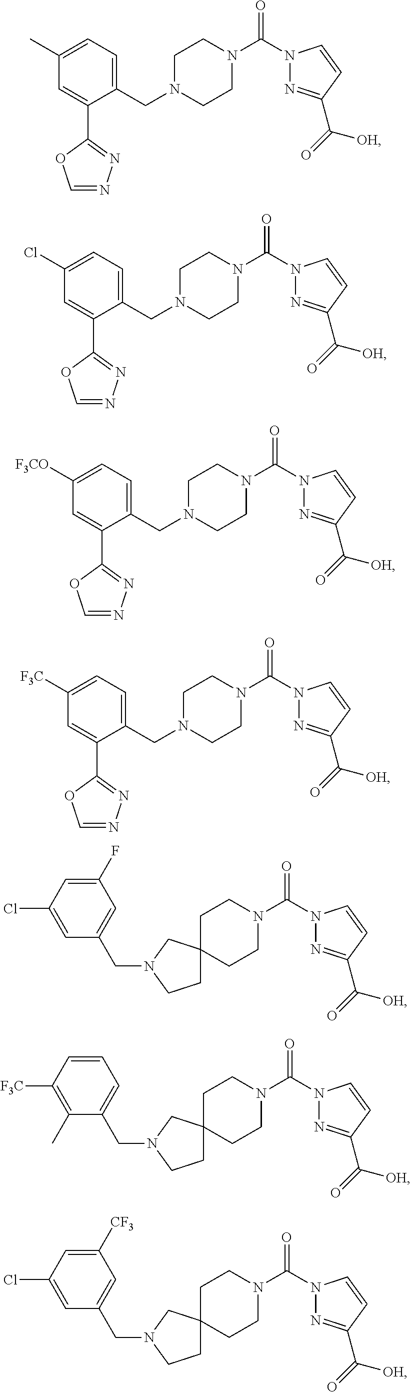

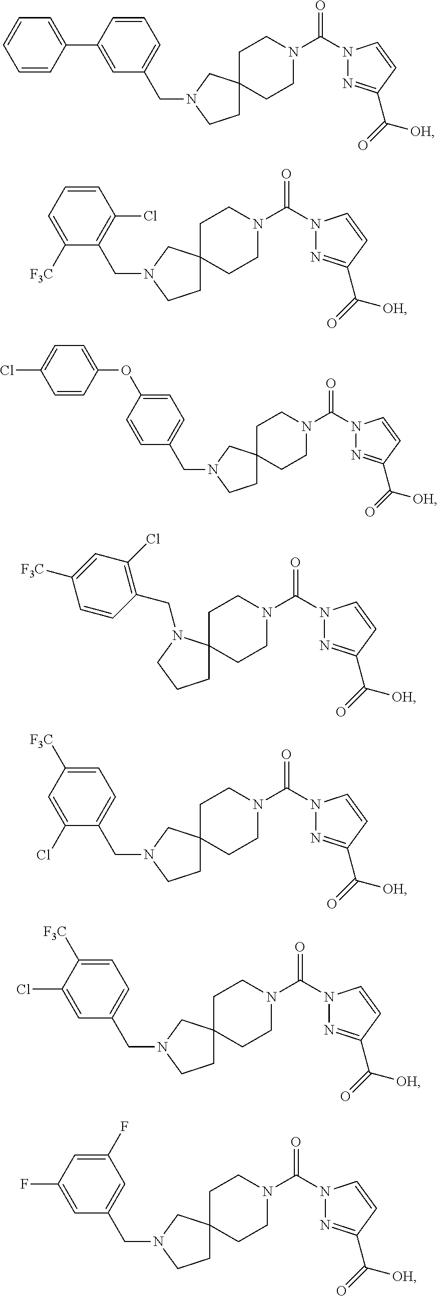

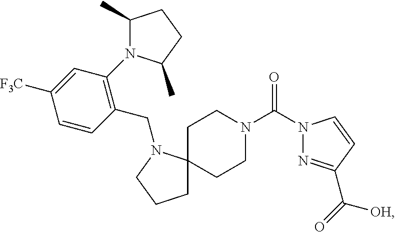 [SMILES: C[C@@H]1CC[C@H](C)N1c1cc(C(F)(F)F)ccc1CN1CCCC12CCN(C(=O)n1ccc(C(=O)O)n1)CC2]

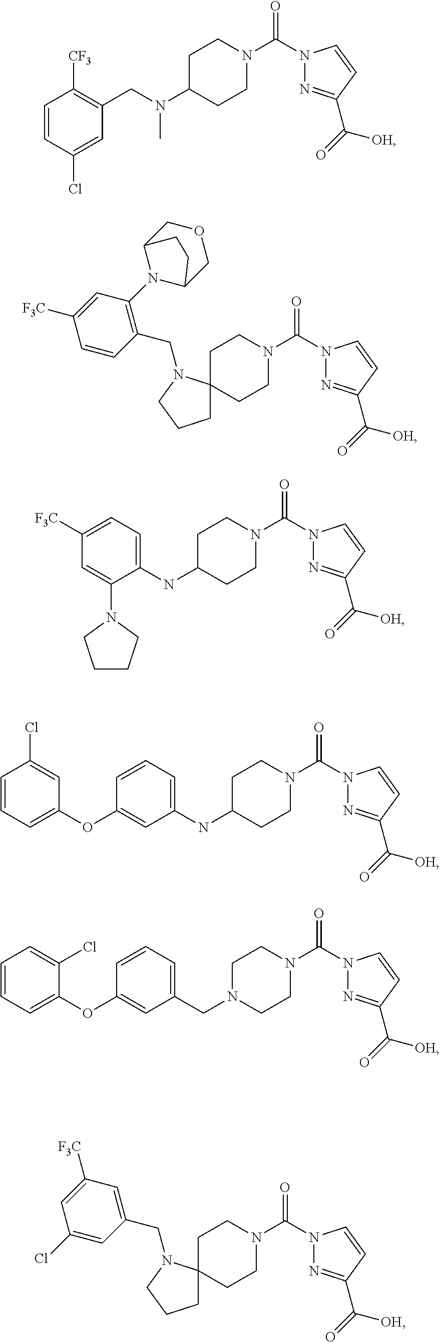 [SMILES: CN(Cc1cc(Cl)ccc1C(F)(F)F)C1CCN(C(=O)n2ccc(C(=O)O)n2)CC1 ; O=C(O)c1ccn(C(=O)N2CCC3(CCCN3Cc3ccc(C(F)(F)F)cc3N3C4CCC3COC4)CC2)n1 ; O=C(O)c1ccn(C(=O)N2CCC(Nc3ccc(C(F)(F)F)cc3N3CCCC3)CC2)n1 ; O=C(O)c1ccn(C(=O)N2CCC(Nc3cccc(Oc4cccc(Cl)c4)c3)CC2)n1 ; O=C(O)c1ccn(C(=O)N2CCN(Cc3cccc(Oc4ccccc4Cl)c3)CC2)n1 ; O=C(O)c1ccn(C(=O)N2CCC3(CCCN3Cc3cc(Cl)cc(C(F)(F)F)c3)CC2)n1]

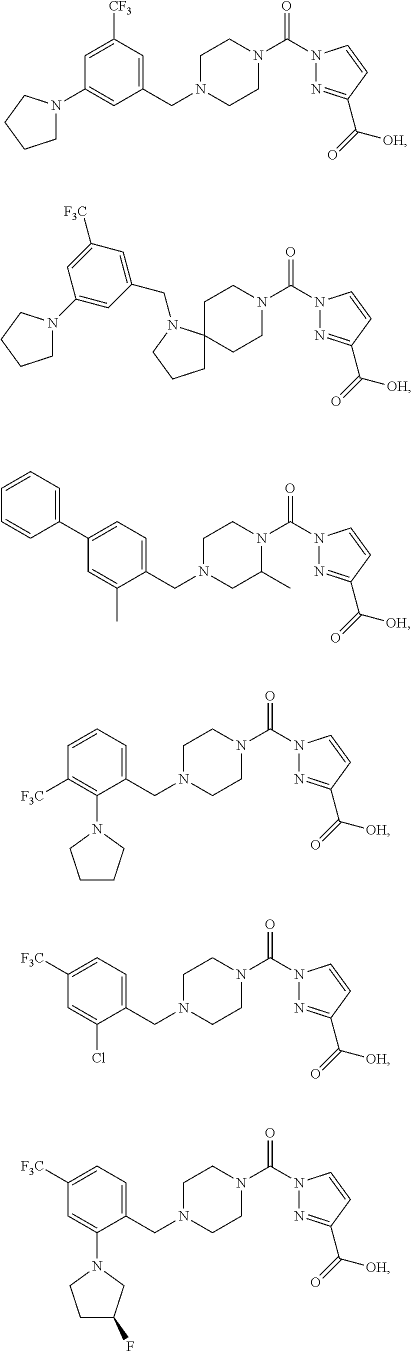 [SMILES: O=C(O)c1ccn(C(=O)N2CCN(Cc3cc(N4CCCC4)cc(C(F)(F)F)c3)CC2)n1 ; O=C(O)c1ccn(C(=O)N2CCC3(CCCN3Cc3cc(N4CCCC4)cc(C(F)(F)F)c3)CC2)n1 ; Cc1cc(-c2ccccc2)ccc1CN1CCN(C(=O)n2ccc(C(=O)O)n2)C(C)C1 ; O=C(O)c1ccn(C(=O)N2CCN(Cc3cccc(C(F)(F)F)c3N3CCCC3)CC2)n1 ; O=C(O)c1ccn(C(=O)N2CCN(Cc3ccc(C(F)(F)F)cc3Cl)CC2)n1 ; O=C(O)c1ccn(C(=O)N2CCN(Cc3ccc(C(F)(F)F)cc3N3CC[C@H](F)C3)CC2)n1]

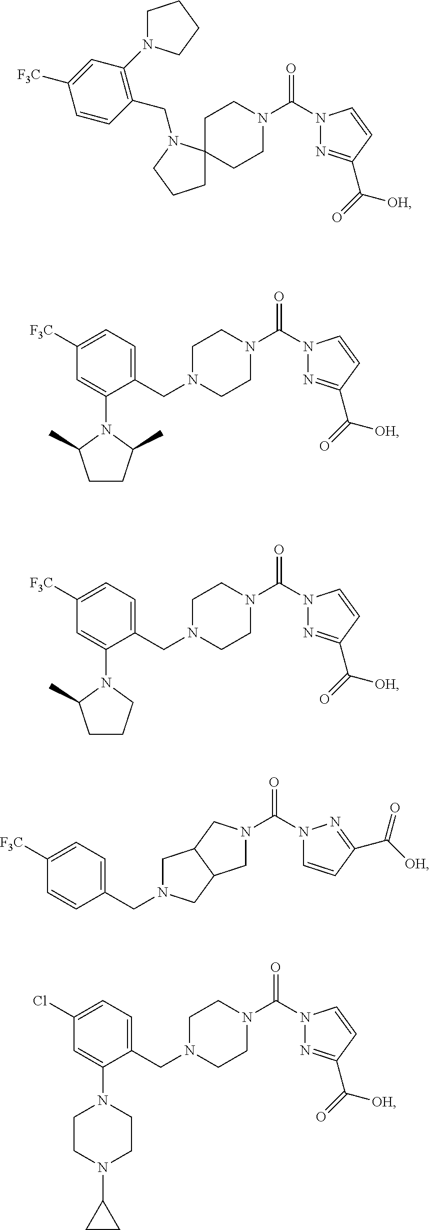 [SMILES: O=C(O)c1ccn(C(=O)N2CCC3(CCCN3Cc3ccc(C(F)(F)F)cc3N3CCCC3)CC2)n1 ; C[C@@H]1CC[C@H](C)N1c1cc(C(F)(F)F)ccc1CN1CCN(C(=O)n2ccc(C(=O)O)n2)CC1 ; C[C@@H]1CCCN1c1cc(C(F)(F)F)ccc1CN1CCN(C(=O)n2ccc(C(=O)O)n2)CC1 ; O=C(O)c1ccn(C(=O)N2CC3CN(Cc4ccc(C(F)(F)F)cc4)CC3C2)n1 ; O=C(O)c1ccn(C(=O)N2CCN(Cc3ccc(Cl)cc3N3CCN(C4CC4)CC3)CC2)n1]

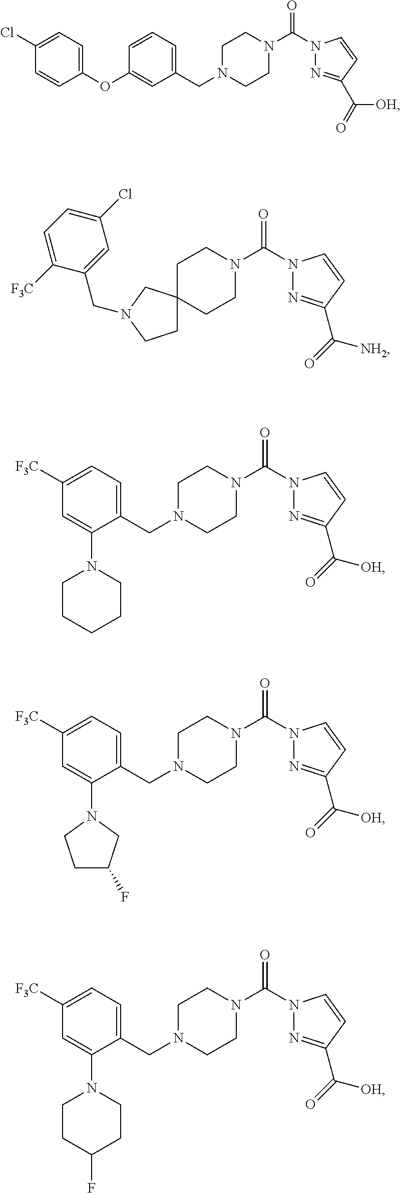 [SMILES: O=C(O)c1ccn(C(=O)N2CCN(Cc3cccc(Oc4ccc(Cl)cc4)c3)CC2)n1 ; NC(=O)c1ccn(C(=O)N2CCC3(CCN(Cc4cc(Cl)ccc4C(F)(F)F)C3)CC2)n1 ; O=C(O)c1ccn(C(=O)N2CCN(Cc3ccc(C(F)(F)F)cc3N3CCCCC3)CC2)n1 ; O=C(O)c1ccn(C(=O)N2CCN(Cc3ccc(C(F)(F)F)cc3N3CC[C@@H](F)C3)CC2)n1 ; O=C(O)c1ccn(C(=O)N2CCN(Cc3ccc(C(F)(F)F)cc3N3CCC(F)CC3)CC2)n1]

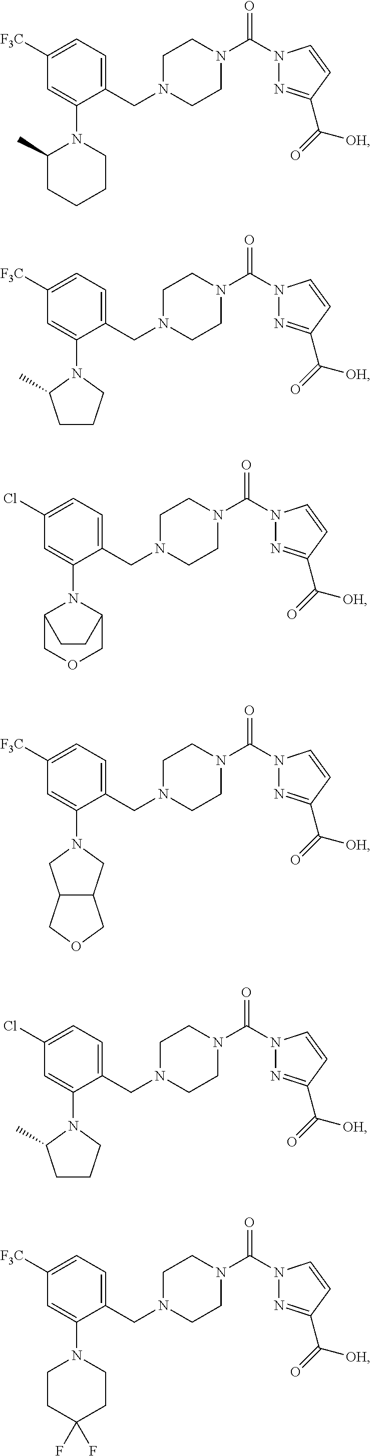 [SMILES: C[C@@H]1CCCCN1c1cc(C(F)(F)F)ccc1CN1CCN(C(=O)n2ccc(C(=O)O)n2)CC1 ; C[C@H]1CCCN1c1cc(C(F)(F)F)ccc1CN1CCN(C(=O)n2ccc(C(=O)O)n2)CC1 ; O=C(O)c1ccn(C(=O)N2CCN(Cc3ccc(Cl)cc3N3C4CCC3COC4)CC2)n1 ; O=C(O)c1ccn(C(=O)N2CCN(Cc3ccc(C(F)(F)F)cc3N3CC4COCC4C3)CC2)n1 ; C[C@H]1CCCN1c1cc(Cl)ccc1CN1CCN(C(=O)n2ccc(C(=O)O)n2)CC1 ; O=C(O)c1ccn(C(=O)N2CCN(Cc3ccc(C(F)(F)F)cc3N3CCC(F)(F)CC3)CC2)n1]

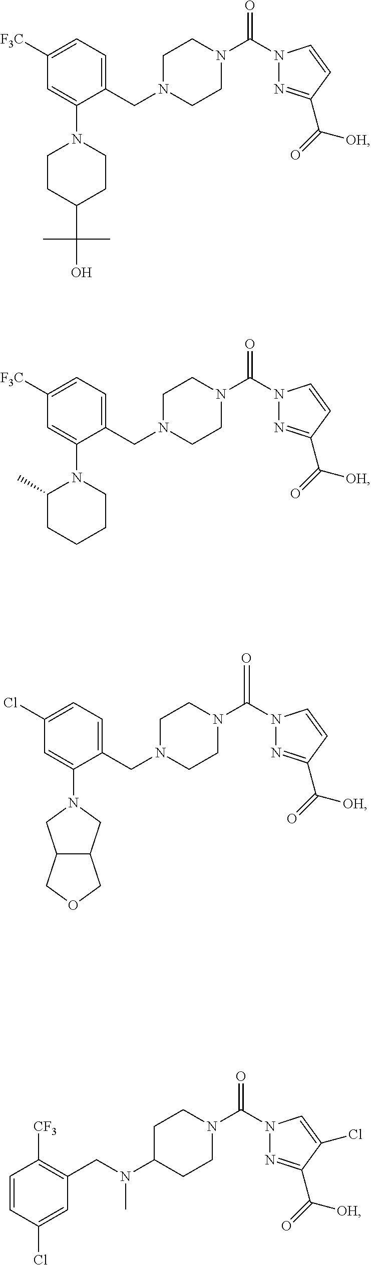 [SMILES: CC(C)(O)C1CCN(c2cc(C(F)(F)F)ccc2CN2CCN(C(=O)n3ccc(C(=O)O)n3)CC2)CC1 ; C[C@H]1CCCCN1c1cc(C(F)(F)F)ccc1CN1CCN(C(=O)n2ccc(C(=O)O)n2)CC1 ; O=C(O)c1ccn(C(=O)N2CCN(Cc3ccc(Cl)cc3N3CC4COCC4C3)CC2)n1 ; CN(Cc1cc(Cl)ccc1C(F)(F)F)C1CCN(C(=O)n2cc(Cl)c(C(=O)O)n2)CC1]

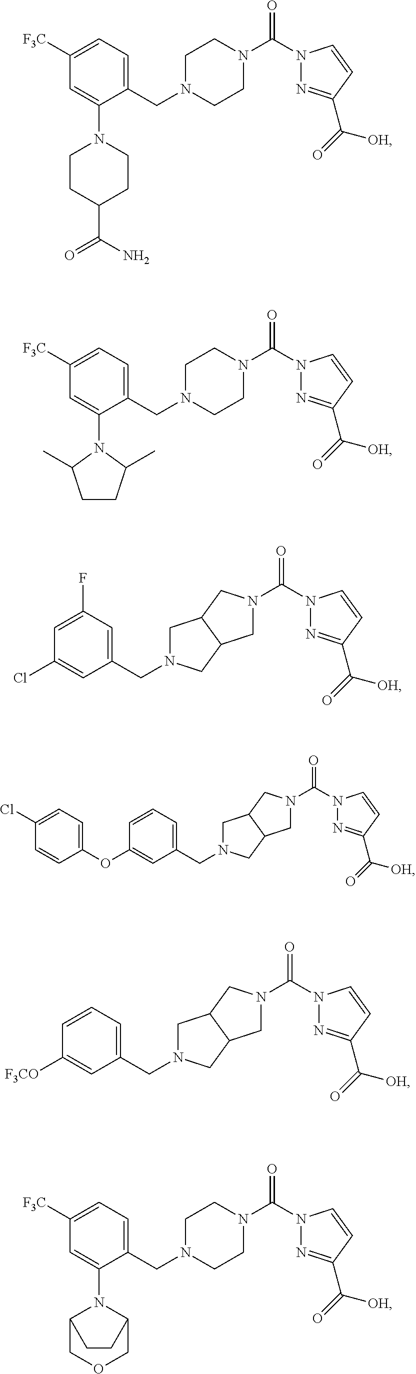 [SMILES: NC(=O)C1CCN(c2cc(C(F)(F)F)ccc2CN2CCN(C(=O)n3ccc(C(=O)O)n3)CC2)CC1 ; CC1CCC(C)N1c1cc(C(F)(F)F)ccc1CN1CCN(C(=O)n2ccc(C(=O)O)n2)CC1 ; O=C(O)c1ccn(C(=O)N2CC3CN(Cc4cc(F)cc(Cl)c4)CC3C2)n1 ; O=C(O)c1ccn(C(=O)N2CC3CN(Cc4cccc(Oc5ccc(Cl)cc5)c4)CC3C2)n1 ; O=C(O)c1ccn(C(=O)N2CC3CN(Cc4cccc(OC(F)(F)F)c4)CC3C2)n1 ; O=C(O)c1ccn(C(=O)N2CCN(Cc3ccc(C(F)(F)F)cc3N3C4CCC3COC4)CC2)n1]

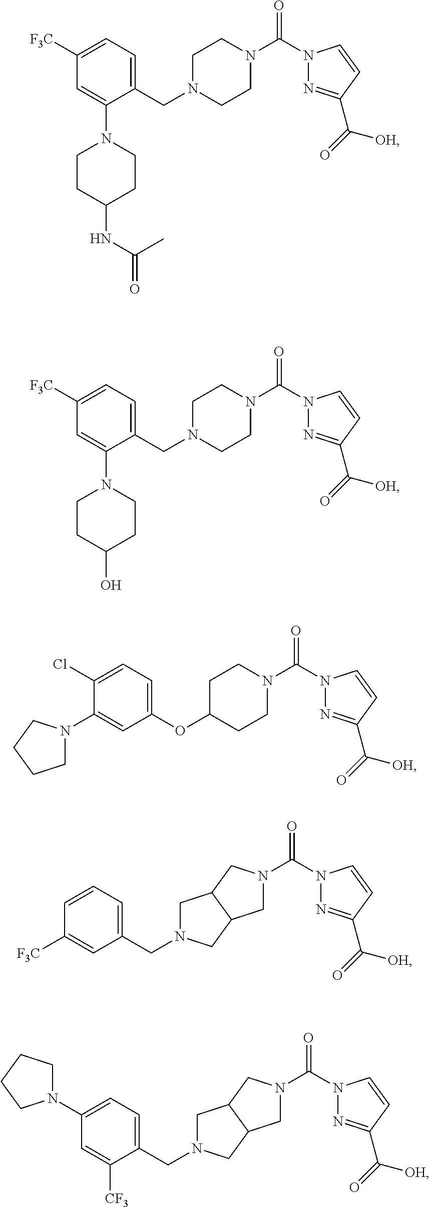 [SMILES: CC(=O)NC1CCN(c2cc(C(F)(F)F)ccc2CN2CCN(C(=O)n3ccc(C(=O)O)n3)CC2)CC1 ; O=C(O)c1ccn(C(=O)N2CCN(Cc3ccc(C(F)(F)F)cc3N3CCC(O)CC3)CC2)n1 ; O=C(O)c1ccn(C(=O)N2CCC(Oc3ccc(Cl)c(N4CCCC4)c3)CC2)n1 ; O=C(O)c1ccn(C(=O)N2CC3CN(Cc4cccc(C(F)(F)F)c4)CC3C2)n1 ; O=C(O)c1ccn(C(=O)N2CC3CN(Cc4ccc(N5CCCC5)cc4C(F)(F)F)CC3C2)n1]

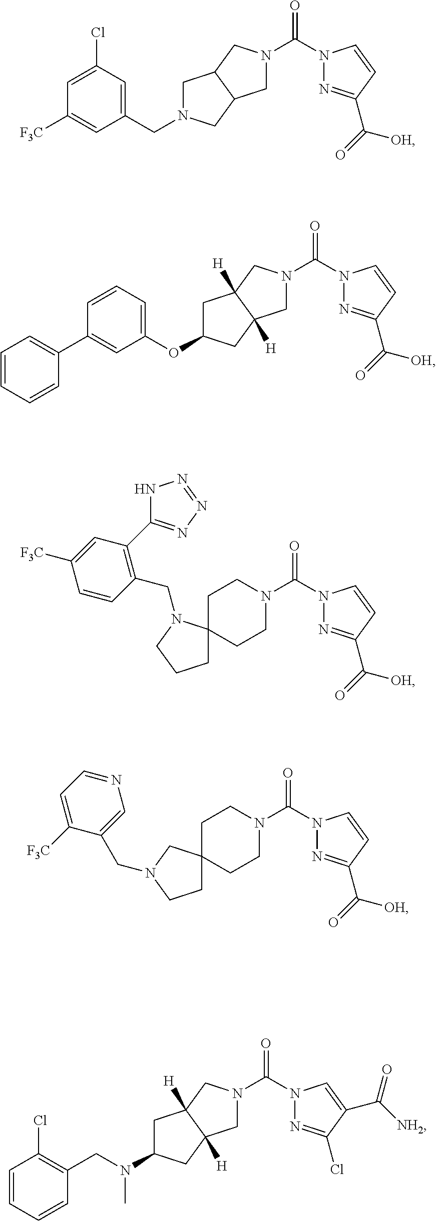 [SMILES: O=C(O)c1ccn(C(=O)N2CC3CN(Cc4cc(Cl)cc(C(F)(F)F)c4)CC3C2)n1 ; O=C(O)c1ccn(C(=O)N2C[C@H]3C[C@H](Oc4cccc(-c5ccccc5)c4)C[C@H]3C2)n1 ; O=C(O)c1ccn(C(=O)N2CCC3(CCCN3Cc3ccc(C(F)(F)F)cc3-c3nnn[nH]3)CC2)n1 ; O=C(O)c1ccn(C(=O)N2CCC3(CCN(Cc4cnccc4C(F)(F)F)C3)CC2)n1 ; CN(Cc1ccccc1Cl)[C@H]1C[C@@H]2CN(C(=O)n3cc(C(N)=O)c(Cl)n3)C[C@@H]2C1]

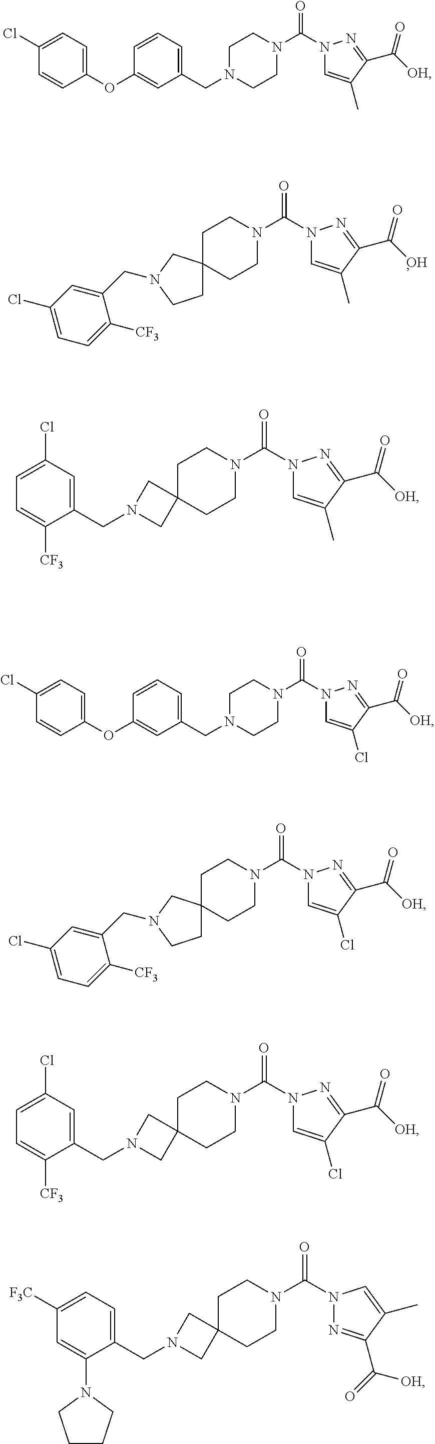

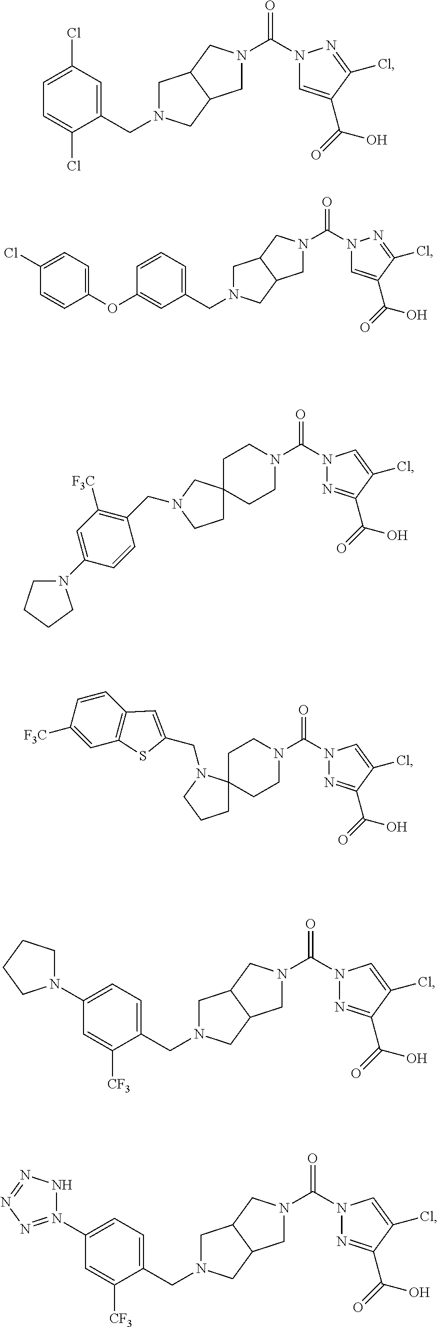

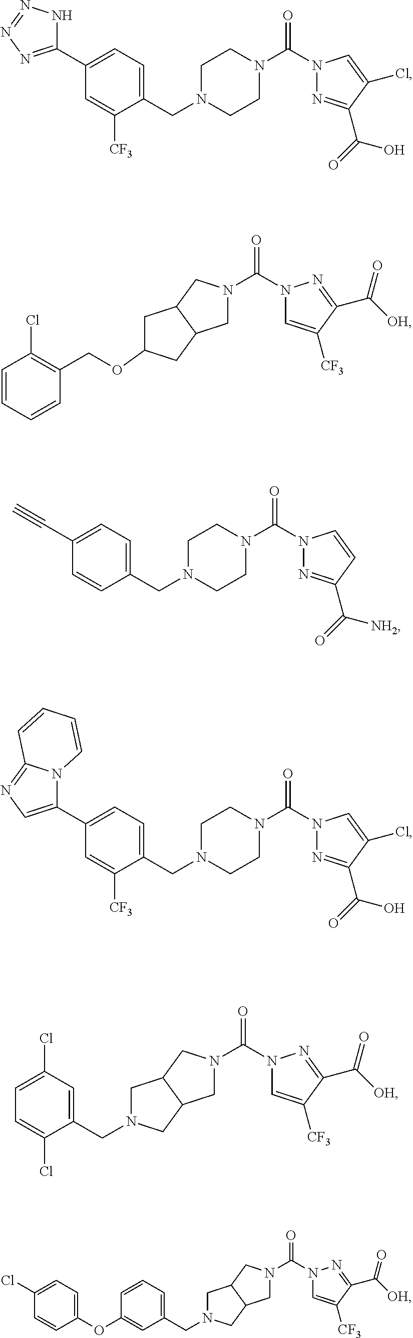 [SMILES: O=C(O)c1nn(C(=O)N2CCN(Cc3ccc(-c4nnn[nH]4)cc3C(F)(F)F)CC2)cc1Cl ; O=C(O)c1nn(C(=O)N2CC3CC(OCc4ccccc4Cl)CC3C2)cc1C(F)(F)F ; C#Cc1ccc(CN2CCN(C(=O)n3ccc(C(N)=O)n3)CC2)cc1 ; O=C(O)c1nn(C(=O)N2CCN(Cc3ccc(-c4cnc5ccccn45)cc3C(F)(F)F)CC2)cc1Cl ; O=C(O)c1nn(C(=O)N2CC3CN(Cc4cc(Cl)ccc4Cl)CC3C2)cc1C(F)(F)F ; O=C(O)c1nn(C(=O)N2CC3CN(Cc4cccc(Oc5ccc(Cl)cc5)c4)CC3C2)cc1C(F)(F)F]

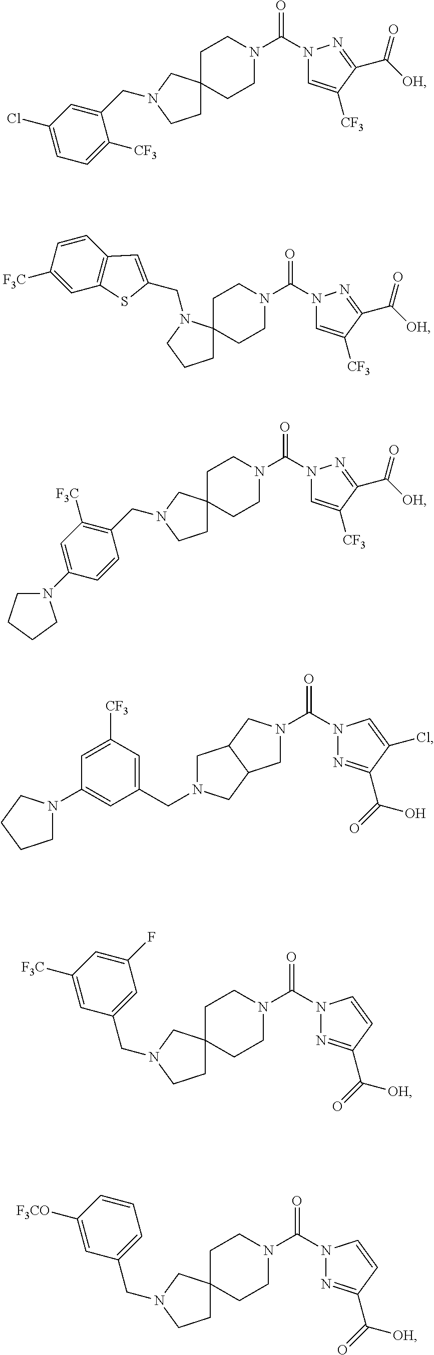 [SMILES: O=C(O)c1nn(C(=O)N2CCC3(CCN(Cc4cc(Cl)ccc4C(F)(F)F)C3)CC2)cc1C(F)(F)F ; O=C(O)c1nn(C(=O)N2CCC3(CCCN3Cc3cc4ccc(C(F)(F)F)cc4s3)CC2)cc1C(F)(F)F ; O=C(O)c1nn(C(=O)N2CCC3(CCN(Cc4ccc(N5CCCC5)cc4C(F)(F)F)C3)CC2)cc1C(F)(F)F ; O=C(O)c1nn(C(=O)N2CC3CN(Cc4cc(N5CCCC5)cc(C(F)(F)F)c4)CC3C2)cc1Cl ; O=C(O)c1ccn(C(=O)N2CCC3(CCN(Cc4cc(F)cc(C(F)(F)F)c4)C3)CC2)n1 ; O=C(O)c1ccn(C(=O)N2CCC3(CCN(Cc4cccc(OC(F)(F)F)c4)C3)CC2)n1]

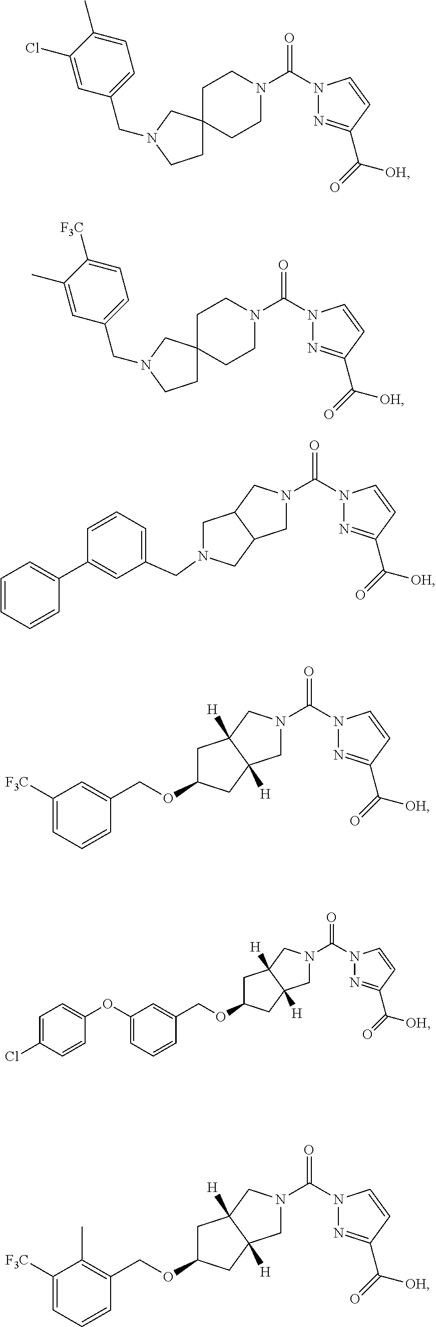

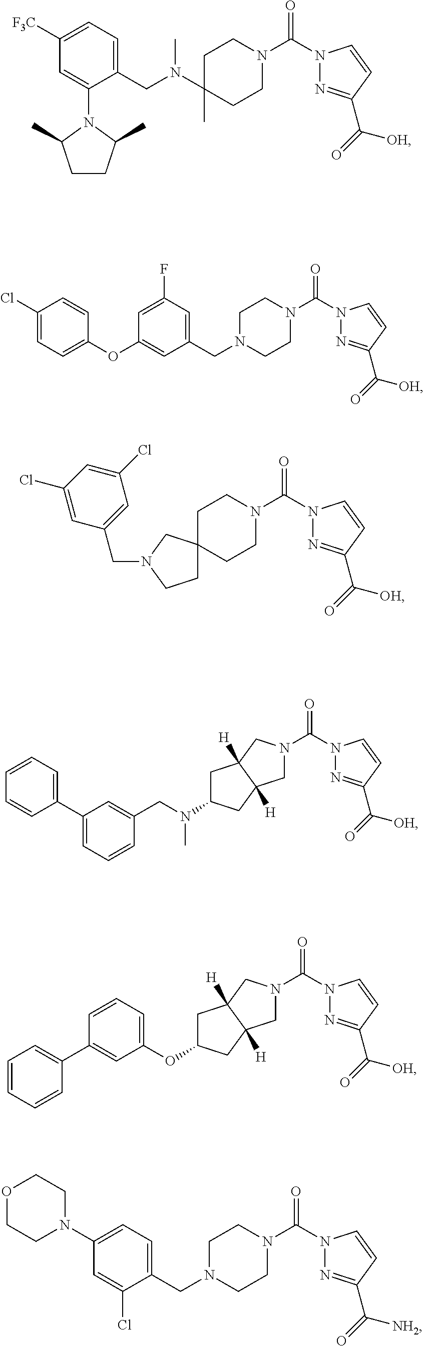 [SMILES: C[C@@H]1CC[C@H](C)N1c1cc(C(F)(F)F)ccc1CN(C)C1(C)CCN(C(=O)n2ccc(C(=O)O)n2)CC1 ; O=C(O)c1ccn(C(=O)N2CCN(Cc3cc(F)cc(Oc4ccc(Cl)cc4)c3)CC2)n1 ; O=C(O)c1ccn(C(=O)N2CCC3(CCN(Cc4cc(Cl)cc(Cl)c4)C3)CC2)n1 ; CN(Cc1cccc(-c2ccccc2)c1)[C@@H]1C[C@@H]2CN(C(=O)n3ccc(C(=O)O)n3)C[C@@H]2C1 ; O=C(O)c1ccn(C(=O)N2C[C@H]3C[C@@H](Oc4cccc(-c5ccccc5)c4)C[C@H]3C2)n1 ; NC(=O)c1ccn(C(=O)N2CCN(Cc3ccc(N4CCOCC4)cc3Cl)CC2)n1]

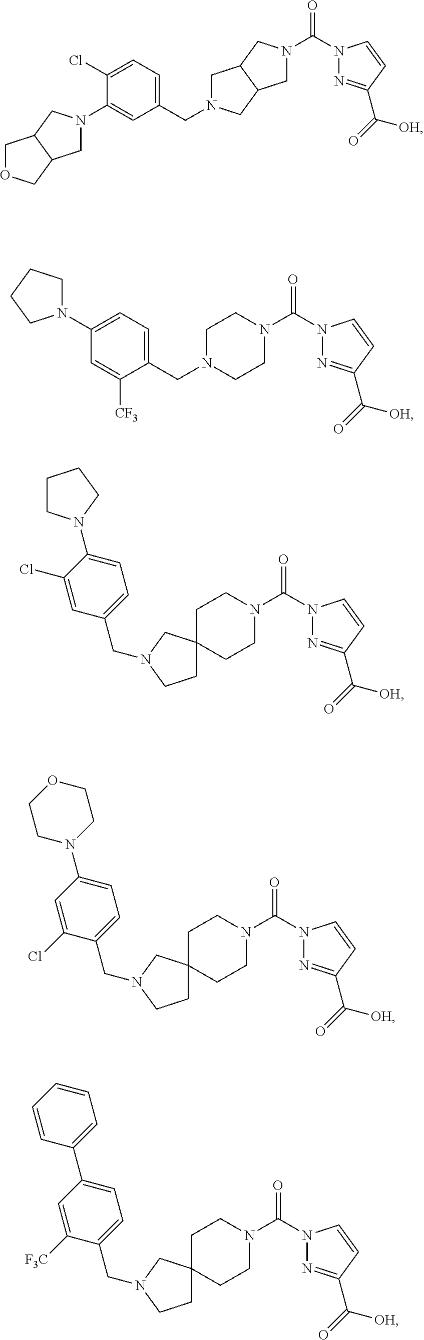 [SMILES: O=C(O)c1ccn(C(=O)N2CC3CN(Cc4ccc(Cl)c(N5CC6COCC6C5)c4)CC3C2)n1 ; O=C(O)c1ccn(C(=O)N2CCN(Cc3ccc(N4CCCC4)cc3C(F)(F)F)CC2)n1 ; O=C(O)c1ccn(C(=O)N2CCC3(CCN(Cc4ccc(N5CCCC5)c(Cl)c4)C3)CC2)n1 ; O=C(O)c1ccn(C(=O)N2CCC3(CCN(Cc4ccc(N5CCOCC5)cc4Cl)C3)CC2)n1 ; O=C(O)c1ccn(C(=O)N2CCC3(CCN(Cc4ccc(-c5ccccc5)cc4C(F)(F)F)C3)CC2)n1]

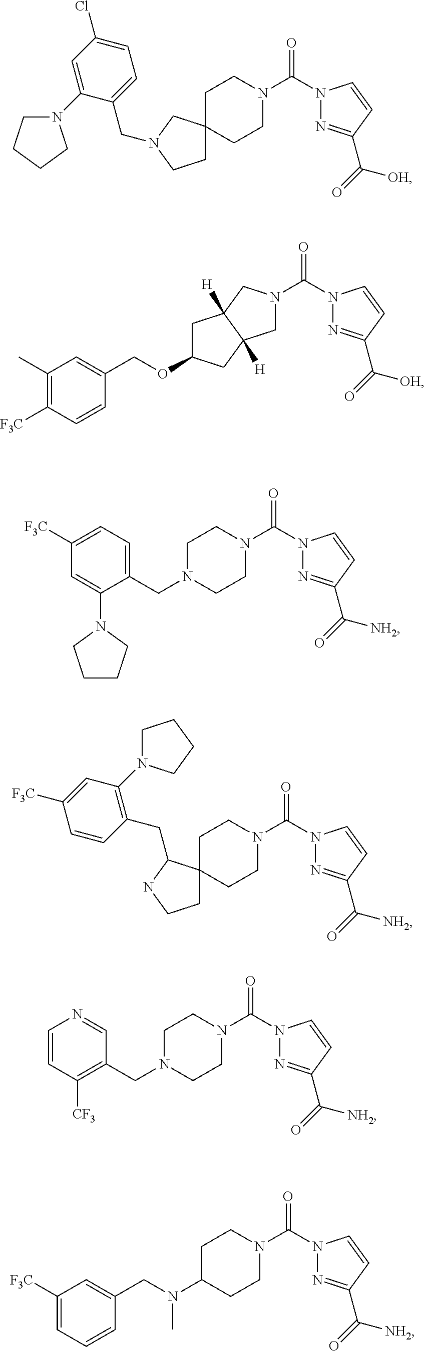 [SMILES: O=C(O)c1ccn(C(=O)N2CCC3(CCN(Cc4ccc(Cl)cc4N4CCCC4)C3)CC2)n1 ; Cc1cc(CO[C@H]2C[C@@H]3CN(C(=O)n4ccc(C(=O)O)n4)C[C@@H]3C2)ccc1C(F)(F)F ; NC(=O)c1ccn(C(=O)N2CCN(Cc3ccc(C(F)(F)F)cc3N3CCCC3)CC2)n1 ; NC(=O)c1ccn(C(=O)N2CCC3(CCNC3Cc3ccc(C(F)(F)F)cc3N3CCCC3)CC2)n1 ; NC(=O)c1ccn(C(=O)N2CCN(Cc3cnccc3C(F)(F)F)CC2)n1 ; CN(Cc1cccc(C(F)(F)F)c1)C1CCN(C(=O)n2ccc(C(N)=O)n2)CC1]

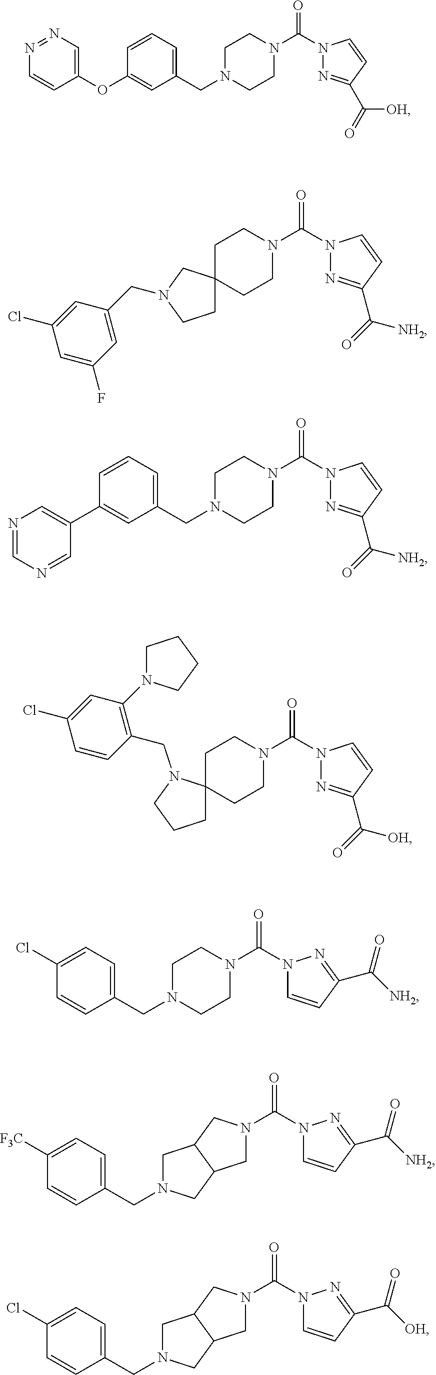

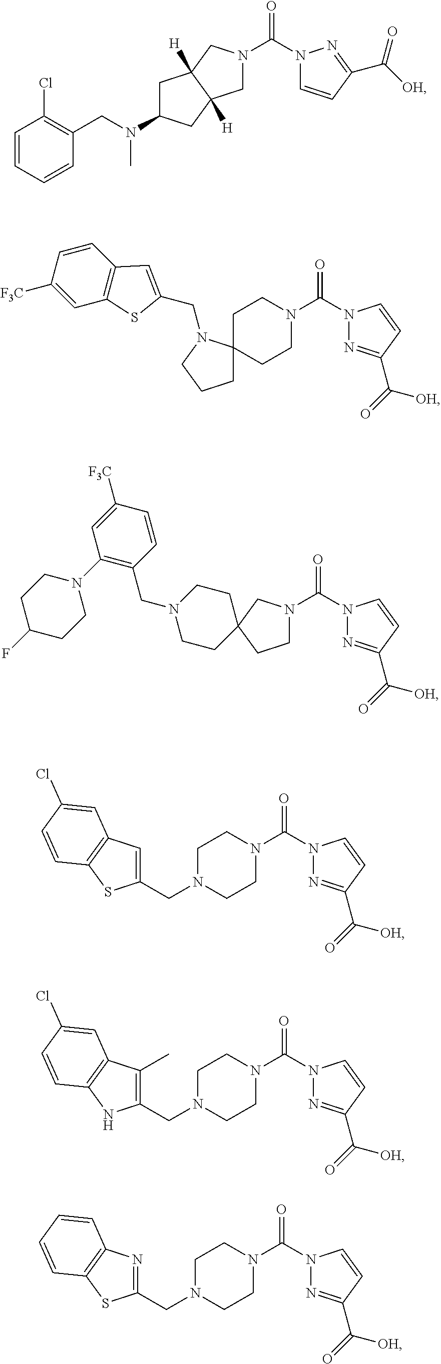 [SMILES: CN(Cc1ccccc1Cl)[C@H]1C[C@@H]2CN(C(=O)n3ccc(C(=O)O)n3)C[C@@H]2C1 ; O=C(O)c1ccn(C(=O)N2CCC3(CCCN3Cc3cc4ccc(C(F)(F)F)cc4s3)CC2)n1 ; O=C(O)c1ccn(C(=O)N2CCC3(CCN(Cc4ccc(C(F)(F)F)cc4N4CCC(F)CC4)CC3)C2)n1 ; O=C(O)c1ccn(C(=O)N2CCN(Cc3cc4cc(Cl)ccc4s3)CC2)n1 ; Cc1c(CN2CCN(C(=O)n3ccc(C(=O)O)n3)CC2)[nH]c2ccc(Cl)cc12 ; O=C(O)c1ccn(C(=O)N2CCN(Cc3nc4ccccc4s3)CC2)n1]

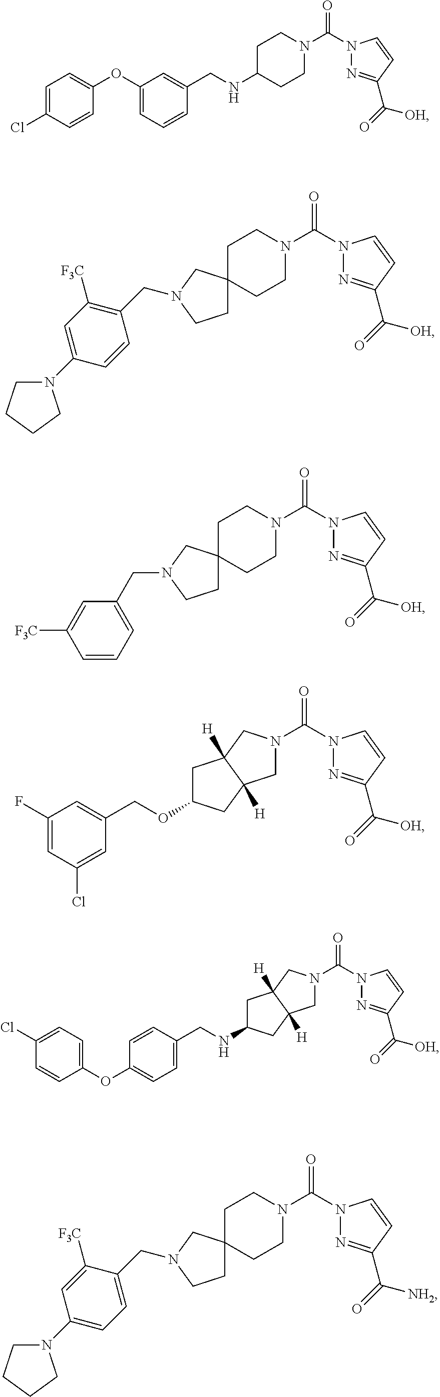 [SMILES: O=C(O)c1ccn(C(=O)N2CCC(NCc3cccc(Oc4ccc(Cl)cc4)c3)CC2)n1 ; O=C(O)c1ccn(C(=O)N2CCC3(CCN(Cc4ccc(N5CCCC5)cc4C(F)(F)F)C3)CC2)n1 ; O=C(O)c1ccn(C(=O)N2CCC3(CCN(Cc4cccc(C(F)(F)F)c4)C3)CC2)n1 ; O=C(O)c1ccn(C(=O)N2C[C@H]3C[C@@H](OCc4cc(F)cc(Cl)c4)C[C@H]3C2)n1 ; O=C(O)c1ccn(C(=O)N2C[C@H]3C[C@H](NCc4ccc(Oc5ccc(Cl)cc5)cc4)C[C@H]3C2)n1 ; NC(=O)c1ccn(C(=O)N2CCC3(CCN(Cc4ccc(N5CCCC5)cc4C(F)(F)F)C3)CC2)n1]

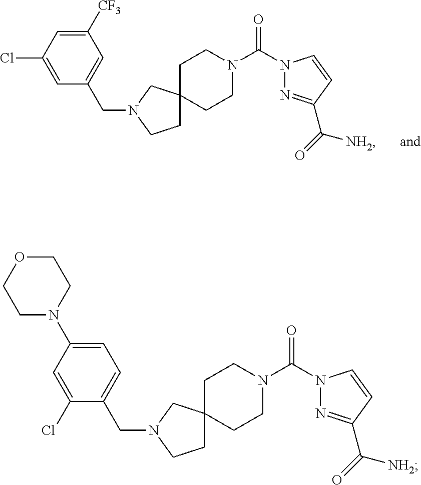 [SMILES: NC(=O)c1ccn(C(=O)N2CCC3(CCN(Cc4cc(Cl)cc(C(F)(F)F)c4)C3)CC2)n1 ; NC(=O)c1ccn(C(=O)N2CCC3(CCN(Cc4ccc(N5CCOCC5)cc4Cl)C3)CC2)n1]

or a solvate, hydrate, tautomer, N-oxide, stereoisomer, or pharmaceutically acceptable salt thereof.

In some embodiments is method for treating a disease or a condition in a patient wherein the disease is selected from atopic dermatitis, bladder dysfunction associated with multiple sclerosis, cardiovascular disease, contact dermatitis, cystic fibrosis, dermatomyositis, eczema, endometriosis, enteritis, fibromyalgia, Tourette syndrome, inflammatory bowel disease, interstitial cystitis, irritable bowel syndrome, ischemia, labor, abdominal pain, abdominal pain associated with irritable bowel syndrome, acute pain, back pain, cancer pain, chest pain, functional chest pain, joint pain, menstrual pain, metabolic disorders, musculoskeletal diseases, neuropathy, osteoarthritis, pancreatitis, pharyngitis, post mastectomy pain syndrome, post trigeminal neuralgia, post operative pain, renal ischemia, rheumatoid arthritis, skeletal muscle contusion, skin diseases, sunburn, systemic lupus erythematosus, toothache, vasoocclusive painful crises in sickle cell disease, and visceral pain, comprising administering to the patient in need thereof a therapeutically effective amount of a compound having the structure:

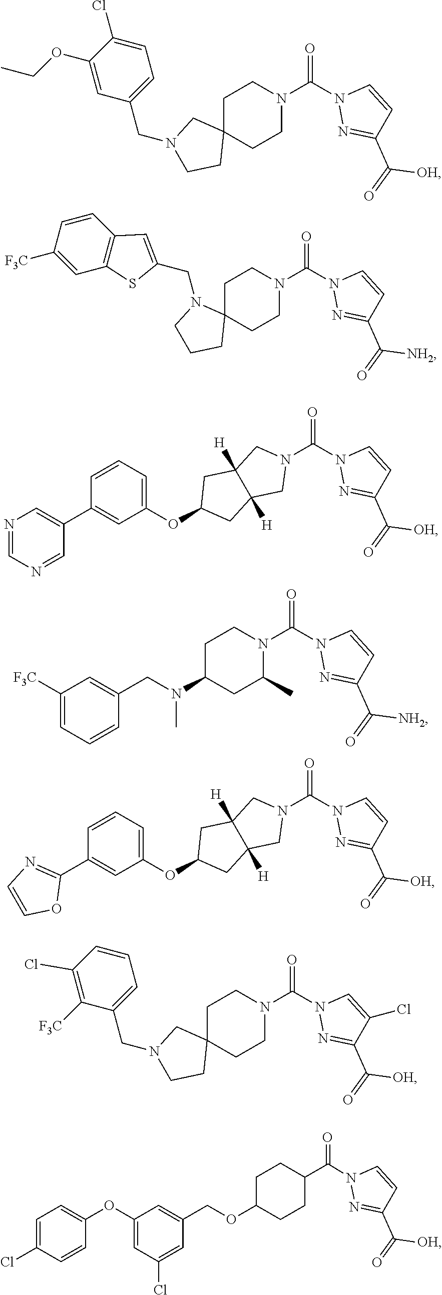

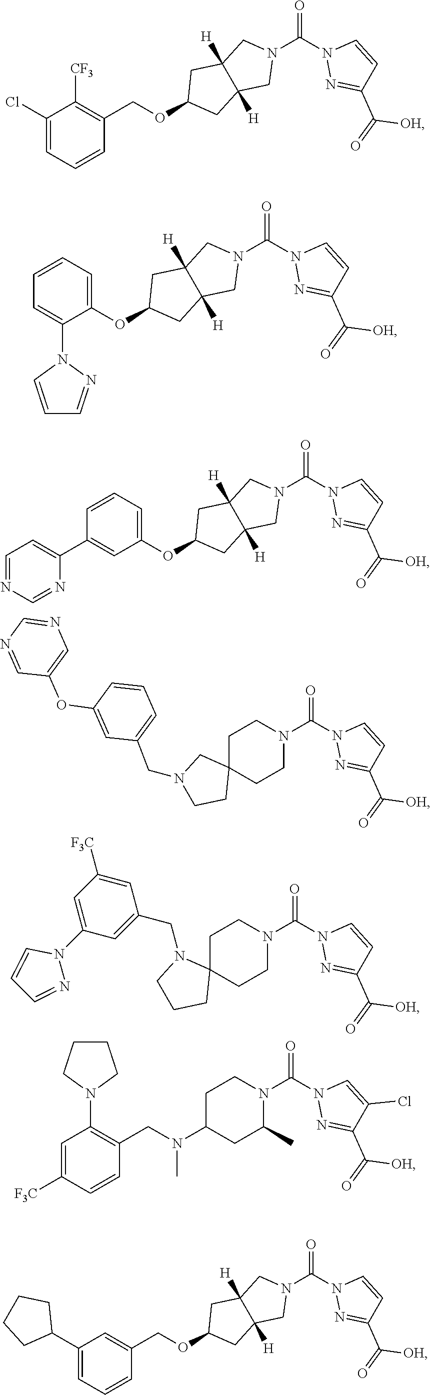

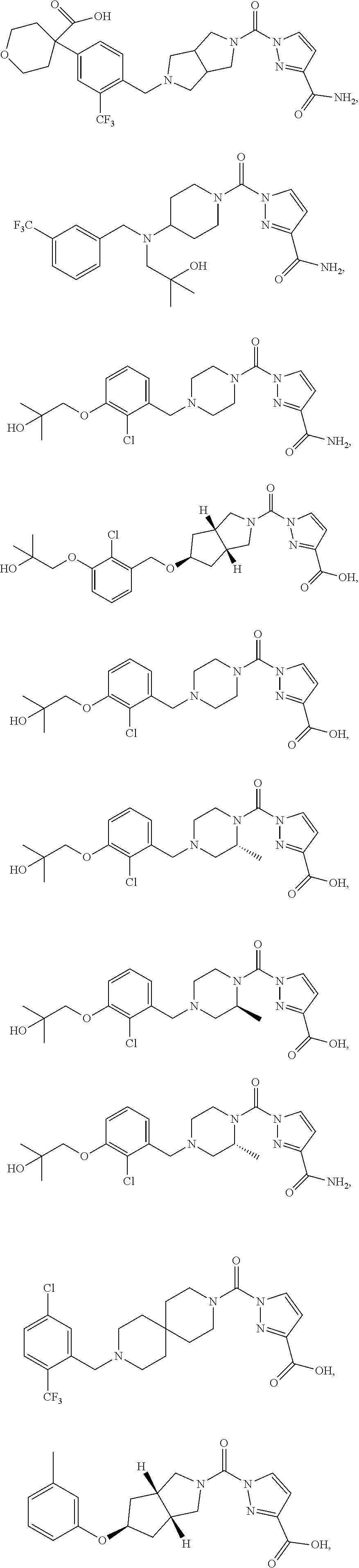

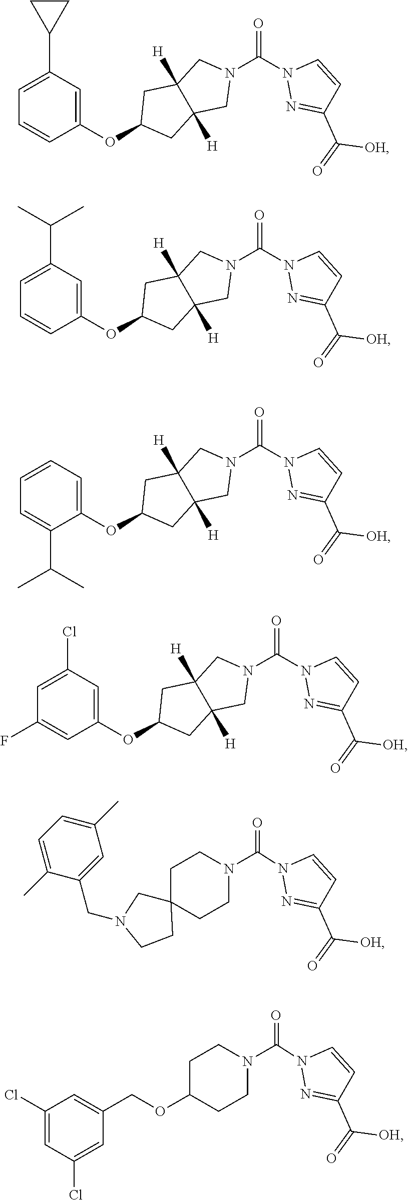

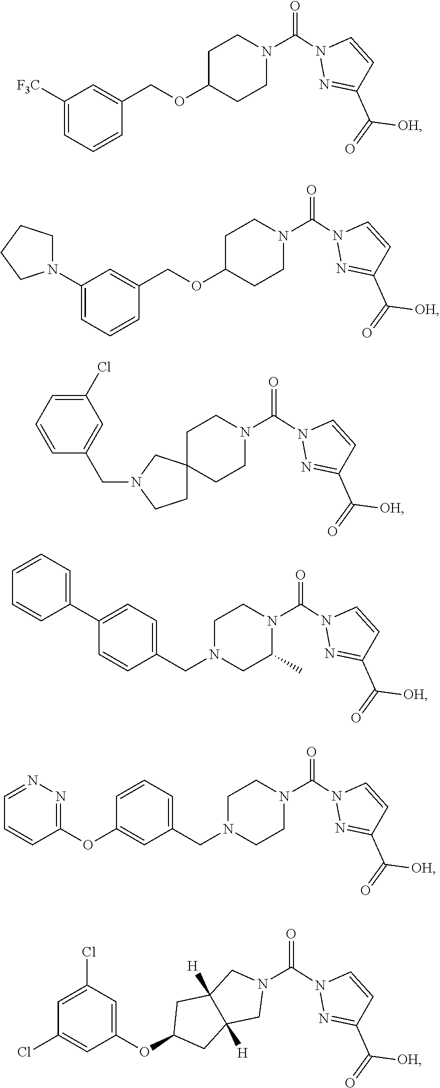

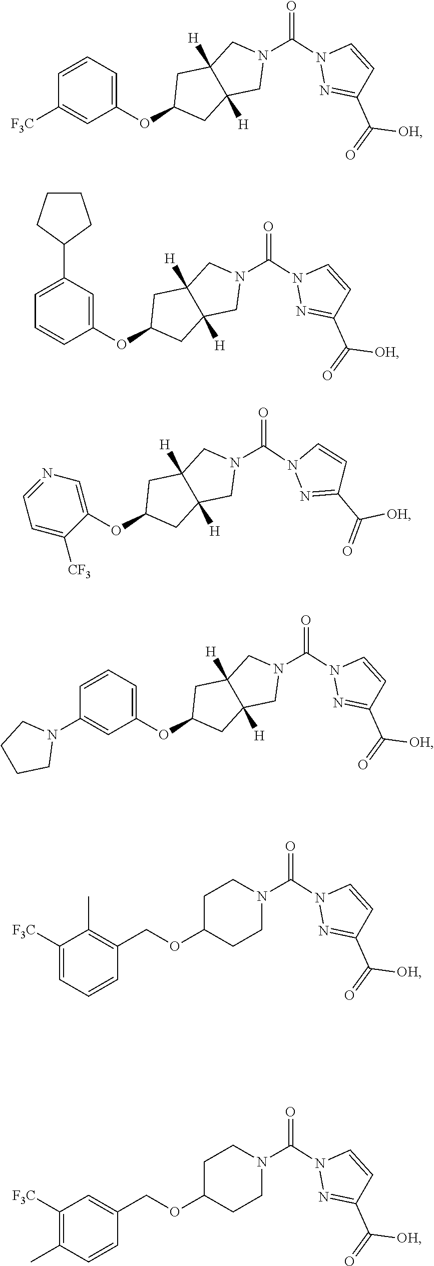 [SMILES: O=C(O)c1ccn(C(=O)N2C[C@H]3C[C@H](Oc4cccc(C(F)(F)F)c4)C[C@H]3C2)n1 ; O=C(O)c1ccn(C(=O)N2C[C@H]3C[C@H](Oc4cccc(C5CCCC5)c4)C[C@H]3C2)n1 ; O=C(O)c1ccn(C(=O)N2C[C@H]3C[C@H](Oc4cnccc4C(F)(F)F)C[C@H]3C2)n1 ; O=C(O)c1ccn(C(=O)N2C[C@H]3C[C@H](Oc4cccc(N5CCCC5)c4)C[C@H]3C2)n1 ; Cc1c(COC2CCN(C(=O)n3ccc(C(=O)O)n3)CC2)cccc1C(F)(F)F ; Cc1ccc(COC2CCN(C(=O)n3ccc(C(=O)O)n3)CC2)cc1C(F)(F)F]

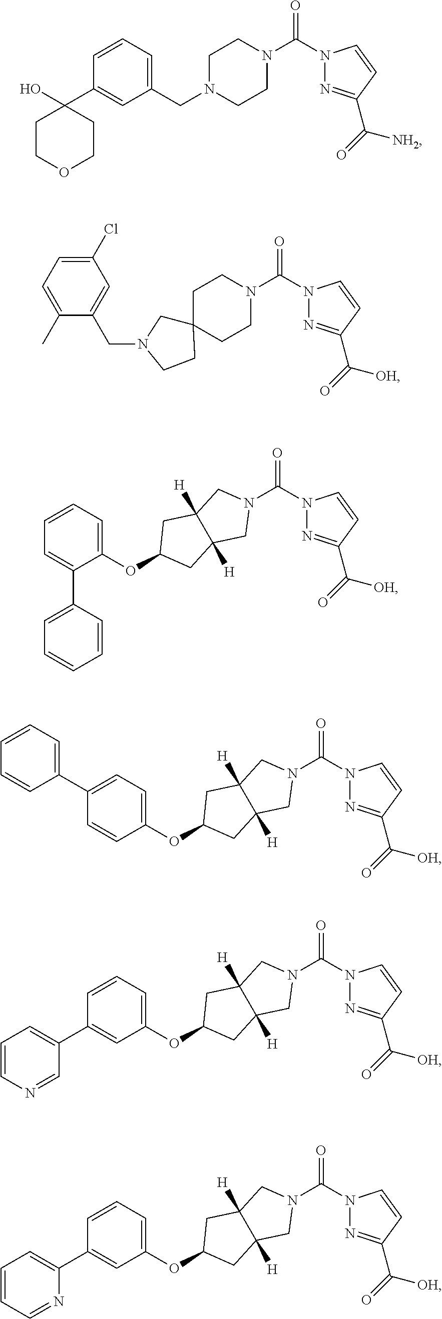 [SMILES: NC(=O)c1ccn(C(=O)N2CCN(Cc3cccc(C4(O)CCOCC4)c3)CC2)n1 ; Cc1ccc(Cl)cc1CN1CCC2(CCN(C(=O)n3ccc(C(=O)O)n3)CC2)C1 ; O=C(O)c1ccn(C(=O)N2C[C@H]3C[C@H](Oc4ccccc4-c4ccccc4)C[C@H]3C2)n1 ; O=C(O)c1ccn(C(=O)N2C[C@H]3C[C@H](Oc4ccc(-c5ccccc5)cc4)C[C@H]3C2)n1 ; O=C(O)c1ccn(C(=O)N2C[C@H]3C[C@H](Oc4cccc(-c5cccnc5)c4)C[C@H]3C2)n1 ; O=C(O)c1ccn(C(=O)N2C[C@H]3C[C@H](Oc4cccc(-c5ccccn5)c4)C[C@H]3C2)n1]

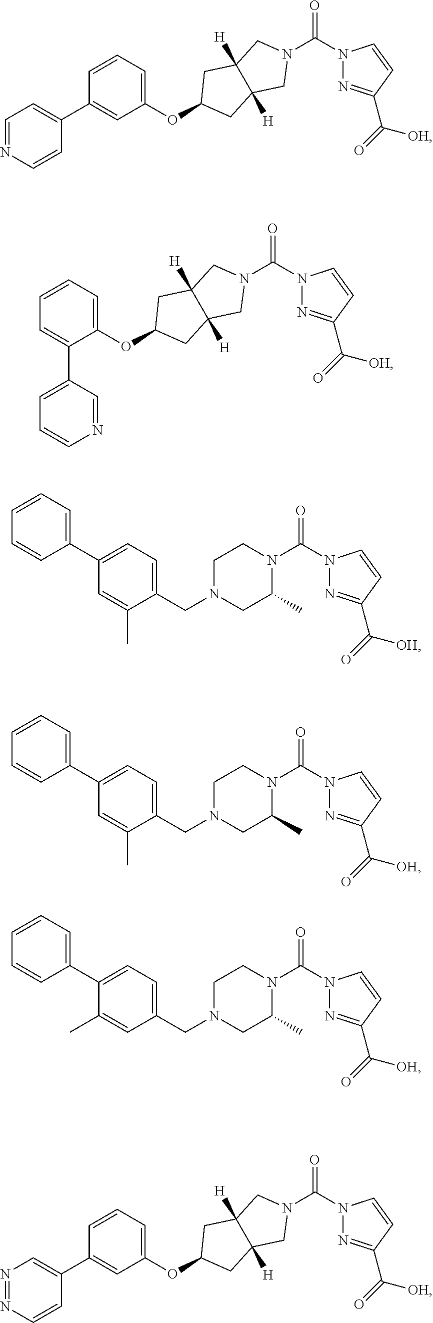 [SMILES: O=C(O)c1ccn(C(=O)N2C[C@H]3C[C@H](Oc4cccc(-c5ccncc5)c4)C[C@H]3C2)n1 ; O=C(O)c1ccn(C(=O)N2C[C@H]3C[C@H](Oc4ccccc4-c4cccnc4)C[C@H]3C2)n1 ; Cc1cc(-c2ccccc2)ccc1CN1CCN(C(=O)n2ccc(C(=O)O)n2)[C@H](C)C1 ; Cc1cc(-c2ccccc2)ccc1CN1CCN(C(=O)n2ccc(C(=O)O)n2)[C@@H](C)C1 ; Cc1cc(CN2CCN(C(=O)n3ccc(C(=O)O)n3)[C@H](C)C2)ccc1-c1ccccc1 ; O=C(O)c1ccn(C(=O)N2C[C@H]3C[C@H](Oc4cccc(-c5ccnnc5)c4)C[C@H]3C2)n1]

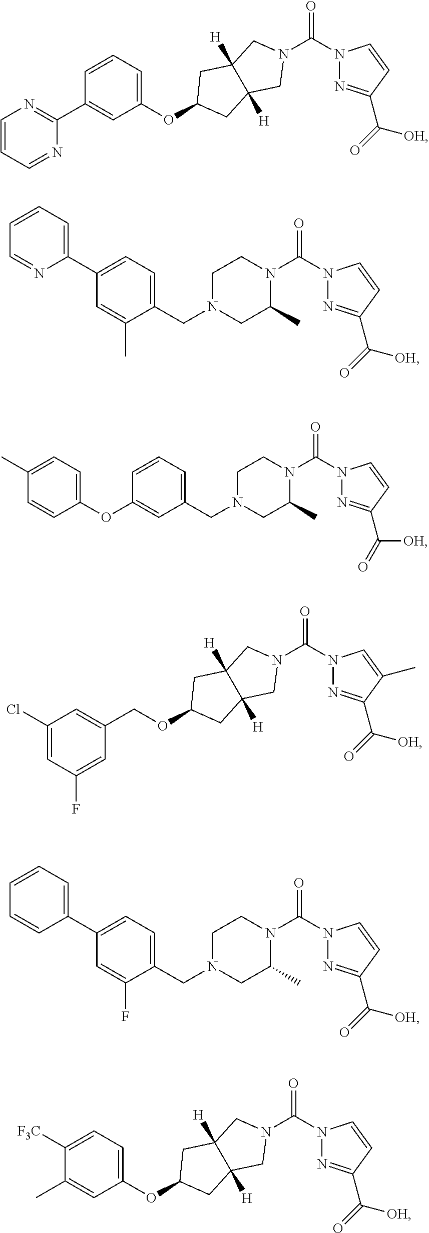

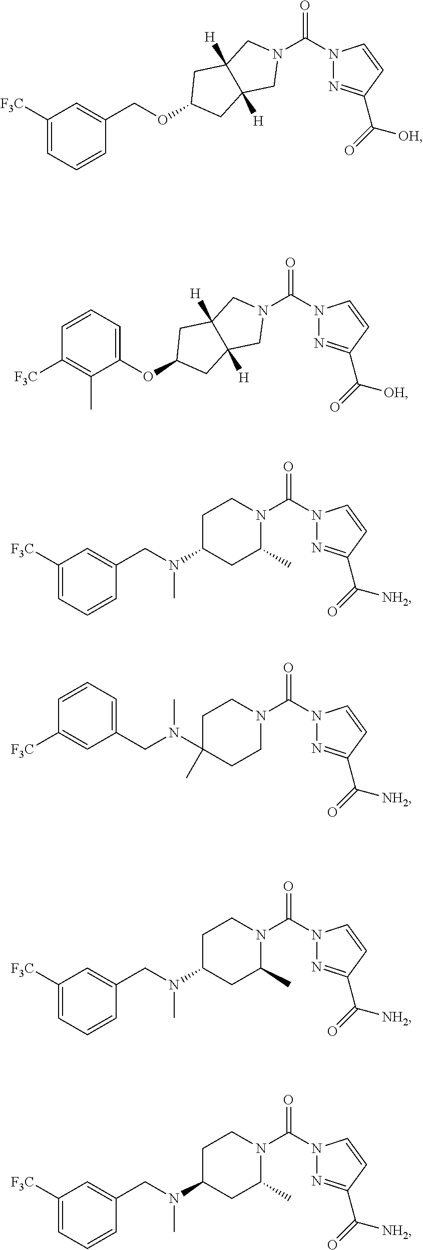 [SMILES: O=C(O)c1ccn(C(=O)N2C[C@H]3C[C@@H](OCc4cccc(C(F)(F)F)c4)C[C@H]3C2)n1 ; Cc1c(O[C@H]2C[C@@H]3CN(C(=O)n4ccc(C(=O)O)n4)C[C@@H]3C2)cccc1C(F)(F)F ; C[C@@H]1C[C@H](N(C)Cc2cccc(C(F)(F)F)c2)CCN1C(=O)n1ccc(C(N)=O)n1 ; CN(Cc1cccc(C(F)(F)F)c1)C1(C)CCN(C(=O)n2ccc(C(N)=O)n2)CC1 ; C[C@H]1C[C@H](N(C)Cc2cccc(C(F)(F)F)c2)CCN1C(=O)n1ccc(C(N)=O)n1 ; C[C@@H]1C[C@@H](N(C)Cc2cccc(C(F)(F)F)c2)CCN1C(=O)n1ccc(C(N)=O)n1]

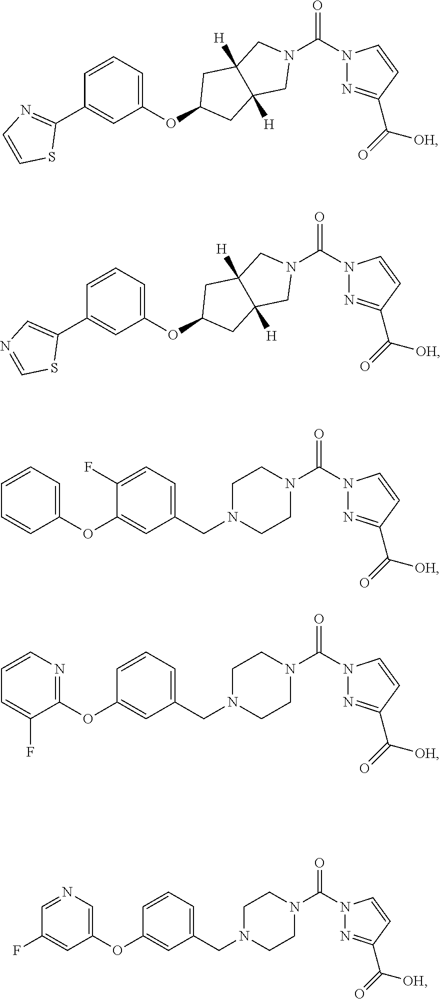 [SMILES: O=C(O)c1ccn(C(=O)N2C[C@H]3C[C@H](Oc4cccc(-c5nccs5)c4)C[C@H]3C2)n1 ; O=C(O)c1ccn(C(=O)N2C[C@H]3C[C@H](Oc4cccc(-c5cncs5)c4)C[C@H]3C2)n1 ; O=C(O)c1ccn(C(=O)N2CCN(Cc3ccc(F)c(Oc4ccccc4)c3)CC2)n1 ; O=C(O)c1ccn(C(=O)N2CCN(Cc3cccc(Oc4ncccc4F)c3)CC2)n1 ; O=C(O)c1ccn(C(=O)N2CCN(Cc3cccc(Oc4cncc(F)c4)c3)CC2)n1]

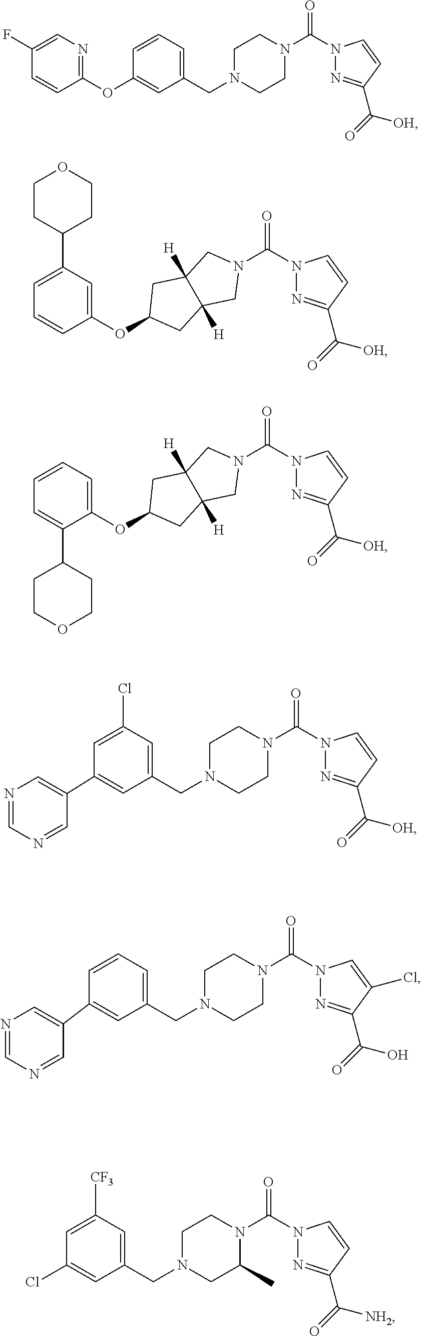 [SMILES: O=C(O)c1ccn(C(=O)N2CCN(Cc3cccc(Oc4ccc(F)cn4)c3)CC2)n1 ; O=C(O)c1ccn(C(=O)N2C[C@H]3C[C@H](Oc4cccc(C5CCOCC5)c4)C[C@H]3C2)n1 ; O=C(O)c1ccn(C(=O)N2C[C@H]3C[C@H](Oc4ccccc4C4CCOCC4)C[C@H]3C2)n1 ; O=C(O)c1ccn(C(=O)N2CCN(Cc3cc(Cl)cc(-c4cncnc4)c3)CC2)n1 ; O=C(O)c1nn(C(=O)N2CCN(Cc3cccc(-c4cncnc4)c3)CC2)cc1Cl ; C[C@H]1CN(Cc2cc(Cl)cc(C(F)(F)F)c2)CCN1C(=O)n1ccc(C(N)=O)n1]

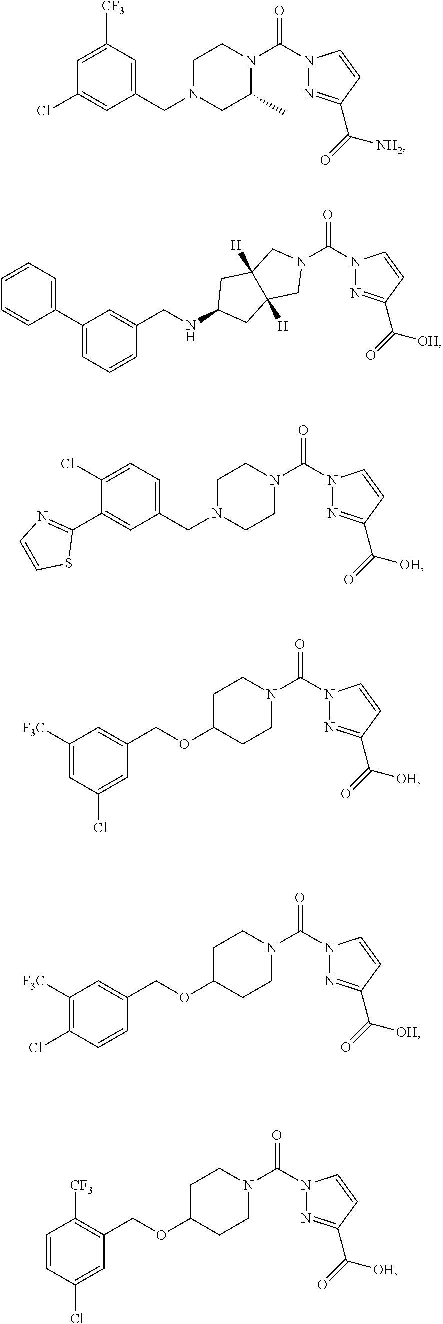 [SMILES: C[C@@H]1CN(Cc2cc(Cl)cc(C(F)(F)F)c2)CCN1C(=O)n1ccc(C(N)=O)n1 ; O=C(O)c1ccn(C(=O)N2C[C@H]3C[C@H](NCc4cccc(-c5ccccc5)c4)C[C@H]3C2)n1 ; O=C(O)c1ccn(C(=O)N2CCN(Cc3ccc(Cl)c(-c4nccs4)c3)CC2)n1 ; O=C(O)c1ccn(C(=O)N2CCC(OCc3cc(Cl)cc(C(F)(F)F)c3)CC2)n1 ; O=C(O)c1ccn(C(=O)N2CCC(OCc3ccc(Cl)c(C(F)(F)F)c3)CC2)n1 ; O=C(O)c1ccn(C(=O)N2CCC(OCc3cc(Cl)ccc3C(F)(F)F)CC2)n1]

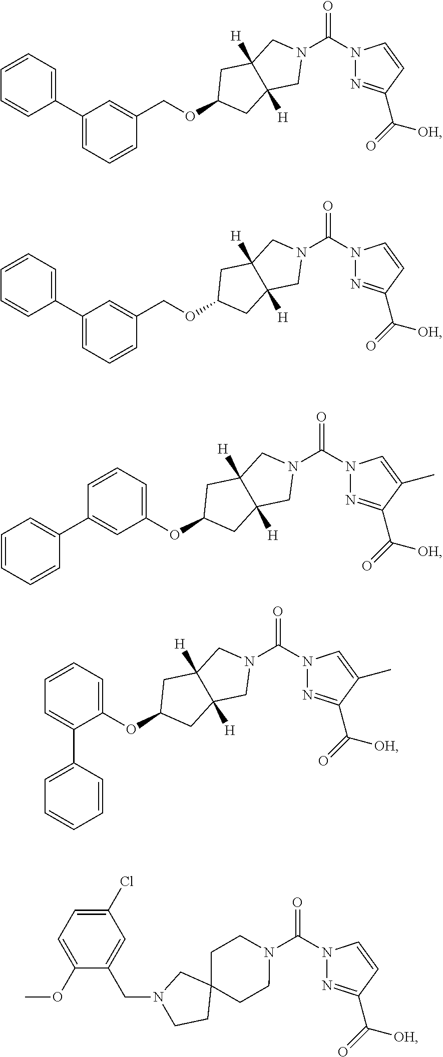

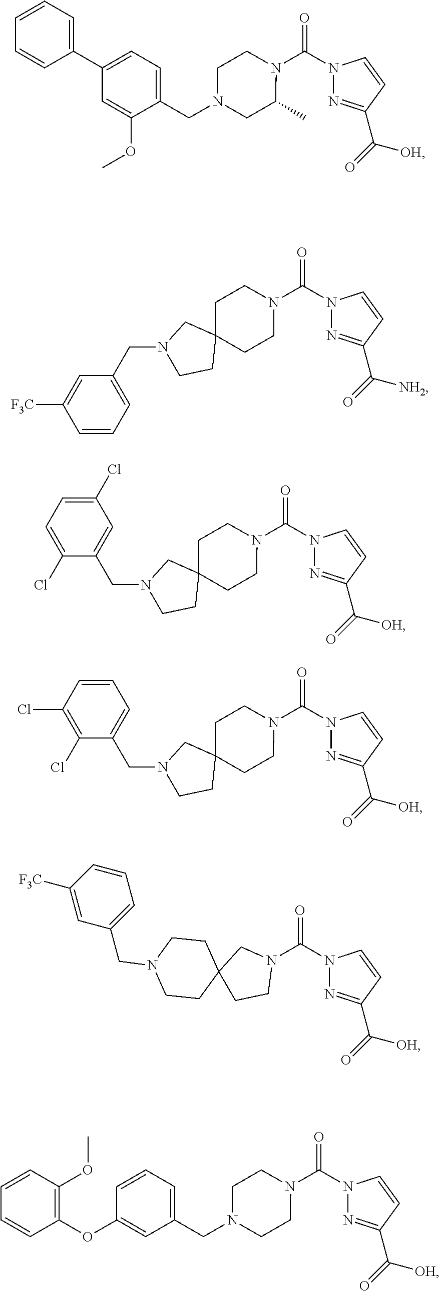

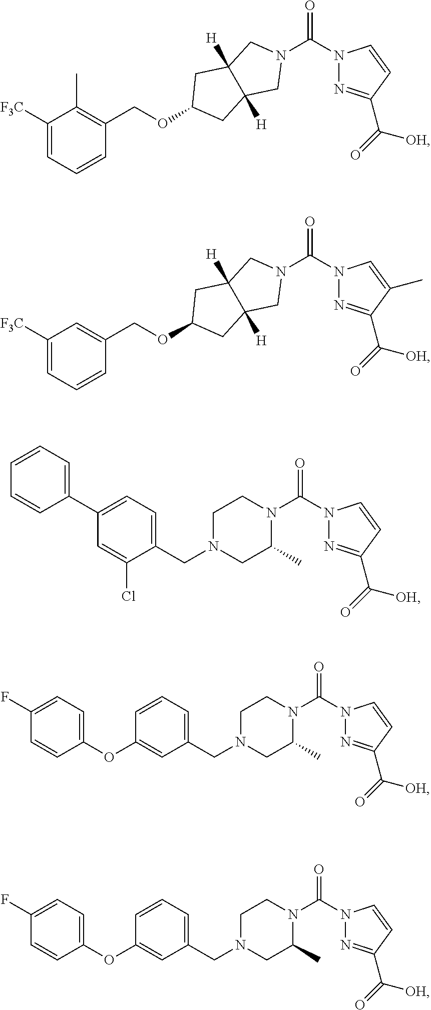 [SMILES: Cc1c(CO[C@@H]2C[C@@H]3CN(C(=O)n4ccc(C(=O)O)n4)C[C@@H]3C2)cccc1C(F)(F)F ; Cc1cn(C(=O)N2C[C@H]3C[C@H](OCc4cccc(C(F)(F)F)c4)C[C@H]3C2)nc1C(=O)O ; C[C@@H]1CN(Cc2ccc(-c3ccccc3)cc2Cl)CCN1C(=O)n1ccc(C(=O)O)n1 ; C[C@@H]1CN(Cc2cccc(Oc3ccc(F)cc3)c2)CCN1C(=O)n1ccc(C(=O)O)n1 ; C[C@H]1CN(Cc2cccc(Oc3ccc(F)cc3)c2)CCN1C(=O)n1ccc(C(=O)O)n1]

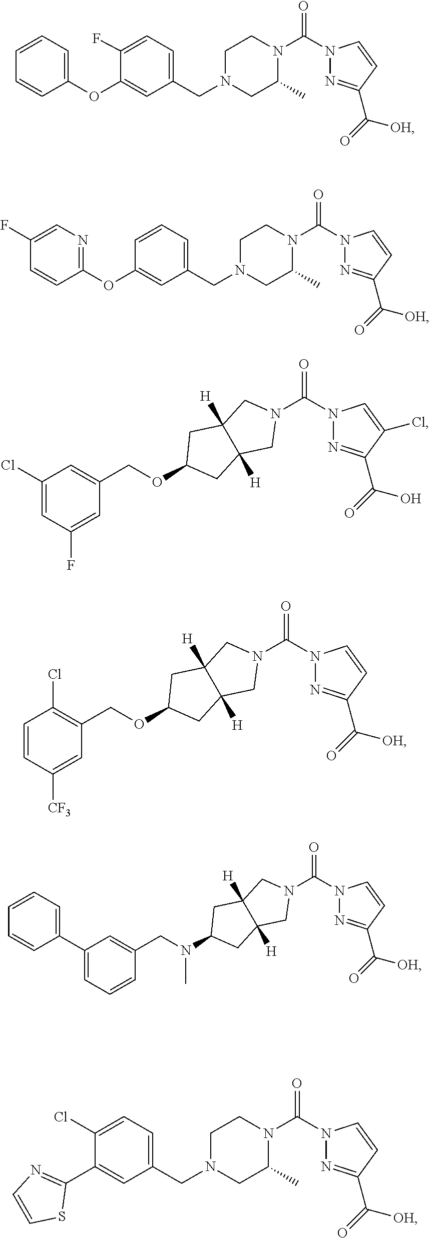 [SMILES: C[C@@H]1CN(Cc2ccc(F)c(Oc3ccccc3)c2)CCN1C(=O)n1ccc(C(=O)O)n1 ; C[C@@H]1CN(Cc2cccc(Oc3ccc(F)cn3)c2)CCN1C(=O)n1ccc(C(=O)O)n1 ; O=C(O)c1nn(C(=O)N2C[C@H]3C[C@H](OCc4cc(F)cc(Cl)c4)C[C@H]3C2)cc1Cl ; O=C(O)c1ccn(C(=O)N2C[C@H]3C[C@H](OCc4cc(C(F)(F)F)ccc4Cl)C[C@H]3C2)n1 ; CN(Cc1cccc(-c2ccccc2)c1)[C@H]1C[C@@H]2CN(C(=O)n3ccc(C(=O)O)n3)C[C@@H]2C1 ; C[C@@H]1CN(Cc2ccc(Cl)c(-c3nccs3)c2)CCN1C(=O)n1ccc(C(=O)O)n1]

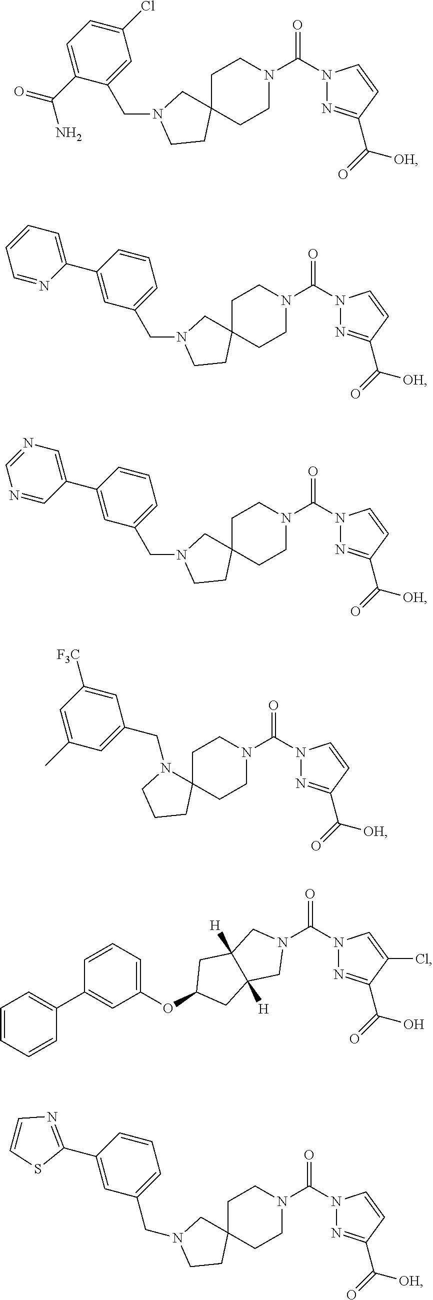 [SMILES: NC(=O)c1ccc(Cl)cc1CN1CCC2(CCN(C(=O)n3ccc(C(=O)O)n3)CC2)C1 ; O=C(O)c1ccn(C(=O)N2CCC3(CCN(Cc4cccc(-c5ccccn5)c4)C3)CC2)n1 ; O=C(O)c1ccn(C(=O)N2CCC3(CCN(Cc4cccc(-c5cncnc5)c4)C3)CC2)n1 ; Cc1cc(CN2CCCC23CCN(C(=O)n2ccc(C(=O)O)n2)CC3)cc(C(F)(F)F)c1 ; O=C(O)c1nn(C(=O)N2C[C@H]3C[C@H](Oc4cccc(-c5ccccc5)c4)C[C@H]3C2)cc1Cl ; O=C(O)c1ccn(C(=O)N2CCC3(CCN(Cc4cccc(-c5nccs5)c4)C3)CC2)n1]

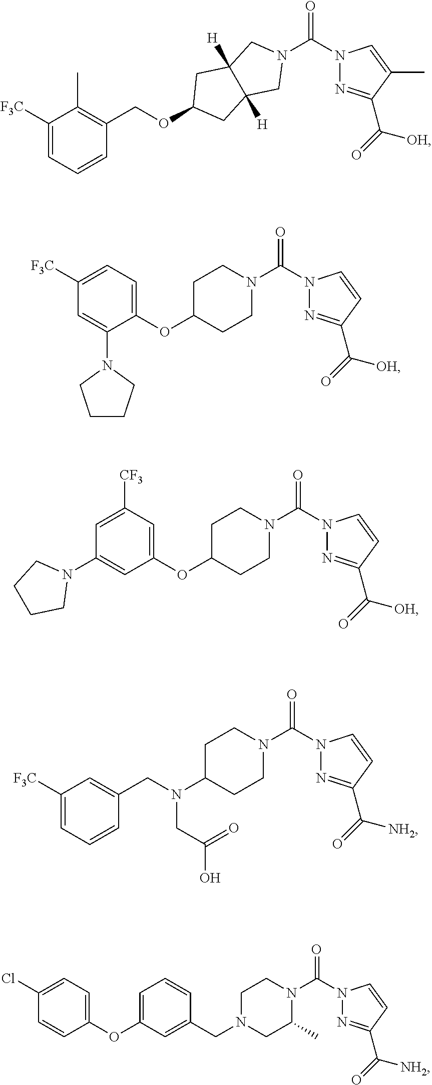 [SMILES: Cc1cn(C(=O)N2C[C@H]3C[C@H](OCc4cccc(C(F)(F)F)c4C)C[C@H]3C2)nc1C(=O)O ; O=C(O)c1ccn(C(=O)N2CCC(Oc3ccc(C(F)(F)F)cc3N3CCCC3)CC2)n1 ; O=C(O)c1ccn(C(=O)N2CCC(Oc3cc(N4CCCC4)cc(C(F)(F)F)c3)CC2)n1 ; NC(=O)c1ccn(C(=O)N2CCC(N(CC(=O)O)Cc3cccc(C(F)(F)F)c3)CC2)n1 ; C[C@@H]1CN(Cc2cccc(Oc3ccc(Cl)cc3)c2)CCN1C(=O)n1ccc(C(N)=O)n1]

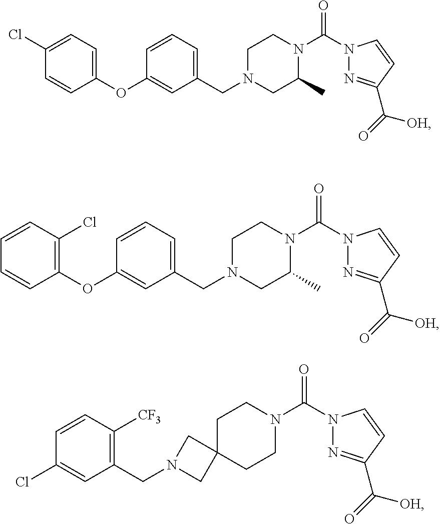 [SMILES: C[C@H]1CN(Cc2cccc(Oc3ccc(Cl)cc3)c2)CCN1C(=O)n1ccc(C(=O)O)n1 ; C[C@@H]1CN(Cc2cccc(Oc3ccccc3Cl)c2)CCN1C(=O)n1ccc(C(=O)O)n1 ; O=C(O)c1ccn(C(=O)N2CCC3(CC2)CN(Cc2cc(Cl)ccc2C(F)(F)F)C3)n1]

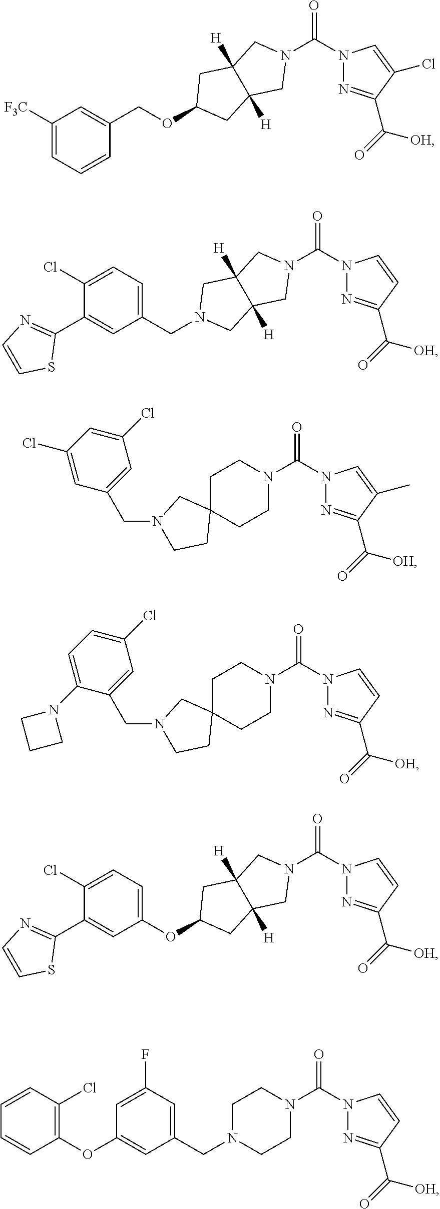 [SMILES: O=C(O)c1nn(C(=O)N2C[C@H]3C[C@H](OCc4cccc(C(F)(F)F)c4)C[C@H]3C2)cc1Cl ; O=C(O)c1ccn(C(=O)N2C[C@H]3CN(Cc4ccc(Cl)c(-c5nccs5)c4)C[C@H]3C2)n1 ; Cc1cn(C(=O)N2CCC3(CCN(Cc4cc(Cl)cc(Cl)c4)C3)CC2)nc1C(=O)O ; O=C(O)c1ccn(C(=O)N2CCC3(CCN(Cc4cc(Cl)ccc4N4CCC4)C3)CC2)n1 ; O=C(O)c1ccn(C(=O)N2C[C@H]3C[C@H](Oc4ccc(Cl)c(-c5nccs5)c4)C[C@H]3C2)n1 ; O=C(O)c1ccn(C(=O)N2CCN(Cc3cc(F)cc(Oc4ccccc4Cl)c3)CC2)n1]

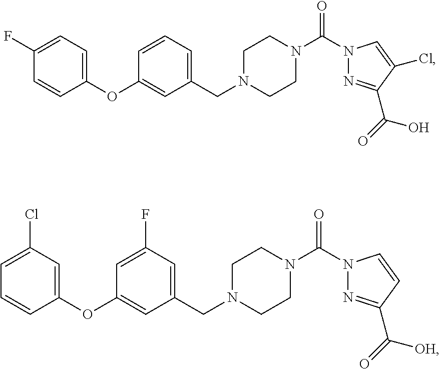 [SMILES: O=C(O)c1nn(C(=O)N2CCN(Cc3cccc(Oc4ccc(F)cc4)c3)CC2)cc1Cl ; O=C(O)c1ccn(C(=O)N2CCN(Cc3cc(F)cc(Oc4cccc(Cl)c4)c3)CC2)n1]

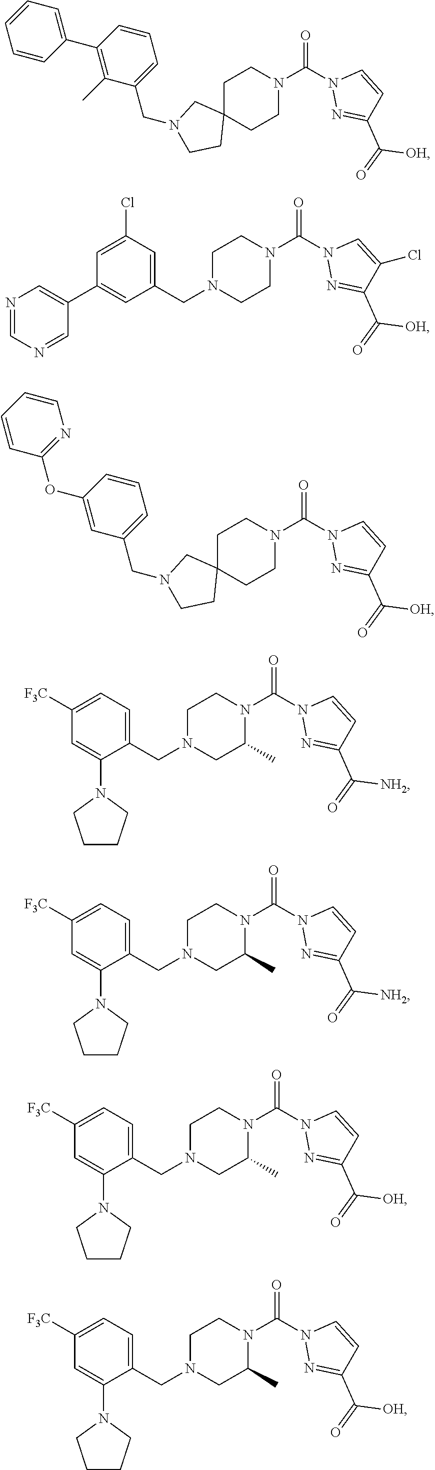

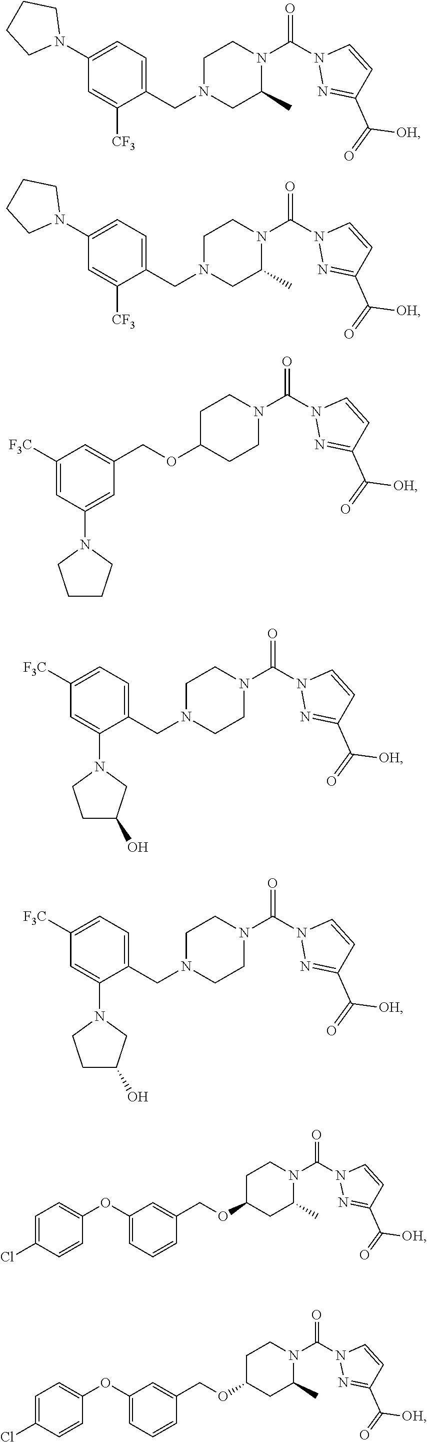

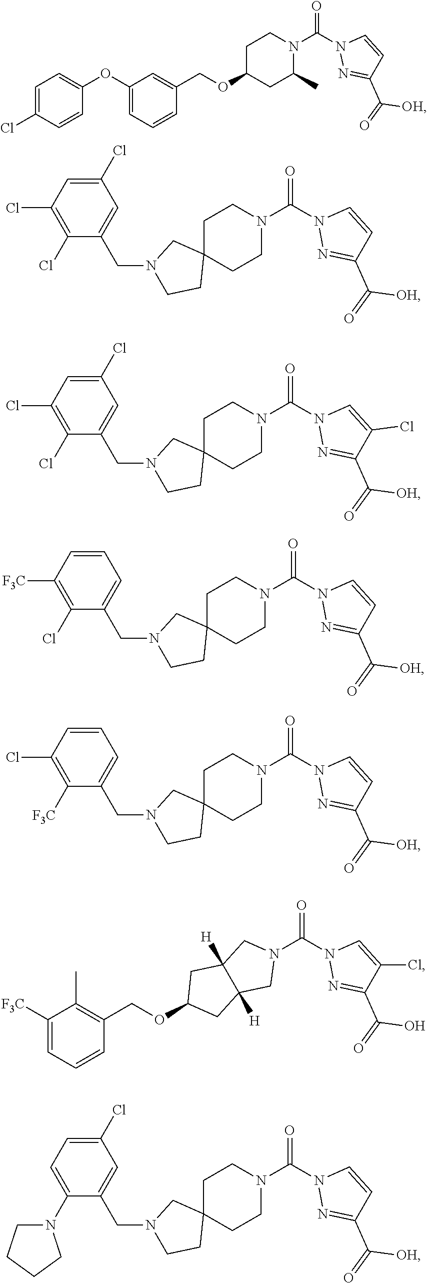

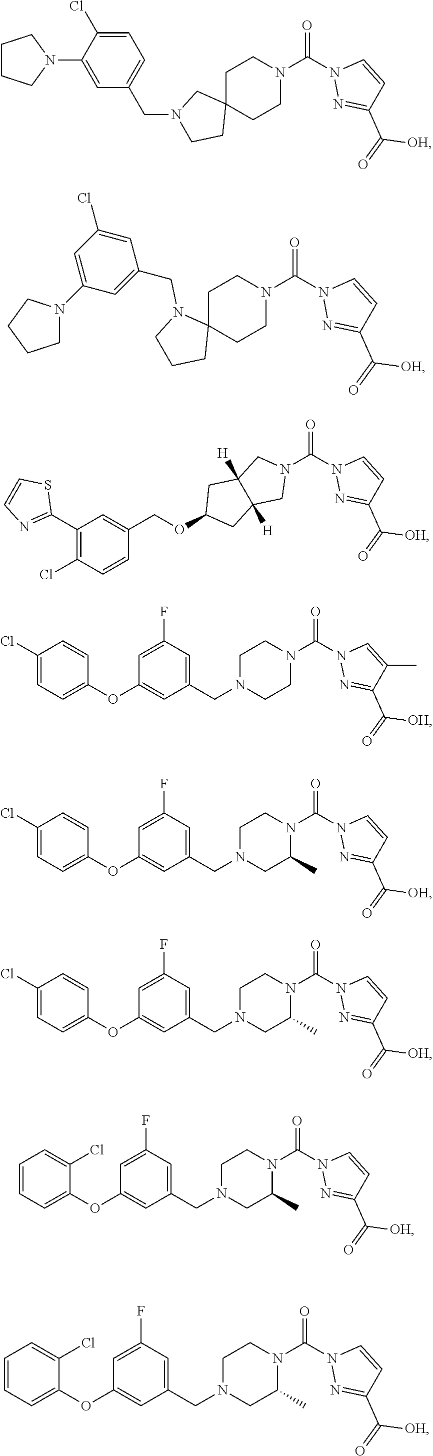

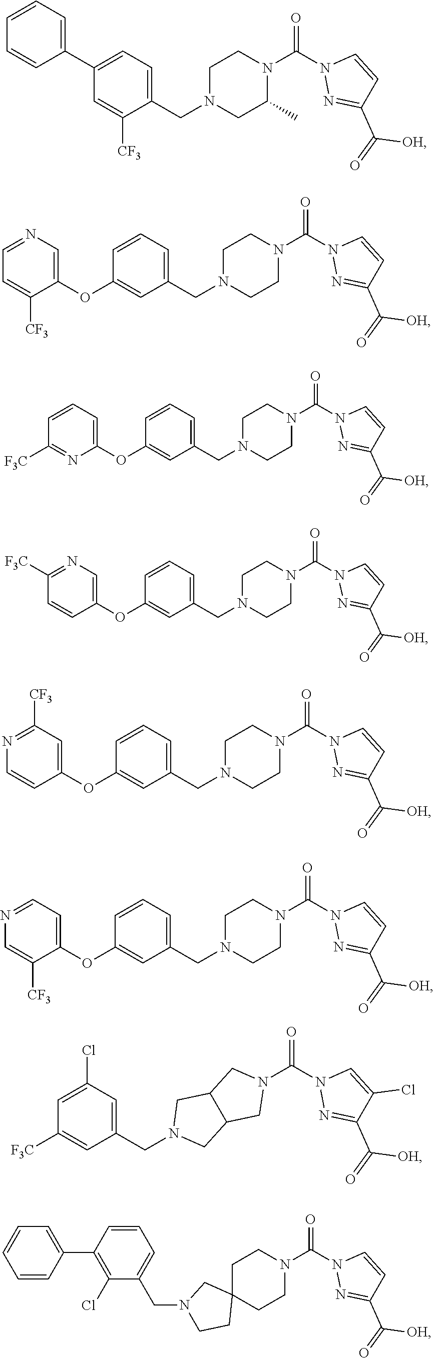

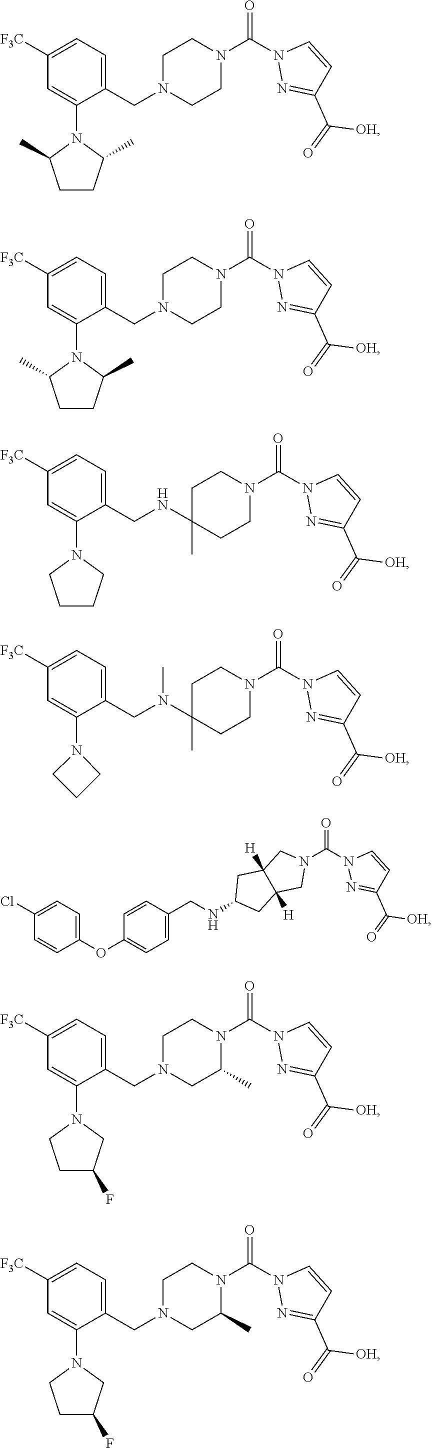

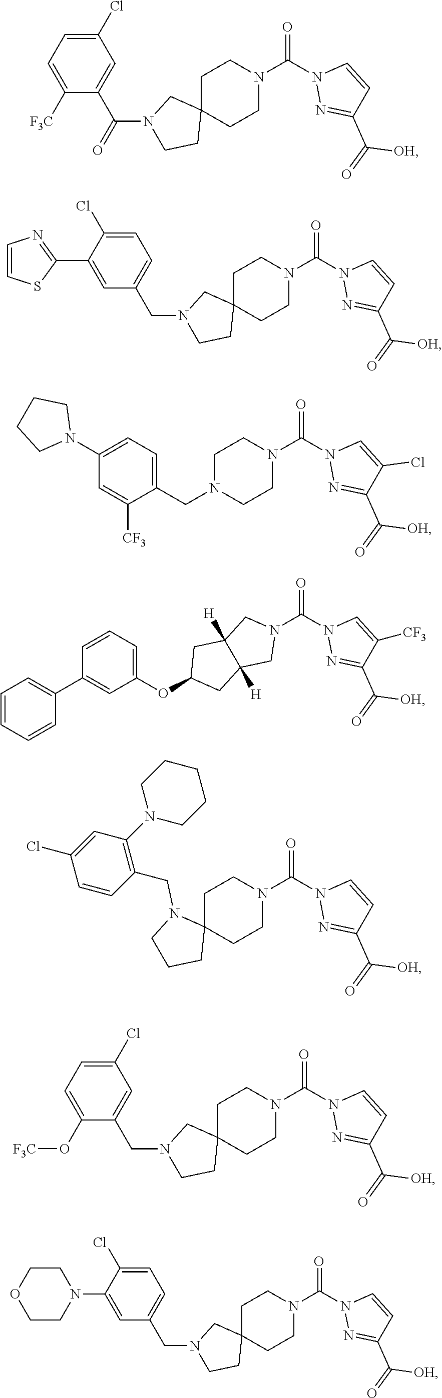

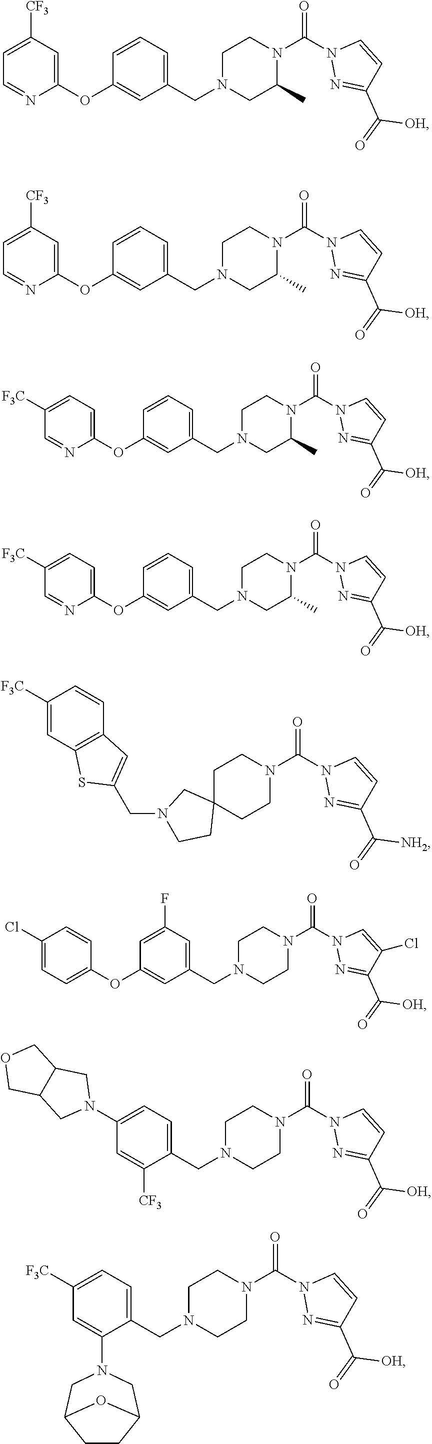

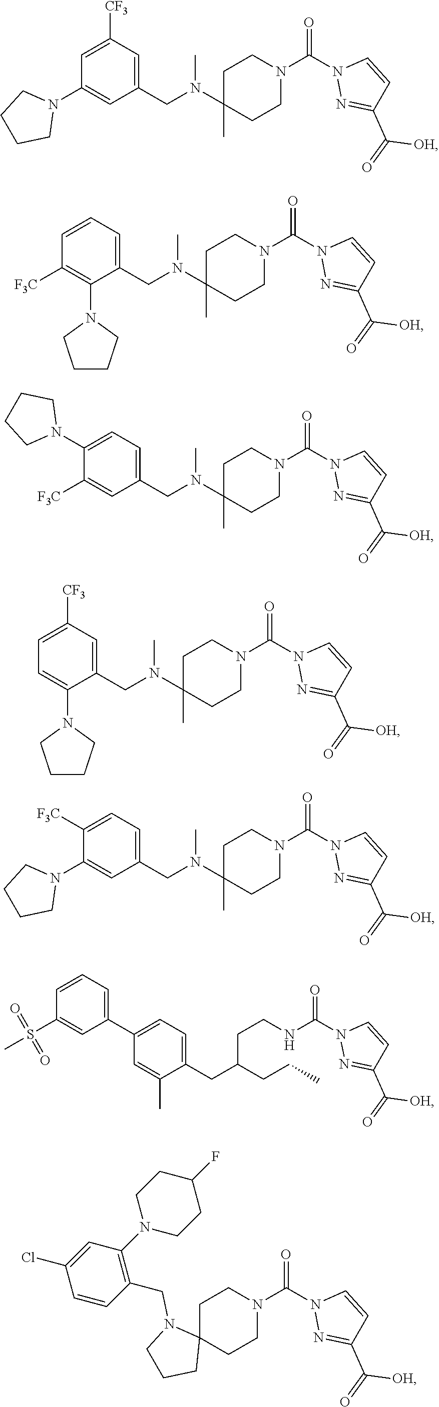

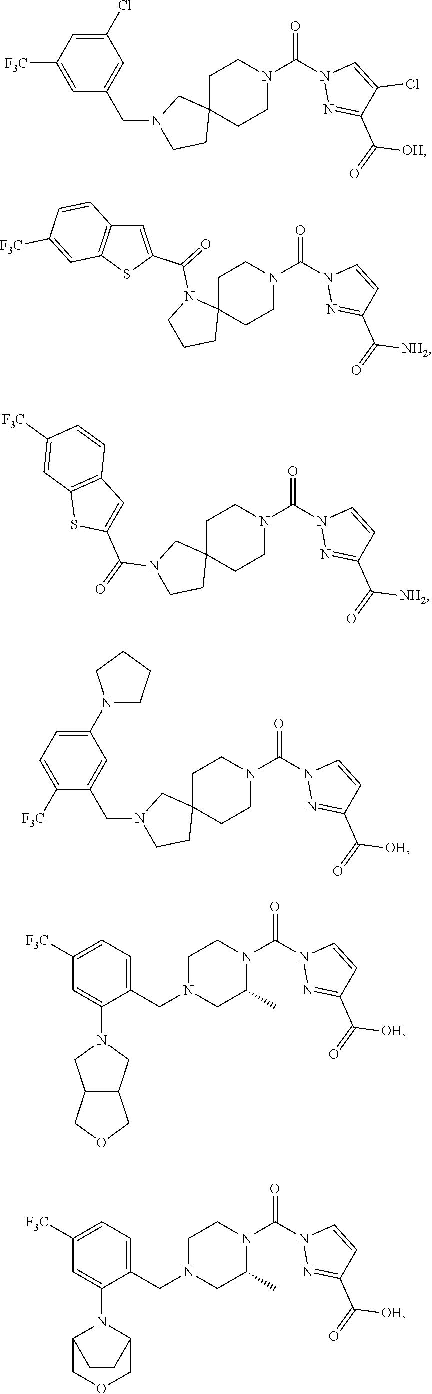 [SMILES: O=C(O)c1nn(C(=O)N2CCC3(CCN(Cc4cc(Cl)cc(C(F)(F)F)c4)C3)CC2)cc1Cl ; NC(=O)c1ccn(C(=O)N2CCC3(CCCN3C(=O)c3cc4ccc(C(F)(F)F)cc4s3)CC2)n1 ; NC(=O)c1ccn(C(=O)N2CCC3(CCN(C(=O)c4cc5ccc(C(F)(F)F)cc5s4)C3)CC2)n1 ; O=C(O)c1ccn(C(=O)N2CCC3(CCN(Cc4cc(N5CCCC5)ccc4C(F)(F)F)C3)CC2)n1 ; C[C@@H]1CN(Cc2ccc(C(F)(F)F)cc2N2CC3COCC3C2)CCN1C(=O)n1ccc(C(=O)O)n1 ; C[C@@H]1CN(Cc2ccc(C(F)(F)F)cc2N2C3CCC2COC3)CCN1C(=O)n1ccc(C(=O)O)n1]

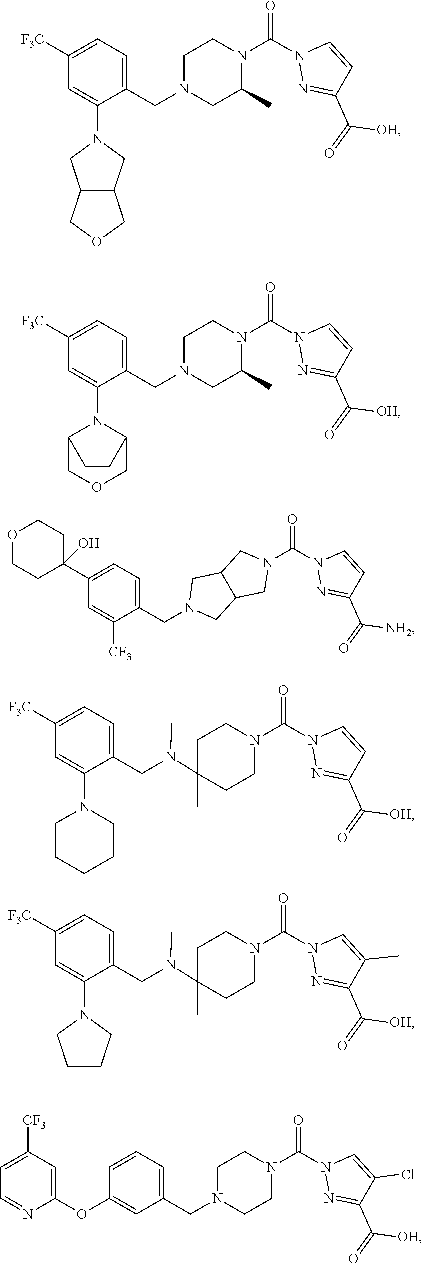

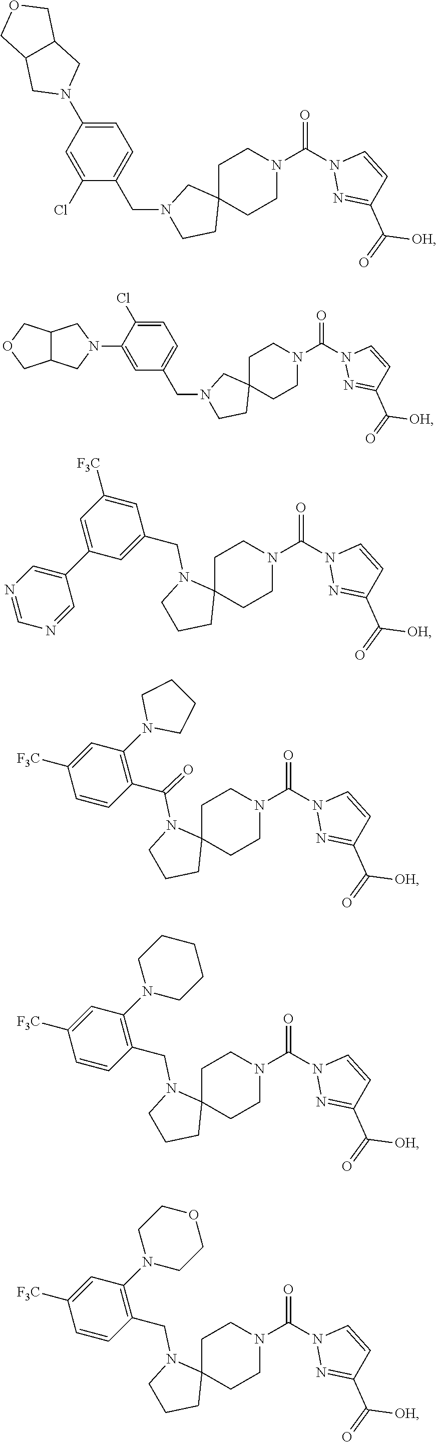 [SMILES: O=C(O)c1ccn(C(=O)N2CCC3(CCN(Cc4ccc(N5CC6COCC6C5)cc4Cl)C3)CC2)n1 ; O=C(O)c1ccn(C(=O)N2CCC3(CCN(Cc4ccc(Cl)c(N5CC6COCC6C5)c4)C3)CC2)n1 ; O=C(O)c1ccn(C(=O)N2CCC3(CCCN3Cc3cc(-c4cncnc4)cc(C(F)(F)F)c3)CC2)n1 ; O=C(O)c1ccn(C(=O)N2CCC3(CCCN3C(=O)c3ccc(C(F)(F)F)cc3N3CCCC3)CC2)n1 ; O=C(O)c1ccn(C(=O)N2CCC3(CCCN3Cc3ccc(C(F)(F)F)cc3N3CCCCC3)CC2)n1 ; O=C(O)c1ccn(C(=O)N2CCC3(CCCN3Cc3ccc(C(F)(F)F)cc3N3CCOCC3)CC2)n1]

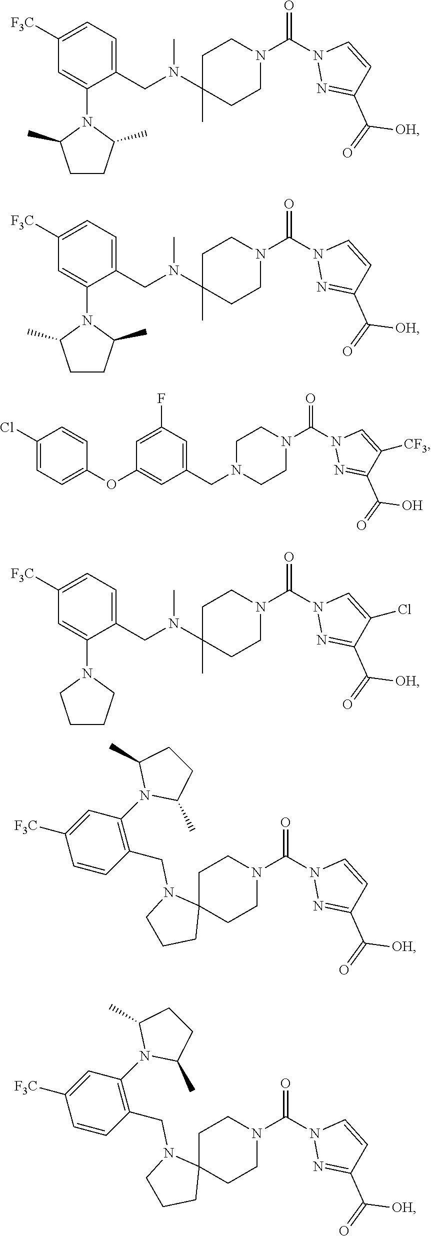

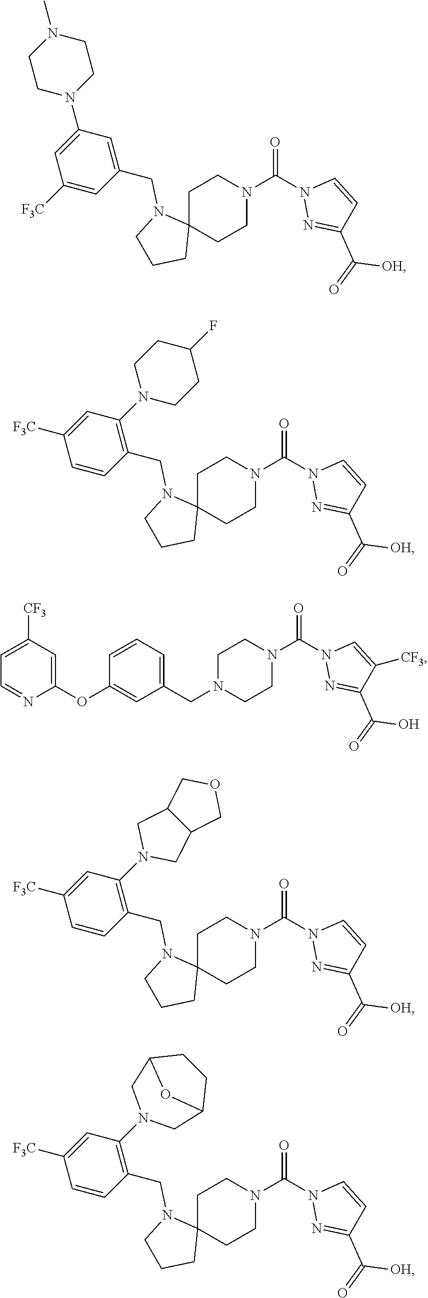 [SMILES: CN1CCN(c2cc(CN3CCCC34CCN(C(=O)n3ccc(C(=O)O)n3)CC4)cc(C(F)(F)F)c2)CC1 ; O=C(O)c1ccn(C(=O)N2CCC3(CCCN3Cc3ccc(C(F)(F)F)cc3N3CCC(F)CC3)CC2)n1 ; O=C(O)c1nn(C(=O)N2CCN(Cc3cccc(Oc4cc(C(F)(F)F)ccn4)c3)CC2)cc1C(F)(F)F ; O=C(O)c1ccn(C(=O)N2CCC3(CCCN3Cc3ccc(C(F)(F)F)cc3N3CC4COCC4C3)CC2)n1 ; O=C(O)c1ccn(C(=O)N2CCC3(CCCN3Cc3ccc(C(F)(F)F)cc3N3CC4CCC(C3)O4)CC2)n1]

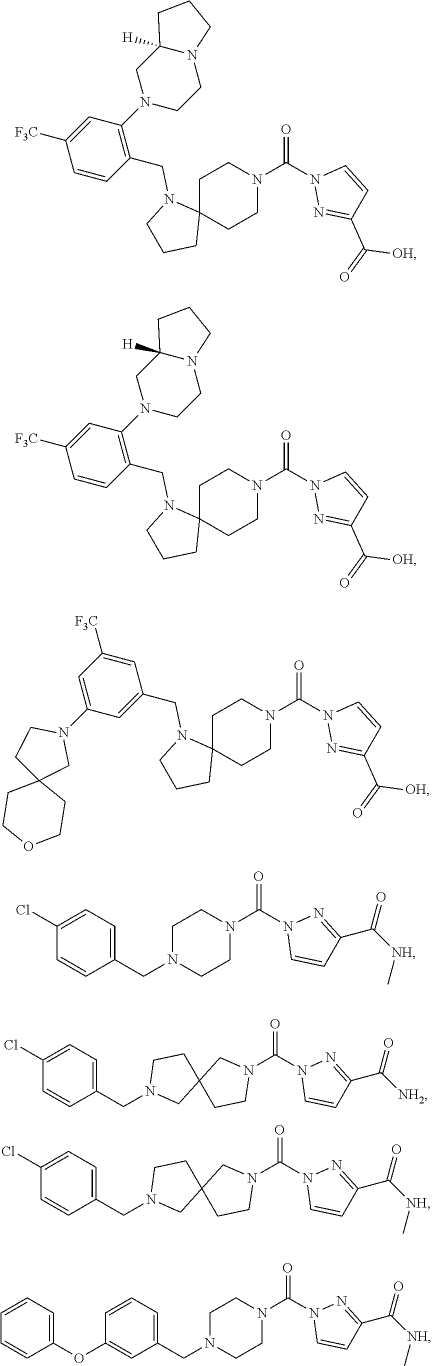

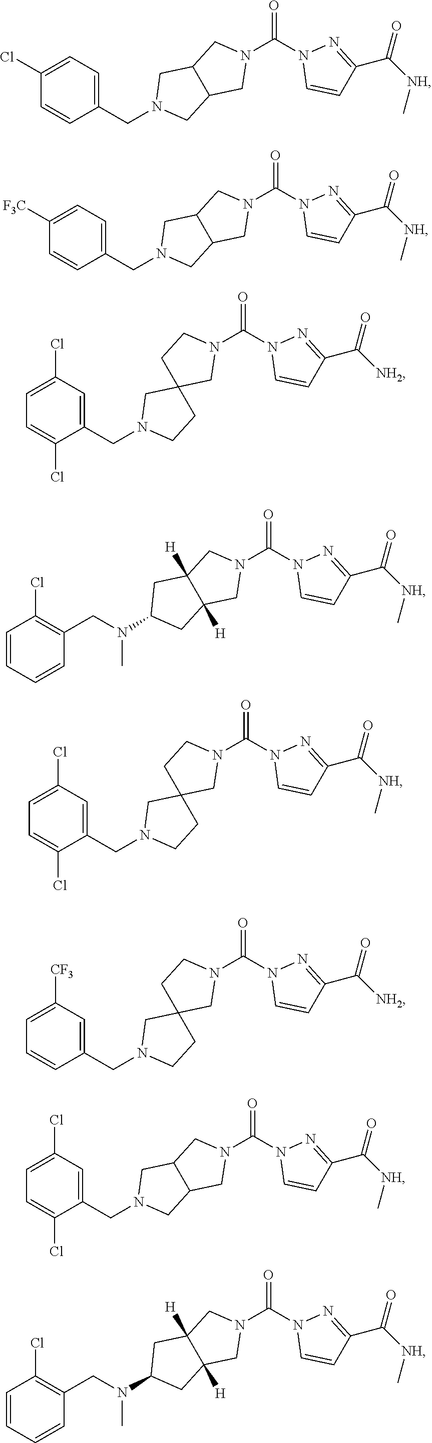

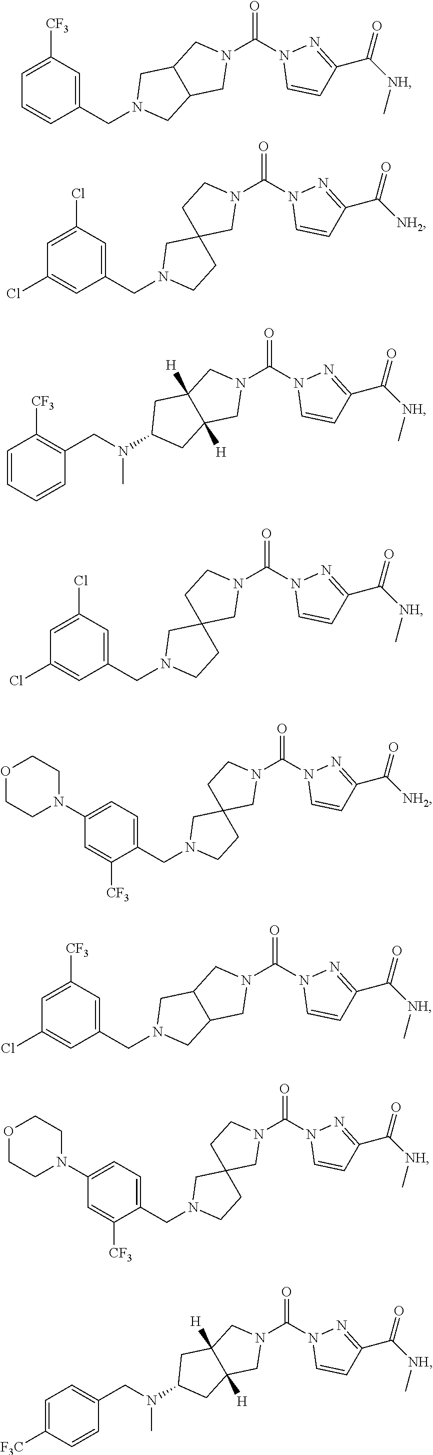

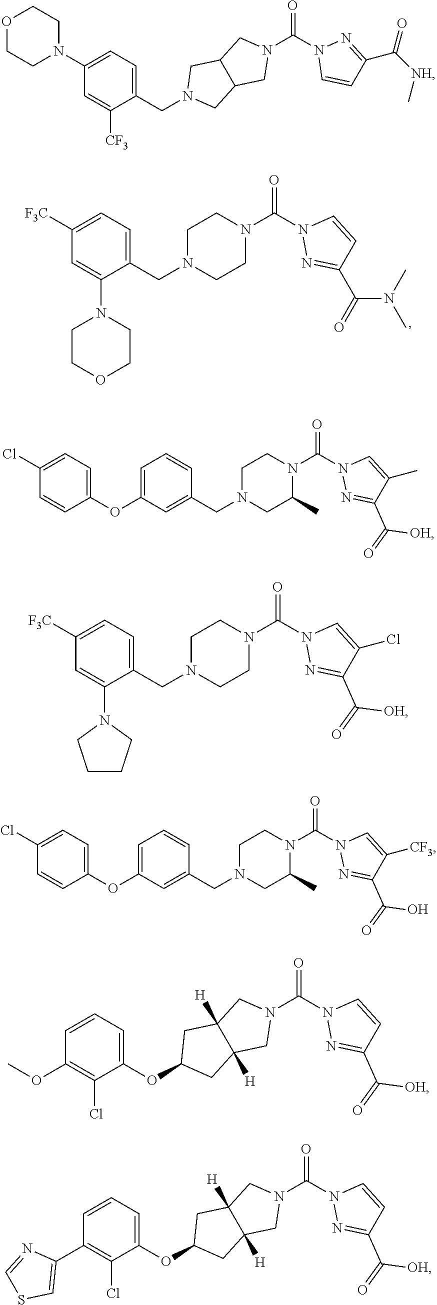

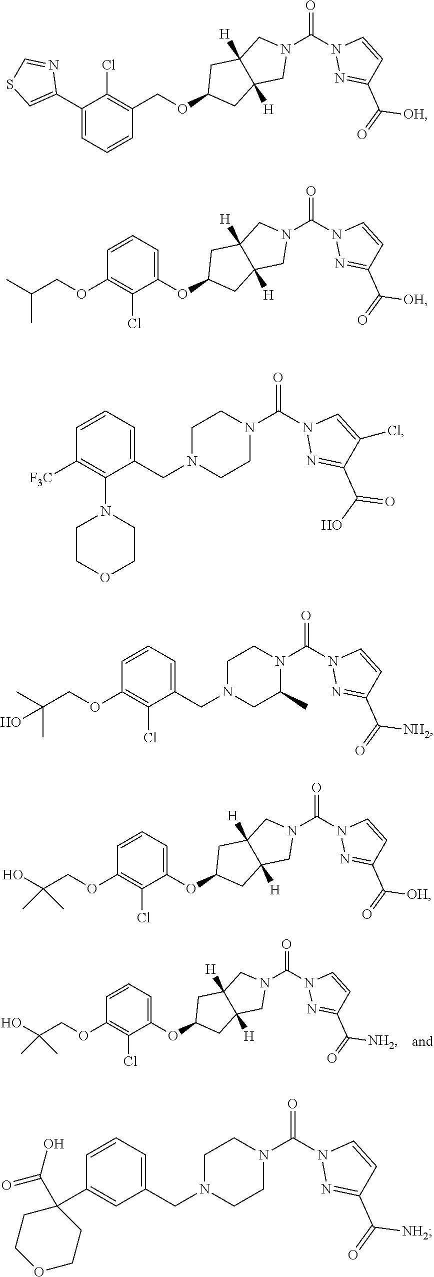

or a solvate, hydrate, tautomer, N-oxide, stereoisomer, or pharmaceutically acceptable salt thereof.

In some embodiments, the disease or condition is atopic dermatitis. In some embodiments, the disease or condition is bladder dysfunction associated with multiple sclerosis. In some embodiments, the disease or condition is cardiovascular disease. In some embodiments, the disease or condition is contact dermatitis. In some embodiments, the disease or condition is cystic fibrosis. In some embodiments, the disease or condition is dermatomyositis. In some embodiments, the disease or condition is eczema. In some embodiments, the disease or condition is endometriosis. In some embodiments, the disease or condition is enteritis. In some embodiments, the disease or condition is fibromyalgia. In some embodiments, the disease or condition is Tourette syndrome. In some embodiments, the disease or condition is inflammatory bowel disease. In some embodiments, the disease or condition is interstitial cystitis. In some embodiments, the disease or condition is irritable bowel syndrome. In some embodiments, the disease or condition is ischemia. In some embodiments, the disease or condition is labor. In some embodiments, the disease or condition is metabolic disorders. In some embodiments, the disease or condition is musculoskeletal diseases. In some embodiments, the disease or condition is neuropathy. In some embodiments, the disease or condition is osteoarthritis. In some embodiments, the disease or condition is pancreatitis. In some embodiments, the disease or condition is pharyngitis. In some embodiments, the disease or condition is post trigeminal neuralgia. In some embodiments, the disease or condition is renal ischemia. In some embodiments, the disease or condition is rheumatoid arthritis. In some embodiments, the disease or condition is skeletal muscle contusion. In some embodiments, the disease or condition is skin diseases. In some embodiments, the disease or condition is sunburn. In some embodiments, the disease or condition is systemic lupus erythematosus. In some embodiments, the disease or condition is pain selected from acute pain, back pain, cancer pain, chest pain, functional chest pain, joint pain, menstrual pain, abdominal pain, abdominal pain associated with irritable bowel syndrome, post mastectomy pain syndrome, post operative pain, toothache, vasoocclusive painful crises in sickle cell disease, and visceral pain.

### DETAILED DESCRIPTION OF THE INVENTION

This disclosure is directed, at least in part, to MAGL modulators or inhibitors. For example, provided herein are methods of treating disease with MAGL modulators or inhibitors.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range varies between 1% and 15% of the stated number or numerical range. The term “comprising” (and related terms such as “comprise” or “comprises” or “having” or “including”) is not intended to exclude that which in other certain embodiments, for example, an embodiment of any composition of matter, composition, method, or process, or the like, described herein, may “consist of” or “consist essentially of” the described features.

### Definitions

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In certain embodiments, an alkyl comprises one to eight carbon atoms (e.g., C₁-C₆ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is hydrogen, alkyl, fluoroalkyl, cycloalkyl, aryl, aralkyl, heterocycloalkyl, heteroaryl or heteroarylalkyl, and each R^{f} is independently alkyl, fluoroalkyl, cycloalkyl, aryl, aralkyl, heterocycloalkyl, heteroaryl or heteroarylalkyl.

“Alkoxy” refers to a radical bonded through an oxygen atom of the formula —O— alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In certain embodiments, an alkenyl comprises two to six carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is hydrogen, alkyl, fluoroalkyl, cycloalkyl, aryl, aralkyl, heterocycloalkyl, heteroaryl or heteroarylalkyl, and each R^{f} is independently alkyl, fluoroalkyl, cycloalkyl, aryl, aralkyl, heterocycloalkyl, heteroaryl, or heteroarylalkyl.

“Alkynyl” refers to a straight or branched hydrocarbon chain radical group consisting solely of carbon and hydrogen atoms, containing at least one carbon-carbon triple bond, and having from two to twelve carbon atoms. In certain embodiments, an alkenyl comprises two to eight carbon atoms. In certain embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is hydrogen, alkyl, fluoroalkyl, cycloalkyl, aryl, aralkyl, heterocycloalkyl, heteroaryl or heteroarylalkyl, and each R^{f} is independently alkyl, fluoroalkyl, cycloalkyl, aryl, aralkyl, heterocycloalkyl, heteroaryl, or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₂-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{f}, —OC(O)— N R^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl, and each R^{f} is independently alkyl, fluoroalkyl, carbocyclyl, carbocyclylalkyl, aryl, aralkyl, heterocyclyl, heterocyclylalkyl, heteroaryl or heteroarylalkyl.

“Aryl” refers to a radical derived from an aromatic monocyclic or multi cyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl,

heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a}) 2, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl (optionally substituted with one or more halo groups), aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenyl ene chain, and where each of the above substituents is unsubstituted unless otherwise indicated.

“Aryloxy” refers to a radical bonded through an oxygen atom of the formula —O-aryl, where aryl is as defined above.

“Aralkyl” refers to a radical of the formula —R^{c}-aryl where R^{c} is an alkyl ene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated, (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. In certain embodiments, a cycloalkyl comprises three to eight carbon atoms (e.g., C₃-C₈ cycloalkyl). In other embodiments, a cycloalkyl comprises three to seven carbon atoms (e.g., C₃-C₇ cycloalkyl). In other embodiments, a cycloalkyl comprises three to six carbon atoms (e.g., C₃-C₆ cycloalkyl). In other embodiments, a cycloalkyl comprises three to five carbon atoms (e.g., C₃-C₅ cycloalkyl). In other embodiments, a cycloalkyl comprises three to four carbon atoms (e.g., C₃-C₄ cycloalkyl). An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbomyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl,

heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a}) 2, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical is optionally substituted as defined above.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, l-fluoromethyl-2-fluoroethyl, and the like. In some embodiments, the alkyl part of the fluoroalkyl radical is optionally substituted as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which includes fused or bridged ring systems. The heteroatoms in the heterocyclyl radical are optionally oxidized. One or more nitrogen atoms, if present, are optionally quaternized. The heterocyclyl radical is partially or fully saturated. In some embodiments, the heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a}) 2, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated. The terms “heterocyclyl”, “heterocycle”, and “heterocycloalkyl” are used interchangeably.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) n-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[6][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5.8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1n-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a}) 2, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroaryloxy” refers to radical bonded through an oxygen atom of the formula —O-heteroaryl, where heteroaryl is as defined above.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

In some embodiments, he compounds disclosed herein contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. In certain embodiments, the compounds presented herein exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

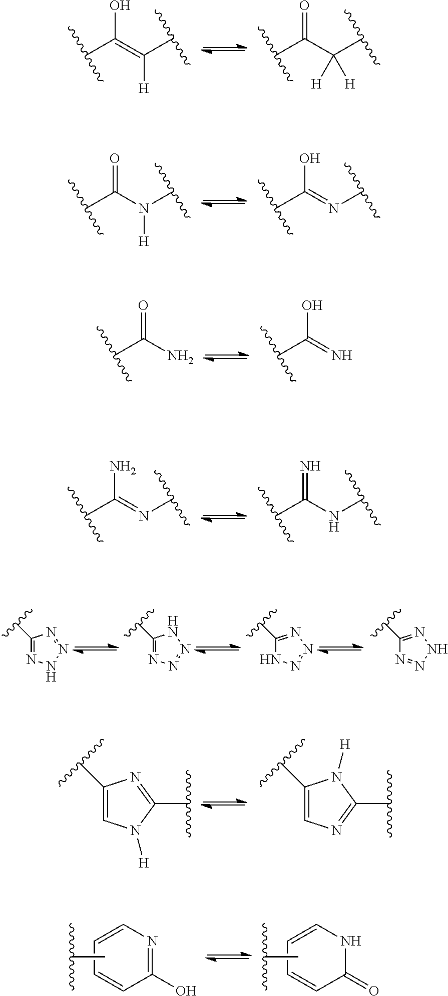

“Optional” or “optionally” means that a subsequently described event or circumstance may or may not occur and that the description includes instances when the event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are prepared by contacting the free base forms with a sufficient amount of the desired acid to produce the salt.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, tri ethyl amine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

As used herein, “treatment” or “treating” or “palliating” or “ameliorating” are used interchangeably herein. These terms refers to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are administered to a patient at risk of developing a particular disease, or to a patient reporting one or more of the physiological symptoms of a disease, even though a diagnosis of this disease has not been made.

### Embodiments of the Invention

In the following, embodiments of the invention are disclosed. The first embodiment is denoted E1, the second embodiment E2 and so forth.

In a first embodiment E1 the present invention relates to a method of treating a disease with a compound of Formula (I).

E1: A method for treating a disease or a condition in a patient wherein the disease or condition is selected from atopic dermatitis, bladder dysfunction associated with multiple sclerosis, cardiovascular disease, contact dermatitis, cystic fibrosis, dermatomyositis, eczema, endometriosis, enteritis, fibromyalgia, Tourette syndrome, inflammatory bowel disease, interstitial cystitis, irritable bowel syndrome, ischemia, labor, abdominal pain, abdominal pain associated with irritable bowel syndrome, acute pain, back pain, cancer pain, chest pain, functional chest pain, joint pain, menstrual pain, metabolic disorders, musculoskeletal diseases, neuropathy, osteoarthritis, pancreatitis, pharyngitis, post mastectomy pain syndrome, post trigeminal neuralgia, post operative pain, renal ischemia, rheumatoid arthritis, skeletal muscle contusion, skin diseases, sunburn, systemic lupus erythematosus, toothache, vasoocclusive painful crises in sickle cell disease, and visceral pain, comprising administering to the patient in need thereof a therapeutically effective amount of a compound of Formula (I) having the structure:

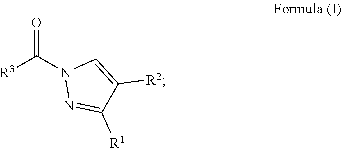

wherein:
R¹ is —C(O)OR¹⁵ or —C(O)NR¹⁰R¹¹;
R² is H, halogen, C₁₋₆alkyl, or C₁₋₆haloalkyl; R³ is

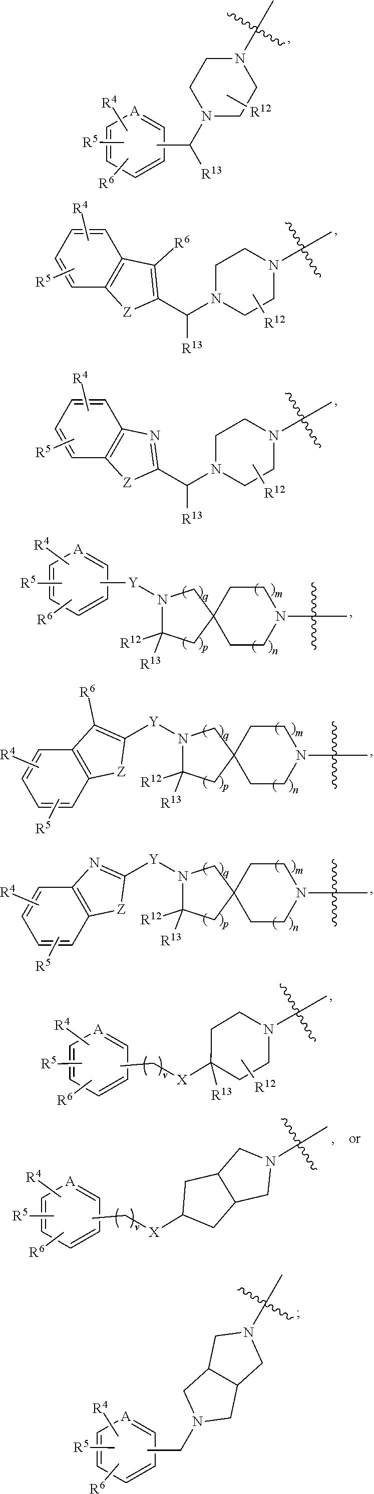

A is N or C(H);
X is —O—, —N(R¹⁶)—, or —CH₂N(R¹⁶)CH₂—;
Y is —CH₂— or —C(O)—;
Z is —S—, —O—, or —N(R¹⁸)—;
R⁴ is H, halogen, —OR⁷, C₁₋₆alkyl, C₂₋₆alkenyl, C₂₋₆alkynyl, C₁₋₆haloalkyl, —C(O)NR⁸R⁹, C₃₋₈cycloalkyl, C₂₋₉heterocycloalkyl, —C₁₋₆alkyl-C₂₋₉heterocycloalkyl, C₆₋₁₀aryl, or C₁₋₉heteroaryl, wherein C₃₋₈cycloalkyl, C₂₋₉heterocycloalkyl, —C₁₋₆alkyl-C₂₋₉ heterocycloalkyl, C₆₋₁₀aryl, or C₁₋₉heteroaryl are optionally substituted with 1 or 2 R¹⁴;
R⁵ is H, halogen, C₁₋₆alkyl, C₁₋₆haloalkyl, C₁₋₆haloalkoxy, or phenyl;
R⁶ is H, halogen or C₁₋₆alkyl;
R⁷ is H, C₁₋₆alkyl, C₁₋₆haloalkyl, —C₁₋₆alkyl-OH, C₂₋₉heterocycloalkyl, C₆₋₁₀aryl, or C₁₋₉heteroaryl, wherein C₂₋₉heterocycloalkyl, C₆₋₁₀aryl, or C₁₋₉heteroaryl are optionally substituted with 1 or 2 R¹⁴;
each R⁸ and each R⁹ are independently selected from H and C₁₋₆alkyl; or R⁸ and R⁹ together with the nitrogen to which they are attached are combined to form a heterocycloalkyl ring;
R¹⁰ and R¹¹ are each independently H or C₁₋₆alkyl;
R¹² is H, halogen, or C₁₋₆alkyl;
R¹³ is H or C₁₋₆alkyl;
each R¹⁴ is independently selected from halogen, —OH, C₁₋₆alkyl, C₁₋₆haloalkyl, C₁₋₆alkoxy, —C₁₋₆alkyl-OH, C₃₋₈cycloalkyl, —C(O)OH, —C(O)NR⁸R⁹, —SO₂—C₁₋₆alkyl, and —N(R¹⁷)C(O)—C₁₋₆alkyl;
R¹⁵ is H or C₁₋₆alkyl;
R¹⁶ is H, C₁₋₆alkyl, —C(O)—C₁₋₆alkyl, —C₁₋₆alkyl-OH, or —CH₂CO₂H;
R¹⁷ is H or C₁₋₆alkyl;
R¹⁸ is H or C₁₋₆alkyl;
v is 0 or 1;
n is 0 or 1;
m is 0 or 1;
p is 0, 1, or 2; and
q is 0, 1 or 2;
or a solvate, hydrate, tautomer, N-oxide, stereoisomer, or pharmaceutically acceptable salt thereof.

E2: A method for treating a disease or a condition in a patient wherein the disease or condition is selected from atopic dermatitis, bladder dysfunction associated with multiple sclerosis, cardiovascular disease, contact dermatitis, cystic fibrosis, dermatomyositis, eczema, endometriosis, enteritis, fibromyalgia, Tourette syndrome, inflammatory bowel disease, interstitial cystitis, irritable bowel syndrome, ischemia, labor, abdominal pain, abdominal pain associated with irritable bowel syndrome, acute pain, back pain, cancer pain, chest pain, functional chest pain, joint pain, menstrual pain, metabolic disorders, musculoskeletal diseases, neuropathy, osteoarthritis, pancreatitis, pharyngitis, post mastectomy pain syndrome, post trigeminal neuralgia, post operative pain, renal ischemia, rheumatoid arthritis, skeletal muscle contusion, skin diseases, sunburn, systemic lupus erythematosus, toothache, vasoocclusive painful crises in sickle cell disease, and visceral pain, comprising administering to the patient in need thereof a therapeutically effective amount of a compound of Formula (I′) having the structure:

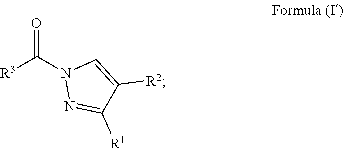

wherein:
R¹ is —C(O)OR¹⁵ or —C(O)NR¹⁰R¹¹;
R² is H, halogen, C₁₋₆alkyl, or C₁₋₆haloalkyl;
R³ is

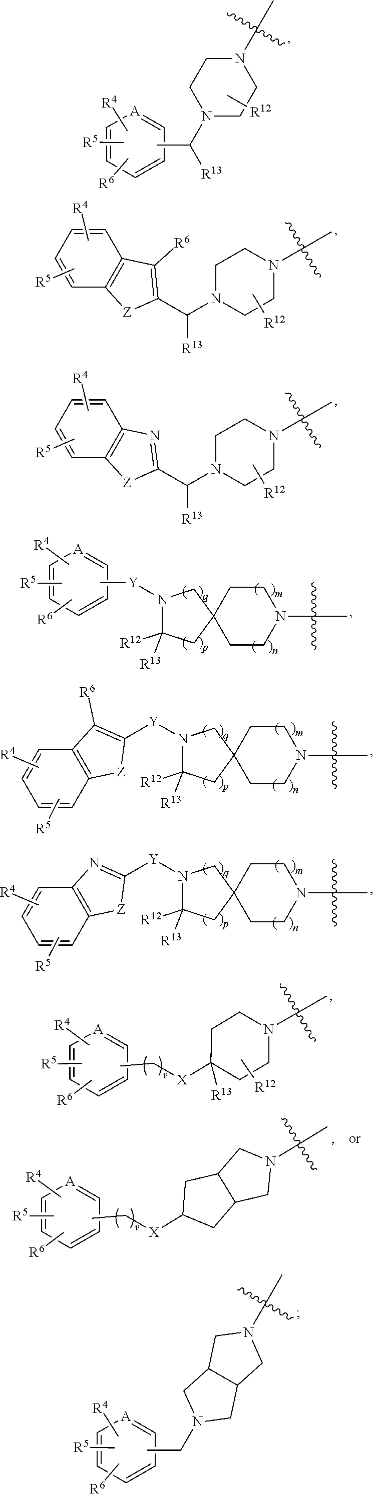

A is N or C(H);
X is —O—, —N(R¹⁶)—, or —CH₂N(R¹⁶)CH₂—;
Y is —CH₂— or —C(O)—;
Z is —S—, —O—, or —N(R¹⁸)—;
R⁴ is H, halogen, —OR⁷, C₁₋₆alkyl, C₂₋₆alkenyl, C₂₋₆alkynyl, C₁₋₆haloalkyl, —C(O)NR⁸R⁹, C₂₋₉heterocycloalkyl, —C₁₋₆alkyl-C₂₋₉heterocycloalkyl, C₆₋₁₀aryl, or C₁₋₉heteroaryl, wherein C₂₋₉heterocycloalkyl, —C₁₋₆alkyl-C₂₋₉heterocycloalkyl, C₆₋₁₀aryl, or C₁₋₉heteroaryl are optionally substituted with 1 or 2 R¹⁴;
R⁵ is H, halogen, C₁₋₆alkyl, C₁₋₆haloalkyl, C₁₋₆haloalkoxy, or phenyl;
R⁶ is H, halogen or C₁₋₆alkyl;
R⁷ is H, C₁₋₆alkyl, C₁₋₆haloalkyl, C₂₋₉heterocycloalkyl, C₆₋₁₀aryl, or C₁₋₉heteroaryl, wherein C₂₋₉heterocycloalkyl, C₆₋₁₀aryl, or C₁₋₉heteroaryl are optionally substituted with 1 or 2 R¹⁴;
each R⁸ and each R⁹ are independently selected from H and C₁₋₆alkyl; or R⁸ and R⁹ together with the nitrogen to which they are attached are combined to form a heterocycloalkyl ring;
R¹⁰ and R¹¹ are each independently H or C₁₋₆alkyl;
R¹² is H, halogen, or C₁₋₆alkyl;
R¹³ is H or C₁₋₆alkyl;
each R¹⁴ is independently selected from halogen, —OH, C₁₋₆alkyl, C₁₋₆haloalkyl, C₁₋₆alkoxy, —C₁₋₆alkyl-OH, C₃₋₈cycloalkyl, —C(O)NR⁸R⁹, —SO₂—C₁₋₆alkyl, and —N(R¹⁷)C(O)—C₁₋₆alkyl;
R¹⁵ is H or C₁₋₆alkyl;
R¹⁶ is H, C₁₋₆alkyl, —C(O)—C₁₋₆alkyl, or —CH₂CO₂H;
R¹⁷ is H or C₁₋₆alkyl;
R¹⁸ is H or C₁₋₆alkyl;
v is 0 or 1;
n is 0 or 1;
m is 0 or 1;
p is 0, 1, or 2; and
q is 0, 1 or 2;
or a solvate, hydrate, tautomer, N-oxide, stereoisomer, or pharmaceutically acceptable salt thereof.

E3: The method of embodiment 1 or 2, or a solvate, hydrate, tautomer, N-oxide, stereoisomer, or pharmaceutically acceptable salt thereof, wherein R³ is

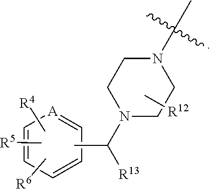

E4: The method of embodiment 3, or a solvate, hydrate, tautomer, N-oxide, stereoisomer, or pharmaceutically acceptable salt thereof, wherein A is C(H).

E5: The method of embodiment 3, or a solvate, hydrate, tautomer, N-oxide, stereoisomer, or pharmaceutically acceptable salt thereof, wherein A is N.

E6: The method of any one of embodiments 3-5, or a solvate, hydrate, tautomer, N-oxide, stereoisomer, or pharmaceutically acceptable salt thereof, wherein R⁶ is H.

E7: The method of embodiment 1 or 2, or a solvate, hydrate, tautomer, N-oxide, stereoisomer, or pharmaceutically acceptable salt thereof, wherein R³ is

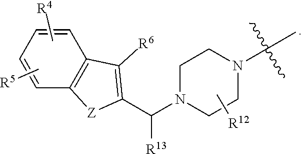

E8: The method of embodiment 1 or 2, or a solvate, hydrate, tautomer, N-oxide, stereoisomer, or pharmaceutically acceptable salt thereof, wherein R³ is

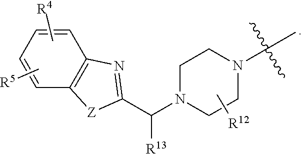

E9: The method of embodiment 7 or embodiment 8, or a solvate, hydrate, tautomer, N-oxide, stereoisomer, or pharmaceutically acceptable salt thereof, wherein Z is —S—.

E10: The method of embodiment 7 or embodiment 8, or a solvate, hydrate, tautomer, N-oxide, stereoisomer, or pharmaceutically acceptable salt thereof, wherein Z is —N(H)—.

E11: The method of embodiment 1 or 2, or a solvate, hydrate, tautomer, N-oxide, stereoisomer, or pharmaceutically acceptable salt thereof, wherein R³ is

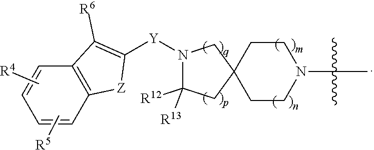

E12: The method of embodiment 11, or a solvate, hydrate, tautomer, N-oxide, stereoisomer, or pharmaceutically acceptable salt thereof, wherein Z is —S—.

E13: The method of embodiment 1 or 2, or a solvate, hydrate, tautomer, N-oxide, stereoisomer, or pharmaceutically acceptable salt thereof, wherein R³ is

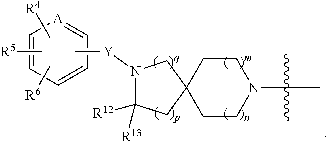

E14: The method of any one of embodiments 11-13, or a solvate, hydrate, tautomer, N-oxide, stereoisomer, or pharmaceutically acceptable salt thereof, wherein m is 1, n is 1, q is 1, and p is 1.

E15: The method of any one of embodiments 11-13, or a solvate, hydrate, tautomer, N-oxide, stereoisomer, or pharmaceutically acceptable salt thereof, wherein m is 0, n is 1, q is 2, and p is 1.

E16: The method of any one of embodiments 11-13, or a solvate, hydrate, tautomer, N-oxide, stereoisomer, or pharmaceutically acceptable salt thereof, wherein m is 1, n is 1, q is 0, and p is 2.

E17: The method of any one of embodiments 11-13, or a solvate, hydrate, tautomer, N-oxide, stereoisomer, or pharmaceutically acceptable salt thereof, wherein m is 1, n is 1, q is 1, and p is 0.

E18: The method of any one of embodiments 11-17, or a solvate, hydrate, tautomer, N-oxide, stereoisomer, or pharmaceutically acceptable salt thereof, wherein Y is —CH₂—.

E19: The method of any one of embodiments 11-17, or a solvate, hydrate, tautomer, N-oxide, stereoisomer, or pharmaceutically acceptable salt thereof, wherein Y is —C(O)—.

E20: The method of any one of embodiments 1-19, or a solvate, hydrate, tautomer, N-oxide, stereoisomer, or pharmaceutically acceptable salt thereof, wherein R¹³ is H.

E21: The method of any one of embodiments 1-20, or a solvate, hydrate, tautomer, N-oxide, stereoisomer, or pharmaceutically acceptable salt thereof, wherein R¹² is H.

E22: The method of embodiment 1 or 2, or a solvate, hydrate, tautomer, N-oxide, stereoisomer, or pharmaceutically acceptable salt thereof, wherein R³ is

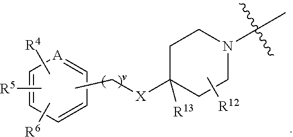

E23: The method of embodiment 22, or a solvate, hydrate, tautomer, N-oxide, stereoisomer, or pharmaceutically acceptable salt thereof, wherein R¹³ is H.

E24: The method of embodiment 22, or a solvate, hydrate, tautomer, N-oxide, stereoisomer, or pharmaceutically acceptable salt thereof, wherein R¹³ is —CH₃.

E25: The method of any one of embodiments 22-24, or a solvate, hydrate, tautomer, N-oxide, stereoisomer, or pharmaceutically acceptable salt thereof, wherein R¹² is H.

E26: The method of embodiment 1 or 2, or a solvate, hydrate, tautomer, N-oxide, stereoisomer, or pharmaceutically acceptable salt thereof, wherein R³ is

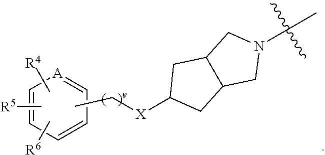

E27: The method of any one of embodiments 22-26, or a solvate, hydrate, tautomer, N-oxide, stereoisomer, or pharmaceutically acceptable salt thereof, wherein v is 0.

E28: The method of any one of embodiments 22-26, or a solvate, hydrate, tautomer, N-oxide, stereoisomer, or pharmaceutically acceptable salt thereof, wherein v is 1.

E29: The method of any one of embodiments 22-28, or a solvate, hydrate, tautomer, N-oxide, stereoisomer, or pharmaceutically acceptable salt thereof, wherein X is —O—.

E30: The method of any one of embodiments 22-28, or a solvate, hydrate, tautomer, N-oxide, stereoisomer, or pharmaceutically acceptable salt thereof, wherein X is —N(CH₃)—.

E31: The method of any one of embodiments 22-28, or a solvate, hydrate, tautomer, N-oxide, stereoisomer, or pharmaceutically acceptable salt thereof, wherein X is —CH₂N(CH₃)CH₂—.

E32: The method of embodiment 1 or 2, or a solvate, hydrate, tautomer, N-oxide, stereoisomer, or pharmaceutically acceptable salt thereof, wherein R³ is

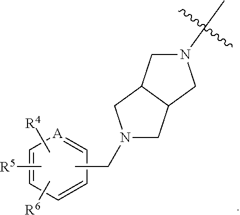

E33: The method of any one of embodiments 13-32, or a solvate, hydrate, tautomer, N-oxide, stereoisomer, or pharmaceutically acceptable salt thereof, wherein A is C(H).

E34: The method of any one of embodiments 13-32, or a solvate, hydrate, tautomer, N-oxide, stereoisomer, or pharmaceutically acceptable salt thereof, wherein A is N.

E35: The method of any one of embodiments 11-34, or a solvate, hydrate, tautomer, N-oxide, stereoisomer, or pharmaceutically acceptable salt thereof, wherein R⁶ is H.

E36: The method of any one of embodiments 1-35, or a solvate, hydrate, tautomer, N-oxide, stereoisomer, or pharmaceutically acceptable salt thereof, wherein R⁴ is halogen, —OR⁷, C₁₋₆alkyl, C₂₋₆alkynyl, C₁₋₆haloalkyl, —C(O)NR⁸R⁹, C₂₋₉ heterocycloalkyl, C₆₋₁₀aryl, or C₁₋₉heteroaryl, wherein C₂₋₉heterocycloalkyl, —C₁₋₆alkyl-C₂₋₉heterocycloalkyl, C₆₋₁₀aryl, C₁₋₉heteroaryl are optionally substituted with 1 or 2 R¹⁴.

E37: The method of any one of embodiments 1-35, or a solvate, hydrate, tautomer, N-oxide, stereoisomer, or pharmaceutically acceptable salt thereof, wherein R⁴ is halogen, —OR⁷, C₁₋₆haloalkyl, C₂₋₉heterocycloalkyl, C₆₋₁₀aryl, or C₁₋₉heteroaryl, wherein C₂₋₉heterocycloalkyl, C₆₋₁₀aryl, or C₁₋₉heteroaryl are optionally substituted with 1 or 2 R¹⁴.

E38: The method of any one of embodiments 1-37, or a solvate, hydrate, tautomer, N-oxide, stereoisomer, or pharmaceutically acceptable salt thereof, wherein R⁴ is halogen.

E39: The method of embodiment 38, or a solvate, hydrate, tautomer, N-oxide, stereoisomer, or pharmaceutically acceptable salt thereof, wherein R⁴ is —C₁.

E40: The method of any one of embodiments 1-37, or a solvate, hydrate, tautomer, N-oxide, stereoisomer, or pharmaceutically acceptable salt thereof, wherein R⁴ is C₁₋₆haloalkyl.

E41: The method of embodiment 40, or a solvate, hydrate, tautomer, N-oxide, stereoisomer, or pharmaceutically acceptable salt thereof, wherein R⁴ is —CF₃.

E42: The method of any one of embodiments 1-37, or a solvate, hydrate, tautomer, N-oxide, stereoisomer, or pharmaceutically acceptable salt thereof, wherein R⁴ is —OR⁷.

E43: The method of embodiment 42, or a solvate, hydrate, tautomer, N-oxide, stereoisomer, or pharmaceutically acceptable salt thereof, wherein R⁷ is C₁₋₆haloalkyl, C₆₋₁₀aryl, or C₁₋₉heteroaryl, wherein C₆₋₁₀aryl or C₁₋₉heteroaryl are optionally substituted with 1 or 2 R¹⁴.

E44: The method of embodiment 42, or a solvate, hydrate, tautomer, N-oxide, stereoisomer, or pharmaceutically acceptable salt thereof, wherein R⁷ is phenyl, pyridyl, pyrimidinyl, pyridizinyl, or pyrazinyl, wherein phenyl, pyridyl, pyrimidinyl, pyridizinyl, or pyrazinyl are optionally substituted with halogen or C₁₋₆haloalkyl.

E45: The method of any one of embodiments 1-37, or a solvate, hydrate, tautomer, N-oxide, stereoisomer, or pharmaceutically acceptable salt thereof, wherein R⁴ is C₂₋₉heterocycloalkyl optionally substituted with 1 or 2 R¹⁴.

E46: The method of embodiment 45, or a solvate, hydrate, tautomer, N-oxide, stereoisomer, or pharmaceutically acceptable salt thereof, wherein R⁴ is an unsubstituted C₂₋₉heterocycloalkyl.

E47: The method of embodiment 45, or a solvate, hydrate, tautomer, N-oxide, stereoisomer, or pharmaceutically acceptable salt thereof, wherein R⁴ is C₂₋₉heterocycloalkyl substituted with 1 or 2 R¹⁴.

E48: The method of embodiment 44, or a solvate, hydrate, tautomer, N-oxide, stereoisomer, or pharmaceutically acceptable salt thereof, wherein R⁴ is

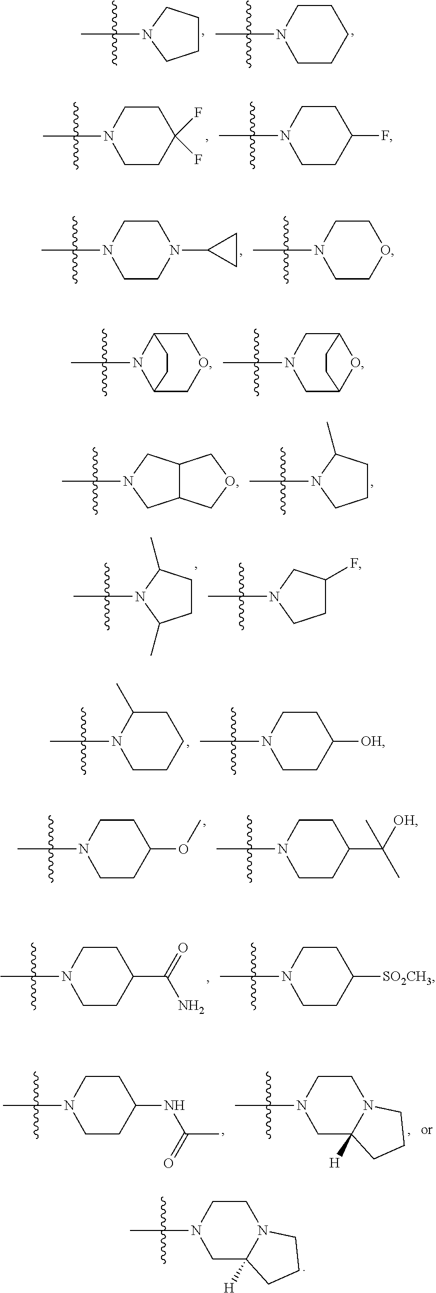

E49: The method of any one of embodiments 1-37, or a solvate, hydrate, tautomer, N-oxide, stereoisomer, or pharmaceutically acceptable salt thereof, wherein R⁴ is C₁₋₉heteroaryl optionally substituted with 1 or 2 R¹⁴.

E50: The method of embodiment 49, or a solvate, hydrate, tautomer, N-oxide, stereoisomer, or pharmaceutically acceptable salt thereof, wherein R⁴ is an unsubstituted C₁₋₉heteroaryl.

E51: The method of embodiment 49, or a solvate, hydrate, tautomer, N-oxide, stereoisomer, or pharmaceutically acceptable salt thereof, wherein R⁴ is C₂₋₉heteroaryl substituted with 1 or 2 R¹⁴.

E52: The method of embodiment 49, or a solvate, hydrate, tautomer, N-oxide, stereoisomer, or pharmaceutically acceptable salt thereof, wherein R⁴ is

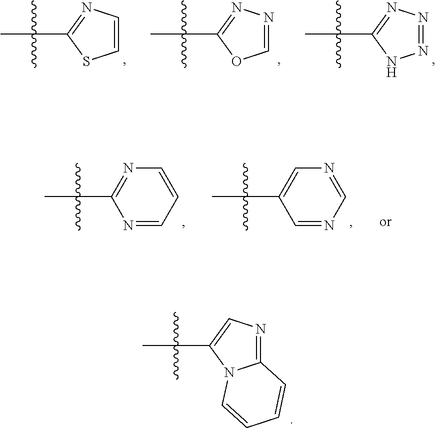 [SMILES: *c1nccs1 ; *c1nnco1 ; *c1nnn[nH]1 ; *c1ncccn1 ; *c1cncnc1 ; *c1cnc2ccccn12]

E53: The method of any one of embodiments 1-52, or a solvate, hydrate, tautomer, N-oxide, stereoisomer, or pharmaceutically acceptable salt thereof, wherein R⁵ is H, halogen, C₁₋₆alkyl, C₁₋₆haloalkyl, or C₁₋₆haloalkoxy.

E54: The method of embodiment 53, or a solvate, hydrate, tautomer, N-oxide, stereoisomer, or pharmaceutically acceptable salt thereof, wherein R⁵ is H.

E55: The method of embodiment 53, or a solvate, hydrate, tautomer, N-oxide, stereoisomer, or pharmaceutically acceptable salt thereof, wherein R⁵ is halogen.

E56: The method of embodiment 55, or a solvate, hydrate, tautomer, N-oxide, stereoisomer, or pharmaceutically acceptable salt thereof, wherein R⁵ is —C₁.

E57: The method of embodiment 53, or a solvate, hydrate, tautomer, N-oxide, stereoisomer, or pharmaceutically acceptable salt thereof, wherein R⁵ is C₁₋₆haloalkyl.

E58: The method of embodiment 57, or a solvate, hydrate, tautomer, N-oxide, stereoisomer, or pharmaceutically acceptable salt thereof, wherein R⁵ is —CF₃.

E59: The method of any one of embodiments 1-58, or a solvate, hydrate, tautomer, N-oxide, stereoisomer, or pharmaceutically acceptable salt thereof, wherein R² is H.

E60: The method of any one of embodiments 1-58, or a solvate, hydrate, tautomer, N-oxide, stereoisomer, or pharmaceutically acceptable salt thereof, wherein R² is halogen.

E61: The method of any one of embodiments 1-58, or a solvate, hydrate, tautomer, N-oxide, stereoisomer, or pharmaceutically acceptable salt thereof, wherein R² is C₁₋₆alkyl.

E62: The method of any one of embodiments 1-61, or a solvate, hydrate, tautomer, N-oxide, stereoisomer, or pharmaceutically acceptable salt thereof, wherein R¹ is —C(O)OR¹⁵.

E63: The method of embodiment 62, or a solvate, hydrate, tautomer, N-oxide, stereoisomer, or pharmaceutically acceptable salt thereof, wherein R¹⁵ is H.

E64: The method of any one of embodiments 1-61, or a solvate, hydrate, tautomer, N-oxide, stereoisomer, or pharmaceutically acceptable salt thereof, wherein R¹ is —C(O)NR¹⁰R¹¹.

E65: The method of embodiment 64, or a solvate, hydrate, tautomer, N-oxide, stereoisomer, or pharmaceutically acceptable salt thereof, wherein R¹⁰ and R¹¹ are each H.

E66: The method of embodiment 1 or 2, wherein the compound of Formula (I) or Formula (I′) is selected from:

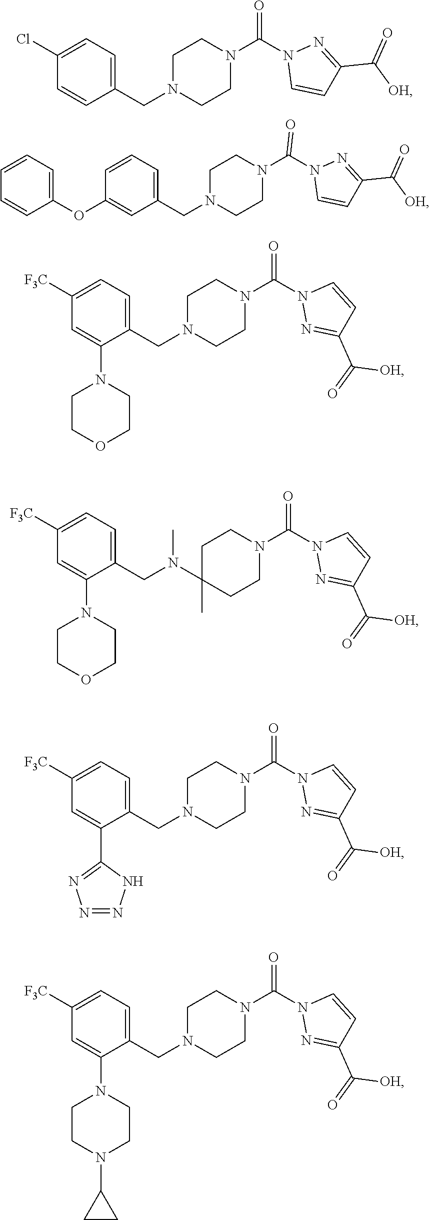

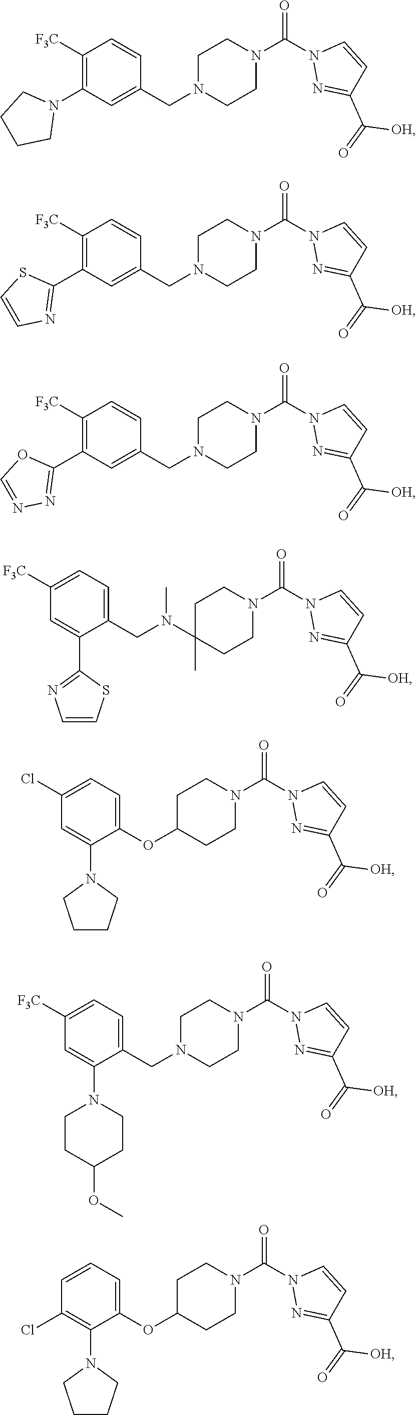

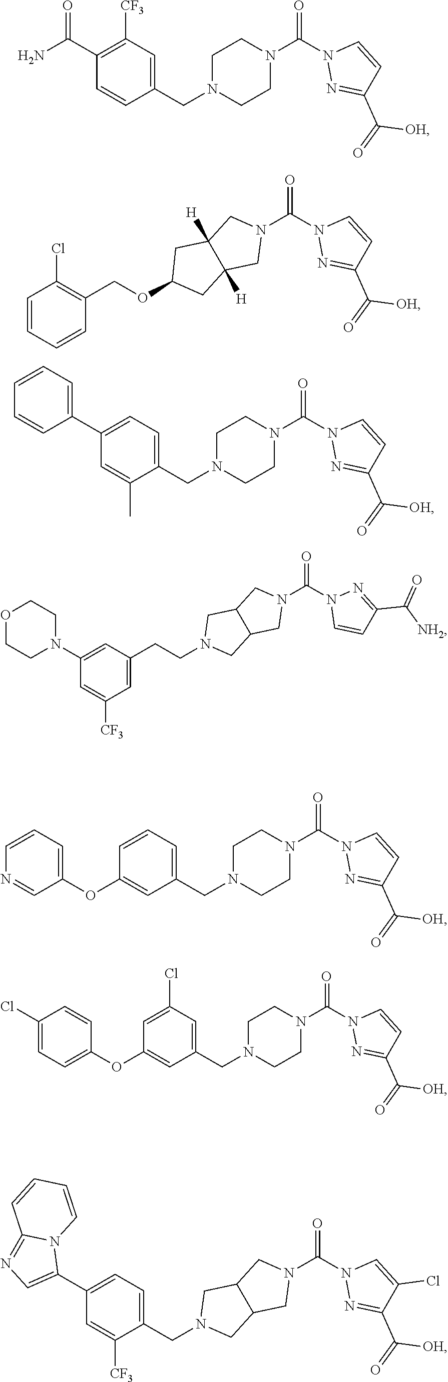

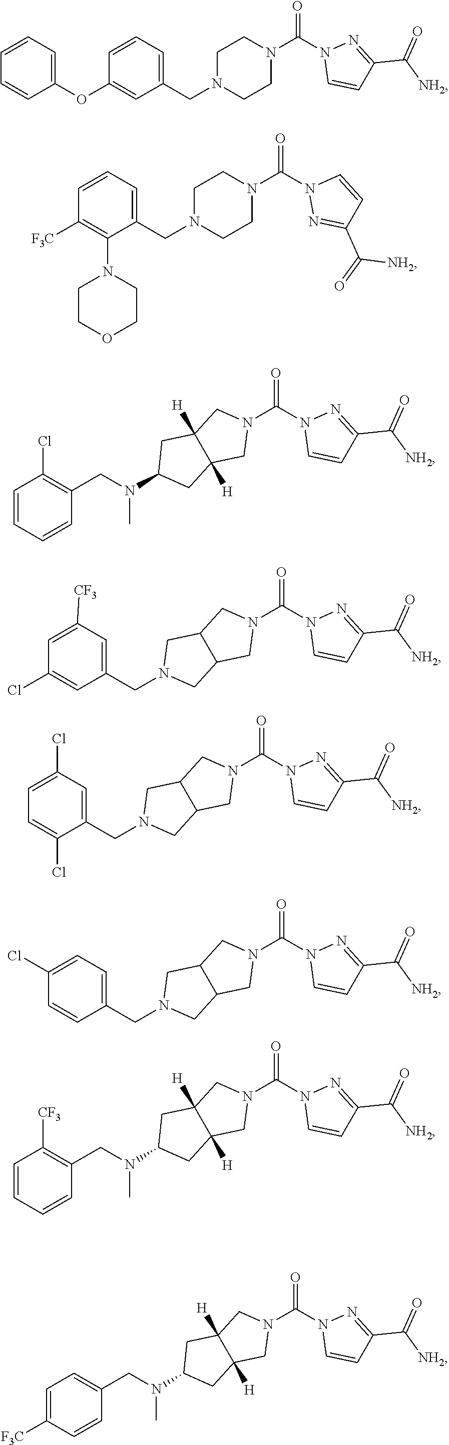

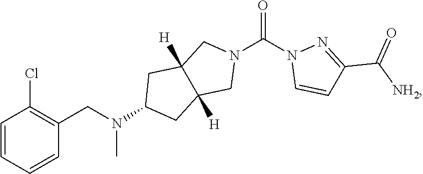 [SMILES: CN(Cc1ccccc1Cl)[C@@H]1C[C@@H]2CN(C(=O)n3ccc(C(N)=O)n3)C[C@@H]2C1]

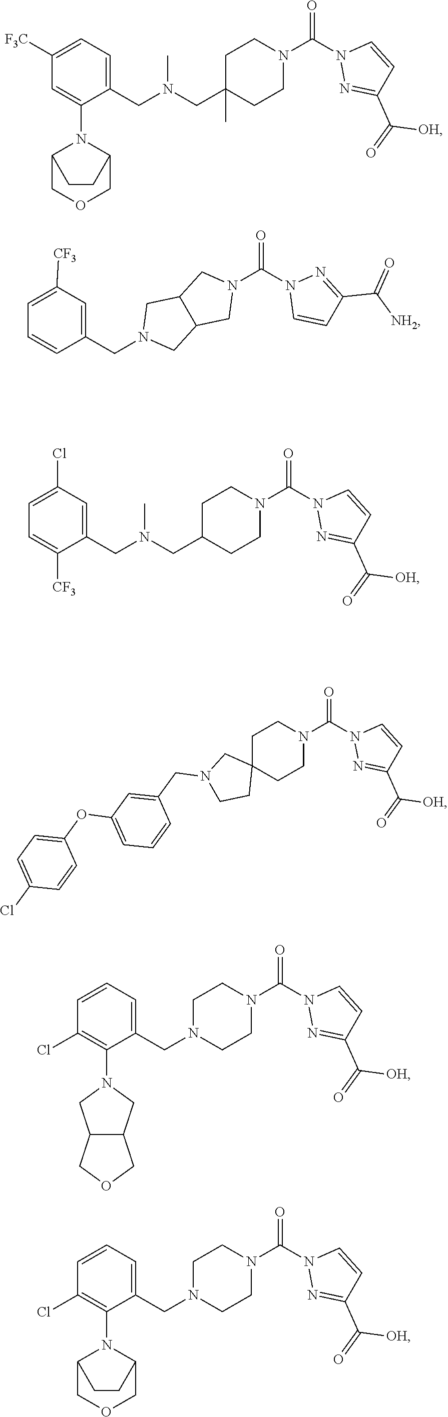 [SMILES: CN(Cc1ccc(C(F)(F)F)cc1N1C2CCC1COC2)CC1(C)CCN(C(=O)n2ccc(C(=O)O)n2)CC1 ; NC(=O)c1ccn(C(=O)N2CC3CN(Cc4cccc(C(F)(F)F)c4)CC3C2)n1 ; CN(Cc1cc(Cl)ccc1C(F)(F)F)CC1CCN(C(=O)n2ccc(C(=O)O)n2)CC1 ; O=C(O)c1ccn(C(=O)N2CCC3(CCN(Cc4cccc(Oc5ccc(Cl)cc5)c4)C3)CC2)n1 ; O=C(O)c1ccn(C(=O)N2CCN(Cc3cccc(Cl)c3N3CC4COCC4C3)CC2)n1 ; O=C(O)c1ccn(C(=O)N2CCN(Cc3cccc(Cl)c3N3C4CCC3COC4)CC2)n1]

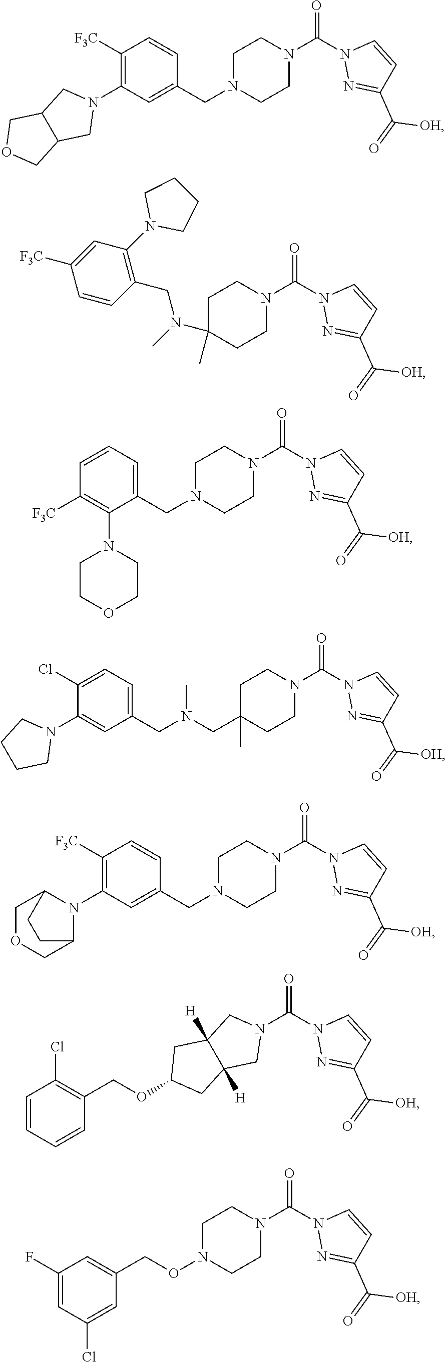

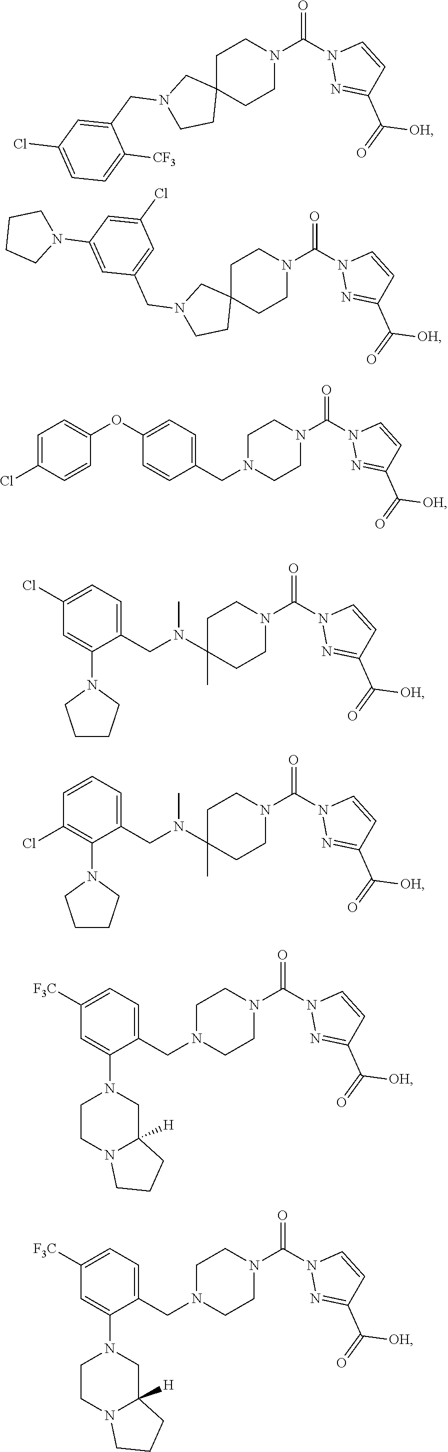

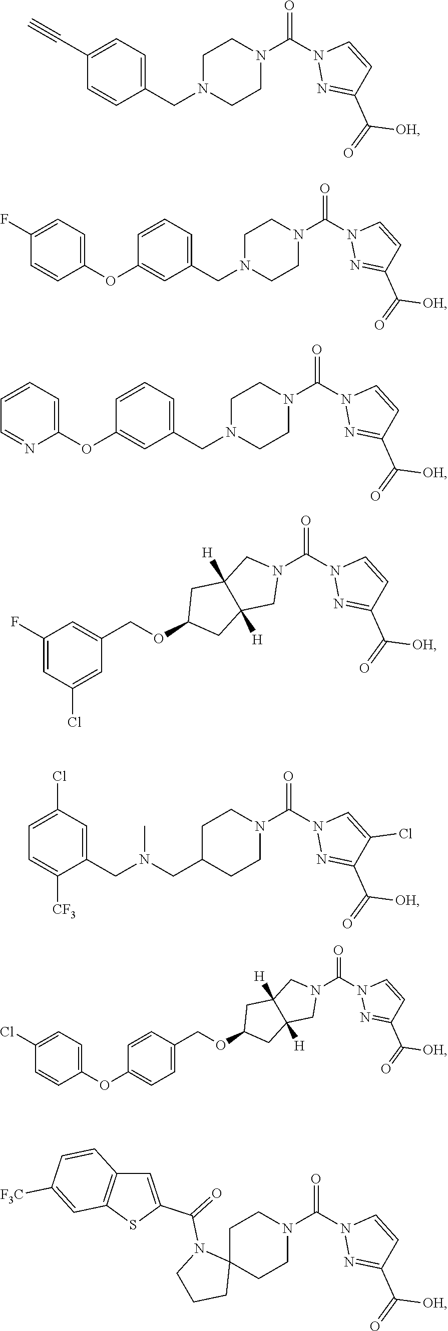

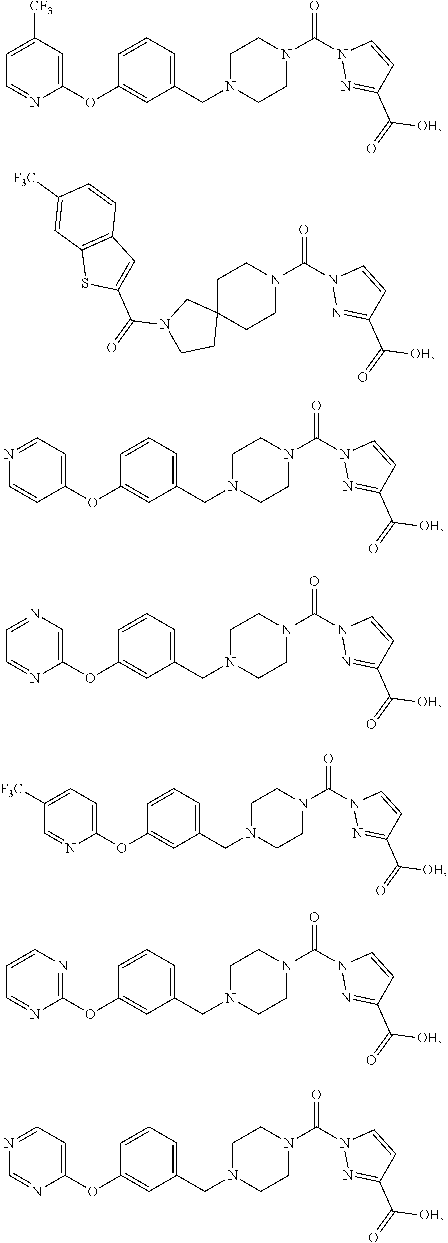

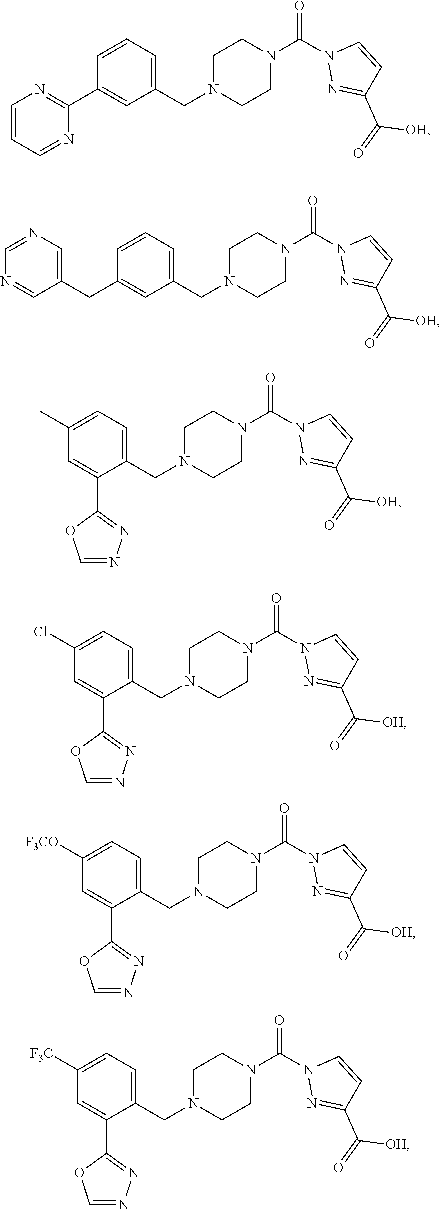 [SMILES: O=C(O)c1ccn(C(=O)N2CCN(Cc3cccc(-c4ncccn4)c3)CC2)n1 ; O=C(O)c1ccn(C(=O)N2CCN(Cc3cccc(Cc4cncnc4)c3)CC2)n1 ; Cc1ccc(CN2CCN(C(=O)n3ccc(C(=O)O)n3)CC2)c(-c2nnco2)c1 ; O=C(O)c1ccn(C(=O)N2CCN(Cc3ccc(Cl)cc3-c3nnco3)CC2)n1 ; O=C(O)c1ccn(C(=O)N2CCN(Cc3ccc(OC(F)(F)F)cc3-c3nnco3)CC2)n1 ; O=C(O)c1ccn(C(=O)N2CCN(Cc3ccc(C(F)(F)F)cc3-c3nnco3)CC2)n1]

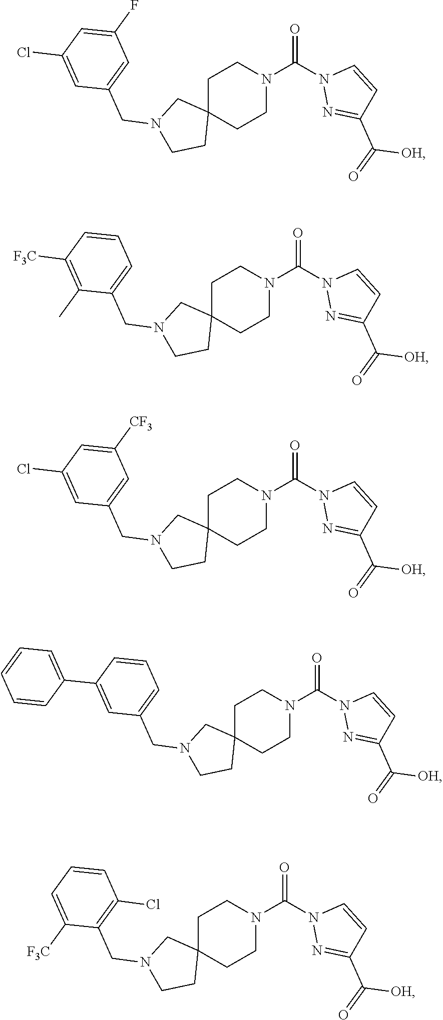 [SMILES: O=C(O)c1ccn(C(=O)N2CCC3(CCN(Cc4cc(F)cc(Cl)c4)C3)CC2)n1 ; Cc1c(CN2CCC3(CCN(C(=O)n4ccc(C(=O)O)n4)CC3)C2)cccc1C(F)(F)F ; O=C(O)c1ccn(C(=O)N2CCC3(CCN(Cc4cc(Cl)cc(C(F)(F)F)c4)C3)CC2)n1 ; O=C(O)c1ccn(C(=O)N2CCC3(CCN(Cc4cccc(-c5ccccc5)c4)C3)CC2)n1 ; O=C(O)c1ccn(C(=O)N2CCC3(CCN(Cc4c(Cl)cccc4C(F)(F)F)C3)CC2)n1]

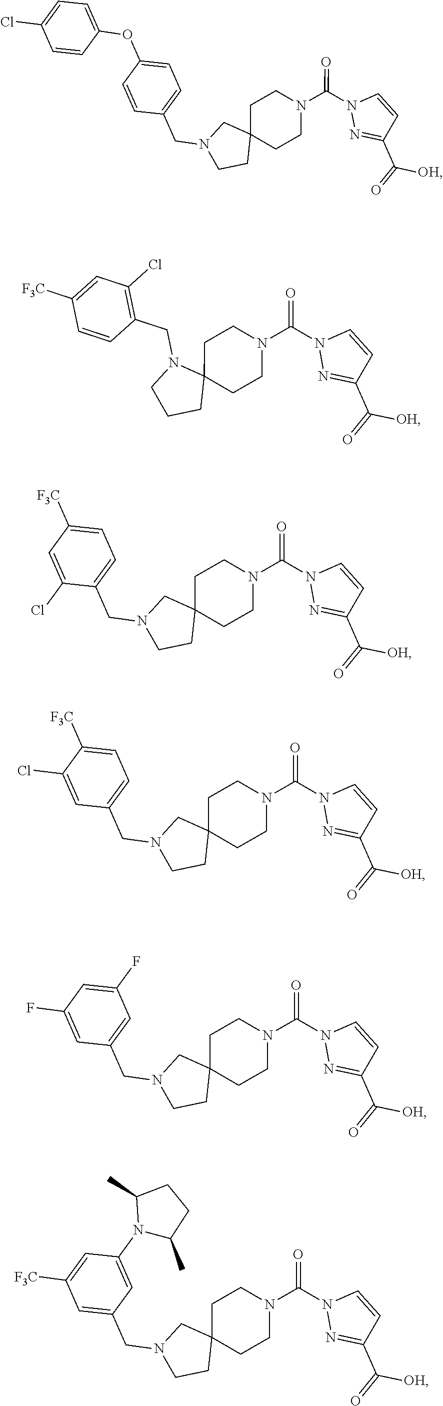 [SMILES: O=C(O)c1ccn(C(=O)N2CCC3(CCN(Cc4ccc(Oc5ccc(Cl)cc5)cc4)C3)CC2)n1 ; O=C(O)c1ccn(C(=O)N2CCC3(CCCN3Cc3ccc(C(F)(F)F)cc3Cl)CC2)n1 ; O=C(O)c1ccn(C(=O)N2CCC3(CCN(Cc4ccc(C(F)(F)F)cc4Cl)C3)CC2)n1 ; O=C(O)c1ccn(C(=O)N2CCC3(CCN(Cc4ccc(C(F)(F)F)c(Cl)c4)C3)CC2)n1 ; O=C(O)c1ccn(C(=O)N2CCC3(CCN(Cc4cc(F)cc(F)c4)C3)CC2)n1 ; C[C@@H]1CC[C@H](C)N1c1cc(CN2CCC3(CCN(C(=O)n4ccc(C(=O)O)n4)CC3)C2)cc(C(F)(F)F)c1]

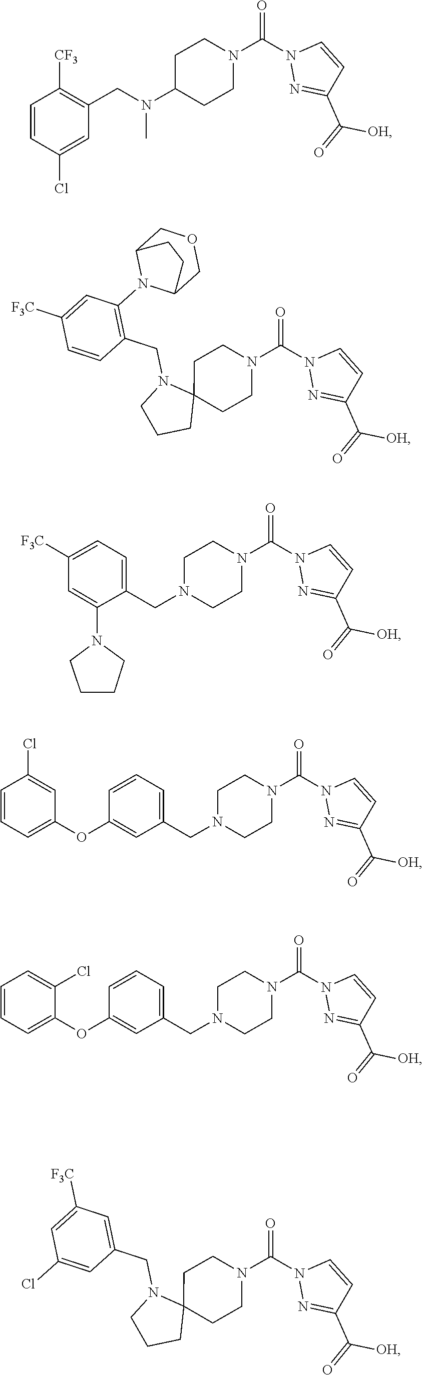 [SMILES: CN(Cc1cc(Cl)ccc1C(F)(F)F)C1CCN(C(=O)n2ccc(C(=O)O)n2)CC1 ; O=C(O)c1ccn(C(=O)N2CCC3(CCCN3Cc3ccc(C(F)(F)F)cc3N3C4CCC3COC4)CC2)n1 ; O=C(O)c1ccn(C(=O)N2CCN(Cc3ccc(C(F)(F)F)cc3N3CCCC3)CC2)n1 ; O=C(O)c1ccn(C(=O)N2CCN(Cc3cccc(Oc4cccc(Cl)c4)c3)CC2)n1 ; O=C(O)c1ccn(C(=O)N2CCN(Cc3cccc(Oc4ccccc4Cl)c3)CC2)n1 ; O=C(O)c1ccn(C(=O)N2CCC3(CCCN3Cc3cc(Cl)cc(C(F)(F)F)c3)CC2)n1]

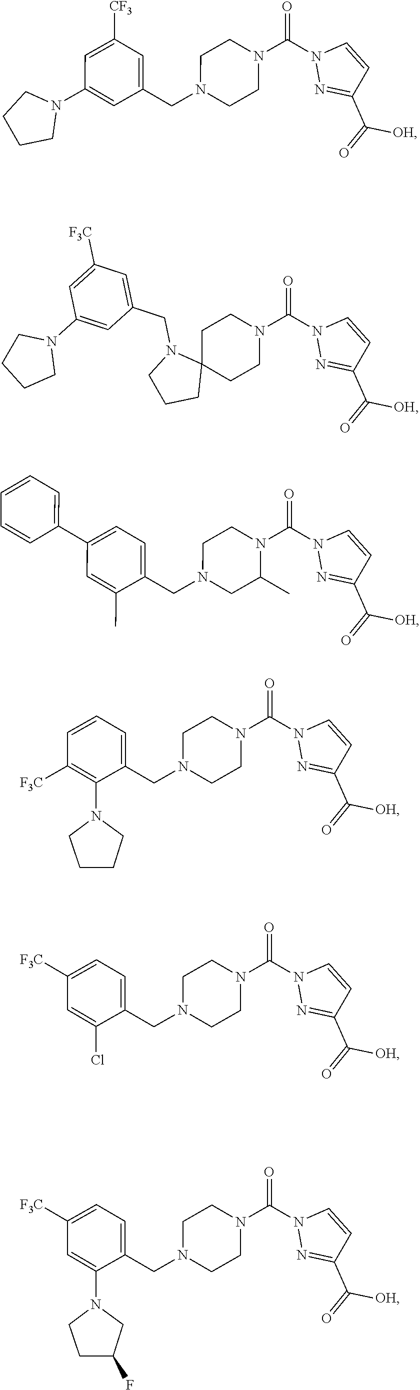 [SMILES: O=C(O)c1ccn(C(=O)N2CCN(Cc3cc(N4CCCC4)cc(C(F)(F)F)c3)CC2)n1 ; O=C(O)c1ccn(C(=O)N2CCC3(CCCN3Cc3cc(N4CCCC4)cc(C(F)(F)F)c3)CC2)n1 ; Cc1cc(-c2ccccc2)ccc1CN1CCN(C(=O)n2ccc(C(=O)O)n2)C(C)C1 ; O=C(O)c1ccn(C(=O)N2CCN(Cc3cccc(C(F)(F)F)c3N3CCCC3)CC2)n1 ; O=C(O)c1ccn(C(=O)N2CCN(Cc3ccc(C(F)(F)F)cc3Cl)CC2)n1 ; O=C(O)c1ccn(C(=O)N2CCN(Cc3ccc(C(F)(F)F)cc3N3CC[C@H](F)C3)CC2)n1]

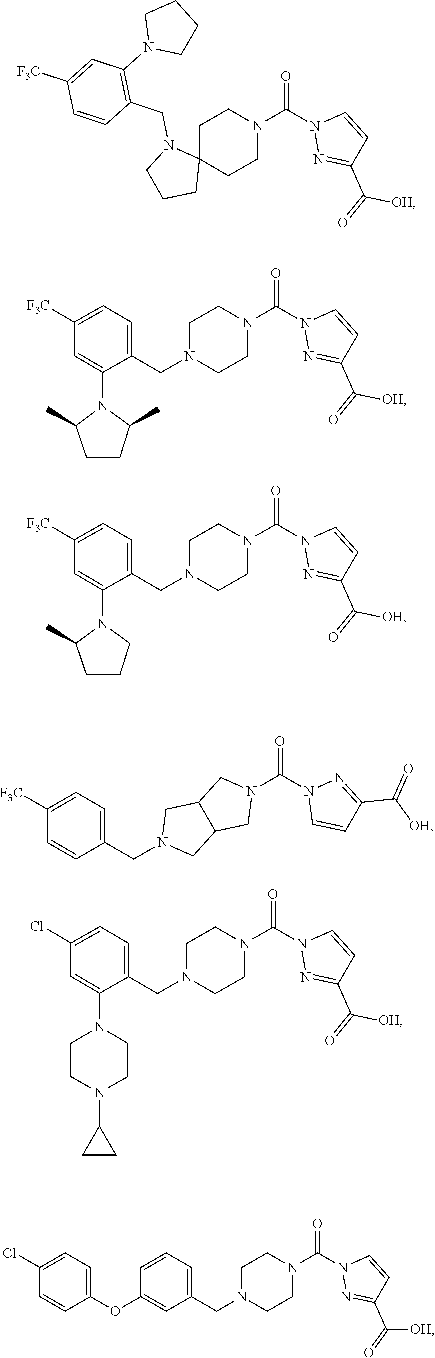 [SMILES: O=C(O)c1ccn(C(=O)N2CCC3(CCCN3Cc3ccc(C(F)(F)F)cc3N3CCCC3)CC2)n1 ; C[C@@H]1CC[C@H](C)N1c1cc(C(F)(F)F)ccc1CN1CCN(C(=O)n2ccc(C(=O)O)n2)CC1 ; C[C@@H]1CCCN1c1cc(C(F)(F)F)ccc1CN1CCN(C(=O)n2ccc(C(=O)O)n2)CC1 ; O=C(O)c1ccn(C(=O)N2CC3CN(Cc4ccc(C(F)(F)F)cc4)CC3C2)n1 ; O=C(O)c1ccn(C(=O)N2CCN(Cc3ccc(Cl)cc3N3CCN(C4CC4)CC3)CC2)n1 ; O=C(O)c1ccn(C(=O)N2CCN(Cc3cccc(Oc4ccc(Cl)cc4)c3)CC2)n1]

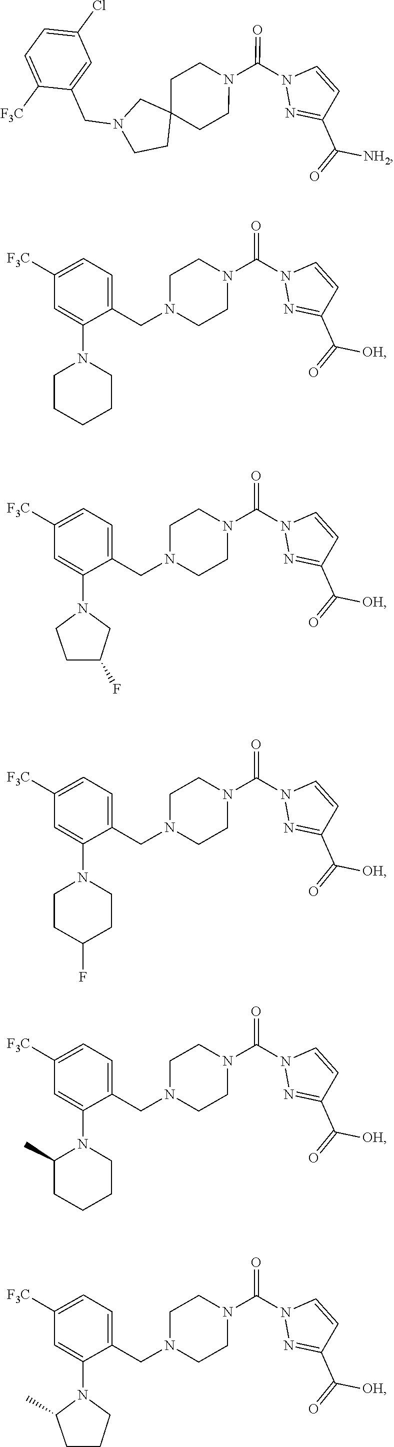 [SMILES: NC(=O)c1ccn(C(=O)N2CCC3(CCN(Cc4cc(Cl)ccc4C(F)(F)F)C3)CC2)n1 ; O=C(O)c1ccn(C(=O)N2CCN(Cc3ccc(C(F)(F)F)cc3N3CCCCC3)CC2)n1 ; O=C(O)c1ccn(C(=O)N2CCN(Cc3ccc(C(F)(F)F)cc3N3CC[C@@H](F)C3)CC2)n1 ; O=C(O)c1ccn(C(=O)N2CCN(Cc3ccc(C(F)(F)F)cc3N3CCC(F)CC3)CC2)n1 ; C[C@@H]1CCCCN1c1cc(C(F)(F)F)ccc1CN1CCN(C(=O)n2ccc(C(=O)O)n2)CC1 ; C[C@H]1CCCN1c1cc(C(F)(F)F)ccc1CN1CCN(C(=O)n2ccc(C(=O)O)n2)CC1]

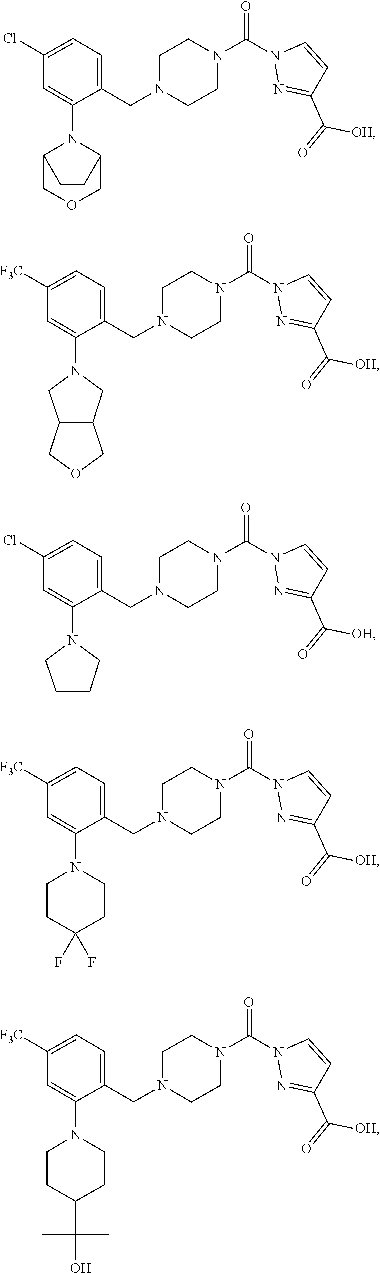 [SMILES: O=C(O)c1ccn(C(=O)N2CCN(Cc3ccc(Cl)cc3N3C4CCC3COC4)CC2)n1 ; O=C(O)c1ccn(C(=O)N2CCN(Cc3ccc(C(F)(F)F)cc3N3CC4COCC4C3)CC2)n1 ; O=C(O)c1ccn(C(=O)N2CCN(Cc3ccc(Cl)cc3N3CCCC3)CC2)n1 ; O=C(O)c1ccn(C(=O)N2CCN(Cc3ccc(C(F)(F)F)cc3N3CCC(F)(F)CC3)CC2)n1 ; CC(C)(O)C1CCN(c2cc(C(F)(F)F)ccc2CN2CCN(C(=O)n3ccc(C(=O)O)n3)CC2)CC1]

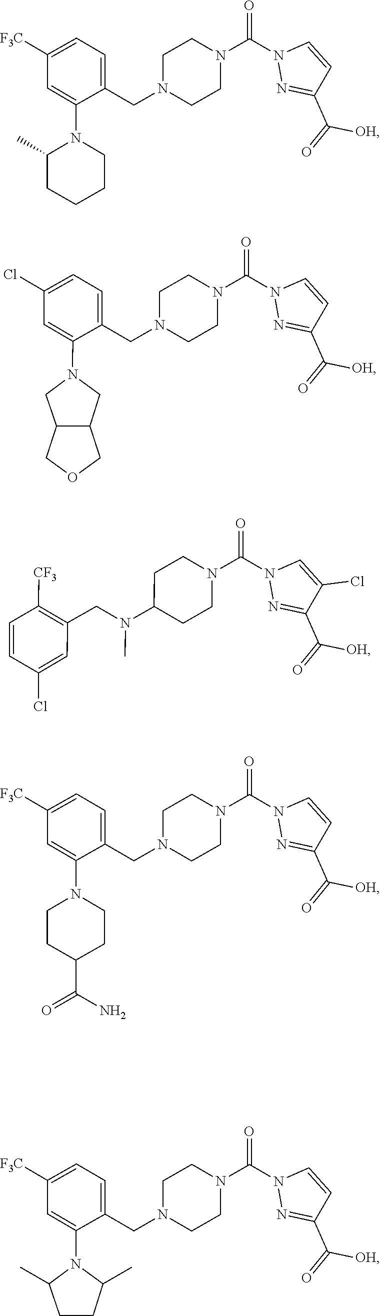 [SMILES: C[C@H]1CCCCN1c1cc(C(F)(F)F)ccc1CN1CCN(C(=O)n2ccc(C(=O)O)n2)CC1 ; O=C(O)c1ccn(C(=O)N2CCN(Cc3ccc(Cl)cc3N3CC4COCC4C3)CC2)n1 ; CN(Cc1cc(Cl)ccc1C(F)(F)F)C1CCN(C(=O)n2cc(Cl)c(C(=O)O)n2)CC1 ; NC(=O)C1CCN(c2cc(C(F)(F)F)ccc2CN2CCN(C(=O)n3ccc(C(=O)O)n3)CC2)CC1 ; CC1CCC(C)N1c1cc(C(F)(F)F)ccc1CN1CCN(C(=O)n2ccc(C(=O)O)n2)CC1]

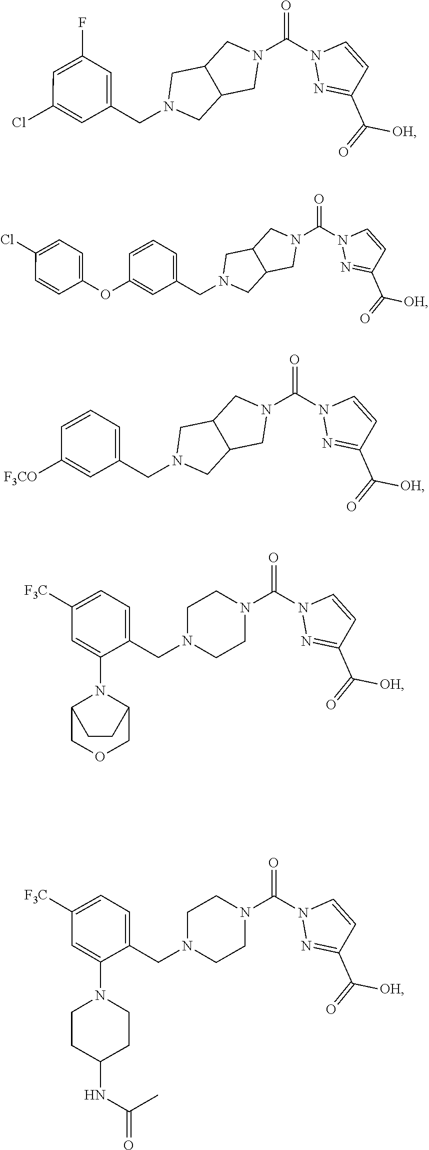 [SMILES: O=C(O)c1ccn(C(=O)N2CC3CN(Cc4cc(F)cc(Cl)c4)CC3C2)n1 ; O=C(O)c1ccn(C(=O)N2CC3CN(Cc4cccc(Oc5ccc(Cl)cc5)c4)CC3C2)n1 ; O=C(O)c1ccn(C(=O)N2CC3CN(Cc4cccc(OC(F)(F)F)c4)CC3C2)n1 ; O=C(O)c1ccn(C(=O)N2CCN(Cc3ccc(C(F)(F)F)cc3N3C4CCC3COC4)CC2)n1 ; CC(=O)NC1CCN(c2cc(C(F)(F)F)ccc2CN2CCN(C(=O)n3ccc(C(=O)O)n3)CC2)CC1]

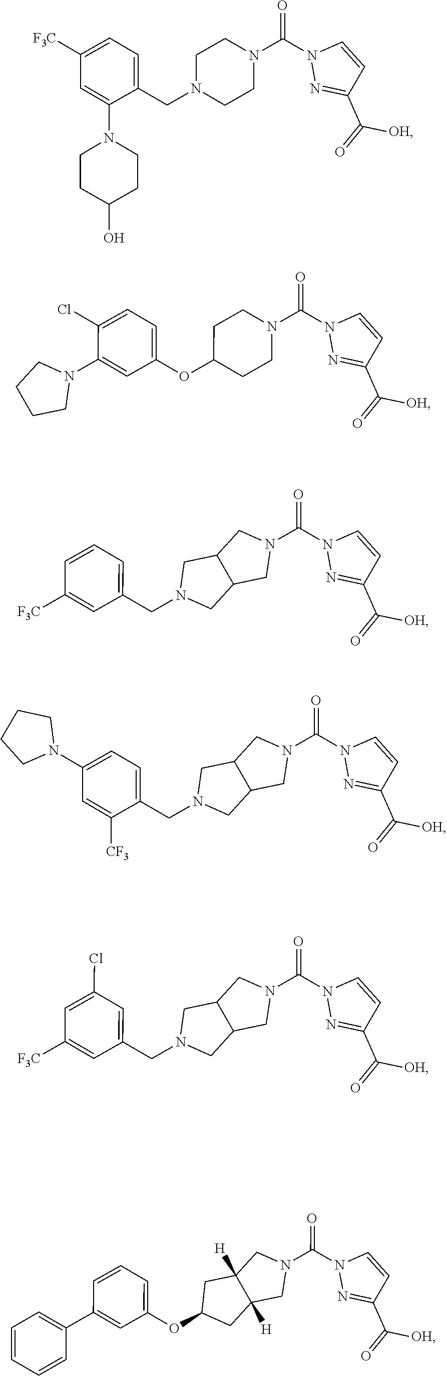 [SMILES: O=C(O)c1ccn(C(=O)N2CCN(Cc3ccc(C(F)(F)F)cc3N3CCC(O)CC3)CC2)n1 ; O=C(O)c1ccn(C(=O)N2CCC(Oc3ccc(Cl)c(N4CCCC4)c3)CC2)n1 ; O=C(O)c1ccn(C(=O)N2CC3CN(Cc4cccc(C(F)(F)F)c4)CC3C2)n1 ; O=C(O)c1ccn(C(=O)N2CC3CN(Cc4ccc(N5CCCC5)cc4C(F)(F)F)CC3C2)n1 ; O=C(O)c1ccn(C(=O)N2CC3CN(Cc4cc(Cl)cc(C(F)(F)F)c4)CC3C2)n1 ; O=C(O)c1ccn(C(=O)N2C[C@H]3C[C@H](Oc4cccc(-c5ccccc5)c4)C[C@H]3C2)n1]

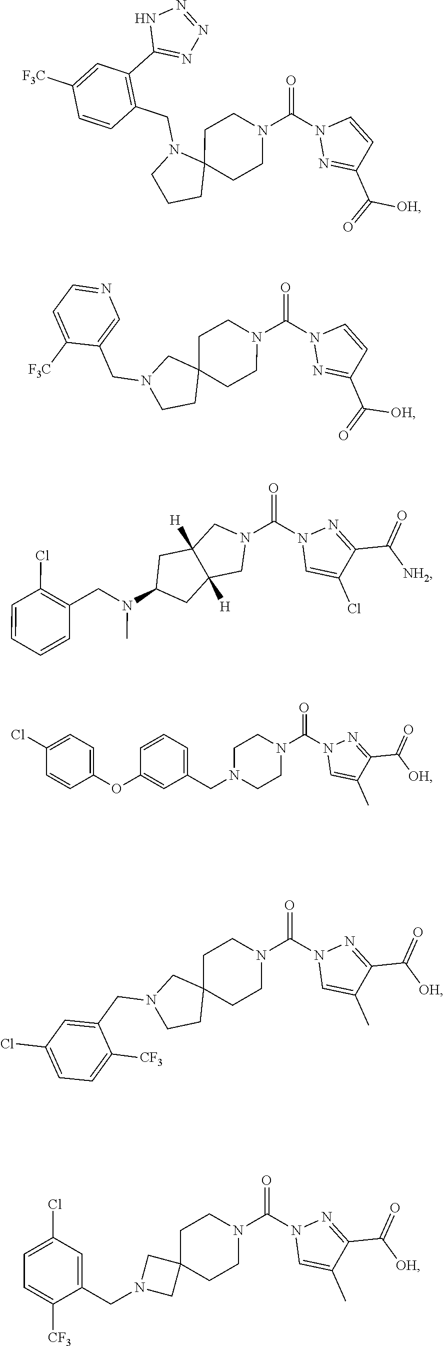 [SMILES: O=C(O)c1ccn(C(=O)N2CCC3(CCCN3Cc3ccc(C(F)(F)F)cc3-c3nnn[nH]3)CC2)n1 ; O=C(O)c1ccn(C(=O)N2CCC3(CCN(Cc4cnccc4C(F)(F)F)C3)CC2)n1 ; CN(Cc1ccccc1Cl)[C@H]1C[C@@H]2CN(C(=O)n3cc(Cl)c(C(N)=O)n3)C[C@@H]2C1 ; Cc1cn(C(=O)N2CCN(Cc3cccc(Oc4ccc(Cl)cc4)c3)CC2)nc1C(=O)O ; Cc1cn(C(=O)N2CCC3(CCN(Cc4cc(Cl)ccc4C(F)(F)F)C3)CC2)nc1C(=O)O ; Cc1cn(C(=O)N2CCC3(CC2)CN(Cc2cc(Cl)ccc2C(F)(F)F)C3)nc1C(=O)O]

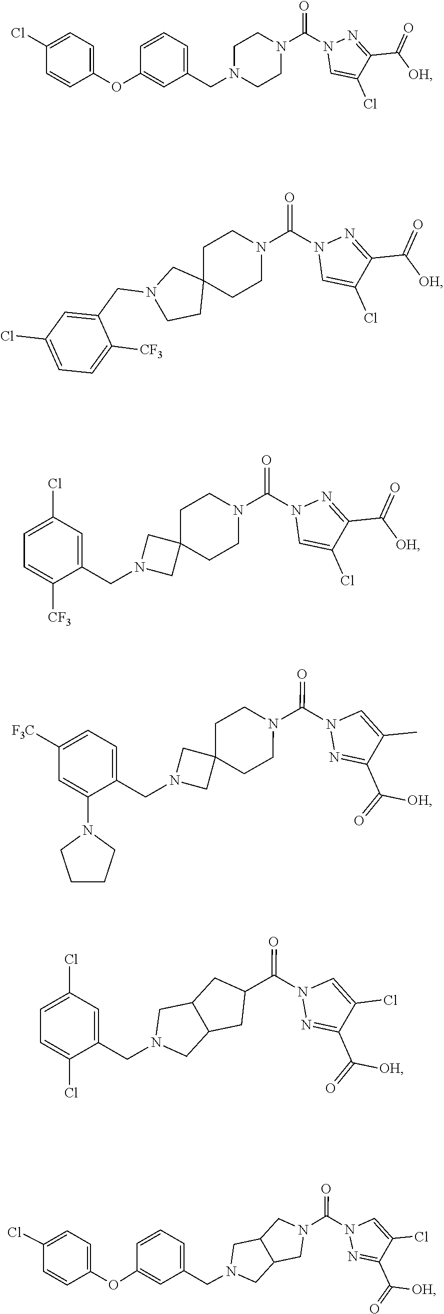 [SMILES: O=C(O)c1nn(C(=O)N2CCN(Cc3cccc(Oc4ccc(Cl)cc4)c3)CC2)cc1Cl ; O=C(O)c1nn(C(=O)N2CCC3(CCN(Cc4cc(Cl)ccc4C(F)(F)F)C3)CC2)cc1Cl ; O=C(O)c1nn(C(=O)N2CCC3(CC2)CN(Cc2cc(Cl)ccc2C(F)(F)F)C3)cc1Cl ; Cc1cn(C(=O)N2CCC3(CC2)CN(Cc2ccc(C(F)(F)F)cc2N2CCCC2)C3)nc1C(=O)O ; O=C(O)c1nn(C(=O)C2CC3CN(Cc4cc(Cl)ccc4Cl)CC3C2)cc1Cl ; O=C(O)c1nn(C(=O)N2CC3CN(Cc4cccc(Oc5ccc(Cl)cc5)c4)CC3C2)cc1Cl]

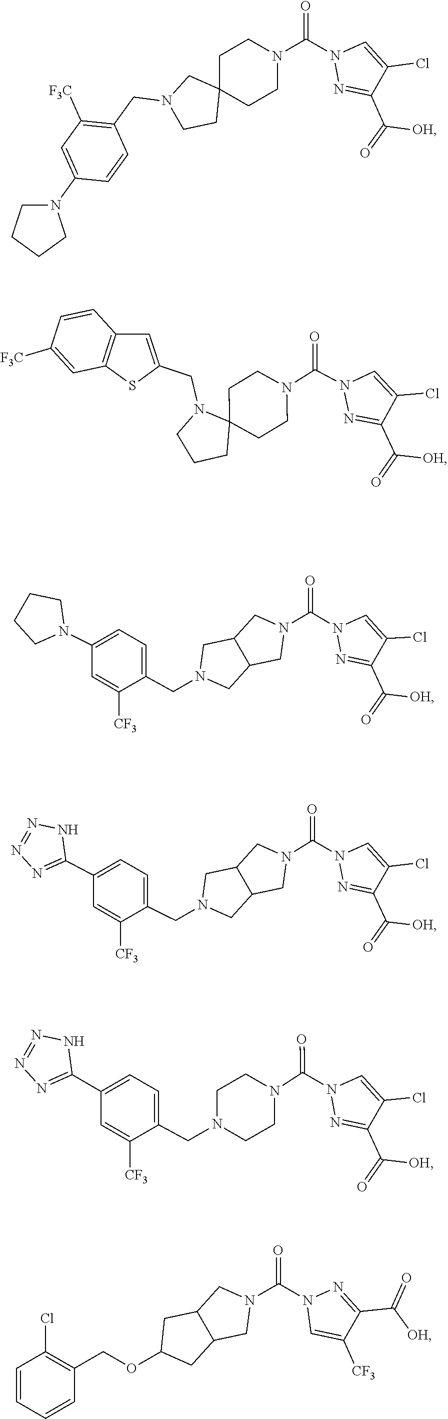 [SMILES: O=C(O)c1nn(C(=O)N2CCC3(CCN(Cc4ccc(N5CCCC5)cc4C(F)(F)F)C3)CC2)cc1Cl ; O=C(O)c1nn(C(=O)N2CCC3(CCCN3Cc3cc4ccc(C(F)(F)F)cc4s3)CC2)cc1Cl ; O=C(O)c1nn(C(=O)N2CC3CN(Cc4ccc(N5CCCC5)cc4C(F)(F)F)CC3C2)cc1Cl ; O=C(O)c1nn(C(=O)N2CC3CN(Cc4ccc(-c5nnn[nH]5)cc4C(F)(F)F)CC3C2)cc1Cl ; O=C(O)c1nn(C(=O)N2CCN(Cc3ccc(-c4nnn[nH]4)cc3C(F)(F)F)CC2)cc1Cl ; O=C(O)c1nn(C(=O)N2CC3CC(OCc4ccccc4Cl)CC3C2)cc1C(F)(F)F]

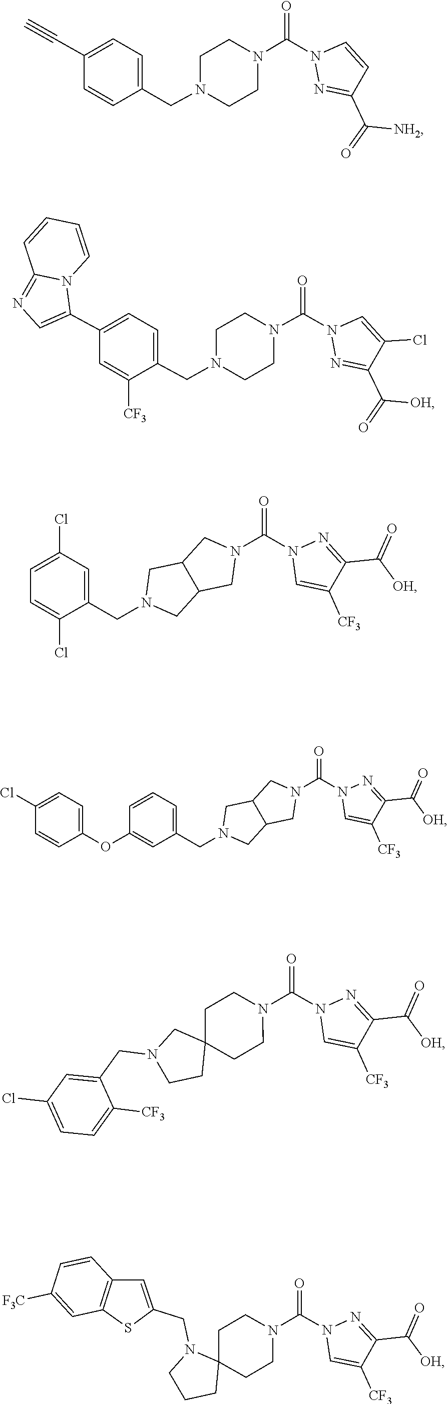 [SMILES: C#Cc1ccc(CN2CCN(C(=O)n3ccc(C(N)=O)n3)CC2)cc1 ; O=C(O)c1nn(C(=O)N2CCN(Cc3ccc(-c4cnc5ccccn45)cc3C(F)(F)F)CC2)cc1Cl ; O=C(O)c1nn(C(=O)N2CC3CN(Cc4cc(Cl)ccc4Cl)CC3C2)cc1C(F)(F)F ; O=C(O)c1nn(C(=O)N2CC3CN(Cc4cccc(Oc5ccc(Cl)cc5)c4)CC3C2)cc1C(F)(F)F ; O=C(O)c1nn(C(=O)N2CCC3(CCN(Cc4cc(Cl)ccc4C(F)(F)F)C3)CC2)cc1C(F)(F)F ; O=C(O)c1nn(C(=O)N2CCC3(CCCN3Cc3cc4ccc(C(F)(F)F)cc4s3)CC2)cc1C(F)(F)F]

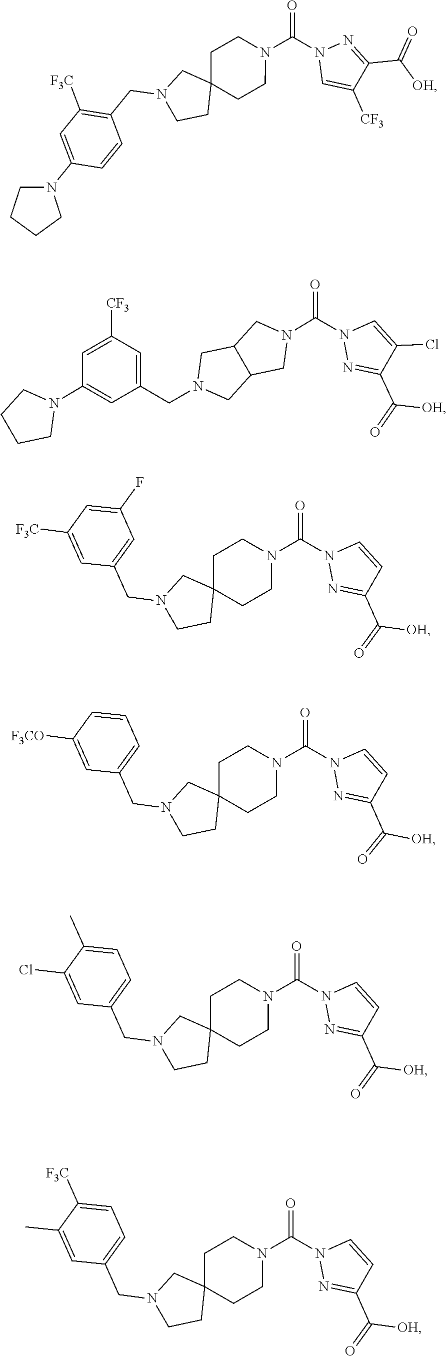 [SMILES: O=C(O)c1nn(C(=O)N2CCC3(CCN(Cc4ccc(N5CCCC5)cc4C(F)(F)F)C3)CC2)cc1C(F)(F)F ; O=C(O)c1nn(C(=O)N2CC3CN(Cc4cc(N5CCCC5)cc(C(F)(F)F)c4)CC3C2)cc1Cl ; O=C(O)c1ccn(C(=O)N2CCC3(CCN(Cc4cc(F)cc(C(F)(F)F)c4)C3)CC2)n1 ; O=C(O)c1ccn(C(=O)N2CCC3(CCN(Cc4cccc(OC(F)(F)F)c4)C3)CC2)n1 ; Cc1ccc(CN2CCC3(CCN(C(=O)n4ccc(C(=O)O)n4)CC3)C2)cc1Cl ; Cc1cc(CN2CCC3(CCN(C(=O)n4ccc(C(=O)O)n4)CC3)C2)ccc1C(F)(F)F]

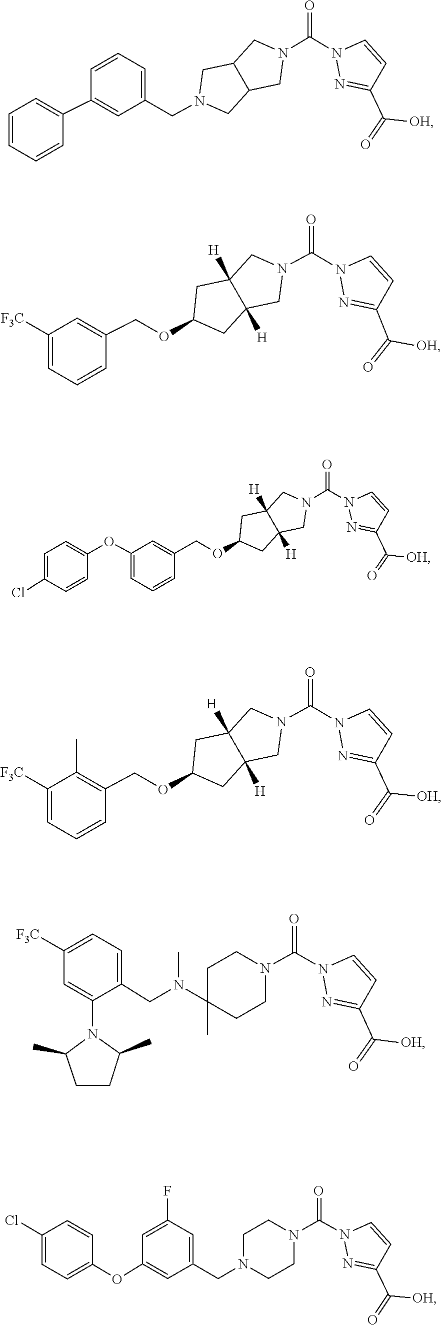 [SMILES: O=C(O)c1ccn(C(=O)N2CC3CN(Cc4cccc(-c5ccccc5)c4)CC3C2)n1 ; O=C(O)c1ccn(C(=O)N2C[C@H]3C[C@H](OCc4cccc(C(F)(F)F)c4)C[C@H]3C2)n1 ; O=C(O)c1ccn(C(=O)N2C[C@H]3C[C@H](OCc4cccc(Oc5ccc(Cl)cc5)c4)C[C@H]3C2)n1 ; Cc1c(CO[C@H]2C[C@@H]3CN(C(=O)n4ccc(C(=O)O)n4)C[C@@H]3C2)cccc1C(F)(F)F ; C[C@@H]1CC[C@H](C)N1c1cc(C(F)(F)F)ccc1CN(C)C1(C)CCN(C(=O)n2ccc(C(=O)O)n2)CC1 ; O=C(O)c1ccn(C(=O)N2CCN(Cc3cc(F)cc(Oc4ccc(Cl)cc4)c3)CC2)n1]

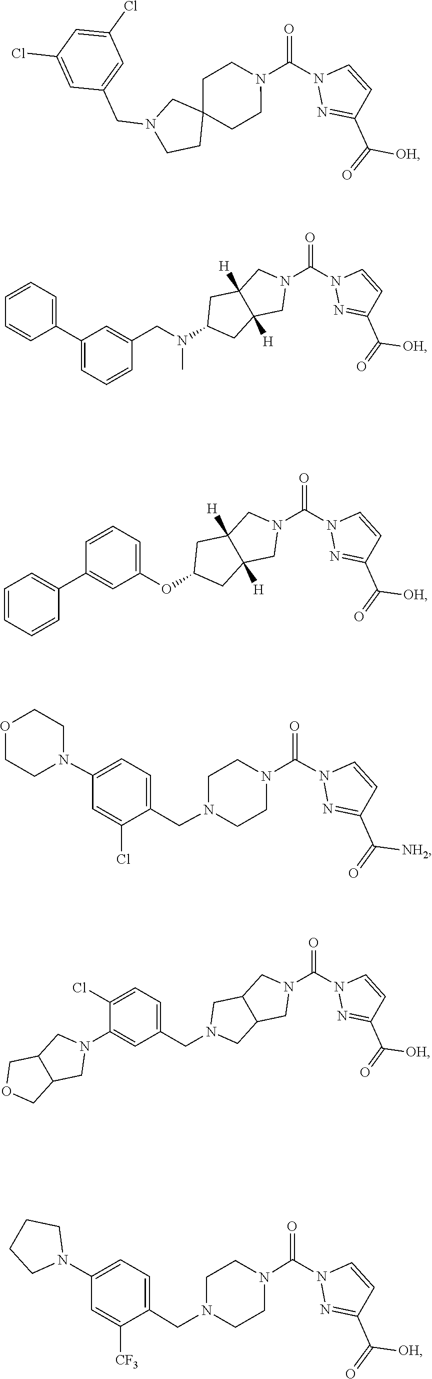 [SMILES: O=C(O)c1ccn(C(=O)N2CCC3(CCN(Cc4cc(Cl)cc(Cl)c4)C3)CC2)n1 ; CN(Cc1cccc(-c2ccccc2)c1)[C@@H]1C[C@@H]2CN(C(=O)n3ccc(C(=O)O)n3)C[C@@H]2C1 ; O=C(O)c1ccn(C(=O)N2C[C@H]3C[C@@H](Oc4cccc(-c5ccccc5)c4)C[C@H]3C2)n1 ; NC(=O)c1ccn(C(=O)N2CCN(Cc3ccc(N4CCOCC4)cc3Cl)CC2)n1 ; O=C(O)c1ccn(C(=O)N2CC3CN(Cc4ccc(Cl)c(N5CC6COCC6C5)c4)CC3C2)n1 ; O=C(O)c1ccn(C(=O)N2CCN(Cc3ccc(N4CCCC4)cc3C(F)(F)F)CC2)n1]

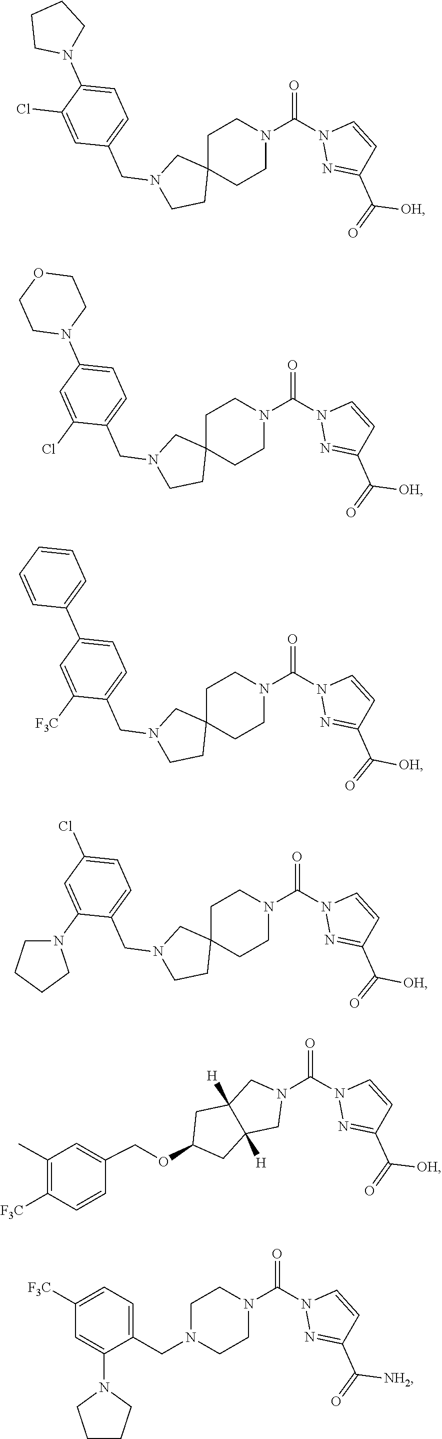 [SMILES: O=C(O)c1ccn(C(=O)N2CCC3(CCN(Cc4ccc(N5CCCC5)c(Cl)c4)C3)CC2)n1 ; O=C(O)c1ccn(C(=O)N2CCC3(CCN(Cc4ccc(N5CCOCC5)cc4Cl)C3)CC2)n1 ; O=C(O)c1ccn(C(=O)N2CCC3(CCN(Cc4ccc(-c5ccccc5)cc4C(F)(F)F)C3)CC2)n1 ; O=C(O)c1ccn(C(=O)N2CCC3(CCN(Cc4ccc(Cl)cc4N4CCCC4)C3)CC2)n1 ; Cc1cc(CO[C@H]2C[C@@H]3CN(C(=O)n4ccc(C(=O)O)n4)C[C@@H]3C2)ccc1C(F)(F)F ; NC(=O)c1ccn(C(=O)N2CCN(Cc3ccc(C(F)(F)F)cc3N3CCCC3)CC2)n1]

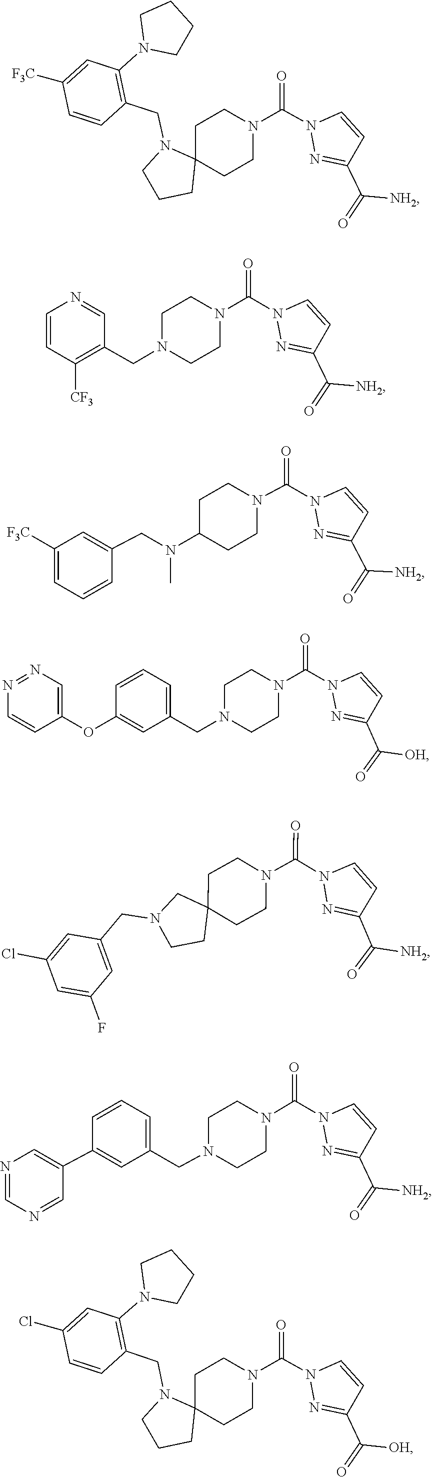

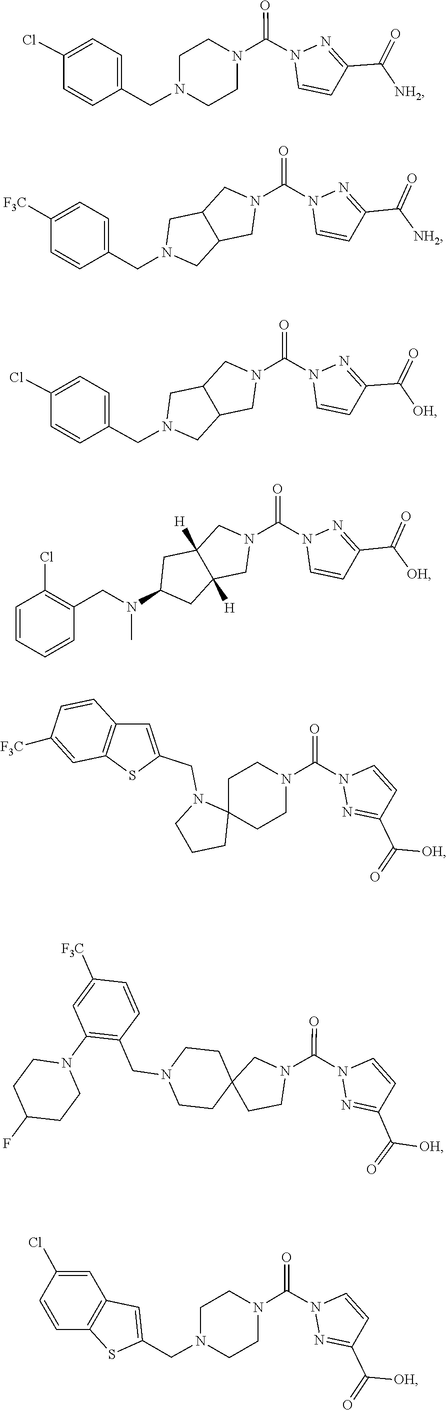

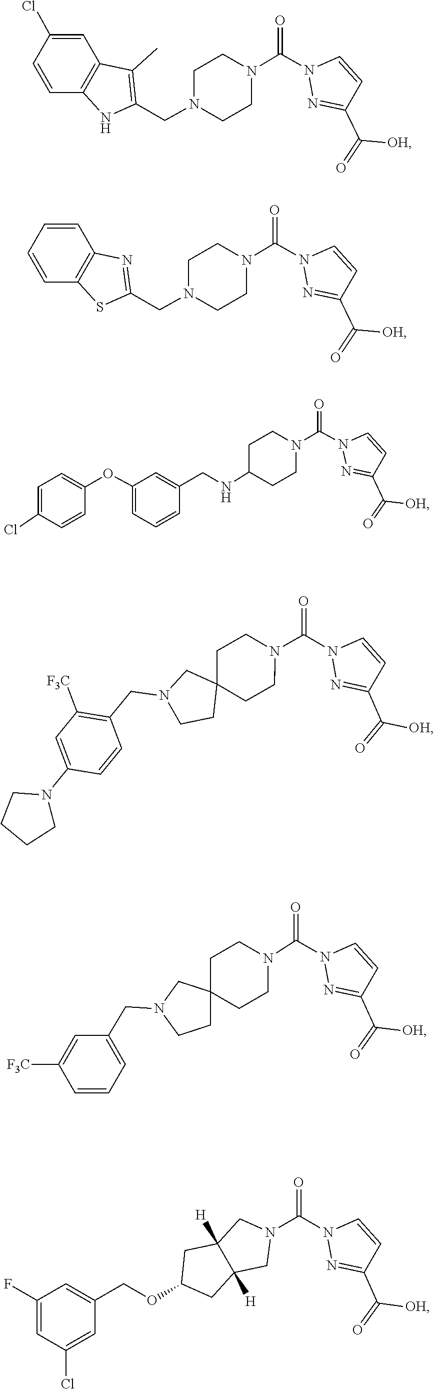 [SMILES: Cc1c(CN2CCN(C(=O)n3ccc(C(=O)O)n3)CC2)[nH]c2ccc(Cl)cc12 ; O=C(O)c1ccn(C(=O)N2CCN(Cc3nc4ccccc4s3)CC2)n1 ; O=C(O)c1ccn(C(=O)N2CCC(NCc3cccc(Oc4ccc(Cl)cc4)c3)CC2)n1 ; O=C(O)c1ccn(C(=O)N2CCC3(CCN(Cc4ccc(N5CCCC5)cc4C(F)(F)F)C3)CC2)n1 ; O=C(O)c1ccn(C(=O)N2CCC3(CCN(Cc4cccc(C(F)(F)F)c4)C3)CC2)n1 ; O=C(O)c1ccn(C(=O)N2C[C@H]3C[C@@H](OCc4cc(F)cc(Cl)c4)C[C@H]3C2)n1]

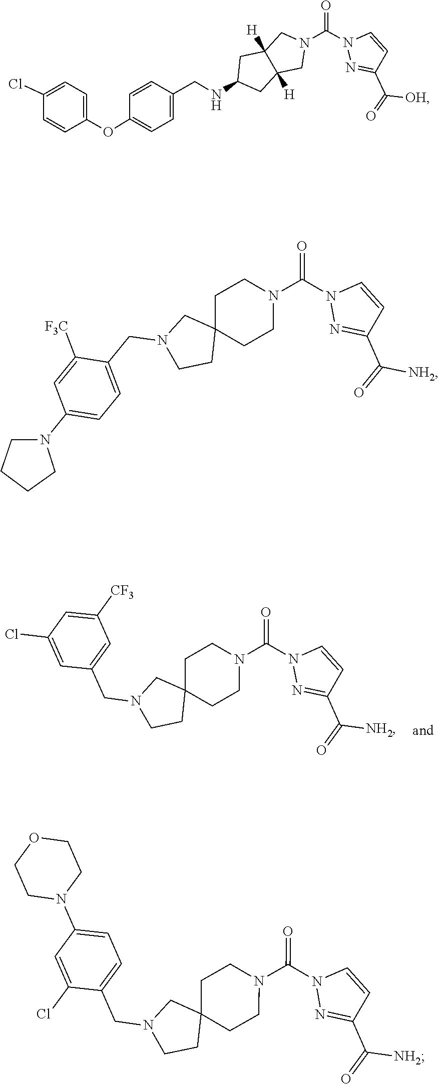 [SMILES: O=C(O)c1ccn(C(=O)N2C[C@H]3C[C@H](NCc4ccc(Oc5ccc(Cl)cc5)cc4)C[C@H]3C2)n1 ; NC(=O)c1ccn(C(=O)N2CCC3(CCN(Cc4ccc(N5CCCC5)cc4C(F)(F)F)C3)CC2)n1 ; NC(=O)c1ccn(C(=O)N2CCC3(CCN(Cc4cc(Cl)cc(C(F)(F)F)c4)C3)CC2)n1 ; NC(=O)c1ccn(C(=O)N2CCC3(CCN(Cc4ccc(N5CCOCC5)cc4Cl)C3)CC2)n1]

or a solvate, hydrate, tautomer, N-oxide, stereoisomer, or pharmaceutically acceptable salt thereof.

E67: The method of embodiment 1 or 2, wherein the compound of Formula (I) or Formula (I′) is selected from:

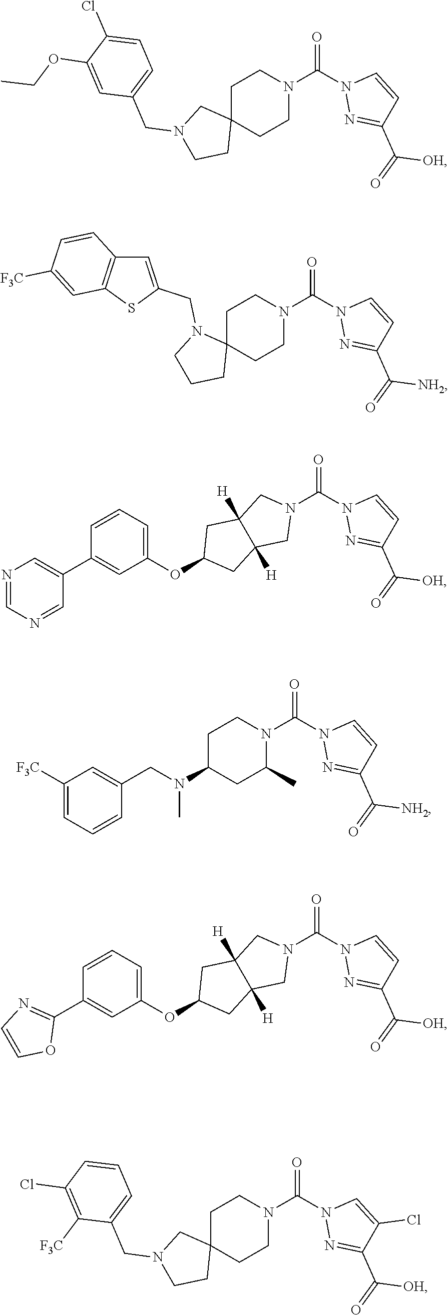 [SMILES: CCOc1cc(CN2CCC3(CCN(C(=O)n4ccc(C(=O)O)n4)CC3)C2)ccc1Cl ; NC(=O)c1ccn(C(=O)N2CCC3(CCCN3Cc3cc4ccc(C(F)(F)F)cc4s3)CC2)n1 ; O=C(O)c1ccn(C(=O)N2C[C@H]3C[C@H](Oc4cccc(-c5cncnc5)c4)C[C@H]3C2)n1 ; C[C@H]1C[C@@H](N(C)Cc2cccc(C(F)(F)F)c2)CCN1C(=O)n1ccc(C(N)=O)n1 ; O=C(O)c1ccn(C(=O)N2C[C@H]3C[C@H](Oc4cccc(-c5ncco5)c4)C[C@H]3C2)n1 ; O=C(O)c1nn(C(=O)N2CCC3(CCN(Cc4cccc(Cl)c4C(F)(F)F)C3)CC2)cc1Cl]

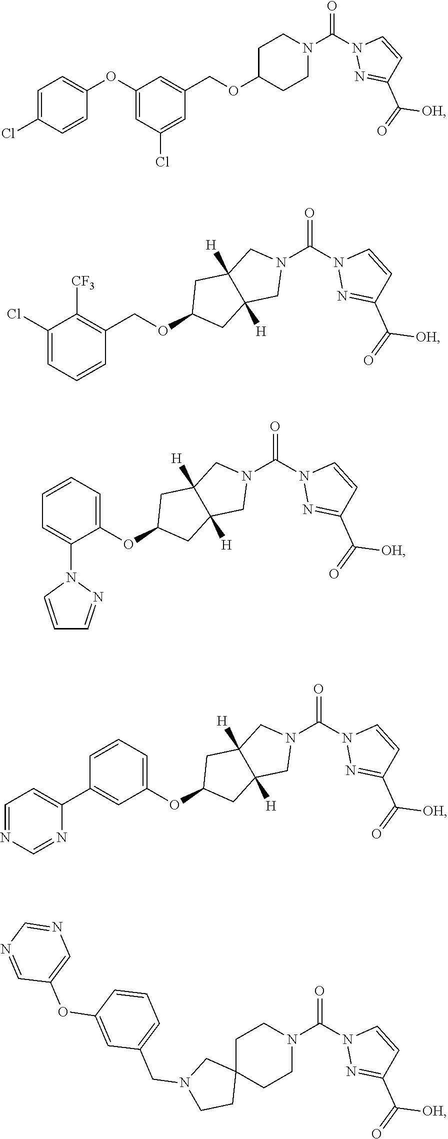 [SMILES: O=C(O)c1ccn(C(=O)N2CCC(OCc3cc(Cl)cc(Oc4ccc(Cl)cc4)c3)CC2)n1 ; O=C(O)c1ccn(C(=O)N2C[C@H]3C[C@H](OCc4cccc(Cl)c4C(F)(F)F)C[C@H]3C2)n1 ; O=C(O)c1ccn(C(=O)N2C[C@H]3C[C@H](Oc4ccccc4-n4cccn4)C[C@H]3C2)n1 ; O=C(O)c1ccn(C(=O)N2C[C@H]3C[C@H](Oc4cccc(-c5ccncn5)c4)C[C@H]3C2)n1 ; O=C(O)c1ccn(C(=O)N2CCC3(CCN(Cc4cccc(Oc5cncnc5)c4)C3)CC2)n1]

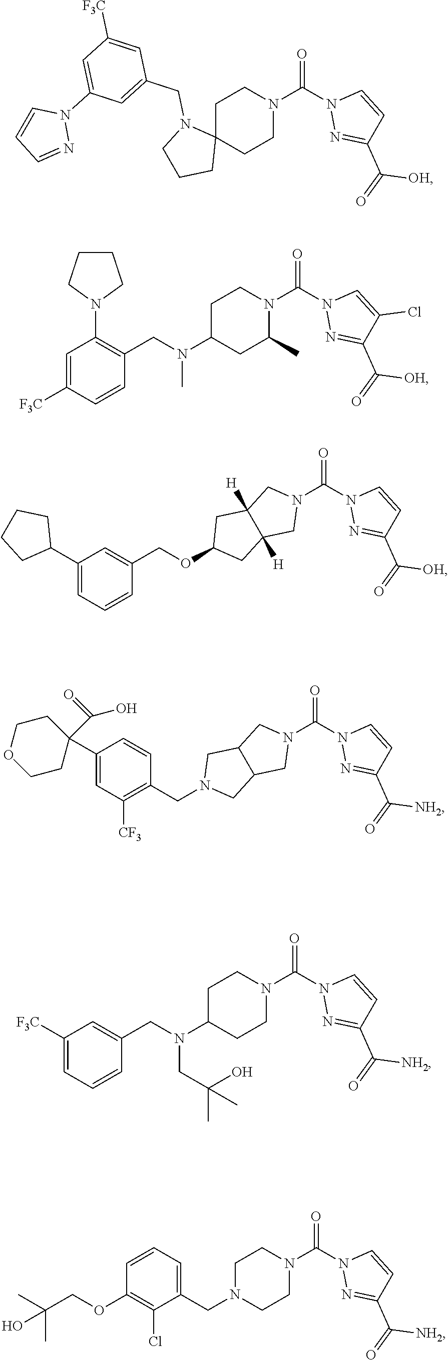 [SMILES: O=C(O)c1ccn(C(=O)N2CCC3(CCCN3Cc3cc(-n4cccn4)cc(C(F)(F)F)c3)CC2)n1 ; C[C@H]1CC(N(C)Cc2ccc(C(F)(F)F)cc2N2CCCC2)CCN1C(=O)n1cc(Cl)c(C(=O)O)n1 ; O=C(O)c1ccn(C(=O)N2C[C@H]3C[C@H](OCc4cccc(C5CCCC5)c4)C[C@H]3C2)n1 ; NC(=O)c1ccn(C(=O)N2CC3CN(Cc4ccc(C5(C(=O)O)CCOCC5)cc4C(F)(F)F)CC3C2)n1 ; CC(C)(O)CN(Cc1cccc(C(F)(F)F)c1)C1CCN(C(=O)n2ccc(C(N)=O)n2)CC1 ; CC(C)(O)COc1cccc(CN2CCN(C(=O)n3ccc(C(N)=O)n3)CC2)c1Cl]

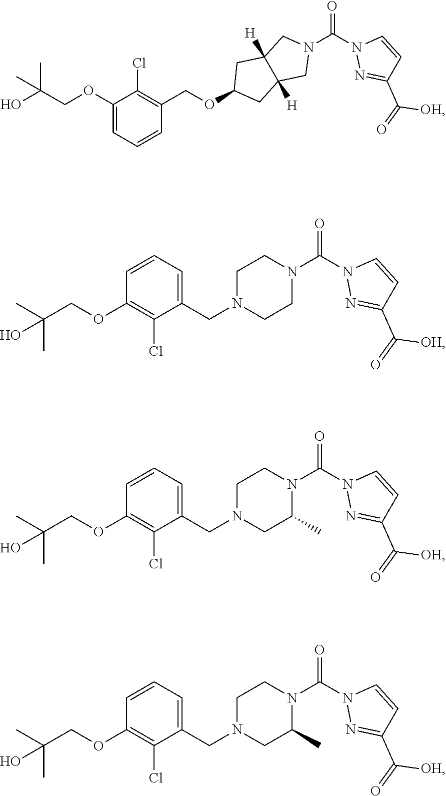 [SMILES: CC(C)(O)COc1cccc(CO[C@H]2C[C@@H]3CN(C(=O)n4ccc(C(=O)O)n4)C[C@@H]3C2)c1Cl ; CC(C)(O)COc1cccc(CN2CCN(C(=O)n3ccc(C(=O)O)n3)CC2)c1Cl ; C[C@@H]1CN(Cc2cccc(OCC(C)(C)O)c2Cl)CCN1C(=O)n1ccc(C(=O)O)n1 ; C[C@H]1CN(Cc2cccc(OCC(C)(C)O)c2Cl)CCN1C(=O)n1ccc(C(=O)O)n1]

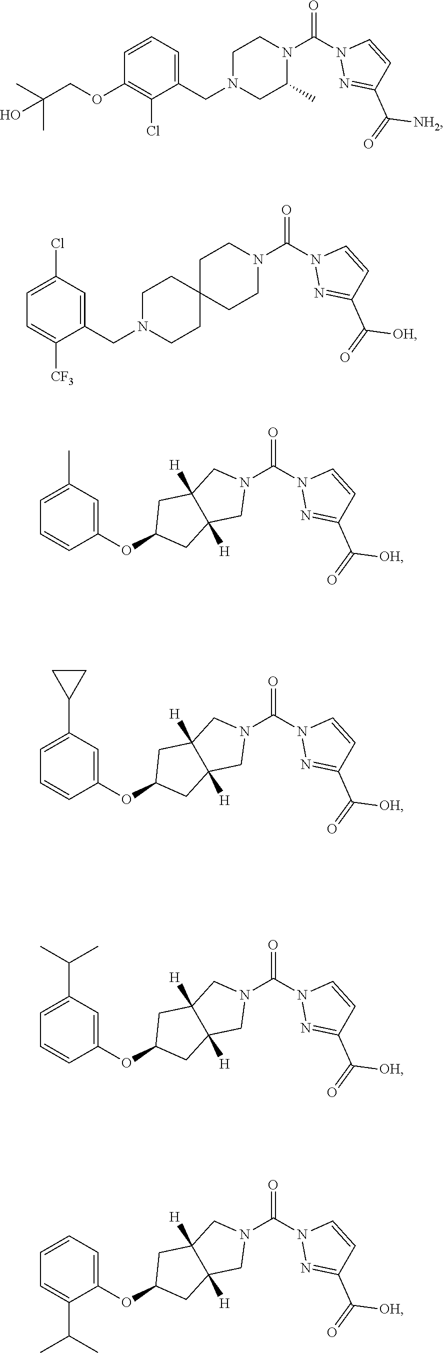 [SMILES: C[C@@H]1CN(Cc2cccc(OCC(C)(C)O)c2Cl)CCN1C(=O)n1ccc(C(N)=O)n1 ; O=C(O)c1ccn(C(=O)N2CCC3(CCN(Cc4cc(Cl)ccc4C(F)(F)F)CC3)CC2)n1 ; Cc1cccc(O[C@H]2C[C@@H]3CN(C(=O)n4ccc(C(=O)O)n4)C[C@@H]3C2)c1 ; O=C(O)c1ccn(C(=O)N2C[C@H]3C[C@H](Oc4cccc(C5CC5)c4)C[C@H]3C2)n1 ; CC(C)c1cccc(O[C@H]2C[C@@H]3CN(C(=O)n4ccc(C(=O)O)n4)C[C@@H]3C2)c1 ; CC(C)c1ccccc1O[C@H]1C[C@@H]2CN(C(=O)n3ccc(C(=O)O)n3)C[C@@H]2C1]

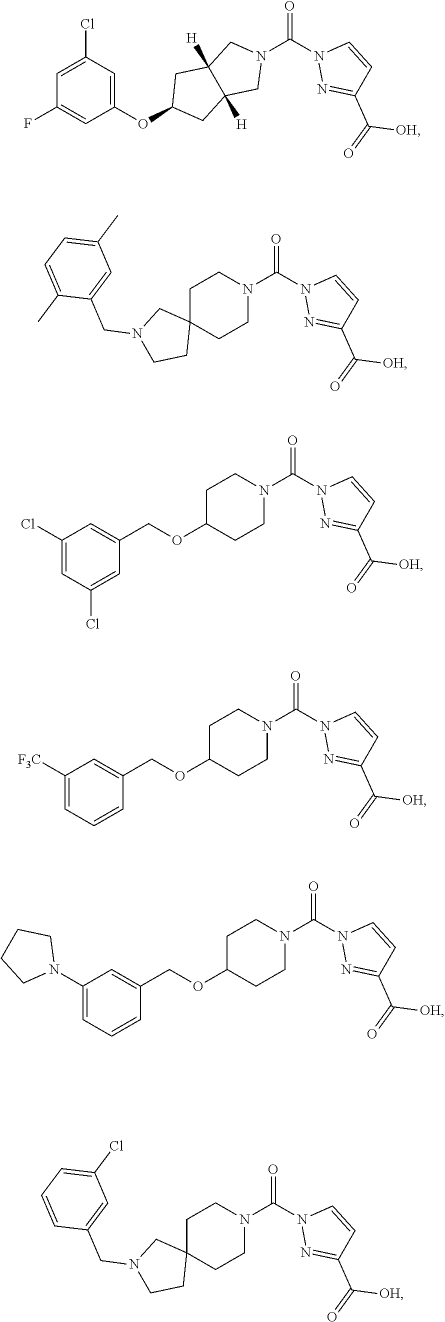 [SMILES: O=C(O)c1ccn(C(=O)N2C[C@H]3C[C@H](Oc4cc(F)cc(Cl)c4)C[C@H]3C2)n1 ; Cc1ccc(C)c(CN2CCC3(CCN(C(=O)n4ccc(C(=O)O)n4)CC3)C2)c1 ; O=C(O)c1ccn(C(=O)N2CCC(OCc3cc(Cl)cc(Cl)c3)CC2)n1 ; O=C(O)c1ccn(C(=O)N2CCC(OCc3cccc(C(F)(F)F)c3)CC2)n1 ; O=C(O)c1ccn(C(=O)N2CCC(OCc3cccc(N4CCCC4)c3)CC2)n1 ; O=C(O)c1ccn(C(=O)N2CCC3(CCN(Cc4cccc(Cl)c4)C3)CC2)n1]

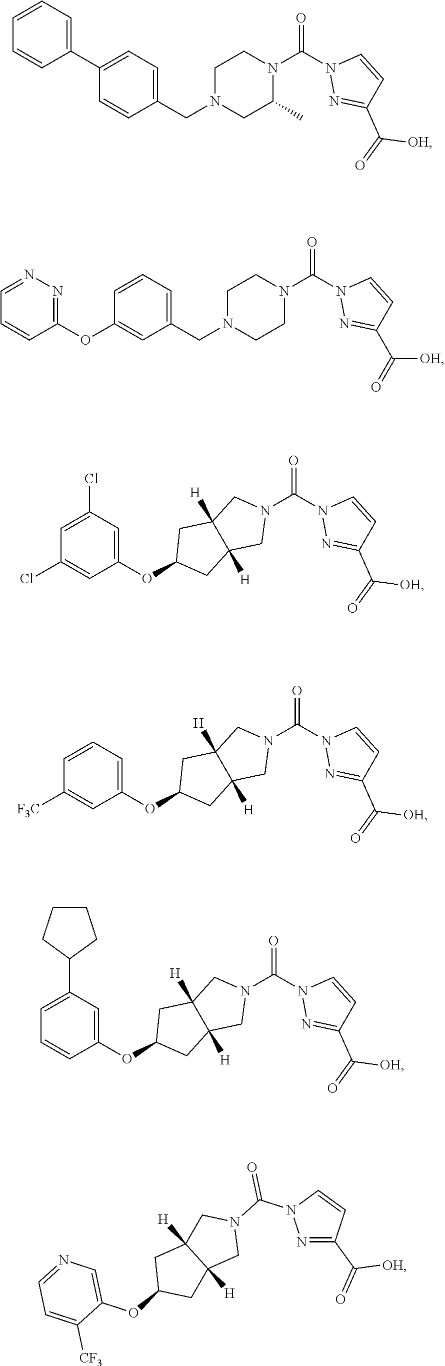 [SMILES: C[C@@H]1CN(Cc2ccc(-c3ccccc3)cc2)CCN1C(=O)n1ccc(C(=O)O)n1 ; O=C(O)c1ccn(C(=O)N2CCN(Cc3cccc(Oc4cccnn4)c3)CC2)n1 ; O=C(O)c1ccn(C(=O)N2C[C@H]3C[C@H](Oc4cc(Cl)cc(Cl)c4)C[C@H]3C2)n1 ; O=C(O)c1ccn(C(=O)N2C[C@H]3C[C@H](Oc4cccc(C(F)(F)F)c4)C[C@H]3C2)n1 ; O=C(O)c1ccn(C(=O)N2C[C@H]3C[C@H](Oc4cccc(C5CCCC5)c4)C[C@H]3C2)n1 ; O=C(O)c1ccn(C(=O)N2C[C@H]3C[C@H](Oc4cnccc4C(F)(F)F)C[C@H]3C2)n1]

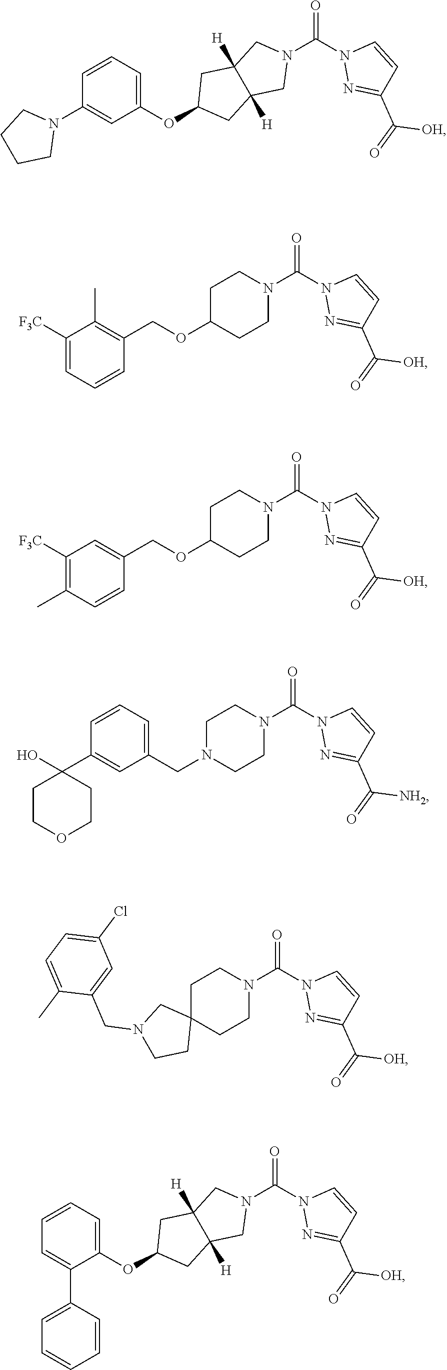 [SMILES: O=C(O)c1ccn(C(=O)N2C[C@H]3C[C@H](Oc4cccc(N5CCCC5)c4)C[C@H]3C2)n1 ; Cc1c(COC2CCN(C(=O)n3ccc(C(=O)O)n3)CC2)cccc1C(F)(F)F ; Cc1ccc(COC2CCN(C(=O)n3ccc(C(=O)O)n3)CC2)cc1C(F)(F)F ; NC(=O)c1ccn(C(=O)N2CCN(Cc3cccc(C4(O)CCOCC4)c3)CC2)n1 ; Cc1ccc(Cl)cc1CN1CCC2(CCN(C(=O)n3ccc(C(=O)O)n3)CC2)C1 ; O=C(O)c1ccn(C(=O)N2C[C@H]3C[C@H](Oc4ccccc4-c4ccccc4)C[C@H]3C2)n1]

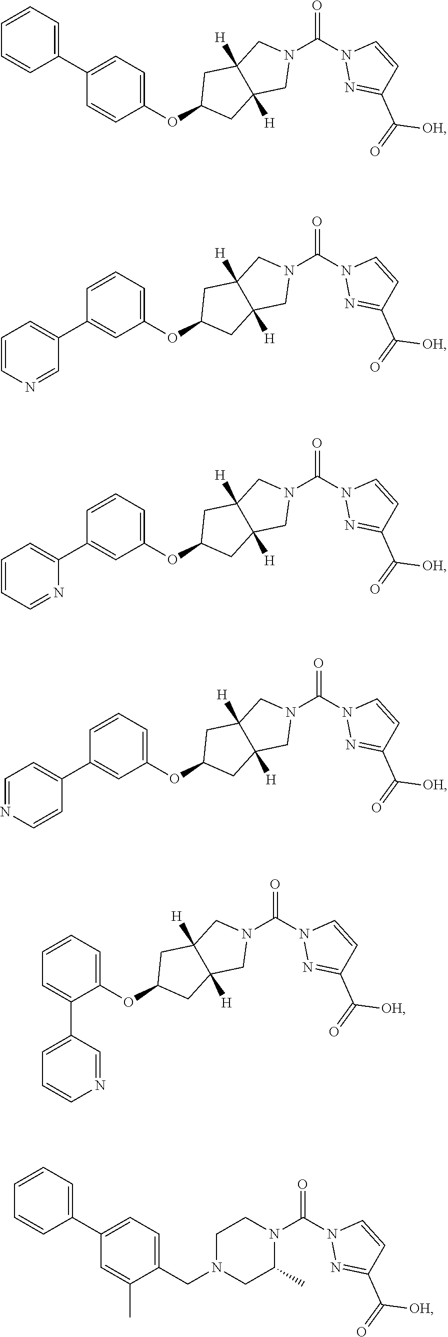 [SMILES: O=C(O)c1ccn(C(=O)N2C[C@H]3C[C@H](Oc4ccc(-c5ccccc5)cc4)C[C@H]3C2)n1 ; O=C(O)c1ccn(C(=O)N2C[C@H]3C[C@H](Oc4cccc(-c5cccnc5)c4)C[C@H]3C2)n1 ; O=C(O)c1ccn(C(=O)N2C[C@H]3C[C@H](Oc4cccc(-c5ccccn5)c4)C[C@H]3C2)n1 ; O=C(O)c1ccn(C(=O)N2C[C@H]3C[C@H](Oc4cccc(-c5ccncc5)c4)C[C@H]3C2)n1 ; O=C(O)c1ccn(C(=O)N2C[C@H]3C[C@H](Oc4ccccc4-c4cccnc4)C[C@H]3C2)n1 ; Cc1cc(-c2ccccc2)ccc1CN1CCN(C(=O)n2ccc(C(=O)O)n2)[C@H](C)C1]

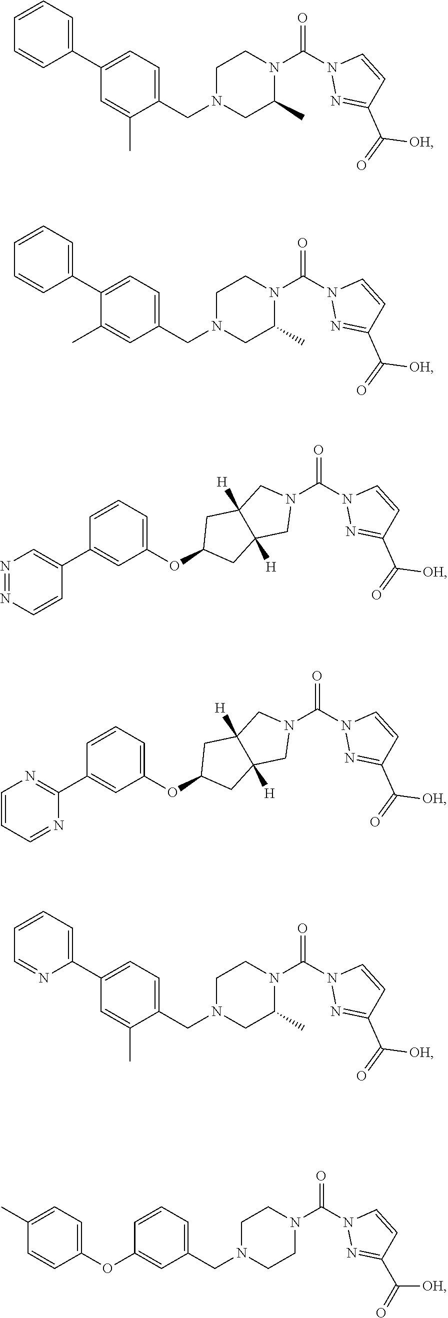 [SMILES: Cc1cc(-c2ccccc2)ccc1CN1CCN(C(=O)n2ccc(C(=O)O)n2)[C@@H](C)C1 ; Cc1cc(CN2CCN(C(=O)n3ccc(C(=O)O)n3)[C@H](C)C2)ccc1-c1ccccc1 ; O=C(O)c1ccn(C(=O)N2C[C@H]3C[C@H](Oc4cccc(-c5ccnnc5)c4)C[C@H]3C2)n1 ; O=C(O)c1ccn(C(=O)N2C[C@H]3C[C@H](Oc4cccc(-c5ncccn5)c4)C[C@H]3C2)n1 ; Cc1cc(-c2ccccn2)ccc1CN1CCN(C(=O)n2ccc(C(=O)O)n2)[C@H](C)C1 ; Cc1ccc(Oc2cccc(CN3CCN(C(=O)n4ccc(C(=O)O)n4)CC3)c2)cc1]

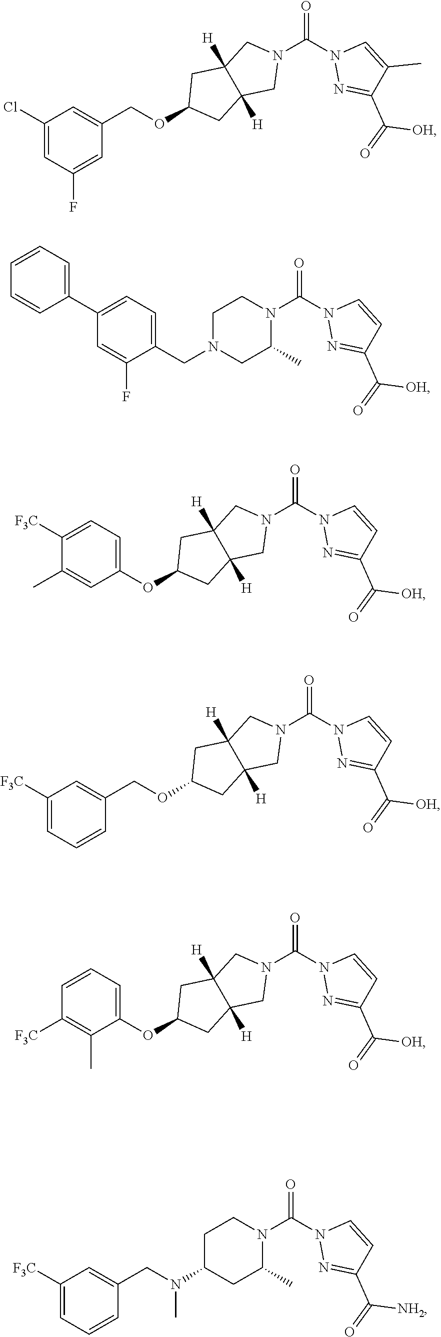

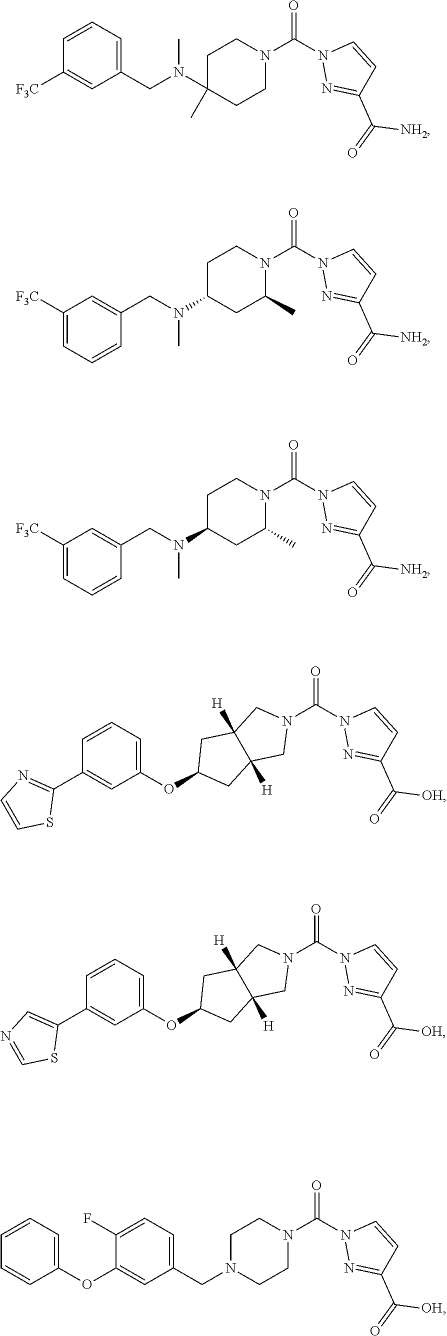 [SMILES: CN(Cc1cccc(C(F)(F)F)c1)C1(C)CCN(C(=O)n2ccc(C(N)=O)n2)CC1 ; C[C@H]1C[C@H](N(C)Cc2cccc(C(F)(F)F)c2)CCN1C(=O)n1ccc(C(N)=O)n1 ; C[C@@H]1C[C@@H](N(C)Cc2cccc(C(F)(F)F)c2)CCN1C(=O)n1ccc(C(N)=O)n1 ; O=C(O)c1ccn(C(=O)N2C[C@H]3C[C@H](Oc4cccc(-c5nccs5)c4)C[C@H]3C2)n1 ; O=C(O)c1ccn(C(=O)N2C[C@H]3C[C@H](Oc4cccc(-c5cncs5)c4)C[C@H]3C2)n1 ; O=C(O)c1ccn(C(=O)N2CCN(Cc3ccc(F)c(Oc4ccccc4)c3)CC2)n1]

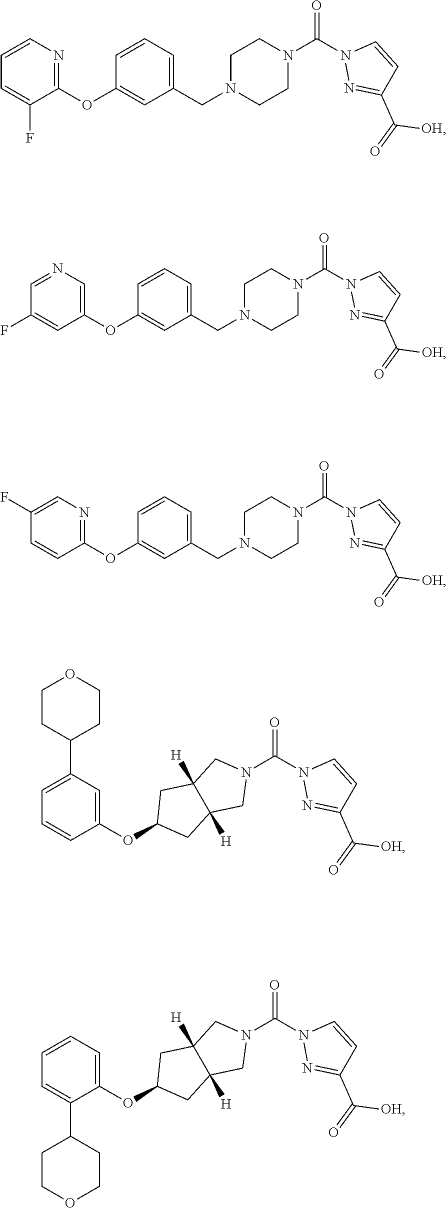 [SMILES: O=C(O)c1ccn(C(=O)N2CCN(Cc3cccc(Oc4ncccc4F)c3)CC2)n1 ; O=C(O)c1ccn(C(=O)N2CCN(Cc3cccc(Oc4cncc(F)c4)c3)CC2)n1 ; O=C(O)c1ccn(C(=O)N2CCN(Cc3cccc(Oc4ccc(F)cn4)c3)CC2)n1 ; O=C(O)c1ccn(C(=O)N2C[C@H]3C[C@H](Oc4cccc(C5CCOCC5)c4)C[C@H]3C2)n1 ; O=C(O)c1ccn(C(=O)N2C[C@H]3C[C@H](Oc4ccccc4C4CCOCC4)C[C@H]3C2)n1]

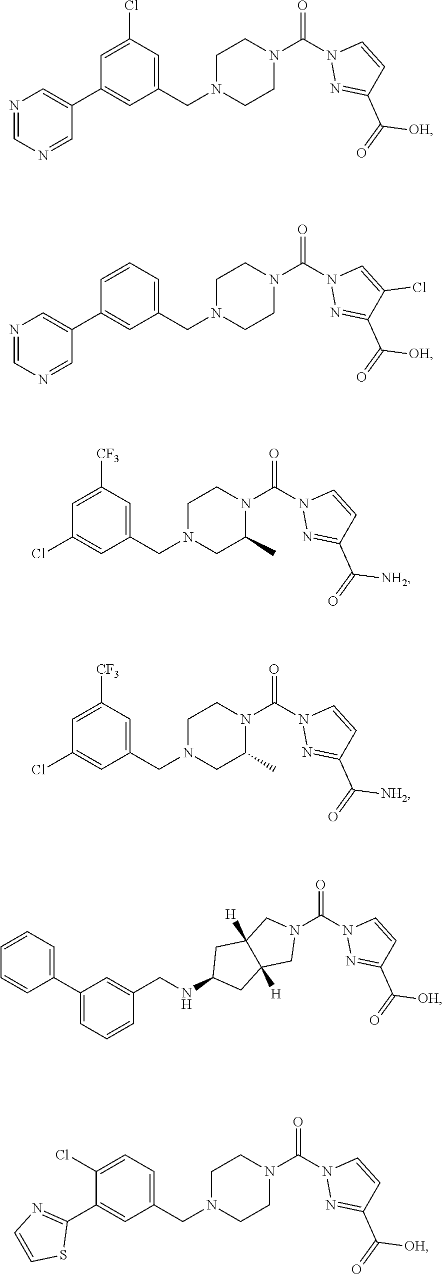 [SMILES: O=C(O)c1ccn(C(=O)N2CCN(Cc3cc(Cl)cc(-c4cncnc4)c3)CC2)n1 ; O=C(O)c1nn(C(=O)N2CCN(Cc3cccc(-c4cncnc4)c3)CC2)cc1Cl ; C[C@H]1CN(Cc2cc(Cl)cc(C(F)(F)F)c2)CCN1C(=O)n1ccc(C(N)=O)n1 ; C[C@@H]1CN(Cc2cc(Cl)cc(C(F)(F)F)c2)CCN1C(=O)n1ccc(C(N)=O)n1 ; O=C(O)c1ccn(C(=O)N2C[C@H]3C[C@H](NCc4cccc(-c5ccccc5)c4)C[C@H]3C2)n1 ; O=C(O)c1ccn(C(=O)N2CCN(Cc3ccc(Cl)c(-c4nccs4)c3)CC2)n1]

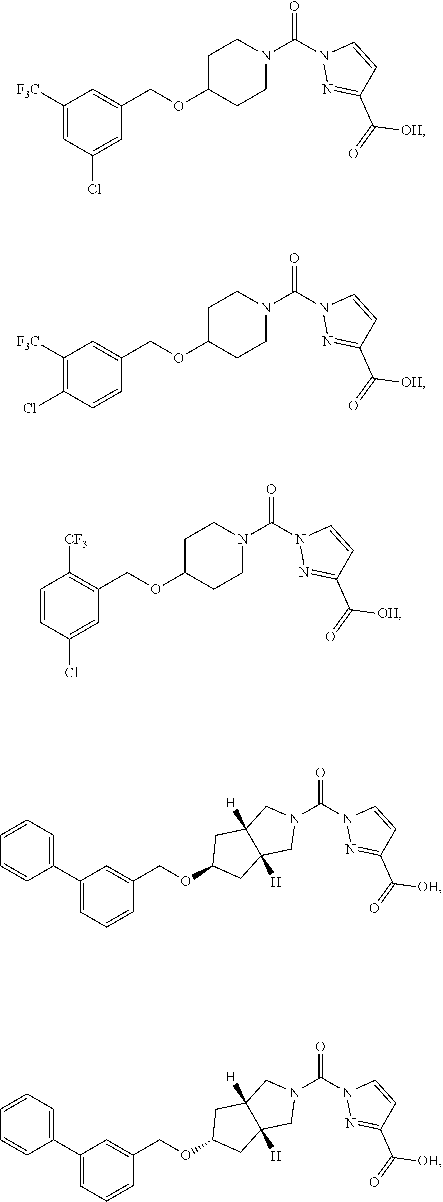 [SMILES: O=C(O)c1ccn(C(=O)N2CCC(OCc3cc(Cl)cc(C(F)(F)F)c3)CC2)n1 ; O=C(O)c1ccn(C(=O)N2CCC(OCc3ccc(Cl)c(C(F)(F)F)c3)CC2)n1 ; O=C(O)c1ccn(C(=O)N2CCC(OCc3cc(Cl)ccc3C(F)(F)F)CC2)n1 ; O=C(O)c1ccn(C(=O)N2C[C@H]3C[C@H](OCc4cccc(-c5ccccc5)c4)C[C@H]3C2)n1 ; O=C(O)c1ccn(C(=O)N2C[C@H]3C[C@@H](OCc4cccc(-c5ccccc5)c4)C[C@H]3C2)n1]

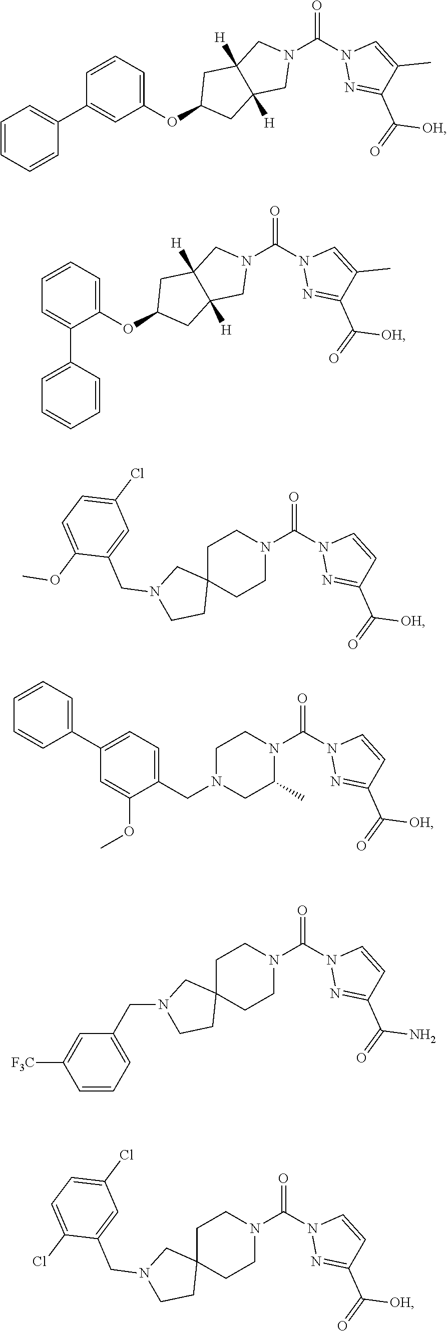

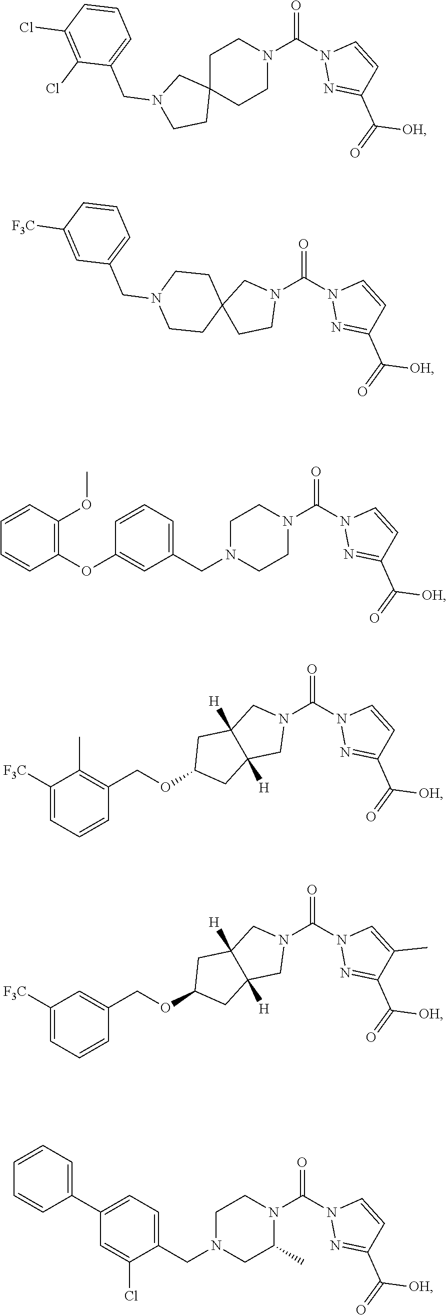 [SMILES: O=C(O)c1ccn(C(=O)N2CCC3(CCN(Cc4cccc(Cl)c4Cl)C3)CC2)n1 ; O=C(O)c1ccn(C(=O)N2CCC3(CCN(Cc4cccc(C(F)(F)F)c4)CC3)C2)n1 ; COc1ccccc1Oc1cccc(CN2CCN(C(=O)n3ccc(C(=O)O)n3)CC2)c1 ; Cc1c(CO[C@@H]2C[C@@H]3CN(C(=O)n4ccc(C(=O)O)n4)C[C@@H]3C2)cccc1C(F)(F)F ; Cc1cn(C(=O)N2C[C@H]3C[C@H](OCc4cccc(C(F)(F)F)c4)C[C@H]3C2)nc1C(=O)O ; C[C@@H]1CN(Cc2ccc(-c3ccccc3)cc2Cl)CCN1C(=O)n1ccc(C(=O)O)n1]

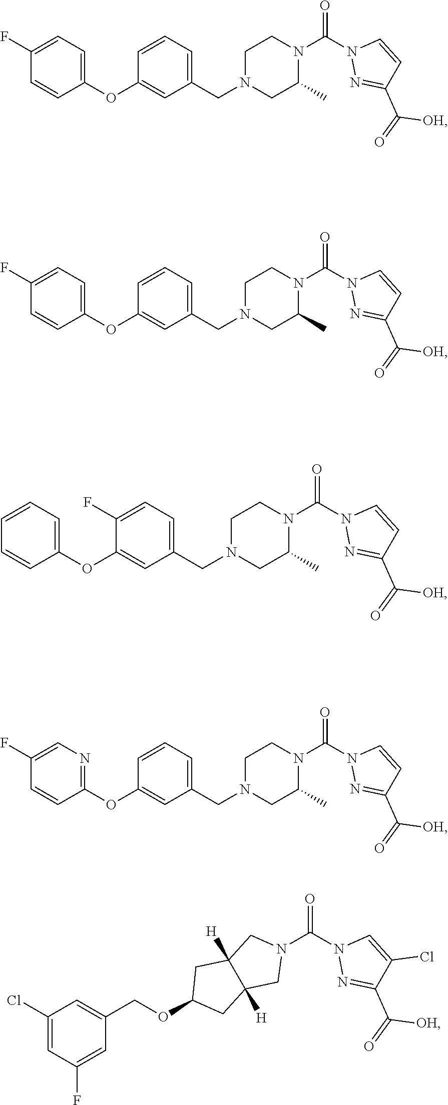 [SMILES: C[C@@H]1CN(Cc2cccc(Oc3ccc(F)cc3)c2)CCN1C(=O)n1ccc(C(=O)O)n1 ; C[C@H]1CN(Cc2cccc(Oc3ccc(F)cc3)c2)CCN1C(=O)n1ccc(C(=O)O)n1 ; C[C@@H]1CN(Cc2ccc(F)c(Oc3ccccc3)c2)CCN1C(=O)n1ccc(C(=O)O)n1 ; C[C@@H]1CN(Cc2cccc(Oc3ccc(F)cn3)c2)CCN1C(=O)n1ccc(C(=O)O)n1 ; O=C(O)c1nn(C(=O)N2C[C@H]3C[C@H](OCc4cc(F)cc(Cl)c4)C[C@H]3C2)cc1Cl]

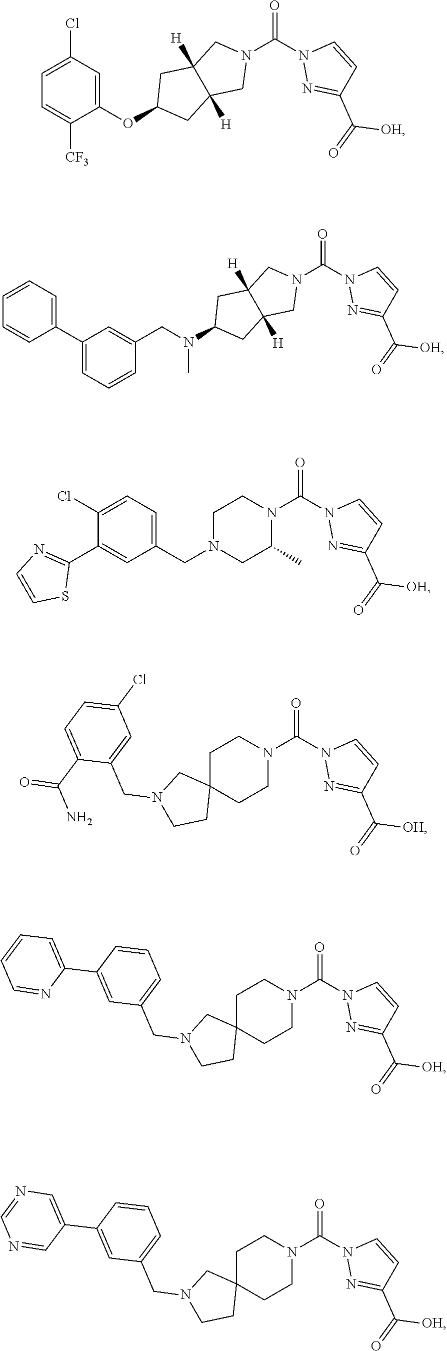 [SMILES: O=C(O)c1ccn(C(=O)N2C[C@H]3C[C@H](Oc4cc(Cl)ccc4C(F)(F)F)C[C@H]3C2)n1 ; CN(Cc1cccc(-c2ccccc2)c1)[C@H]1C[C@@H]2CN(C(=O)n3ccc(C(=O)O)n3)C[C@@H]2C1 ; C[C@@H]1CN(Cc2ccc(Cl)c(-c3nccs3)c2)CCN1C(=O)n1ccc(C(=O)O)n1 ; NC(=O)c1ccc(Cl)cc1CN1CCC2(CCN(C(=O)n3ccc(C(=O)O)n3)CC2)C1 ; O=C(O)c1ccn(C(=O)N2CCC3(CCN(Cc4cccc(-c5ccccn5)c4)C3)CC2)n1 ; O=C(O)c1ccn(C(=O)N2CCC3(CCN(Cc4cccc(-c5cncnc5)c4)C3)CC2)n1]

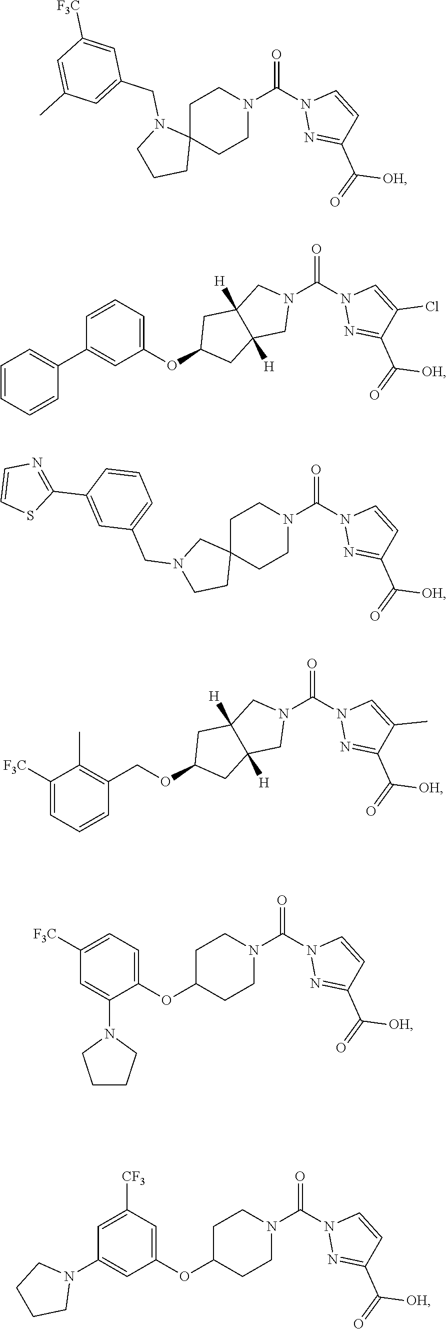 [SMILES: Cc1cc(CN2CCCC23CCN(C(=O)n2ccc(C(=O)O)n2)CC3)cc(C(F)(F)F)c1 ; O=C(O)c1nn(C(=O)N2C[C@H]3C[C@H](Oc4cccc(-c5ccccc5)c4)C[C@H]3C2)cc1Cl ; O=C(O)c1ccn(C(=O)N2CCC3(CCN(Cc4cccc(-c5nccs5)c4)C3)CC2)n1 ; Cc1cn(C(=O)N2C[C@H]3C[C@H](OCc4cccc(C(F)(F)F)c4C)C[C@H]3C2)nc1C(=O)O ; O=C(O)c1ccn(C(=O)N2CCC(Oc3ccc(C(F)(F)F)cc3N3CCCC3)CC2)n1 ; O=C(O)c1ccn(C(=O)N2CCC(Oc3cc(N4CCCC4)cc(C(F)(F)F)c3)CC2)n1]

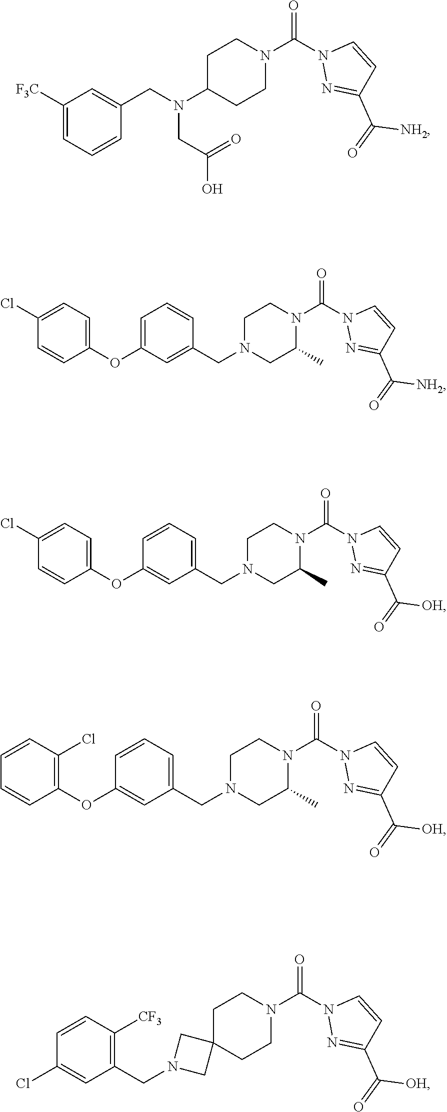 [SMILES: NC(=O)c1ccn(C(=O)N2CCC(N(CC(=O)O)Cc3cccc(C(F)(F)F)c3)CC2)n1 ; C[C@@H]1CN(Cc2cccc(Oc3ccc(Cl)cc3)c2)CCN1C(=O)n1ccc(C(N)=O)n1 ; C[C@H]1CN(Cc2cccc(Oc3ccc(Cl)cc3)c2)CCN1C(=O)n1ccc(C(=O)O)n1 ; C[C@@H]1CN(Cc2cccc(Oc3ccccc3Cl)c2)CCN1C(=O)n1ccc(C(=O)O)n1 ; O=C(O)c1ccn(C(=O)N2CCC3(CC2)CN(Cc2cc(Cl)ccc2C(F)(F)F)C3)n1]

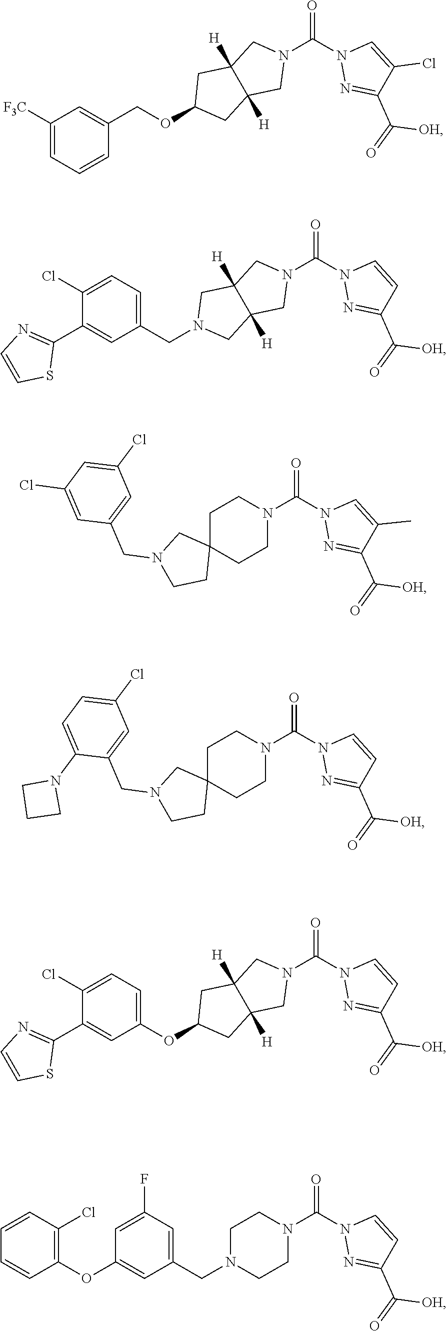 [SMILES: O=C(O)c1nn(C(=O)N2C[C@H]3C[C@H](OCc4cccc(C(F)(F)F)c4)C[C@H]3C2)cc1Cl ; O=C(O)c1ccn(C(=O)N2C[C@H]3CN(Cc4ccc(Cl)c(-c5nccs5)c4)C[C@H]3C2)n1 ; Cc1cn(C(=O)N2CCC3(CCN(Cc4cc(Cl)cc(Cl)c4)C3)CC2)nc1C(=O)O ; O=C(O)c1ccn(C(=O)N2CCC3(CCN(Cc4cc(Cl)ccc4N4CCC4)C3)CC2)n1 ; O=C(O)c1ccn(C(=O)N2C[C@H]3C[C@H](Oc4ccc(Cl)c(-c5nccs5)c4)C[C@H]3C2)n1 ; O=C(O)c1ccn(C(=O)N2CCN(Cc3cc(F)cc(Oc4ccccc4Cl)c3)CC2)n1]

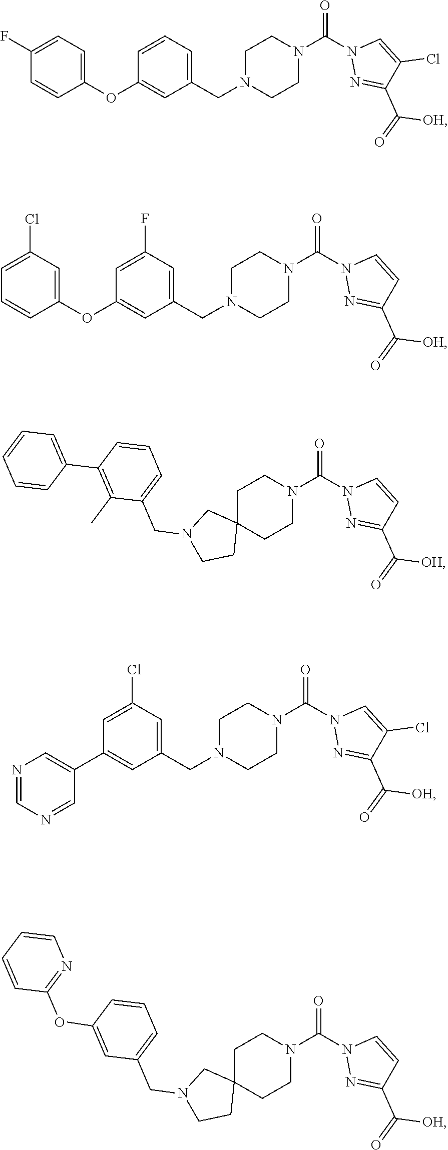 [SMILES: O=C(O)c1nn(C(=O)N2CCN(Cc3cccc(Oc4ccc(F)cc4)c3)CC2)cc1Cl ; O=C(O)c1ccn(C(=O)N2CCN(Cc3cc(F)cc(Oc4cccc(Cl)c4)c3)CC2)n1 ; Cc1c(CN2CCC3(CCN(C(=O)n4ccc(C(=O)O)n4)CC3)C2)cccc1-c1ccccc1 ; O=C(O)c1nn(C(=O)N2CCN(Cc3cc(Cl)cc(-c4cncnc4)c3)CC2)cc1Cl ; O=C(O)c1ccn(C(=O)N2CCC3(CCN(Cc4cccc(Oc5ccccn5)c4)C3)CC2)n1]

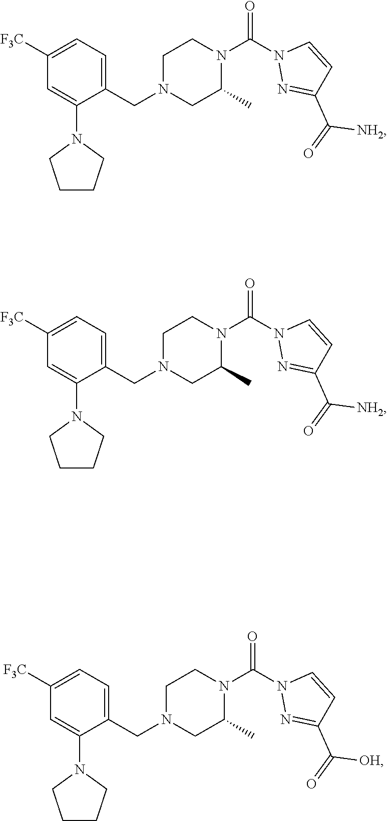 [SMILES: C[C@@H]1CN(Cc2ccc(C(F)(F)F)cc2N2CCCC2)CCN1C(=O)n1ccc(C(N)=O)n1 ; C[C@H]1CN(Cc2ccc(C(F)(F)F)cc2N2CCCC2)CCN1C(=O)n1ccc(C(N)=O)n1 ; C[C@@H]1CN(Cc2ccc(C(F)(F)F)cc2N2CCCC2)CCN1C(=O)n1ccc(C(=O)O)n1]

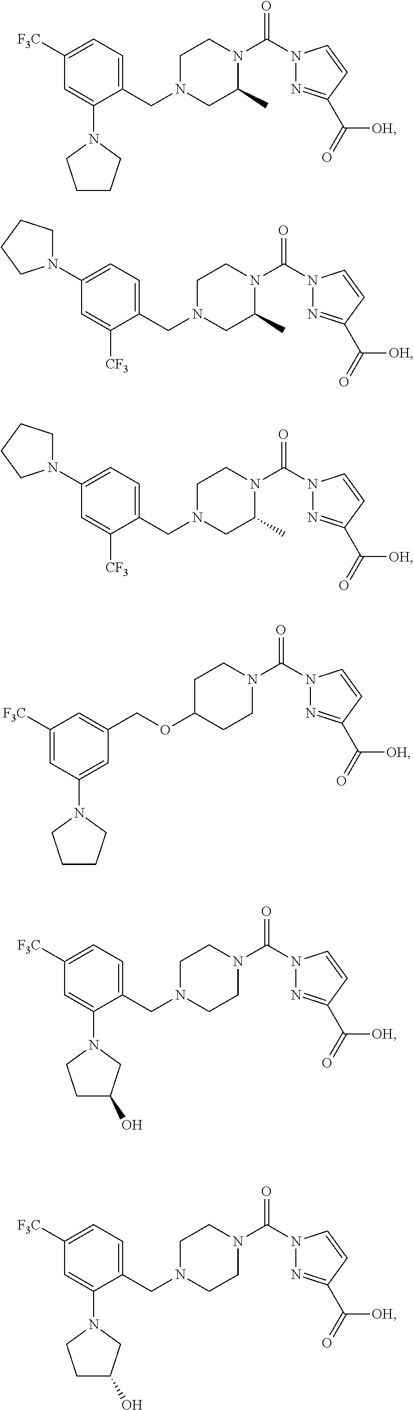 [SMILES: C[C@H]1CN(Cc2ccc(C(F)(F)F)cc2N2CCCC2)CCN1C(=O)n1ccc(C(=O)O)n1 ; C[C@H]1CN(Cc2ccc(N3CCCC3)cc2C(F)(F)F)CCN1C(=O)n1ccc(C(=O)O)n1 ; C[C@@H]1CN(Cc2ccc(N3CCCC3)cc2C(F)(F)F)CCN1C(=O)n1ccc(C(=O)O)n1 ; O=C(O)c1ccn(C(=O)N2CCC(OCc3cc(N4CCCC4)cc(C(F)(F)F)c3)CC2)n1 ; O=C(O)c1ccn(C(=O)N2CCN(Cc3ccc(C(F)(F)F)cc3N3CC[C@H](O)C3)CC2)n1 ; O=C(O)c1ccn(C(=O)N2CCN(Cc3ccc(C(F)(F)F)cc3N3CC[C@@H](O)C3)CC2)n1]

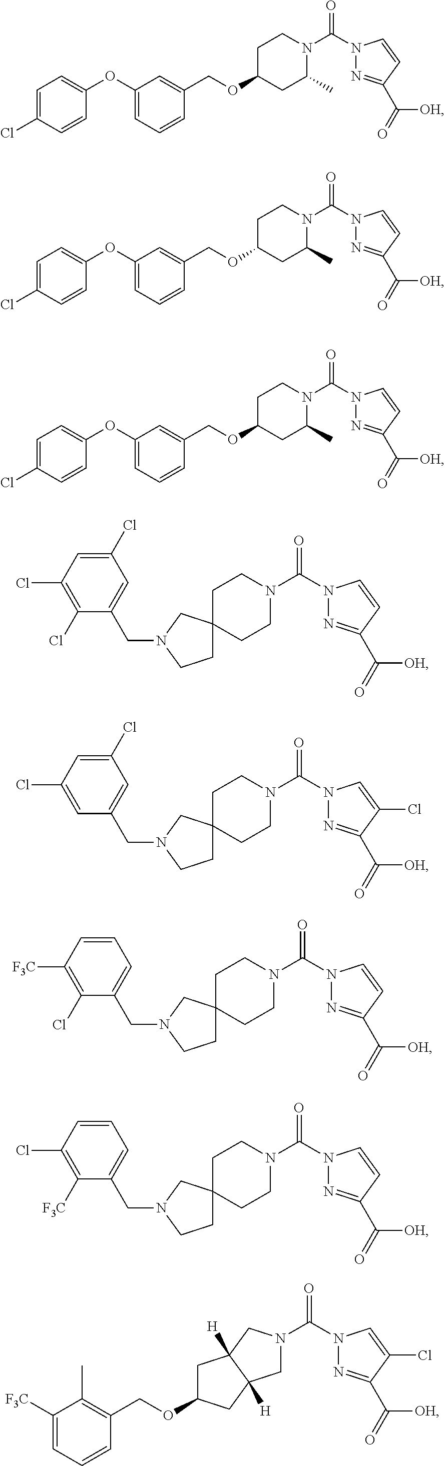

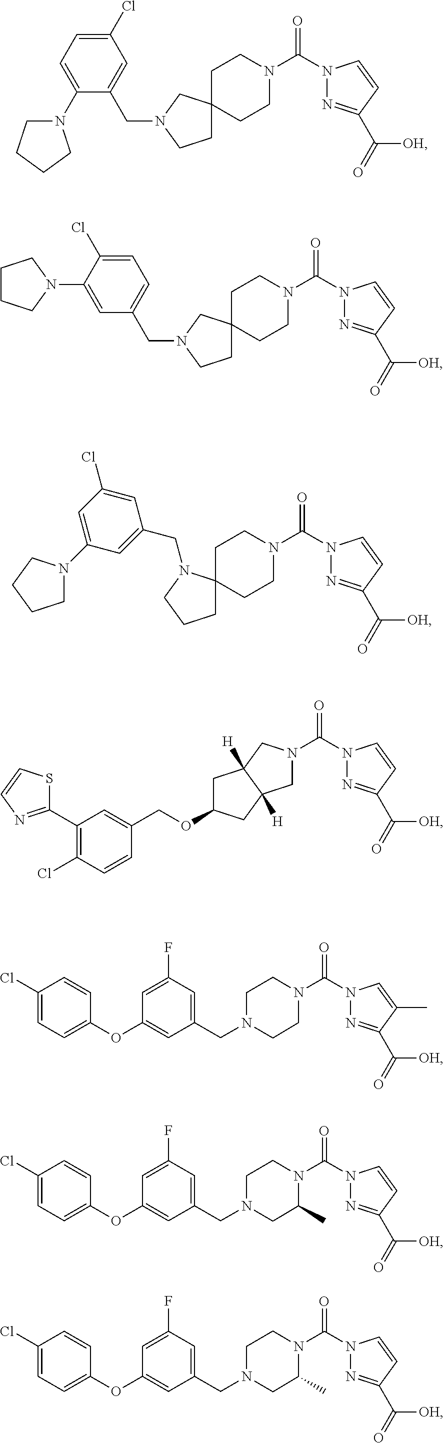

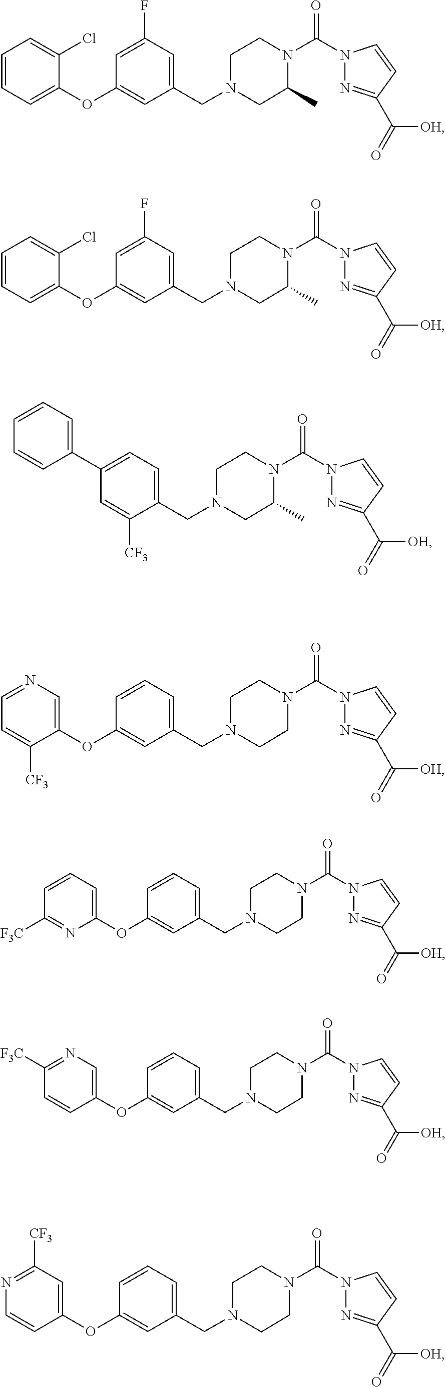

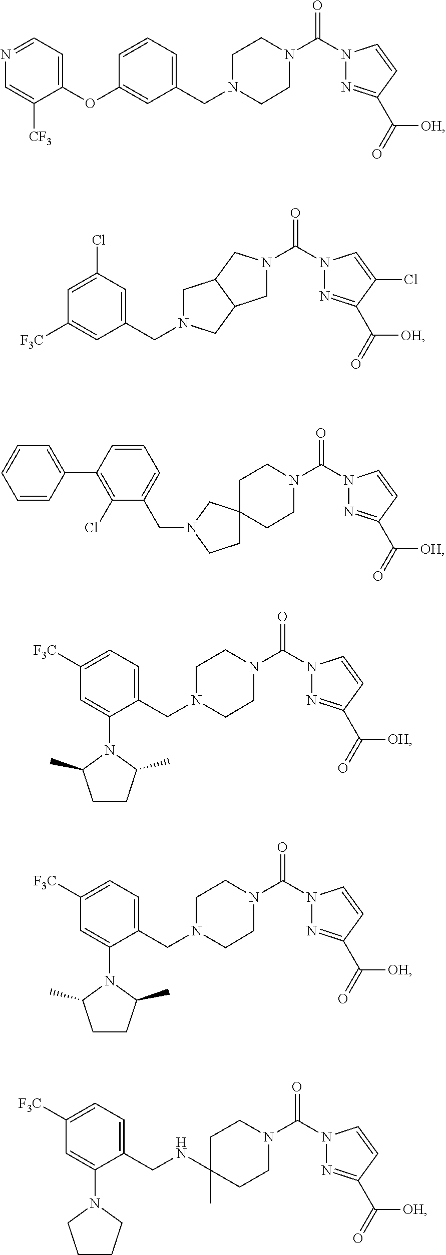 [SMILES: O=C(O)c1ccn(C(=O)N2CCN(Cc3cccc(Oc4ccncc4C(F)(F)F)c3)CC2)n1 ; O=C(O)c1nn(C(=O)N2CC3CN(Cc4cc(Cl)cc(C(F)(F)F)c4)CC3C2)cc1Cl ; O=C(O)c1ccn(C(=O)N2CCC3(CCN(Cc4cccc(-c5ccccc5)c4Cl)C3)CC2)n1 ; C[C@@H]1CC[C@@H](C)N1c1cc(C(F)(F)F)ccc1CN1CCN(C(=O)n2ccc(C(=O)O)n2)CC1 ; C[C@H]1CC[C@H](C)N1c1cc(C(F)(F)F)ccc1CN1CCN(C(=O)n2ccc(C(=O)O)n2)CC1 ; CC1(NCc2ccc(C(F)(F)F)cc2N2CCCC2)CCN(C(=O)n2ccc(C(=O)O)n2)CC1]

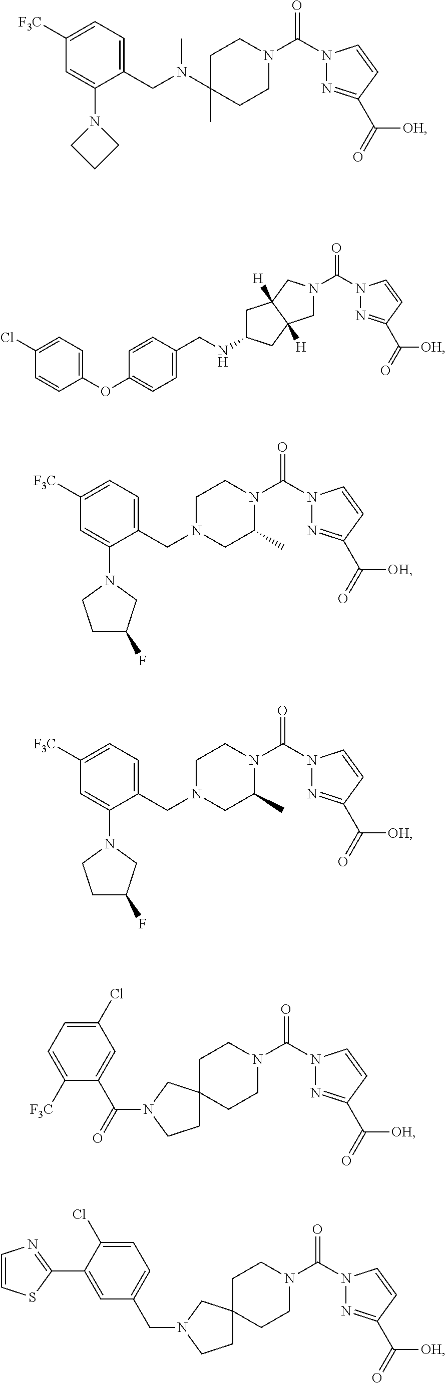 [SMILES: CN(Cc1ccc(C(F)(F)F)cc1N1CCC1)C1(C)CCN(C(=O)n2ccc(C(=O)O)n2)CC1 ; O=C(O)c1ccn(C(=O)N2C[C@H]3C[C@@H](NCc4ccc(Oc5ccc(Cl)cc5)cc4)C[C@H]3C2)n1 ; C[C@@H]1CN(Cc2ccc(C(F)(F)F)cc2N2CC[C@H](F)C2)CCN1C(=O)n1ccc(C(=O)O)n1 ; C[C@H]1CN(Cc2ccc(C(F)(F)F)cc2N2CC[C@H](F)C2)CCN1C(=O)n1ccc(C(=O)O)n1 ; O=C(O)c1ccn(C(=O)N2CCC3(CCN(C(=O)c4cc(Cl)ccc4C(F)(F)F)C3)CC2)n1 ; O=C(O)c1ccn(C(=O)N2CCC3(CCN(Cc4ccc(Cl)c(-c5nccs5)c4)C3)CC2)n1]

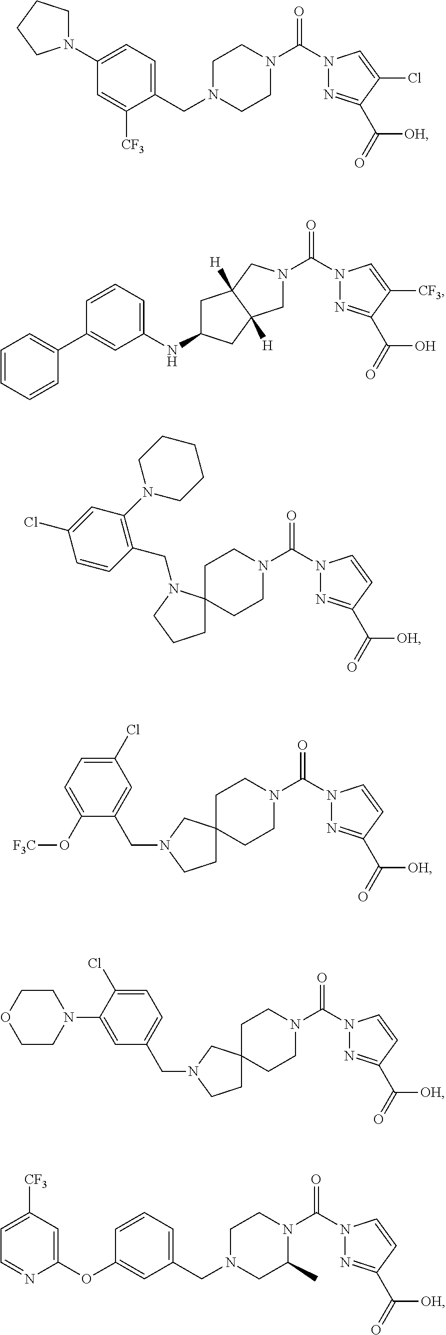 [SMILES: O=C(O)c1nn(C(=O)N2CCN(Cc3ccc(N4CCCC4)cc3C(F)(F)F)CC2)cc1Cl ; O=C(O)c1nn(C(=O)N2C[C@H]3C[C@H](Nc4cccc(-c5ccccc5)c4)C[C@H]3C2)cc1C(F)(F)F ; O=C(O)c1ccn(C(=O)N2CCC3(CCCN3Cc3ccc(Cl)cc3N3CCCCC3)CC2)n1 ; O=C(O)c1ccn(C(=O)N2CCC3(CCN(Cc4cc(Cl)ccc4OC(F)(F)F)C3)CC2)n1 ; O=C(O)c1ccn(C(=O)N2CCC3(CCN(Cc4ccc(Cl)c(N5CCOCC5)c4)C3)CC2)n1 ; C[C@H]1CN(Cc2cccc(Oc3cc(C(F)(F)F)ccn3)c2)CCN1C(=O)n1ccc(C(=O)O)n1]

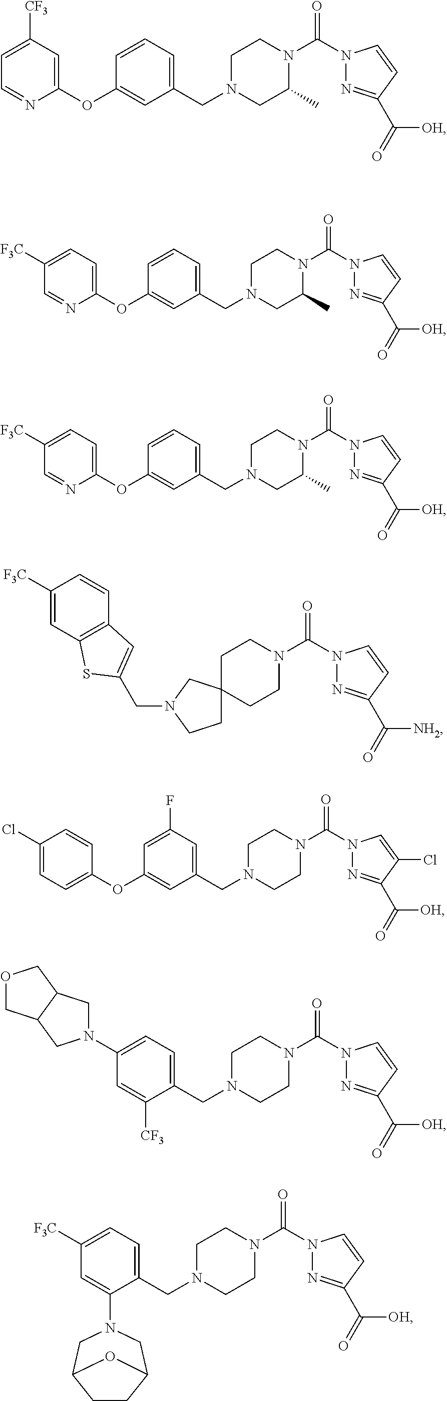

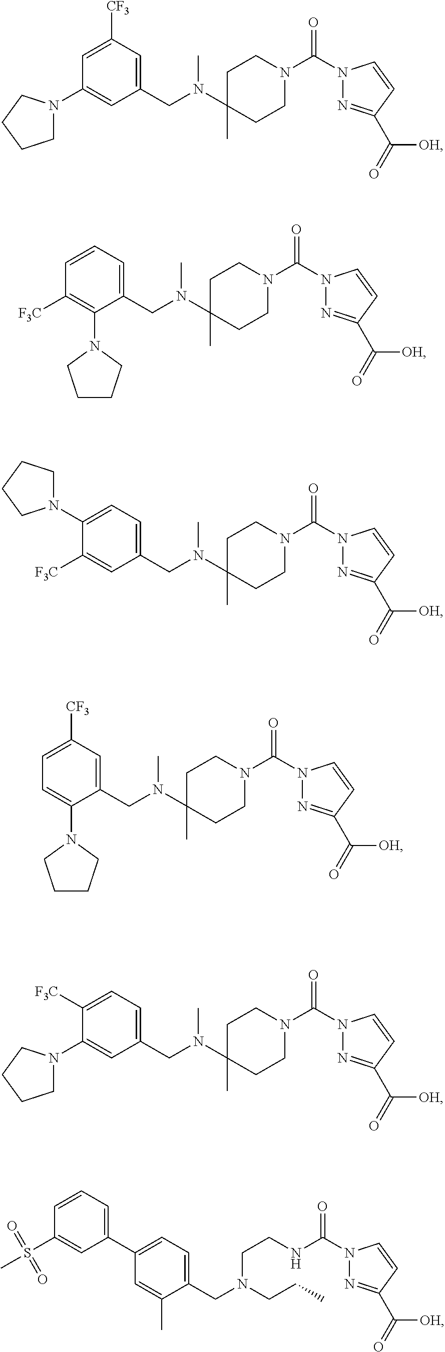

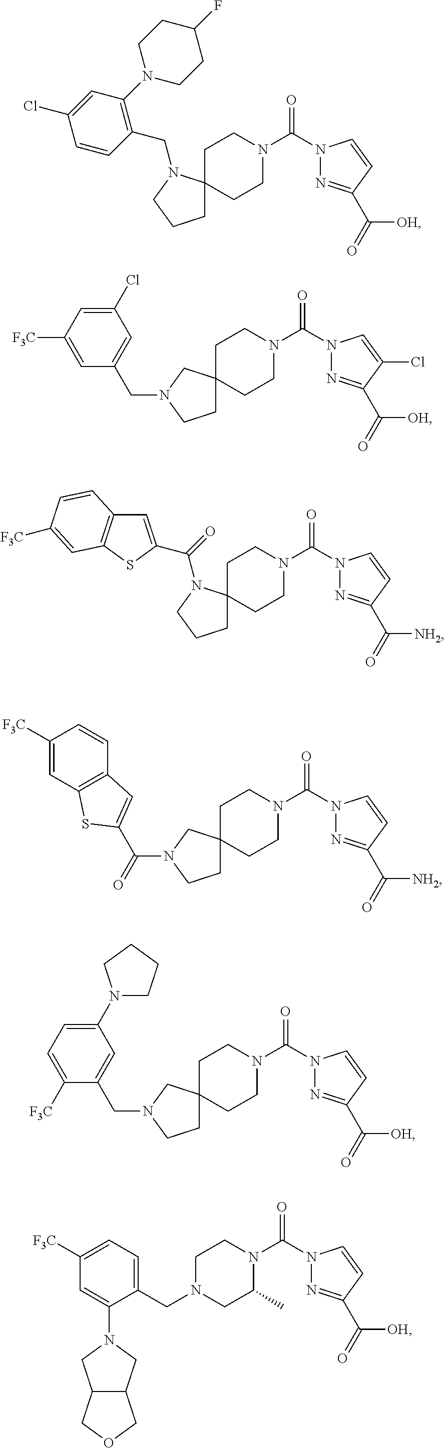 [SMILES: O=C(O)c1ccn(C(=O)N2CCC3(CCCN3Cc3ccc(Cl)cc3N3CCC(F)CC3)CC2)n1 ; O=C(O)c1nn(C(=O)N2CCC3(CCN(Cc4cc(Cl)cc(C(F)(F)F)c4)C3)CC2)cc1Cl ; NC(=O)c1ccn(C(=O)N2CCC3(CCCN3C(=O)c3cc4ccc(C(F)(F)F)cc4s3)CC2)n1 ; NC(=O)c1ccn(C(=O)N2CCC3(CCN(C(=O)c4cc5ccc(C(F)(F)F)cc5s4)C3)CC2)n1 ; O=C(O)c1ccn(C(=O)N2CCC3(CCN(Cc4cc(N5CCCC5)ccc4C(F)(F)F)C3)CC2)n1 ; C[C@@H]1CN(Cc2ccc(C(F)(F)F)cc2N2CC3COCC3C2)CCN1C(=O)n1ccc(C(=O)O)n1]

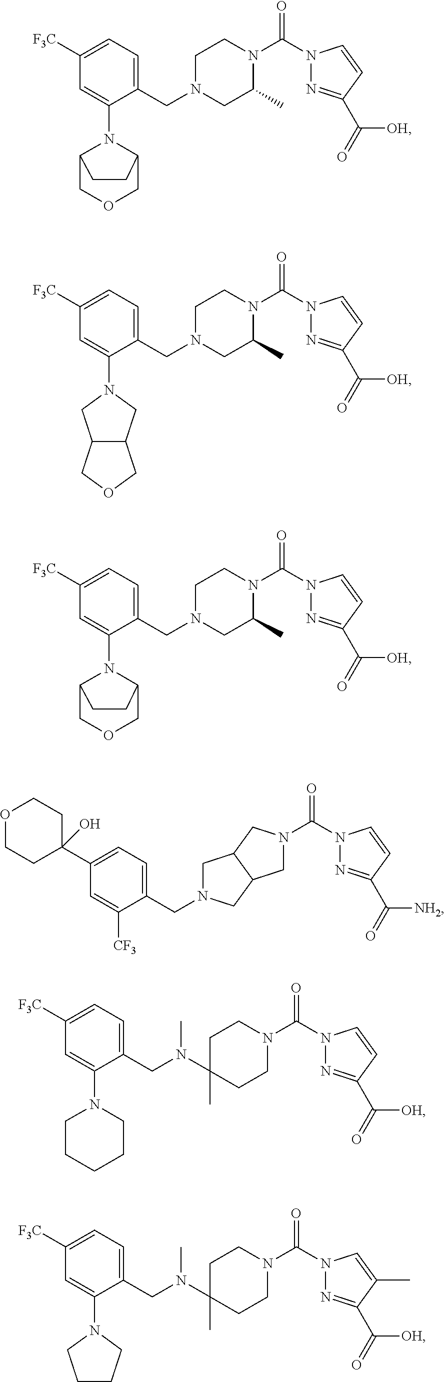 [SMILES: C[C@@H]1CN(Cc2ccc(C(F)(F)F)cc2N2C3CCC2COC3)CCN1C(=O)n1ccc(C(=O)O)n1 ; C[C@H]1CN(Cc2ccc(C(F)(F)F)cc2N2CC3COCC3C2)CCN1C(=O)n1ccc(C(=O)O)n1 ; C[C@H]1CN(Cc2ccc(C(F)(F)F)cc2N2C3CCC2COC3)CCN1C(=O)n1ccc(C(=O)O)n1 ; NC(=O)c1ccn(C(=O)N2CC3CN(Cc4ccc(C5(O)CCOCC5)cc4C(F)(F)F)CC3C2)n1 ; CN(Cc1ccc(C(F)(F)F)cc1N1CCCCC1)C1(C)CCN(C(=O)n2ccc(C(=O)O)n2)CC1 ; Cc1cn(C(=O)N2CCC(C)(N(C)Cc3ccc(C(F)(F)F)cc3N3CCCC3)CC2)nc1C(=O)O]

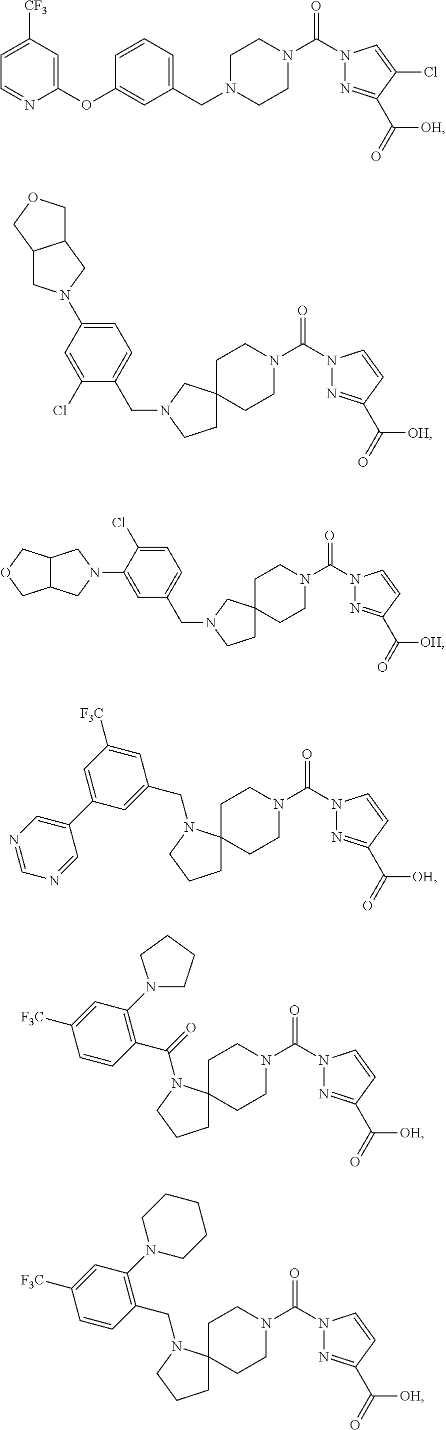 [SMILES: O=C(O)c1nn(C(=O)N2CCN(Cc3cccc(Oc4cc(C(F)(F)F)ccn4)c3)CC2)cc1Cl ; O=C(O)c1ccn(C(=O)N2CCC3(CCN(Cc4ccc(N5CC6COCC6C5)cc4Cl)C3)CC2)n1 ; O=C(O)c1ccn(C(=O)N2CCC3(CCN(Cc4ccc(Cl)c(N5CC6COCC6C5)c4)C3)CC2)n1 ; O=C(O)c1ccn(C(=O)N2CCC3(CCCN3Cc3cc(-c4cncnc4)cc(C(F)(F)F)c3)CC2)n1 ; O=C(O)c1ccn(C(=O)N2CCC3(CCCN3C(=O)c3ccc(C(F)(F)F)cc3N3CCCC3)CC2)n1 ; O=C(O)c1ccn(C(=O)N2CCC3(CCCN3Cc3ccc(C(F)(F)F)cc3N3CCCCC3)CC2)n1]

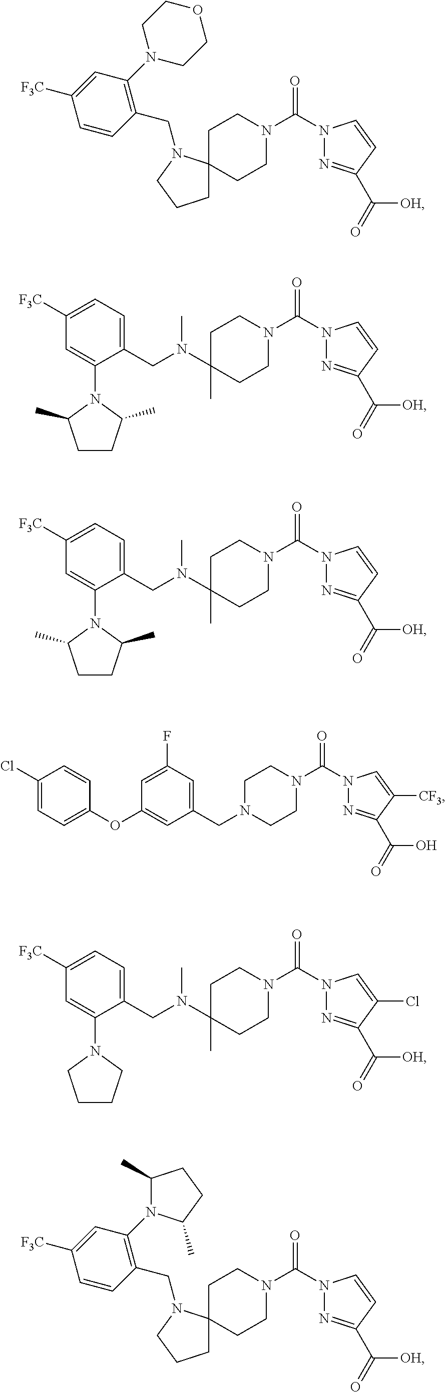 [SMILES: O=C(O)c1ccn(C(=O)N2CCC3(CCCN3Cc3ccc(C(F)(F)F)cc3N3CCOCC3)CC2)n1 ; C[C@@H]1CC[C@@H](C)N1c1cc(C(F)(F)F)ccc1CN(C)C1(C)CCN(C(=O)n2ccc(C(=O)O)n2)CC1 ; C[C@H]1CC[C@H](C)N1c1cc(C(F)(F)F)ccc1CN(C)C1(C)CCN(C(=O)n2ccc(C(=O)O)n2)CC1 ; O=C(O)c1nn(C(=O)N2CCN(Cc3cc(F)cc(Oc4ccc(Cl)cc4)c3)CC2)cc1C(F)(F)F ; CN(Cc1ccc(C(F)(F)F)cc1N1CCCC1)C1(C)CCN(C(=O)n2cc(Cl)c(C(=O)O)n2)CC1 ; C[C@H]1CC[C@H](C)N1c1cc(C(F)(F)F)ccc1CN1CCCC12CCN(C(=O)n1ccc(C(=O)O)n1)CC2]

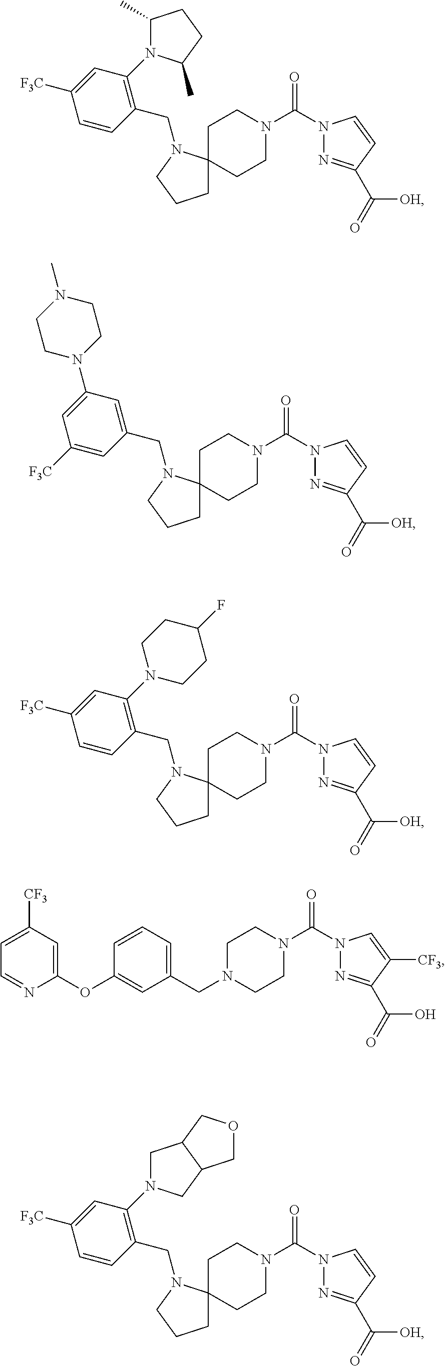 [SMILES: C[C@@H]1CC[C@@H](C)N1c1cc(C(F)(F)F)ccc1CN1CCCC12CCN(C(=O)n1ccc(C(=O)O)n1)CC2 ; CN1CCN(c2cc(CN3CCCC34CCN(C(=O)n3ccc(C(=O)O)n3)CC4)cc(C(F)(F)F)c2)CC1 ; O=C(O)c1ccn(C(=O)N2CCC3(CCCN3Cc3ccc(C(F)(F)F)cc3N3CCC(F)CC3)CC2)n1 ; O=C(O)c1nn(C(=O)N2CCN(Cc3cccc(Oc4cc(C(F)(F)F)ccn4)c3)CC2)cc1C(F)(F)F ; O=C(O)c1ccn(C(=O)N2CCC3(CCCN3Cc3ccc(C(F)(F)F)cc3N3CC4COCC4C3)CC2)n1]

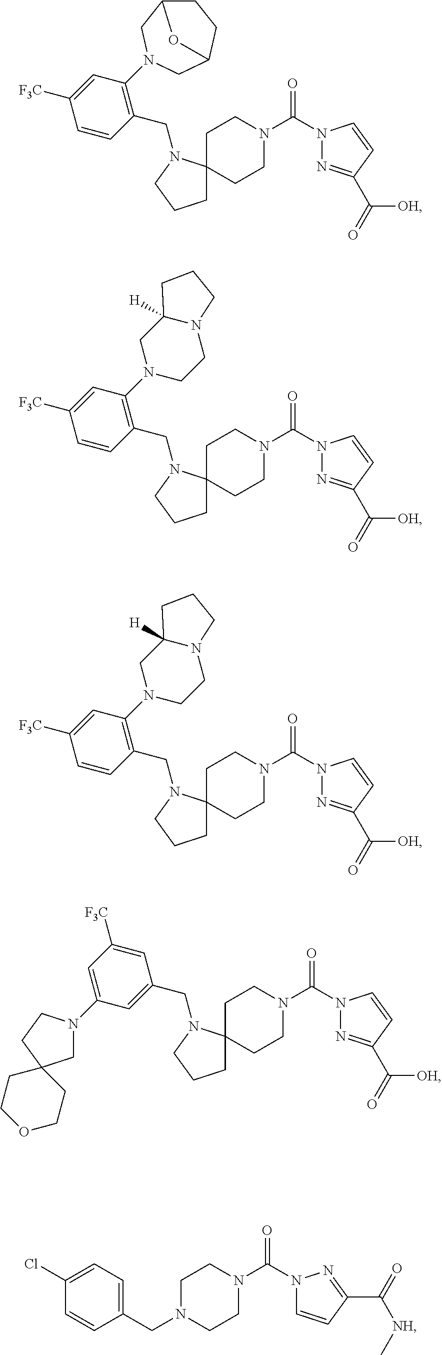 [SMILES: O=C(O)c1ccn(C(=O)N2CCC3(CCCN3Cc3ccc(C(F)(F)F)cc3N3CC4CCC(C3)O4)CC2)n1 ; O=C(O)c1ccn(C(=O)N2CCC3(CCCN3Cc3ccc(C(F)(F)F)cc3N3CCN4CCC[C@@H]4C3)CC2)n1 ; O=C(O)c1ccn(C(=O)N2CCC3(CCCN3Cc3ccc(C(F)(F)F)cc3N3CCN4CCC[C@H]4C3)CC2)n1 ; O=C(O)c1ccn(C(=O)N2CCC3(CCCN3Cc3cc(N4CCC5(CCOCC5)C4)cc(C(F)(F)F)c3)CC2)n1 ; CNC(=O)c1ccn(C(=O)N2CCN(Cc3ccc(Cl)cc3)CC2)n1]

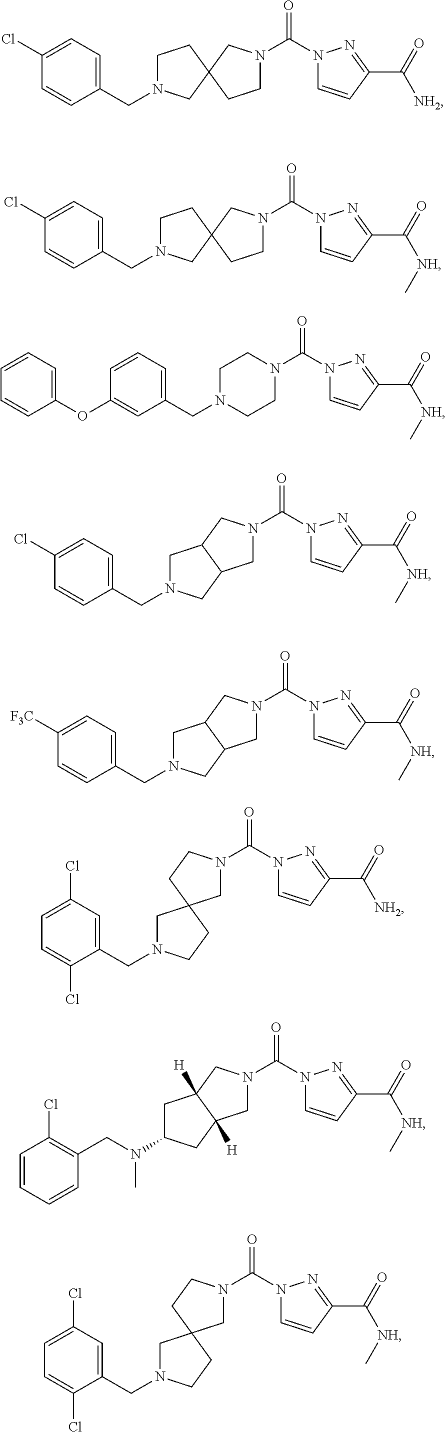

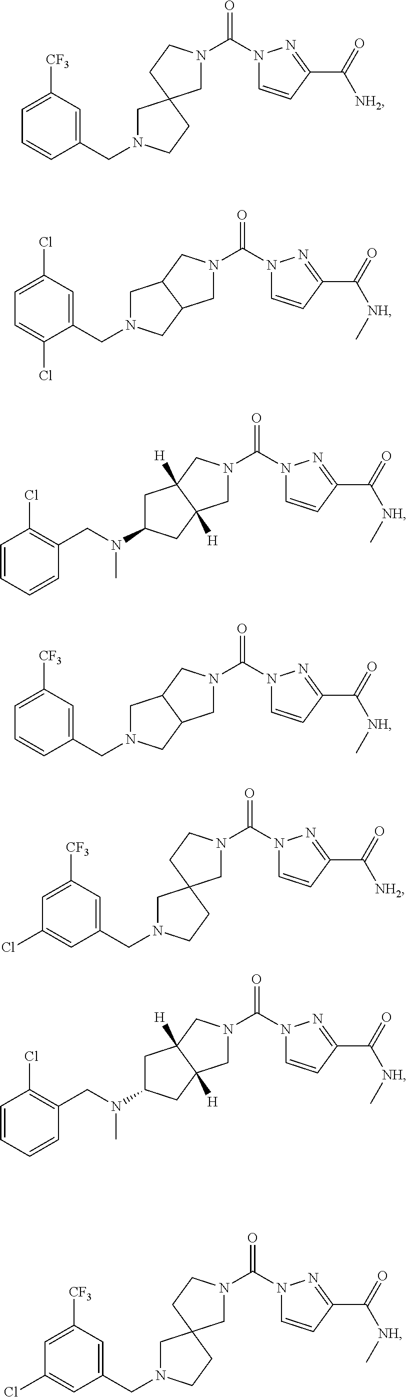

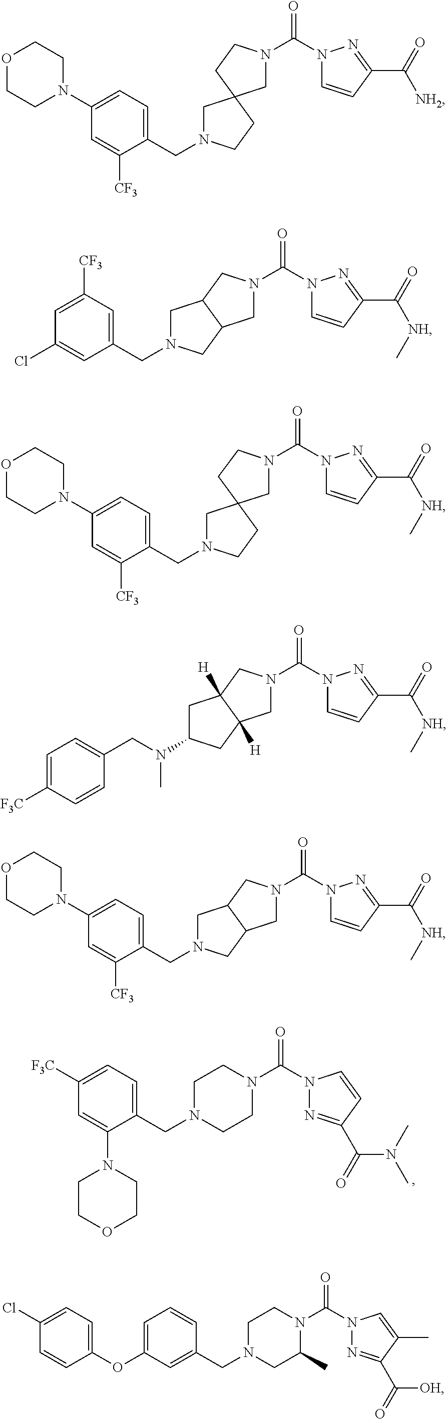

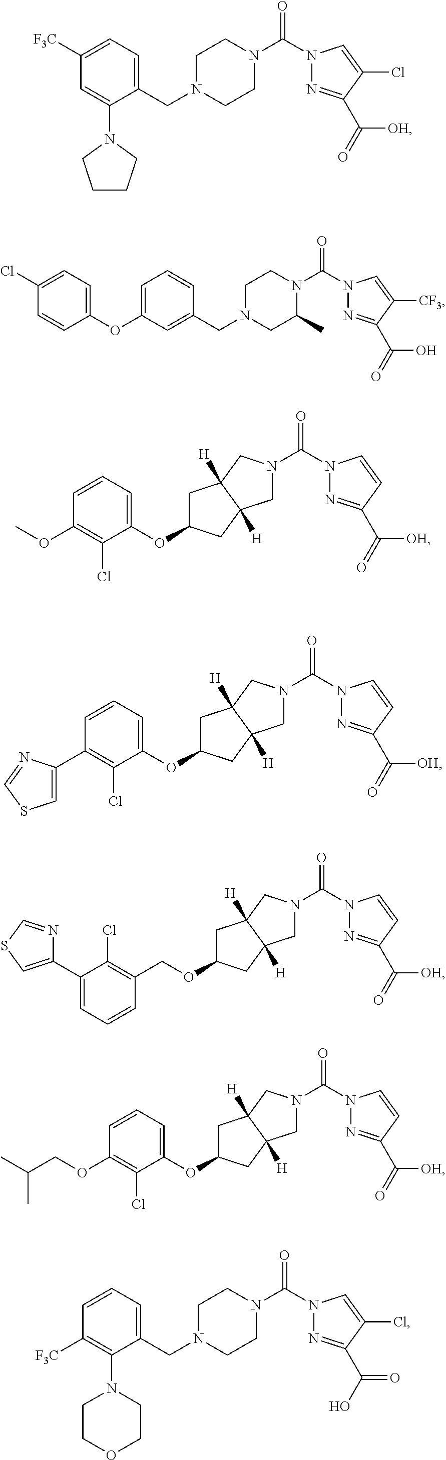

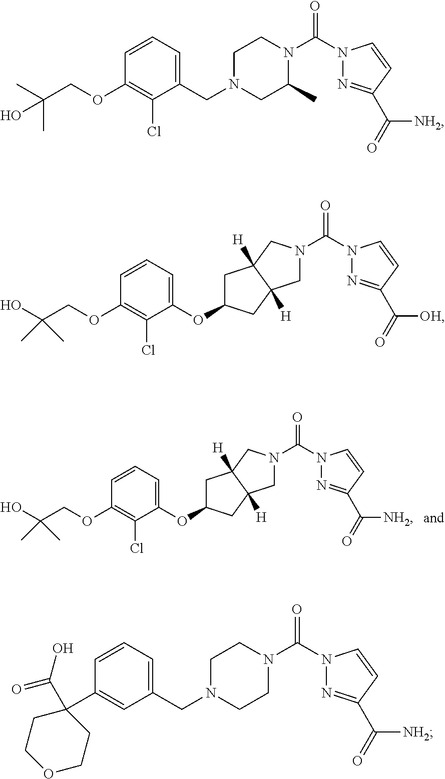 [SMILES: C[C@H]1CN(Cc2cccc(OCC(C)(C)O)c2Cl)CCN1C(=O)n1ccc(C(N)=O)n1 ; CC(C)(O)COc1cccc(O[C@H]2C[C@@H]3CN(C(=O)n4ccc(C(=O)O)n4)C[C@@H]3C2)c1Cl ; CC(C)(O)COc1cccc(O[C@H]2C[C@@H]3CN(C(=O)n4ccc(C(N)=O)n4)C[C@@H]3C2)c1Cl ; NC(=O)c1ccn(C(=O)N2CCN(Cc3cccc(C4(C(=O)O)CCOCC4)c3)CC2)n1]

or a solvate, hydrate, tautomer, N-oxide, stereoisomer, or pharmaceutically acceptable salt thereof.

E68: The method of any one of embodiments 1-67, wherein the disease or condition is atopic dermatitis.

E69: The method of any one of embodiments 1-67, wherein the disease or condition is bladder dysfunction associated with multiple sclerosis.

E70: The method of any one of embodiments 1-67, wherein the disease or condition is cardiovascular disease.

E71: The method of any one of embodiments 1-67, wherein the disease or condition is contact dermatitis.

E72: The method of any one of embodiments 1-67, wherein the disease or condition is cystic fibrosis.

E73: The method of any one of embodiments 1-67, wherein the disease or condition is dermatomyositis.

E74: The method of any one of embodiments 1-67, wherein the disease or condition is eczema.

E75: The method of any one of embodiments 1-67, wherein the disease or condition is endometriosis.

E76: The method of any one of embodiments 1-67, wherein the disease or condition is enteritis.

E77: The method of any one of embodiments 1-67, wherein the disease or condition is fibromyalgia.

E78: The method of any one of embodiments 1-67, wherein the disease or condition is Tourette syndrome.

E79: The method of any one of embodiments 1-67, wherein the disease or condition is inflammatory bowel disease.

E80: The method of any one of embodiments 1-67, wherein the disease or condition is interstitial cystitis.

E81: The method of any one of embodiments 1-67, wherein the disease or condition is irritable bowel syndrome.

E82: The method of any one of embodiments 1-67, wherein the disease or condition is ischemia.

E83: The method of any one of embodiments 1-67, wherein the disease or condition is labor.

E84: The method of any one of embodiments 1-67, wherein the disease or condition is metabolic disorders.

E85: The method of any one of embodiments 1-67, wherein the disease or condition is musculoskeletal diseases.

E86: The method of any one of embodiments 1-67, wherein the disease or condition is neuropathy.

E87: The method of any one of embodiments 1-67, wherein the disease or condition is osteoarthritis.

E88: The method of any one of embodiments 1-67, wherein the disease or condition is pancreatitis.

E89: The method of any one of embodiments 1-67, wherein the disease or condition is pharyngitis.

E90: The method of any one of embodiments 1-67, wherein the disease or condition is post trigeminal neuralgia.

E91: The method of any one of embodiments 1-67, wherein the disease or condition is renal ischemia.

E92: The method of any one of embodiments 1-67, wherein the disease or condition is rheumatoid arthritis.

E93: The method of any one of embodiments 1-67, wherein the disease or condition is skeletal muscle contusion.

E94: The method of any one of embodiments 1-67, wherein the disease or condition is skin diseases.

E95: The method of any one of embodiments 1-67, wherein the disease or condition is sunburn.

E96: The method of any one of embodiments 1-67, wherein the disease or condition is systemic lupus erythematosus.

E97: The method of any one of embodiments 1-67, wherein the disease or condition is pain selected from acute pain, back pain, cancer pain, chest pain, functional chest pain, joint pain, menstrual pain, abdominal pain, abdominal pain associated with irritable bowel syndrome, post mastectomy pain syndrome, post operative pain, toothache, vasoocclusive painful crises in sickle cell disease, and visceral pain.

### Methods

In some embodiments disclosed herein are methods of modulating the activity of MAGL. Contemplated methods, for example, comprise exposing said enzyme to a compound described herein. The ability of compounds described herein to modulate or inhibit MAGL is evaluated by procedures known in the art and/or described herein. Another aspect of this disclosure provides methods of treating a disease associated with expression or activity of MAGL in a patient.

Compounds described herein which are modulators of MAGL. In some embodiments, these compounds and pharmaceutical compositions comprising these compounds, are useful for the treatment of atopic dermatitis, bladder dysfunction associated with multiple sclerosis, cardiovascular disease, contact dermatitis, cystic fibrosis, dermatomyositis, eczema, endometriosis, enteritis, fibromyalgia, Tourette syndrome, inflammatory bowel disease, interstitial cystitis, irritable bowel syndrome, ischemia, labor, abdominal pain, abdominal pain associated with irritable bowel syndrome, acute pain, back pain, cancer pain, chest pain, functional chest pain, joint pain, menstrual pain, metabolic disorders, musculoskeletal diseases, neuropathy, osteoarthritis, pancreatitis, pharyngitis, post mastectomy pain syndrome, post trigeminal neuralgia, post operative pain, renal ischemia, rheumatoid arthritis, skeletal muscle contusion, skin diseases, sunburn, systemic lupus erythematosus, toothache, vasoocclusive painful crises in sickle cell disease, and visceral pain.

In some embodiments is a method for treating a disease or a condition in a patient wherein the disease or condition is selected from atopic dermatitis, bladder dysfunction associated with multiple sclerosis, cardiovascular disease, contact dermatitis, cystic fibrosis, dermatomyositis, eczema, endometriosis, enteritis, fibromyalgia, Tourette syndrome, inflammatory bowel disease, interstitial cystitis, irritable bowel syndrome, ischemia, labor, abdominal pain, abdominal pain associated with irritable bowel syndrome, acute pain, back pain, cancer pain, chest pain, functional chest pain, joint pain, menstrual pain, metabolic disorders, musculoskeletal diseases, neuropathy, osteoarthritis, pancreatitis, pharyngitis, post mastectomy pain syndrome, post trigeminal neuralgia, post operative pain, renal ischemia, rheumatoid arthritis, skeletal muscle contusion, skin diseases, sunburn, systemic lupus erythematosus, toothache, vasoocclusive painful crises in sickle cell disease, and visceral pain, comprising administering to the patient in need thereof a therapeutically effective amount of a compound of Formula (I) having the structure:

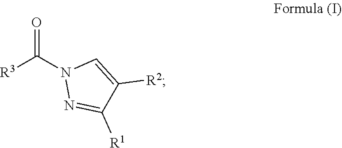

wherein:
R¹ is —C(O)OR¹⁵ or —C(O)NR¹⁰R¹¹;
R² is H, halogen, C₁₋₆alkyl, or C₁₋₆haloalkyl;
R³ is

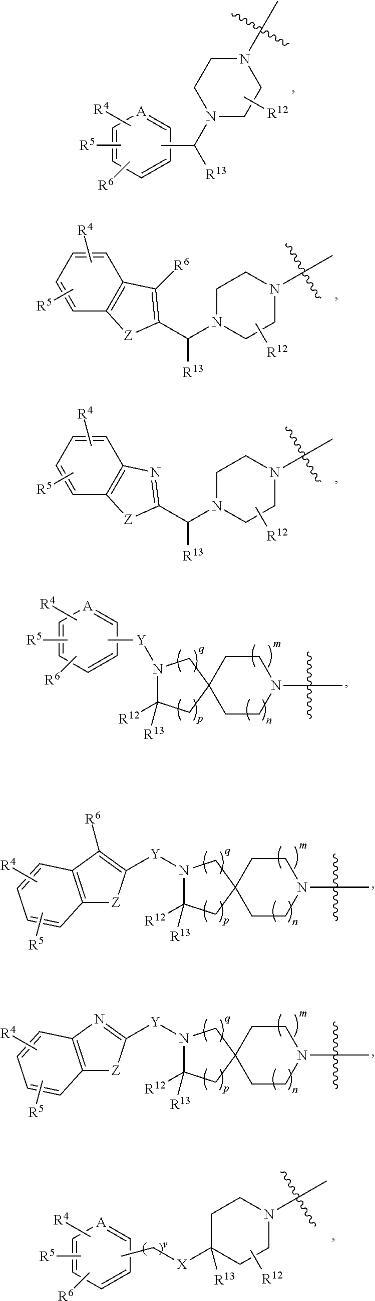

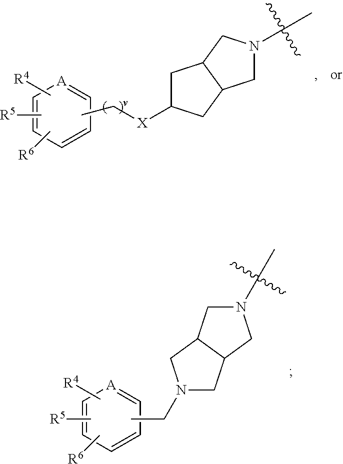

A is N or C(H);
X is —O—, —N(R¹⁶)—, or —CH₂N(R¹⁶)CH₂—;
Y is —CH₂— or —C(O)—;
Z is —S—, —O—, or —N(R¹⁸)—;
R⁴ is H, halogen, —OR⁷, C₁₋₆alkyl, C₂₋₆alkenyl, C₂₋₆alkynyl, C₁₋₆haloalkyl, —C(O)NR⁸R⁹, C₃₋₈cycloalkyl, C₂₋₉heterocycloalkyl, —C₁₋₆alkyl-C₂₋₉heterocycloalkyl, C₆₋₁₀aryl, or C₁₋₉heteroaryl, wherein C₃₋₈cycloalkyl, C₂₋₉heterocycloalkyl, —C₁₋₆alkyl-C₂₋₉ heterocycloalkyl, C₆₋₁₀aryl, or C₁₋₉heteroaryl are optionally substituted with 1 or 2 R¹⁴;
R⁵ is H, halogen, C₁₋₆alkyl, C₁₋₆haloalkyl, C₁₋₆haloalkoxy, or phenyl;
R⁶ is H, halogen or C₁₋₆alkyl;
R⁷ is H, C₁₋₆alkyl, C₁₋₆haloalkyl, —C₁₋₆alkyl-OH, C₂₋₉heterocycloalkyl, C₆₋₁₀aryl, or C₁₋₉heteroaryl, wherein C₂₋₉heterocycloalkyl, C₆₋₁₀aryl, or C₁₋₉heteroaryl are optionally substituted with 1 or 2 R¹⁴;
each R⁸ and each R⁹ are independently selected from H and C₁₋₆alkyl; or R⁸ and R⁹ together with the nitrogen to which they are attached are combined to form a heterocycloalkyl ring;
R¹⁰ and R¹¹ are each independently H or C₁₋₆alkyl;
R¹² is H, halogen, or C₁₋₆alkyl;
R¹³ is H or C₁₋₆alkyl;
each R¹⁴ is independently selected from halogen, —OH, C₁₋₆alkyl, C₁₋₆haloalkyl, C₁₋₆alkoxy, —C₁₋₆alkyl-OH, C₃₋₈cycloalkyl, —C(O)OH, —C(O)NR⁸R⁹, —SO₂—C₁₋₆alkyl, and —N(R¹⁷)C(O)—C₁₋₆alkyl;
R¹⁵ is H or C₁₋₆alkyl;
R¹⁶ is H, C₁₋₆alkyl, —C(O)—C₁₋₆alkyl, —C₁₋₆alkyl-OH, or —CH₂CO₂H;
R¹⁷ is H or C₁₋₆alkyl;
R¹⁸ is H or C₁₋₆alkyl;
v is 0 or 1;
n is 0 or 1;
m is 0 or 1;
p is 0, 1, or 2; and
q is 0, 1 or 2;
or a solvate, hydrate, tautomer, N-oxide, stereoisomer, or pharmaceutically acceptable salt thereof.

In some embodiments of the methods for treating a disease or condition with a compound of Formula (I), R¹ is —C(O)OR¹⁵. In some embodiments of the methods for treating a disease or condition with a compound of Formula (I), R¹ is —C(O)OR¹⁵ and R¹⁵ is H. In some embodiments of the methods for treating a disease or condition with a compound of Formula (I), R¹ is —C(O)OR¹⁵ and R¹⁵ is C₁₋₆alkyl. In some embodiments of the methods for treating a disease or condition with a compound of Formula (I), R¹ is —C(O)OR¹⁵ and R¹⁵ is —CH₃. In some embodiments of the methods for treating a disease or condition with a compound of Formula (I), R¹ is —C(O)NR¹⁰R¹¹. In some embodiments of the methods for treating a disease or condition with a compound of Formula (I), R¹ is —C(O)NR¹⁰R¹¹, R¹⁰ is H, and R¹¹ is H. In some embodiments of the methods for treating a disease or condition with a compound of Formula (I), R¹ is —C(O)NR¹⁰R¹¹, R¹⁰ is H, and R¹¹ is C₁₋₆alkyl. In some embodiments of the methods for treating a disease or condition with a compound of Formula (I), R¹ is —C(O)NR¹⁰R¹¹, R¹⁰ is H, and R¹¹ is —CH₃. In some embodiments of the methods for treating a disease or condition with a compound of Formula (I), R¹ is —C(O)NR¹⁰R¹¹, R¹⁰ is C₁₋₆alkyl, and R¹¹ is C₁₋₆alkyl. In some embodiments of the methods for treating a disease or condition with a compound of Formula (I), R¹ is —C(O)NR¹⁰R¹¹, R¹⁰ is —CH₃, and R¹¹ is —CH₃.

In some embodiments of the methods for treating a disease or condition with a compound of Formula (I), R² is H. In some embodiments of the methods for treating a disease or condition with a compound of Formula (I), R² is halogen. In some embodiments of the methods for treating a disease or condition with a compound of Formula (I), R² is —Cl. In some embodiments of the methods for treating a disease or condition with a compound of Formula (I), R² is C₁₋₆alkyl. In some embodiments of the methods for treating a disease or condition with a compound of Formula (I), R² is —CH₃. In some embodiments of the methods for treating a disease or condition with a compound of Formula (I), R² is C₁₋₆haloalkyl. In some embodiments of the methods for treating a disease or condition with a compound of Formula (I), R² is —CF₃.

In some embodiments of the methods for treating a disease or condition with a compound of Formula (I), R³ is

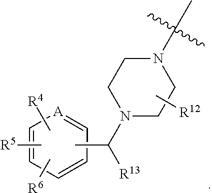

In some embodiments of the methods for treating a disease or condition with a compound of Formula (I), R³ is

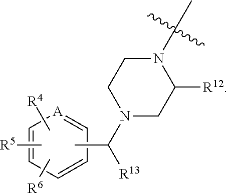

In some embodiments of the methods for treating a disease or condition with a compound of Formula (I), R³ is

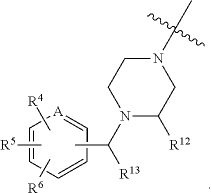

In some embodiments of the methods for treating a disease or condition with a compound of Formula (I), A is N. In some embodiments of the methods for treating a disease or condition with a compound of Formula (I), A is C(H). In some embodiments of the methods for treating a disease or condition with a compound of Formula (I), R³ is

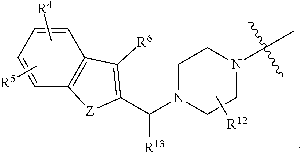

In some embodiments of the methods for treating a disease or condition with a compound of Formula (I), R³ is

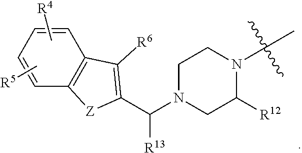

In some embodiments of the methods for treating a disease or condition with a compound of Formula (I), R³ is

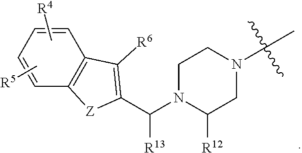

In some embodiments of the methods for treating a disease or condition with a compound of Formula (I), R³ is

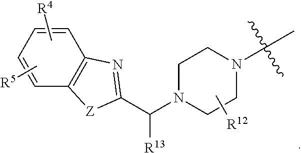

In some embodiments of the methods for treating a disease or condition with a compound of Formula (I), R³ is

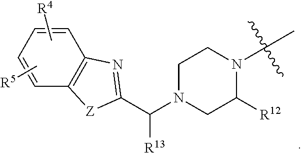

In some embodiments of the methods for treating a disease or condition with a compound of Formula (I), R³ is

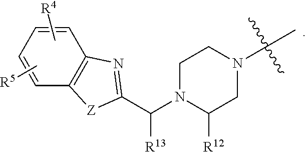

In some embodiments of the methods for treating a disease or condition with a compound of Formula (I), Z is —S—. In some embodiments of the methods for treating a disease or condition with a compound of Formula (I), Z is —O—. In some embodiments of the methods for treating a disease or condition with a compound of Formula (I), Z is —N(R¹⁸)—. In some embodiments of the methods for treating a disease or condition with a compound of Formula (I), Z is —N(H)—. In some embodiments of the methods for treating a disease or condition with a compound of Formula (I), Z is —N(CH₃)—. In some embodiments of the methods for treating a disease or condition with a compound of Formula (I), R¹² is H and R¹³ is H. In some embodiments of the methods for treating a disease or condition with a compound of Formula (I), R¹² is C₁₋₆alkyl and R¹³ is H. In some embodiments of the methods for treating a disease or condition with a compound of Formula (I), R¹² is —CH₃ and R¹³ is H. In some embodiments of the methods for treating a disease or condition with a compound of Formula (I), R¹² is H and R¹³ is C₁₋₆alkyl. In some embodiments of the methods for treating a disease or condition with a compound of Formula (I), R¹² is H and R¹³ is —CH₃. In some embodiments of the methods for treating a disease or condition with a compound of Formula (I), R¹² is C₁₋₆alkyl and R¹³ is C₁₋₆alkyl. In some embodiments of the methods for treating a disease or condition with a compound of Formula (I), R¹² is —CH₃ and R¹³ is —CH₃.

In some embodiments of the methods for treating a disease or condition with a compound of Formula (I), R is

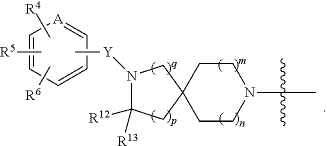

In some embodiments of the methods for treating a disease or condition with a compound of Formula (I), A is N. In some embodiments of the methods for treating a disease or condition with a compound of Formula (I), A is C(H). In some embodiments of the methods for treating a disease or condition with a compound of Formula (I), R³ is

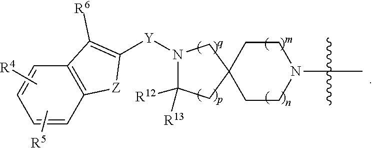

In some embodiments of the methods for treating a disease or condition with a compound of Formula (I), R³ is

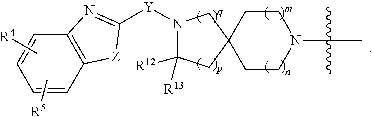

In some embodiments of the methods for treating a disease or condition with a compound of Formula (I), Z is —S—. In some embodiments of the methods for treating a disease or condition with a compound of Formula (I), Z is —O—. In some embodiments of the methods for treating a disease or condition with a compound of Formula (I), Z is —N(R¹⁸)—. In some embodiments of the methods for treating a disease or condition with a compound of Formula (I), Z is —N(H)—. In some embodiments of the methods for treating a disease or condition with a compound of Formula (I), Z is —N(CH₃)—. In some embodiments of the methods for treating a disease or condition with a compound of Formula (I), Y is —CH₂—. In some embodiments of the methods for treating a disease or condition with a compound of Formula (I), Y is —C(O)—. In some embodiments of the methods for treating a disease or condition with a compound of Formula (I), m is 1, n is 1, q is 1, and p is 1. In some embodiments of the methods for treating a disease or condition with a compound of Formula (I), m is 0, n is 1, q is 2, and p is 1. In some embodiments of the methods for treating a disease or condition with a compound of Formula (I), m is 1, n is 1, q is 0, and p is 2. In some embodiments of the methods for treating a disease or condition with a compound of Formula (I), m is 1, n is 1, q is 1, and p is 0. In some embodiments of the methods for treating a disease or condition with a compound of Formula (I), m is 0, n is 0, q is 2, and p is 1. In some embodiments of the methods for treating a disease or condition with a compound of Formula (I), R¹² is H and R¹³ is H. In some embodiments of the methods for treating a disease or condition with a compound of Formula (I), R¹² is C₁₋₆alkyl and R¹³ is H. In some embodiments of the methods for treating a disease or condition with a compound of Formula (I), R¹² is —CH₃ and R¹³ is H. In some embodiments of the methods for treating a disease or condition with a compound of Formula (I), R¹² is H and R¹³ is C₁₋₆alkyl. In some embodiments of the methods for treating a disease or condition with a compound of Formula (I), R¹² is H and R¹³ is —CH₃. In some embodiments of the methods for treating a disease or condition with a compound of Formula (I), R¹² is C₁₋₆alkyl and R¹³ is C₁₋₆alkyl. In some embodiments of the methods for treating a disease or condition with a compound of Formula (I), R¹² is —CH₃ and R¹³ is-CH₃.

In some embodiments of the methods for treating a disease or condition with a compound of Formula (I), R³ is

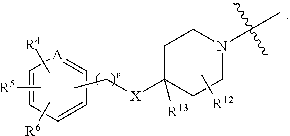

In some embodiments of the methods for treating a disease or condition with a compound of Formula (I), R¹² is H. In some embodiments of the methods for treating a disease or condition with a compound of Formula (I), R¹² is halogen. In some embodiments of the methods for treating a disease or condition with a compound of Formula (I), R¹² is F. In some embodiments of the methods for treating a disease or condition with a compound of Formula (I), R¹² is C₁₋₆alkyl. In some embodiments of the methods for treating a disease or condition with a compound of Formula (I), R¹² is —CH₃. In some embodiments of the methods for treating a disease or condition with a compound of Formula (I), R¹² is H and R¹³ is H. In some embodiments of the methods for treating a disease or condition with a compound of Formula (I), R¹² is C₁₋₆alkyl and R¹³ is H. In some embodiments of the methods for treating a disease or condition with a compound of Formula (I), R¹² is —CH₃ and R¹³ is H. In some embodiments of the methods for treating a disease or condition with a compound of Formula (I), R¹² is H and R¹³ is C₁₋₆alkyl. In some embodiments of the methods for treating a disease or condition with a compound of Formula (I), R¹² is H and R¹³ is —CH₃. In some embodiments of the methods for treating a disease or condition with a compound of Formula (I), R¹² is C₁₋₆alkyl and R¹³ is C₁₋₆alkyl. In some embodiments of the methods for treating a disease or condition with a compound of Formula (I), R¹² is —CH₃ and R¹³ is —CH₃. In some embodiments of the methods for treating a disease or condition with a compound of Formula (I), R³ is

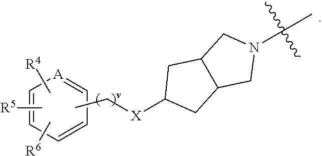

In some embodiments of the methods for treating a disease or condition with a compound of Formula (I), X is —O—. In some embodiments of the methods for treating a disease or condition with a compound of Formula (I), X is —N(R¹⁶)—. In some embodiments of the methods for treating a disease or condition with a compound of Formula (I), X is —N(H)—. In some embodiments of the methods for treating a disease or condition with a compound of Formula (I), X is —N(CH₃)—. In some embodiments of the methods for treating a disease or condition with a compound of Formula (I), X is —N(R¹⁶)— and R¹⁶ is —C₁₋₆alkyl-OH. In some embodiments of the methods for treating a disease or condition with a compound of Formula (I), X is —CH₂N(R¹⁶)CH₂—. In some embodiments of the methods for treating a disease or condition with a compound of Formula (I), X is —CH₂N(H)CH₂—. In some embodiments of the methods for treating a disease or condition with a compound of Formula (I), X is —CH₂N(CH₃)CH₂—. In some embodiments of the methods for treating a disease or condition with a compound of Formula (I), A is N. In some embodiments of the methods for treating a disease or condition with a compound of Formula (I), A is C(H). In some embodiments of the methods for treating a disease or condition with a compound of Formula (I), v is 0. In some embodiments of the methods for treating a disease or condition with a compound of Formula (I), v is 1. In some embodiments of the methods for treating a disease or condition with a compound of Formula (I), R³ is

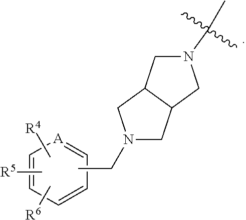

In some embodiments of the methods for treating a disease or condition with a compound of Formula (I), R⁴ is halogen, —OR⁷, C₁₋₆alkyl, C₂₋₆alkynyl, C₁₋₆haloalkyl, —C(O)NR⁸R⁹, C₃₋₈cycloalkyl, C₂₋₉heterocycloalkyl, C₆₋₁₀aryl, or C₁₋₉heteroaryl, wherein C₂₋₉heterocycloalkyl, —C₁₋₆alkyl-C₂₋₉heterocycloalkyl, C₆₋₁₀aryl, or C₁₋₉heteroaryl are optionally substituted with 1 or 2 R¹⁴. In some embodiments of the methods for treating a disease or condition with a compound of Formula (I), R⁴ is halogen, —OR⁷, C₁₋₆haloalkyl, C₂₋₉heterocycloalkyl, C₆₋₁₀aryl, or C₁₋₉heteroaryl, wherein C₂₋₉ heterocycloalkyl, C₆₋₁₀aryl, or C₁₋₉heteroaryl are optionally substituted with 1 or 2 R¹⁴. In some embodiments of the methods for treating a disease or condition with a compound of Formula (I), R⁴ is halogen. In some embodiments of the methods for treating a disease or condition with a compound of Formula (I), R⁴ is —Cl. In some embodiments of the methods for treating a disease or condition with a compound of Formula (I), R⁴ is C₁₋₆haloalkyl. In some embodiments of the methods for treating a disease or condition with a compound of Formula (I), R⁴ is —CF₃. In some embodiments of the methods for treating a disease or condition with a compound of Formula (I), R⁴ is —OR⁷. In some embodiments of the methods for treating a disease or condition with a compound of Formula (I), R⁴ is —OR⁷ and R⁷ is C₁₋₆haloalkyl, —C₁₋₆alkyl-OH, C₆₋₁₀aryl, or C₁₋₉heteroaryl, wherein C₆₋₁₀aryl or C₁₋₉heteroaryl are optionally substituted with 1 or 2 R¹⁴. In some embodiments of the methods for treating a disease or condition with a compound of Formula (I), R⁴ is —OR⁷ and R⁷ is C₁₋₆haloalkyl, C₆₋₁₀aryl, or C₁₋₉heteroaryl, wherein C₆₋₁₀aryl or C₁₋₉heteroaryl are optionally substituted with 1 or 2 R¹⁴. In some embodiments of the methods for treating a disease or condition with a compound of Formula (I), R⁴ is —OR⁷ and R⁷ is C₁₋₆haloalkyl. In some embodiments of the methods for treating a disease or condition with a compound of Formula (I), R⁴ is —OR⁷ and R⁷ is —CF₃. In some embodiments of the methods for treating a disease or condition with a compound of Formula (I), R⁴ is —OR⁷ and R⁷ is —C₁₋₆alkyl-OH. In some embodiments of the methods for treating a disease or condition with a compound of Formula (I), R⁴ is —OR⁷ and R⁷ is C₆₋₁₀aryl or C₁₋₉heteroaryl, wherein C₆₋₁₀aryl or C₁₋₉heteroaryl are optionally substituted with 1 or 2 R¹⁴. In some embodiments of the methods for treating a disease or condition with a compound of Formula (I), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are optionally substituted with halogen or C₁₋₆haloalkyl. In some embodiments of the methods for treating a disease or condition with a compound of Formula (I), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are unsubstituted. In some embodiments of the methods for treating a disease or condition with a compound of Formula (I), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are substituted with halogen or C₁₋₆haloalkyl. In some embodiments of the methods for treating a disease or condition with a compound of Formula (I), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are substituted with —Cl or —CF₃. In some embodiments of the methods for treating a disease or condition with a compound of Formula (I), R⁴ is C₃₋₈cycloalkyl optionally substituted with 1 or 2 R¹⁴. In some embodiments of the methods for treating a disease or condition with a compound of Formula (I), R⁴ is an unsubstituted C₃₋₈cycloalkyl. In some embodiments of the methods for treating a disease or condition with a compound of Formula (I), R⁴ is C₂₋₉heterocycloalkyl optionally substituted with 1 or 2 R¹⁴. In some embodiments of the methods for treating a disease or condition with a compound of Formula (I), R⁴ is an unsubstituted C₂₋₉heterocycloalkyl. In some embodiments of the methods for treating a disease or condition with a compound of Formula (I), R⁴ is C₂₋₉heterocycloalkyl substituted with 1 or 2 R¹⁴. In some embodiments of the methods for treating a disease or condition with a compound of Formula (I), R⁴ is

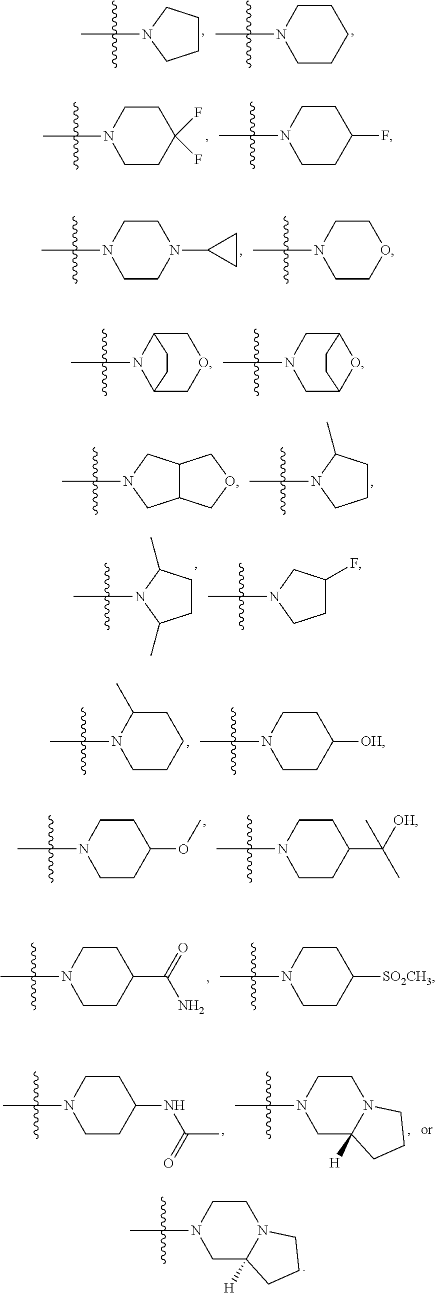

In some embodiments of the methods for treating a disease or condition with a compound of Formula (I), R⁴ is C₁₋₉heteroaryl optionally substituted with 1 or 2 R¹⁴. In some embodiments of the methods for treating a disease or condition with a compound of Formula (I), R⁴ is an unsubstituted C₁₋₉heteroaryl. In some embodiments of the methods for treating a disease or condition with a compound of Formula (I), R⁴ is C₂₋₉heteroaryl substituted with 1 or 2 R¹⁴. In some embodiments of the methods for treating a disease or condition with a compound of Formula (I), R⁴ is

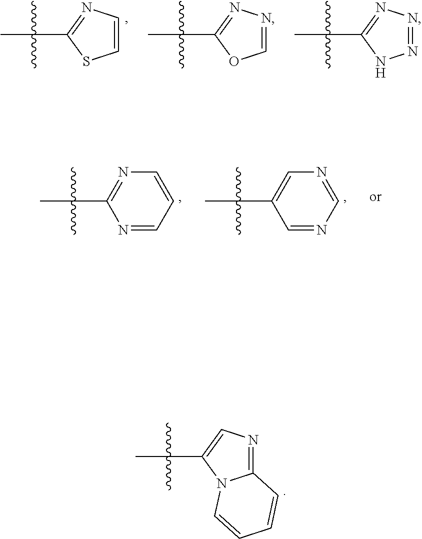 [SMILES: *c1nccs1 ; *c1nnco1 ; *c1nnn[nH]1 ; *c1ncccn1 ; *c1cncnc1 ; *c1cnc2ccccn12]

In some embodiments of the methods for treating a disease or condition with a compound of Formula (I), R⁵ is H, halogen, C₁₋₆alkyl, C₁₋₆haloalkyl, C₁₋₆haloalkoxy. In some embodiments of the methods for treating a disease or condition with a compound of Formula (I), R⁵ is H. In some embodiments of the methods for treating a disease or condition with a compound of Formula (I), R⁵ is halogen. In some embodiments of the methods for treating a disease or condition with a compound of Formula (I), R⁵ is —Cl. In some embodiments of the methods for treating a disease or condition with a compound of Formula (I), R⁵ is —F. In some embodiments of the methods for treating a disease or condition with a compound of Formula (I), R⁵ is C₁₋₆alkyl. In some embodiments of the methods for treating a disease or condition with a compound of Formula (I), R⁵ is —CH₃. In some embodiments of the methods for treating a disease or condition with a compound of Formula (I), R⁵ is C₁₋₆haloalkyl. In some embodiments of the methods for treating a disease or condition with a compound of Formula (I), R⁵ is —CF₃. In some embodiments of the methods for treating a disease or condition with a compound of Formula (I), R⁵ is C₁₋₆haloalkoxy. In some embodiments of the methods for treating a disease or condition with a compound of Formula (I), R⁵ is —OCF₃.

In some embodiments of the methods for treating a disease or condition with a compound of Formula (I), R⁶ is H. In some embodiments of the methods for treating a disease or condition with a compound of Formula (I), R⁶ is halogen. In some embodiments of the methods for treating a disease or condition with a compound of Formula (I), R⁶ is —Cl. In some embodiments of the methods for treating a disease or condition with a compound of Formula (I), R⁶ is —F. In some embodiments of the methods for treating a disease or condition with a compound of Formula (I), R⁶ is C₁₋₆alkyl. In some embodiments of the methods for treating a disease or condition with a compound of Formula (I), R⁶ is —CH₃.

In some embodiments is a method for treating a disease or a condition in a patient wherein the disease or condition is selected from atopic dermatitis, bladder dysfunction associated with multiple sclerosis, cardiovascular disease, contact dermatitis, cystic fibrosis, dermatomyositis, eczema, endometriosis, enteritis, fibromyalgia, Tourette syndrome, inflammatory bowel disease, interstitial cystitis, irritable bowel syndrome, ischemia, labor, abdominal pain, abdominal pain associated with irritable bowel syndrome, acute pain, back pain, cancer pain, chest pain, functional chest pain, joint pain, menstrual pain, metabolic disorders, musculoskeletal diseases, neuropathy, osteoarthritis, pancreatitis, pharyngitis, post mastectomy pain syndrome, post trigeminal neuralgia, post operative pain, renal ischemia, rheumatoid arthritis, skeletal muscle contusion, skin diseases, sunburn, systemic lupus erythematosus, toothache, vasoocclusive painful crises in sickle cell disease, and visceral pain, comprising administering to the patient in need thereof a therapeutically effective amount of a compound of Formula (I′) having the structure:

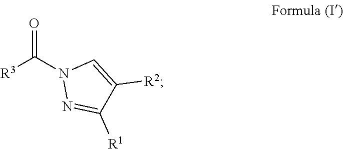

wherein:
R¹ is —C(O)OR¹⁵ or —C(O)NR¹⁰R¹¹;
R² is H, halogen, C₁₋₆alkyl, or C₁₋₆haloalkyl; R³ is

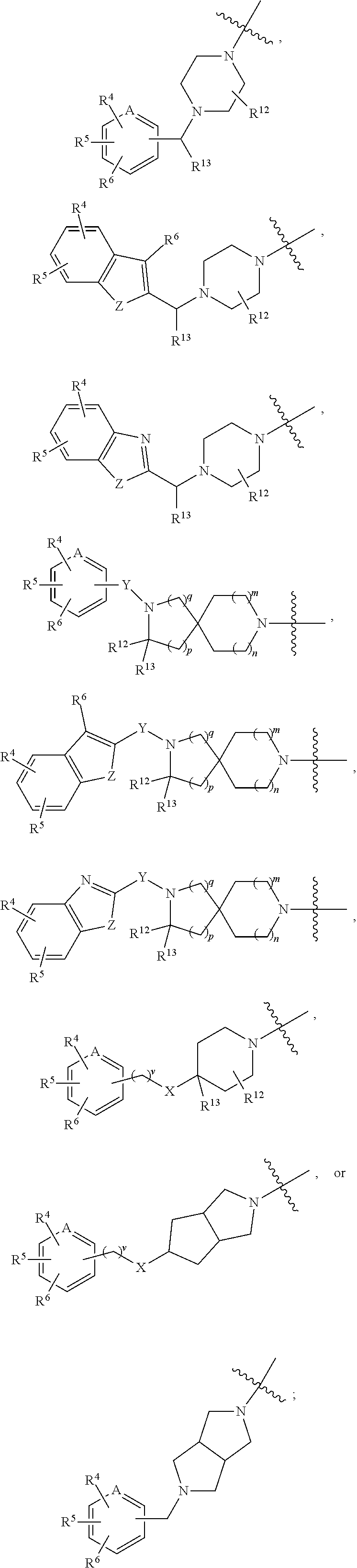

A is N or C(H);
X is —O—, —N(R¹⁶)—, or —CH₂N(R¹⁶)CH₂₋;
Y is —CH₂— or —C(O)—;
Z is —S—, —O—, or —N(R¹⁸)—;
R⁴ is H, halogen, —OR⁷, C₁₋₆alkyl, C₂₋₆alkenyl, C₂₋₆alkynyl, C₁₋₆haloalkyl, —C(O)NR⁸R⁹, C₂₋₉heterocycloalkyl, —C₁₋₆alkyl-C₂₋₉heterocycloalkyl, C₆₋₁₀aryl, or C₁₋₉heteroaryl, wherein C₂₋₉heterocycloalkyl, —C₁₋₆alkyl-C₂₋₉heterocycloalkyl, C₆₋₁₀aryl, or C₁₋₉heteroaryl are optionally substituted with 1 or 2 R¹⁴;
R⁵ is H, halogen, C₁₋₆alkyl, C₁₋₆haloalkyl, C₁₋₆haloalkoxy, or phenyl;
R⁶ is H, halogen or C₁₋₆alkyl;
R⁷ is H, C₁₋₆alkyl, C₁₋₆haloalkyl, C₂₋₉heterocycloalkyl, C₆₋₁₀aryl, or C₁₋₉heteroaryl, wherein C₂₋₉heterocycloalkyl, C₆₋₁₀aryl, or C₁₋₉heteroaryl are optionally substituted with 1 or 2 R¹⁴;
each R⁸ and each R⁹ are independently selected from H and C₁₋₆alkyl; or R⁸ and R⁹ together with the nitrogen to which they are attached are combined to form a heterocycloalkyl ring;
R¹⁰ and R¹¹ are each independently H or C₁₋₆alkyl;
R¹² is H, halogen, or C₁₋₆alkyl;
R¹³ is H or C₁₋₆alkyl;
each R¹⁴ is independently selected from halogen, —OH, C₁₋₆alkyl, C₁₋₆haloalkyl, C₁₋₆alkoxy, —C₁₋₆alkyl-OH, C₃₋₈cycloalkyl, —C(O)NR⁸R⁹, —SO₂—C₁₋₆alkyl, and —N(R¹⁷)C(O)—C₁₋₆alkyl;
R¹⁵ is H or C₁₋₆alkyl;
R¹⁶ is H, C₁₋₆alkyl, —C(O)—C₁₋₆alkyl, or —CH₂CO₂H;
R¹⁷ is H or C₁₋₆alkyl;
R¹⁸ is H or C₁₋₆alkyl;
v is 0 or 1;
n is 0 or 1;
m is 0 or 1;
p is 0, 1, or 2; and
q is 0, 1 or 2;
or a solvate, hydrate, tautomer, N-oxide, stereoisomer, or pharmaceutically acceptable salt thereof.

In some embodiments of the methods for treating a disease or condition with a compound of Formula (I′), R¹ is —C(O)OR¹⁵. In some embodiments of the methods for treating a disease or condition with a compound of Formula (I′), R¹ is —C(O)OR¹⁵ and R¹⁵ is H. In some embodiments of the methods for treating a disease or condition with a compound of Formula (I′), R¹ is —C(O)OR¹⁵ and R¹⁵ is C₁₋₆alkyl. In some embodiments of the methods for treating a disease or condition with a compound of Formula (I′), R¹ is —C(O)OR¹⁵ and R¹⁵ is —CH₃. In some embodiments of the methods for treating a disease or condition with a compound of Formula (I′), R¹ is —C(O)NR¹⁰R¹¹. In some embodiments of the methods for treating a disease or condition with a compound of Formula (I′), R¹ is —C(O)NR¹⁰R¹¹, R¹⁰ is H, and R¹¹ is H. In some embodiments of the methods for treating a disease or condition with a compound of Formula (I′), R¹ is —C(O)NR¹⁰R¹¹, R¹⁰ is H, and R¹¹ is C₁₋₆alkyl. In some embodiments of the methods for treating a disease or condition with a compound of Formula (I′), R¹ is —C(O)NR¹⁰R¹¹, R¹⁰ is H, and R¹¹ is —CH₃. In some embodiments of the methods for treating a disease or condition with a compound of Formula (I′), R¹ is —C(O)NR¹⁰R¹¹, R¹⁰ is C₁₋₆alkyl, and R¹¹ is C₁₋₆alkyl. In some embodiments of the methods for treating a disease or condition with a compound of Formula (I′), R¹ is —C(O)NR¹⁰R¹¹, R¹⁰ is —CH₃, and R¹¹ is —CH₃.

In some embodiments of the methods for treating a disease or condition with a compound of Formula (I′), R² is H. In some embodiments of the methods for treating a disease or condition with a compound of Formula (I′), R² is halogen. In some embodiments of the methods for treating a disease or condition with a compound of Formula (I′), R² is —Cl. In some embodiments of the methods for treating a disease or condition with a compound of Formula (I′), R² is C₁₋₆alkyl. In some embodiments of the methods for treating a disease or condition with a compound of Formula (I′), R² is —CH₃. In some embodiments of the methods for treating a disease or condition with a compound of Formula (I′), R² is C₁₋₆haloalkyl. In some embodiments of the methods for treating a disease or condition with a compound of Formula (I′), R² is —CF₃.

In some embodiments of the methods for treating a disease or condition with a compound of Formula (I′), R³ is

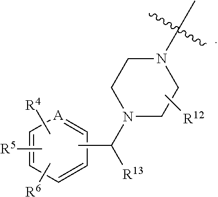

In some embodiments of the methods for treating a disease or condition with a compound of Formula (I′), R³ is

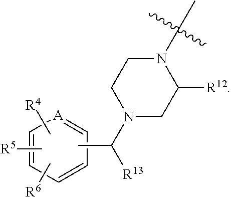

In some embodiments of the methods for treating a disease or condition with a compound of Formula (I′), R³ is

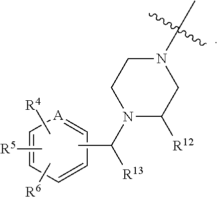

In some embodiments of the methods for treating a disease or condition with a compound of Formula (I′), A is N. In some embodiments of the methods for treating a disease or condition with a compound of Formula (I′), A is C(H). In some embodiments of the methods for treating a disease or condition with a compound of Formula (I′), R³ is

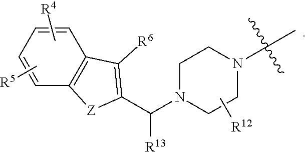

In some embodiments of the methods for treating a disease or condition with a compound of Formula (I′), R³ is

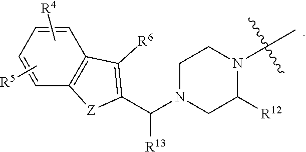

In some embodiments of the methods for treating a disease or condition with a compound of Formula (I′), R³ is

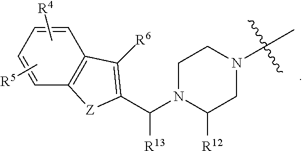

In some embodiments of the methods for treating a disease or condition with a compound of Formula (I′), R³ is

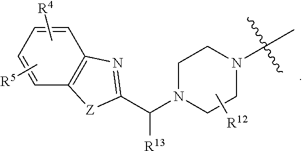

In some embodiments of the methods for treating a disease or condition with a compound of Formula (I′), R³ is

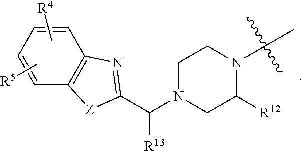

In some embodiments of the methods for treating a disease or condition with a compound of Formula (I′), R³ is

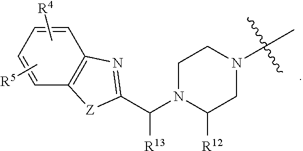

In some embodiments of the methods for treating a disease or condition with a compound of Formula (I′), Z is —S—. In some embodiments of the methods for treating a disease or condition with a compound of Formula (I′), Z is —O—. In some embodiments of the methods for treating a disease or condition with a compound of Formula (I′), Z is —N(R¹⁸)—. In some embodiments of the methods for treating a disease or condition with a compound of Formula (I′), Z is —N(H)—. In some embodiments of the methods for treating a disease or condition with a compound of Formula (I′), Z is —N(CH₃)—. In some embodiments of the methods for treating a disease or condition with a compound of Formula (I′), R¹² is H and R¹³ is H. In some embodiments of the methods for treating a disease or condition with a compound of Formula (I′), R¹² is C₁₋₆alkyl and R¹³ is H. In some embodiments of the methods for treating a disease or condition with a compound of Formula (I′), R¹² is —CH₃ and R¹³ is H. In some embodiments of the methods for treating a disease or condition with a compound of Formula (I′), R¹² is H and R¹³ is C₁₋₆alkyl. In some embodiments of the methods for treating a disease or condition with a compound of Formula (I′), R¹² is H and R¹³ is —CH₃. In some embodiments of the methods for treating a disease or condition with a compound of Formula (I′), R¹² is C₁₋₆alkyl and R¹³ is C₁₋₆alkyl. In some embodiments of the methods for treating a disease or condition with a compound of Formula (I′), R¹² is —CH₃ and R¹³ is —CH₃.

In some embodiments of the methods for treating a disease or condition with a compound of Formula (I′), R is

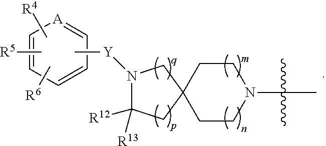

In some embodiments of the methods for treating a disease or condition with a compound of Formula (I′), A is N. In some embodiments of the methods for treating a disease or condition with a compound of Formula (I′), A is C(H). In some embodiments of the methods for treating a disease or condition with a compound of Formula (I′), R³ is

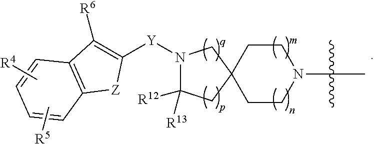

In some embodiments of the methods for treating a disease or condition with a compound of Formula (I′), R³ is

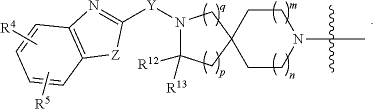

In some embodiments of the methods for treating a disease or condition with a compound of Formula (I′), Z is —S—. In some embodiments of the methods for treating a disease or condition with a compound of Formula (I′), Z is —O—. In some embodiments of the methods for treating a disease or condition with a compound of Formula (I′), Z is —N(R¹⁸)—. In some embodiments of the methods for treating a disease or condition with a compound of Formula (I′), Z is —N(H)—. In some embodiments of the methods for treating a disease or condition with a compound of Formula (I′), Z is —N(CH₃)—. In some embodiments of the methods for treating a disease or condition with a compound of Formula (I′), Y is —CH₂—. In some embodiments of the methods for treating a disease or condition with a compound of Formula (I′), Y is —C(O)—. In some embodiments of the methods for treating a disease or condition with a compound of Formula (I′), m is 1, n is 1, q is 1, and p is 1. In some embodiments of the methods for treating a disease or condition with a compound of Formula (I′), m is 0, n is 1, q is 2, and p is 1. In some embodiments of the methods for treating a disease or condition with a compound of Formula (I′), m is 1, n is 1, q is 0, and p is 2. In some embodiments of the methods for treating a disease or condition with a compound of Formula (I′), m is 1, n is 1, q is 1, and p is 0. In some embodiments of the methods for treating a disease or condition with a compound of Formula (I′), m is 0, n is 0, q is 2, and p is 1. In some embodiments of the methods for treating a disease or condition with a compound of Formula (I′), R¹² is H and R¹³ is H. In some embodiments of the methods for treating a disease or condition with a compound of Formula (I′), R¹² is C₁₋₆alkyl and R¹³ is H. In some embodiments of the methods for treating a disease or condition with a compound of Formula (I′), R¹² is —CH₃ and R¹³ is H. In some embodiments of the methods for treating a disease or condition with a compound of Formula (I′), R¹² is H and R¹³ is C₁₋₆alkyl. In some embodiments of the methods for treating a disease or condition with a compound of Formula (I′), R¹² is H and R¹³ is —CH₃. In some embodiments of the methods for treating a disease or condition with a compound of Formula (I′), R¹² is C₁₋₆alkyl and R¹³ is C₁₋₆alkyl. In some embodiments of the methods for treating a disease or condition with a compound of Formula (I′), R¹² is —CH₃ and R¹³ is-CH₃.

In some embodiments of the methods for treating a disease or condition with a compound of Formula (I′), R is

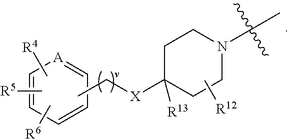

In some embodiments of the methods for treating a disease or condition with a compound of Formula (I′), R¹² is H. In some embodiments of the methods for treating a disease or condition with a compound of Formula (I′), R¹² is halogen. In some embodiments of the methods for treating a disease or condition with a compound of Formula (I′), R¹² is F. In some embodiments of the methods for treating a disease or condition with a compound of Formula (I′), R¹² is C₁₋₆alkyl. In some embodiments of the methods for treating a disease or condition with a compound of Formula (I′), R¹² is —CH₃. In some embodiments of the methods for treating a disease or condition with a compound of Formula (I′), R¹² is H and R¹³ is H. In some embodiments of the methods for treating a disease or condition with a compound of Formula (I′), R¹² is C₁₋₆alkyl and R¹³ is H. In some embodiments of the methods for treating a disease or condition with a compound of Formula (I′), R¹² is —CH₃ and R¹³ is H. In some embodiments of the methods for treating a disease or condition with a compound of Formula (I′), R¹² is H and R¹³ is C₁₋₆alkyl. In some embodiments of the methods for treating a disease or condition with a compound of Formula (I′), R¹² is H and R¹³ is —CH₃. In some embodiments of the methods for treating a disease or condition with a compound of Formula (I′), R¹² is C₁₋₆alkyl and R¹³ is C₁₋₆alkyl. In some embodiments of the methods for treating a disease or condition with a compound of Formula (I′), R¹² is —CH₃ and R¹³ is —CH₃. In some embodiments of the methods for treating a disease or condition with a compound of Formula (I′), R³ is

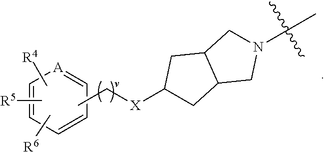

In some embodiments of the methods for treating a disease or condition with a compound of Formula (I′), X is —O—. In some embodiments of the methods for treating a disease or condition with a compound of Formula (I′), X is —N(R¹⁶)—. In some embodiments of the methods for treating a disease or condition with a compound of Formula (I′), X is —N(H)—. In some embodiments of the methods for treating a disease or condition with a compound of Formula (I′), X is —N(CH₃)—. In some embodiments of the methods for treating a disease or condition with a compound of Formula (I′), X is —CH₂N(R¹⁶)CH₂—. In some embodiments of the methods for treating a disease or condition with a compound of Formula (I′), X is —CH₂N(H)CH₂—. In some embodiments of the methods for treating a disease or condition with a compound of Formula (I′), X is —CH₂N(CH₃)CH₂—. In some embodiments of the methods for treating a disease or condition with a compound of Formula (I′), A is N. In some embodiments of the methods for treating a disease or condition with a compound of Formula (I′), A is C(H). In some embodiments of the methods for treating a disease or condition with a compound of Formula (I′), v is 0. In some embodiments of the methods for treating a disease or condition with a compound of Formula (I′), v is 1. In some embodiments of the methods for treating a disease or condition with a compound of Formula (I′), R³ is

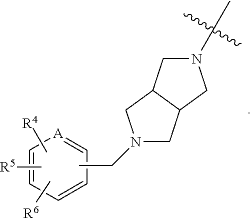

In some embodiments of the methods for treating a disease or condition with a compound of Formula (I′), R⁴ is halogen, —OR⁷, C₁₋₆alkyl, C₂₋₆alkynyl, C₁₋₆haloalkyl, —C(O)NR⁸R⁹, C₂₋₉heterocycloalkyl, C₆₋₁₀aryl, or C₁₋₉heteroaryl, wherein C₂₋₉ heterocycloalkyl, —C₁₋₆alkyl-C₂₋₉heterocycloalkyl, C₆₋₁₀aryl, or C₁₋₉heteroaryl are optionally substituted with 1 or 2 R¹⁴. In some embodiments of the methods for treating a disease or condition with a compound of Formula (I′), R⁴ is halogen, —OR⁷, C₁₋₆haloalkyl, C₂₋₉heterocycloalkyl, C₆₋₁₀aryl, or C₁₋₉heteroaryl, wherein C₂₋₉ heterocycloalkyl, C₆₋₁₀aryl, or C₁₋₉heteroaryl are optionally substituted with 1 or 2 R¹⁴. In some embodiments of the methods for treating a disease or condition with a compound of Formula (I′), R⁴ is halogen. In some embodiments of the methods for treating a disease or condition with a compound of Formula (I′), R⁴ is —Cl. In some embodiments of the methods for treating a disease or condition with a compound of Formula (I′), R⁴ is C₁₋₆haloalkyl. In some embodiments of the methods for treating a disease or condition with a compound of Formula (I′), R⁴ is —CF₃. In some embodiments of the methods for treating a disease or condition with a compound of Formula (I′), R⁴ is —OR⁷. In some embodiments of the methods for treating a disease or condition with a compound of Formula (I′), R⁴ is —OR⁷ and R⁷ is C₁₋₆haloalkyl, C₆₋₁₀aryl, or C₁₋₉heteroaryl, wherein C₆₋₁₀aryl or C₁₋₉heteroaryl are optionally substituted with 1 or 2 R¹⁴. In some embodiments of the methods for treating a disease or condition with a compound of Formula (I′), R⁴ is —OR⁷ and R⁷ is C₁₋₆haloalkyl. In some embodiments of the methods for treating a disease or condition with a compound of Formula (I′), R⁴ is —OR⁷ and R⁷ is —CF₃. In some embodiments of the methods for treating a disease or condition with a compound of Formula (I′), R⁴ is —OR⁷ and R⁷ is C₆₋₁₀aryl or C₁₋₉heteroaryl, wherein C₆₋₁₀aryl or C₁₋₉heteroaryl are optionally substituted with 1 or 2 R¹⁴. In some embodiments of the methods for treating a disease or condition with a compound of Formula (I′), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are optionally substituted with halogen or C₁₋₆haloalkyl. In some embodiments of the methods for treating a disease or condition with a compound of Formula (I′), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are unsubstituted. In some embodiments of the methods for treating a disease or condition with a compound of Formula (I′), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are substituted with halogen or C₁₋₆haloalkyl. In some embodiments of the methods for treating a disease or condition with a compound of Formula (I′), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are substituted with —Cl or —CF₃. In some embodiments of the methods for treating a disease or condition with a compound of Formula (I′), R⁴ is C₂₋₉heterocycloalkyl optionally substituted with 1 or 2 R¹⁴. In some embodiments of the methods for treating a disease or condition with a compound of Formula (I′), R⁴ is an unsubstituted C₂₋₉heterocycloalkyl. In some embodiments of the methods for treating a disease or condition with a compound of Formula (I′), R⁴ is C₂₋₉heterocycloalkyl substituted with 1 or 2 R¹⁴. In some embodiments of the methods for treating a disease or condition with a compound of Formula (I′), R⁴ is

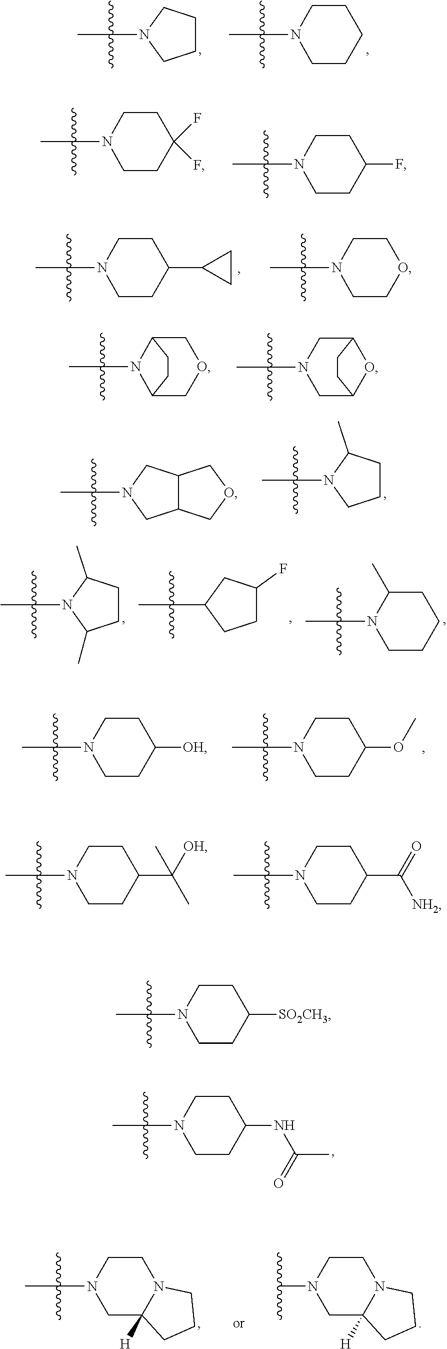

In some embodiments of the methods for treating a disease or condition with a compound of Formula (I′), R⁴ is C₁₋₉heteroaryl optionally substituted with 1 or 2 R¹⁴. In some embodiments of the methods for treating a disease or condition with a compound of Formula (I′), R⁴ is an unsubstituted C₁₋₉heteroaryl. In some embodiments of the methods for treating a disease or condition with a compound of Formula (I′), R⁴ is C₂₋₉heteroaryl substituted with 1 or 2 R¹⁴. In some embodiments of the methods for treating a disease or condition with a compound of Formula (I′), R⁴ is

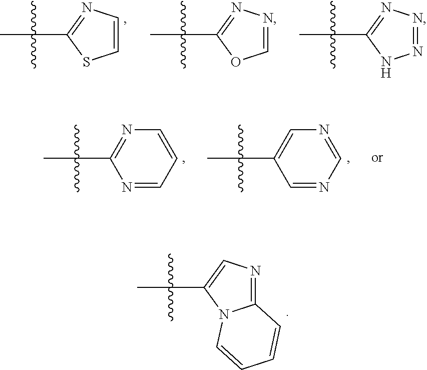 [SMILES: *c1nccs1 ; *c1nnco1 ; *c1nnn[nH]1 ; *c1ncccn1 ; *c1cncnc1 ; *c1cnc2ccccn12]

In some embodiments of the methods for treating a disease or condition with a compound of Formula (I′), R⁵ is H, halogen, C₁₋₆alkyl, C₁₋₆haloalkyl, C₁₋₆haloalkoxy. In some embodiments of the methods for treating a disease or condition with a compound of Formula (I′), R⁵ is H. In some embodiments of the methods for treating a disease or condition with a compound of Formula (I′), R⁵ is halogen. In some embodiments of the methods for treating a disease or condition with a compound of Formula (I′), R⁵ is —Cl. In some embodiments of the methods for treating a disease or condition with a compound of Formula (I′), R⁵ is —F. In some embodiments of the methods for treating a disease or condition with a compound of Formula (I′), R⁵ is C₁₋₆alkyl. In some embodiments of the methods for treating a disease or condition with a compound of Formula (I′), R⁵ is —CH₃. In some embodiments of the methods for treating a disease or condition with a compound of Formula (I′), R⁵ is C₁₋₆haloalkyl. In some embodiments of the methods for treating a disease or condition with a compound of Formula (I′), R⁵ is —CF₃. In some embodiments of the methods for treating a disease or condition with a compound of Formula (I′), R⁵ is C₁₋₆haloalkoxy. In some embodiments of the methods for treating a disease or condition with a compound of Formula (I′), R⁵ is —OCF₃.

In some embodiments of the methods for treating a disease or condition with a compound of Formula (I′), R⁶ is H. In some embodiments of the methods for treating a disease or condition with a compound of Formula (I′), R⁶ is halogen. In some embodiments of the methods for treating a disease or condition with a compound of Formula (I′), R⁶ is —Cl. In some embodiments of the methods for treating a disease or condition with a compound of Formula (I′), R⁶ is —F. In some embodiments of the methods for treating a disease or condition with a compound of Formula (I′), R⁶ is C₁₋₆alkyl. In some embodiments of the methods for treating a disease or condition with a compound of Formula (I′), R⁶ is —CH₃.

In some embodiments is a method for treating a disease or a condition in a patient wherein the disease or condition is selected from atopic dermatitis, bladder dysfunction associated with multiple sclerosis, cardiovascular disease, contact dermatitis, cystic fibrosis, dermatomyositis, eczema, endometriosis, enteritis, fibromyalgia, Tourette syndrome, inflammatory bowel disease, interstitial cystitis, irritable bowel syndrome, ischemia, labor, abdominal pain, abdominal pain associated with irritable bowel syndrome, acute pain, back pain, cancer pain, chest pain, functional chest pain, joint pain, menstrual pain, metabolic disorders, musculoskeletal diseases, neuropathy, osteoarthritis, pancreatitis, pharyngitis, post mastectomy pain syndrome, post trigeminal neuralgia, post operative pain, renal ischemia, rheumatoid arthritis, skeletal muscle contusion, skin diseases, sunburn, systemic lupus erythematosus, toothache, vasoocclusive painful crises in sickle cell disease, and visceral pain, comprising administering to the patient in need thereof a therapeutically effective amount of a compound of Formula (Ia) having the structure:

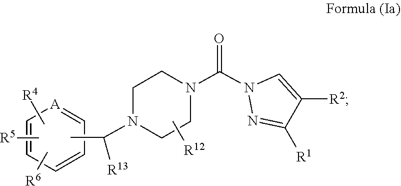

wherein:
R¹ is —C(O)OR¹⁵ or —C(O)NR¹⁰R¹¹;
R² is H, halogen, C₁₋₆alkyl, or C₁₋₆haloalkyl;
A is N or C(H);
R⁴ is H, halogen, —OR⁷, C₁₋₆alkyl, C₂₋₆alkenyl, C₂₋₆alkynyl, C₁₋₆haloalkyl, —C(O)NR⁸R⁹, C₂₋₉heterocycloalkyl, —C₁₋₆alkyl-C₂₋₉heterocycloalkyl, C₆₋₁₀aryl, or C₁₋₉heteroaryl, wherein C₂₋₉heterocycloalkyl, —C₁₋₆alkyl-C₂₋₉heterocycloalkyl, C₆₋₁₀aryl, or C₁₋₉heteroaryl are optionally substituted with 1 or 2 R¹⁴;
R⁵ is H, halogen, C₁₋₆alkyl, C₁₋₆haloalkyl, C₁₋₆haloalkoxy, or phenyl;
R⁶ is H, halogen or C₁₋₆alkyl;
R⁷ is H, C₁₋₆alkyl, C₁₋₆haloalkyl, C₂₋₉heterocycloalkyl, C₆₋₁₀aryl, or C₁₋₉heteroaryl, wherein C₂₋₉heterocycloalkyl, C₆₋₁₀aryl, or C₁₋₉heteroaryl are optionally substituted with 1 or 2 R¹⁴;
each R⁸ and each R⁹ are independently selected from H and C₁₋₆alkyl; or R⁸ and R⁹ together with the nitrogen to which they are attached are combined to form a heterocycloalkyl ring;
R¹⁰ and R¹¹ are each independently H or C₁₋₆alkyl;
R¹² is H, halogen, or C₁₋₆alkyl;
R¹³ is H or C₁₋₆alkyl;
each R¹⁴ is independently selected from halogen, —OH, C₁₋₆alkyl, C₁₋₆haloalkyl, C₁₋₆alkoxy, —C₁₋₆alkyl-OH, C₃₋₈cycloalkyl, —C(O)NR⁸R⁹, —SO₂—C₁₋₆alkyl, and —N(R¹⁷)C(O)—C₁₋₆alkyl;
R¹⁵ is H or C₁₋₆alkyl; and
R¹⁷ is H or C₁₋₆alkyl;
or a solvate, hydrate, tautomer, N-oxide, stereoisomer, or pharmaceutically acceptable salt thereof.

In some embodiments of the methods for treating a disease or condition with a compound of Formula (Ia), R³ is

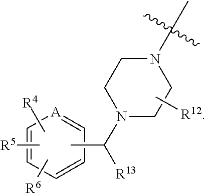

In some embodiments of the methods for treating a disease or condition with a compound of Formula (Ia), R³ is

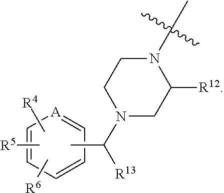

In some embodiments of the methods for treating a disease or condition with a compound of Formula (Ia), R³ is

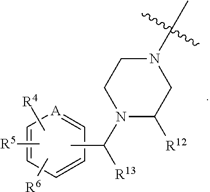

In some embodiments of the methods for treating a disease or condition with a compound of Formula (Ia), R¹ is —C(O)OR¹⁵. In some embodiments of the methods for treating a disease or condition with a compound of Formula (Ia), R¹ is —C(O)OR¹⁵ and R¹⁵ is H. In some embodiments of the methods for treating a disease or condition with a compound of Formula (Ia), R¹ is —C(O)OR¹⁵ and R¹⁵ is C₁₋₆alkyl. In some embodiments of the methods for treating a disease or condition with a compound of Formula (Ia), R¹ is —C(O)OR¹⁵ and R¹⁵ is —CH₃. In some embodiments of the methods for treating a disease or condition with a compound of Formula (Ia), R¹ is —C(O)NR¹⁰R¹¹. In some embodiments of the methods for treating a disease or condition with a compound of Formula (Ia), R¹ is —C(O)NR¹⁰R¹¹, R¹⁰ is H, and R¹¹ is H. In some embodiments of the methods for treating a disease or condition with a compound of Formula (Ia), R¹ is —C(O)NR¹⁰R¹¹, R¹⁰ is H, and R¹¹ is C₁₋₆alkyl. In some embodiments of the methods for treating a disease or condition with a compound of Formula (Ia), R¹ is —C(O)NR¹⁰R¹¹, R¹⁰ is H, and R¹¹ is —CH₃. In some embodiments of the methods for treating a disease or condition with a compound of Formula (Ia), R¹ is —C(O)NR¹⁰R¹¹, R¹⁰ is C₁₋₆alkyl, and R¹¹ is C₁₋₆alkyl. In some embodiments of the methods for treating a disease or condition with a compound of Formula (Ia), R¹ is —C(O)NR¹⁰R¹¹, R¹⁰ is —CH₃, and R¹¹ is —CH₃.

In some embodiments of the methods for treating a disease or condition with a compound of Formula (Ia), R² is H. In some embodiments of the methods for treating a disease or condition with a compound of Formula (Ia), R² is halogen. In some embodiments of the methods for treating a disease or condition with a compound of Formula (Ia), R² is —Cl. In some embodiments of the methods for treating a disease or condition with a compound of Formula (Ia), R² is C₁₋₆alkyl. In some embodiments of the methods for treating a disease or condition with a compound of Formula (Ia), R² is —CH₃. In some embodiments of the methods for treating a disease or condition with a compound of Formula (Ia), R² is C₁₋₆haloalkyl. In some embodiments of the methods for treating a disease or condition with a compound of Formula (Ia), R² is —CF₃.

In some embodiments of the methods for treating a disease or condition with a compound of Formula (Ia), R¹² is H. In some embodiments of the methods for treating a disease or condition with a compound of Formula (Ia), R¹² is halogen. In some embodiments of the methods for treating a disease or condition with a compound of Formula (Ia), R¹² is F. In some embodiments of the methods for treating a disease or condition with a compound of Formula (Ia), R¹² is C₁₋₆alkyl. In some embodiments of the methods for treating a disease or condition with a compound of Formula (Ia), R¹² is —CH₃. In some embodiments of the methods for treating a disease or condition with a compound of Formula (Ia), R¹³ is H. In some embodiments of the methods for treating a disease or condition with a compound of Formula (Ia), R¹³ is C₁₋₆alkyl. In some embodiments of the methods for treating a disease or condition with a compound of Formula (Ia), R¹³ is —CH₃. In some embodiments of the methods for treating a disease or condition with a compound of Formula (Ia), R¹² is H and R¹³ is H. In some embodiments of the methods for treating a disease or condition with a compound of Formula (Ia), R¹² is H and R¹³ is C₁₋₆alkyl. In some embodiments of the methods for treating a disease or condition with a compound of Formula (Ia), R¹² is H and R¹³ is —CH₃. In some embodiments of the methods for treating a disease or condition with a compound of Formula (Ia), R¹² is C₁₋₆alkyl and R¹³ is H. In some embodiments of the methods for treating a disease or condition with a compound of Formula (Ia), R¹² is —CH₃ and R¹³ is H. In some embodiments of the methods for treating a disease or condition with a compound of Formula (Ia), R¹² is C₁₋₆alkyl and R¹³ is C₁₋₆alkyl. In some embodiments of the methods for treating a disease or condition with a compound of Formula (Ia), R¹² is —CH₃ and R¹³ is —CH₃.

In some embodiments of the methods for treating a disease or condition with a compound of Formula (Ia), A is C(H). In some embodiments of the methods for treating a disease or condition with a compound of Formula (Ia), A is N.

In some embodiments of the methods for treating a disease or condition with a compound of Formula (Ia), R⁴ is halogen, —OR⁷, C₁₋₆alkyl, C₂₋₆alkynyl, C₁₋₆haloalkyl, —C(O)NR⁸R⁹, C₂₋₉heterocycloalkyl, C₆₋₁₀aryl, or C₁₋₉heteroaryl, wherein C₂₋₉ heterocycloalkyl, —C₁₋₆alkyl-C₂₋₉heterocycloalkyl, C₆₋₁₀aryl, or C₁₋₉heteroaryl are optionally substituted with 1 or 2 R¹⁴. In some embodiments of the methods for treating a disease or condition with a compound of Formula (Ia), R⁴ is halogen, —OR⁷, C₁₋₆haloalkyl, C₂₋₉heterocycloalkyl, C₆₋₁₀aryl, or C₁₋₉heteroaryl, wherein C₂₋₉heterocycloalkyl, C₆₋₁₀aryl, or C₁₋₉heteroaryl are optionally substituted with 1 or 2 R¹⁴. In some embodiments of the methods for treating a disease or condition with a compound of Formula (Ia), R⁴ is halogen. In some embodiments of the methods for treating a disease or condition with a compound of Formula (Ia), R⁴ is —Cl. In some embodiments of the methods for treating a disease or condition with a compound of Formula (Ia), R⁴ is C₁₋₆haloalkyl. In some embodiments of the methods for treating a disease or condition with a compound of Formula (Ia), R⁴ is —CF₃. In some embodiments of the methods for treating a disease or condition with a compound of Formula (Ia), R⁴ is —OR⁷. In some embodiments of the methods for treating a disease or condition with a compound of Formula (Ia), R⁴ is —OR⁷ and R⁷ is C₁₋₆haloalkyl, C₆₋₁₀aryl, or C₁₋₉heteroaryl, wherein C₆₋₁₀aryl or C₁₋₉heteroaryl are optionally substituted with 1 or 2 R¹⁴. In some embodiments of the methods for treating a disease or condition with a compound of Formula (Ia), R⁴ is —OR⁷ and R⁷ is C₁₋₆haloalkyl. In some embodiments of the methods for treating a disease or condition with a compound of Formula (Ia), R⁴ is —OR⁷ and R⁷ is —CF₃. In some embodiments of the methods for treating a disease or condition with a compound of Formula (Ia), R⁴ is —OR⁷ and R⁷ is C₆₋₁₀aryl or C₁₋₉heteroaryl, wherein C₆₋₁₀aryl or C₁₋₉heteroaryl are optionally substituted with 1 or 2 R¹⁴. In some embodiments of the methods for treating a disease or condition with a compound of Formula (Ia), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are optionally substituted with halogen or C₁₋₆haloalkyl. In some embodiments of the methods for treating a disease or condition with a compound of Formula (Ia), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are unsubstituted. In some embodiments of the methods for treating a disease or condition with a compound of Formula (Ia), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are substituted with halogen or C₁₋₆haloalkyl. In some embodiments of the methods for treating a disease or condition with a compound of Formula (Ia), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are substituted with —Cl or —CF₃. In some embodiments of the methods for treating a disease or condition with a compound of Formula (Ia), R⁴ is C₂₋₉heterocycloalkyl optionally substituted with 1 or 2 R¹⁴. In some embodiments of the methods for treating a disease or condition with a compound of Formula (Ia), R⁴ is an unsubstituted C₂₋₉ heterocycloalkyl. In some embodiments of the methods for treating a disease or condition with a compound of Formula (Ia), R⁴ is C₂₋₉heterocycloalkyl substituted with 1 or 2 R¹⁴. In some embodiments of the methods for treating a disease or condition with a compound of Formula (Ia), R⁴ is

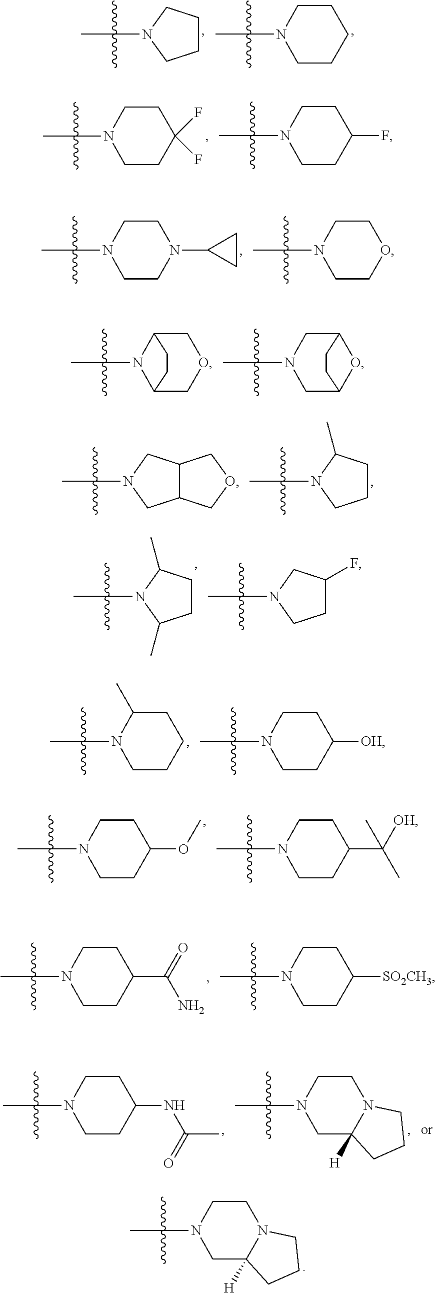

In some embodiments of the methods for treating a disease or condition with a compound of Formula (Ia), R⁴ is C₁₋₉ heteroaryl optionally substituted with 1 or 2 R¹⁴. In some embodiments of the methods for treating a disease or condition with a compound of Formula (Ia), R⁴ is an unsubstituted C₁₋₉heteroaryl. In some embodiments of the methods for treating a disease or condition with a compound of Formula (Ia), R⁴ is C₂₋₉heteroaryl substituted with 1 or 2 R¹⁴. In some embodiments of the methods for treating a disease or condition with a compound of Formula (Ia), R⁴ is

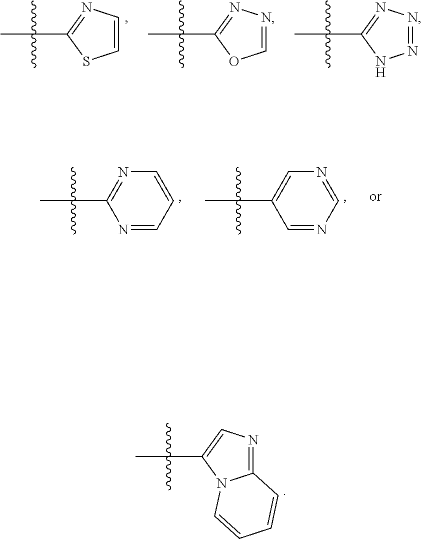 [SMILES: *c1nccs1 ; *c1nnco1 ; *c1nnn[nH]1 ; *c1ncccn1 ; *c1cncnc1 ; *c1cnc2ccccn12]

In some embodiments of the methods for treating a disease or condition with a compound of Formula (Ia), R⁵ is H, halogen, C₁₋₆alkyl, C₁₋₆haloalkyl, C₁₋₆haloalkoxy. In some embodiments of the methods for treating a disease or condition with a compound of Formula (Ia), R⁵ is H. In some embodiments of the methods for treating a disease or condition with a compound of Formula (Ia), R⁵ is halogen. In some embodiments of the methods for treating a disease or condition with a compound of Formula (Ia), R⁵ is —Cl. In some embodiments of the methods for treating a disease or condition with a compound of Formula (Ia), R⁵ is —F. In some embodiments of the methods for treating a disease or condition with a compound of Formula (Ia), R⁵ is C₁₋₆alkyl. In some embodiments of the methods for treating a disease or condition with a compound of Formula (Ia), R⁵ is —CH₃. In some embodiments of the methods for treating a disease or condition with a compound of Formula (Ia), R⁵ is C₁₋₆haloalkyl. In some embodiments of the methods for treating a disease or condition with a compound of Formula (Ia), R⁵ is —CF₃. In some embodiments of the methods for treating a disease or condition with a compound of Formula (Ia), R⁵ is C₁₋₆haloalkoxy. In some embodiments of the methods for treating a disease or condition with a compound of Formula (Ia), R⁵ is —OCF₃.

In some embodiments of the methods for treating a disease or condition with a compound of Formula (Ia), R⁶ is H. In some embodiments of the methods for treating a disease or condition with a compound of Formula (Ia), R⁶ is halogen. In some embodiments of the methods for treating a disease or condition with a compound of Formula (Ia), R⁶ is —Cl. In some embodiments of the methods for treating a disease or condition with a compound of Formula (Ia), R⁶ is —F. In some embodiments of the methods for treating a disease or condition with a compound of Formula (Ia), R⁶ is C₁₋₆alkyl. In some embodiments of the methods for treating a disease or condition with a compound of Formula (Ia), R⁶ is —CH₃.

In some embodiments is a method for treating a disease or a condition in a patient wherein the disease or condition is selected from atopic dermatitis, bladder dysfunction associated with multiple sclerosis, cardiovascular disease, contact dermatitis, cystic fibrosis, dermatomyositis, eczema, endometriosis, enteritis, fibromyalgia, Tourette syndrome, inflammatory bowel disease, interstitial cystitis, irritable bowel syndrome, ischemia, labor, abdominal pain, abdominal pain associated with irritable bowel syndrome, acute pain, back pain, cancer pain, chest pain, functional chest pain, joint pain, menstrual pain, metabolic disorders, musculoskeletal diseases, neuropathy, osteoarthritis, pancreatitis, pharyngitis, post mastectomy pain syndrome, post trigeminal neuralgia, post operative pain, renal ischemia, rheumatoid arthritis, skeletal muscle contusion, skin diseases, sunburn, systemic lupus erythematosus, toothache, vasoocclusive painful crises in sickle cell disease, and visceral pain, comprising administering to the patient in need thereof a therapeutically effective amount of a compound of Formula (Ib) having the structure:

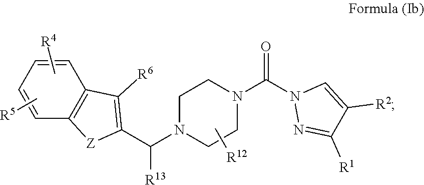

wherein:
R¹ is —C(O)OR¹⁵ or —C(O)NR¹⁰R¹¹;
R² is H, halogen, C₁₋₆alkyl, or C₁₋₆haloalkyl;
Z is —S—, —O—, or —N(R¹⁸)—;
R⁴ is H, halogen, —OR⁷, C₁₋₆alkyl, C₂₋₆alkenyl, C₂₋₆alkynyl, C₁₋₆haloalkyl, —C(O)NR⁸R⁹, C₂₋₉heterocycloalkyl, —C₁₋₆alkyl-C₂₋₉heterocycloalkyl, C₆₋₁₀aryl, or C₁₋₉heteroaryl, wherein C₂₋₉heterocycloalkyl, —C₁₋₆alkyl-C₂₋₉heterocycloalkyl, C₆₋₁₀aryl, C₁₋₉heteroaryl are optionally substituted with 1 or 2 R¹⁴;
R⁵ is H, halogen, C₁₋₆alkyl, C₁₋₆haloalkyl, C₁₋₆haloalkoxy, or phenyl;
R⁶ is H, halogen or C₁₋₆alkyl;
R⁷ is H, C₁₋₆alkyl, C₁₋₆haloalkyl, C₂₋₉heterocycloalkyl, C₆₋₁₀aryl, or C₁₋₉heteroaryl, wherein C₂₋₉heterocycloalkyl, C₆₋₁₀aryl, or C₁₋₉heteroaryl are optionally substituted with 1 or 2 R¹⁴;
each R⁸ and each R⁹ are independently selected from H and C₁₋₆alkyl; or R⁸ and R⁹ together with the nitrogen to which they are attached are combined to form a heterocycloalkyl ring;
R¹⁰ and R¹¹ are each independently H or C₁₋₆alkyl;
R¹² is H, halogen, or C₁₋₆alkyl;
R¹³ is H or C₁₋₆alkyl;
each R¹⁴ is independently selected from halogen, —OH, C₁₋₆alkyl, C₁₋₆haloalkyl, C₁₋₆alkoxy, —C₁₋₆alkyl-OH, C₃₋₈cycloalkyl, —C(O)NR⁸R⁹, —SO₂—C₁₋₆alkyl, and —N(R¹⁷)C(O)—C₁₋₆alkyl;
R¹⁵ is H or C₁₋₆alkyl;
R¹⁷ is H or C₁₋₆alkyl; and
R¹⁸ is H or C₁₋₆alkyl;
or a solvate, hydrate, tautomer, N-oxide, stereoisomer, or pharmaceutically acceptable salt thereof.

In some embodiments of the methods for treating a disease or condition with a compound of Formula (Ib), R³ is

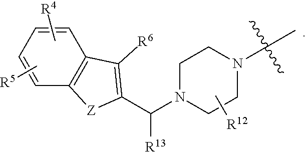

In some embodiments of the methods for treating a disease or condition with a compound of Formula (Ib), R³ is

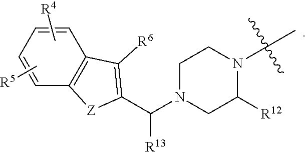

In some embodiments of the methods for treating a disease or condition with a compound of Formula (Ib), R³ is

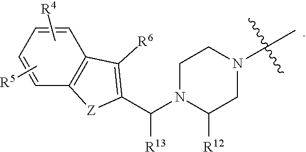

In some embodiments of the methods for treating a disease or condition with a compound of Formula (Ib), R¹ is —C(O)OR¹⁵. In some embodiments of the methods for treating a disease or condition with a compound of Formula (Ib), R¹ is —C(O)OR¹⁵ and R¹⁵ is H. In some embodiments of the methods for treating a disease or condition with a compound of Formula (Ib), R¹ is —C(O)OR¹⁵ and R¹⁵ is C₁₋₆alkyl. In some embodiments of the methods for treating a disease or condition with a compound of Formula (Ib), R¹ is —C(O)OR¹⁵ and R¹⁵ is —CH₃. In some embodiments of the methods for treating a disease or condition with a compound of Formula (Ib), R¹ is —C(O)NR¹⁰R¹¹. In some embodiments of the methods for treating a disease or condition with a compound of Formula (Ib), R¹ is —C(O)NR¹⁰R¹¹, R¹⁰ is H, and R¹¹ is H. In some embodiments of the methods for treating a disease or condition with a compound of Formula (Ib), R¹ is —C(O)NR¹⁰R¹¹, R¹⁰ is H, and R¹¹ is C₁₋₆alkyl. In some embodiments of the methods for treating a disease or condition with a compound of Formula (Ib), R¹ is —C(O)NR¹⁰R¹¹, R¹⁰ is H, and R¹¹ is —CH₃. In some embodiments of the methods for treating a disease or condition with a compound of Formula (Ib), R¹ is —C(O)NR¹⁰R¹¹, R¹⁰ is C₁₋₆alkyl, and R¹¹ is C₁₋₆alkyl. In some embodiments of the methods for treating a disease or condition with a compound of Formula (Ib), R¹ is —C(O)NR¹⁰R¹¹, R¹⁰ is —CH₃, and R¹¹ is —CH₃.

In some embodiments of the methods for treating a disease or condition with a compound of Formula (Ib), R² is H. In some embodiments of the methods for treating a disease or condition with a compound of Formula (Ib), R² is halogen. In some embodiments of the methods for treating a disease or condition with a compound of Formula (Ib), R² is —Cl. In some embodiments of the methods for treating a disease or condition with a compound of Formula (Ib), R² is C₁₋₆alkyl. In some embodiments of the methods for treating a disease or condition with a compound of Formula (Ib), R² is —CH₃. In some embodiments of the methods for treating a disease or condition with a compound of Formula (Ib), R² is C₁₋₆haloalkyl. In some embodiments of the methods for treating a disease or condition with a compound of Formula (Ib), R² is —CF₃.

In some embodiments of the methods for treating a disease or condition with a compound of Formula (Ib), R¹² is H. In some embodiments of the methods for treating a disease or condition with a compound of Formula (Ib), R¹² is halogen. In some embodiments of the methods for treating a disease or condition with a compound of Formula (Ib), R¹² is F. In some embodiments of the methods for treating a disease or condition with a compound of Formula (Ib), R¹² is C₁₋₆alkyl. In some embodiments of the methods for treating a disease or condition with a compound of Formula (Ib), R¹² is —CH₃. In some embodiments of the methods for treating a disease or condition with a compound of Formula (Ib), R¹³ is H. In some embodiments of the methods for treating a disease or condition with a compound of Formula (Ib), R¹³ is C₁₋₆alkyl. In some embodiments of the methods for treating a disease or condition with a compound of Formula (Ib), R¹³ is —CH₃. In some embodiments of the methods for treating a disease or condition with a compound of Formula (Ib), R¹² is H and R¹³ is H. In some embodiments of the methods for treating a disease or condition with a compound of Formula (Ib), R¹² is H and R¹³ is C₁₋₆alkyl. In some embodiments of the methods for treating a disease or condition with a compound of Formula (Ib), R¹² is H and R¹³ is —CH₃. In some embodiments of the methods for treating a disease or condition with a compound of Formula (Ib), R¹² is C₁₋₆alkyl and R¹³ is H. In some embodiments of the methods for treating a disease or condition with a compound of Formula (Ib), R¹² is —CH₃ and R¹³ is H. In some embodiments of the methods for treating a disease or condition with a compound of Formula (Ib), R¹² is C₁₋₆alkyl and R¹³ is C₁₋₆alkyl. In some embodiments of the methods for treating a disease or condition with a compound of Formula (Ib), R¹² is —CH₃ and R¹³ is —CH₃.

In some embodiments of the methods for treating a disease or condition with a compound of Formula (Ib), Z is —S—. In some embodiments of the methods for treating a disease or condition with a compound of Formula (Ib), Z is —O—. In some embodiments of the methods for treating a disease or condition with a compound of Formula (Ib), Z is —N(R¹⁸)—. In some embodiments of the methods for treating a disease or condition with a compound of Formula (Ib), Z is —N(H)—. In some embodiments of the methods for treating a disease or condition with a compound of Formula (Ib), Z is —N(CH₃)—.

In some embodiments of the methods for treating a disease or condition with a compound of Formula (Ib), R⁴ is halogen, —OR⁷, C₁₋₆alkyl, C₂₋₆alkynyl, C₁₋₆haloalkyl, —C(O)NR⁸R⁹, C₂₋₉heterocycloalkyl, C₆₋₁₀aryl, or C₁₋₉heteroaryl, wherein C₂₋₉ heterocycloalkyl, —C₁₋₆alkyl-C₂₋₉heterocycloalkyl, C₆₋₁₀aryl, C₁₋₉heteroaryl are optionally substituted with 1 or 2 R¹⁴. In some embodiments of the methods for treating a disease or condition with a compound of Formula (Ib), R⁴ is halogen, —OR⁷, C₁₋₆haloalkyl, C₂₋₉heterocycloalkyl, C₆₋₁₀aryl, or C₁₋₉heteroaryl, wherein C₂₋₉heterocycloalkyl, C₆₋₁₀aryl, or C₁₋₉heteroaryl are optionally substituted with 1 or 2 R¹⁴. In some embodiments of the methods for treating a disease or condition with a compound of Formula (Ib), R⁴ is halogen. In some embodiments of the methods for treating a disease or condition with a compound of Formula (Ib), R⁴ is —Cl. In some embodiments of the methods for treating a disease or condition with a compound of Formula (Ib), R⁴ is C₁₋₆haloalkyl. In some embodiments of the methods for treating a disease or condition with a compound of Formula (Ib), R⁴ is —CF₃. In some embodiments of the methods for treating a disease or condition with a compound of Formula (Ib), R⁴ is —OR⁷. In some embodiments of the methods for treating a disease or condition with a compound of Formula (Ib), R⁴ is —OR⁷ and R⁷ is C₁₋₆haloalkyl, C₆₋₁₀aryl, or C₁₋₉heteroaryl, wherein C₆₋₁₀aryl or C₁₋₉heteroaryl are optionally substituted with 1 or 2 R¹⁴. In some embodiments of the methods for treating a disease or condition with a compound of Formula (Ib), R⁴ is —OR⁷ and R⁷ is C₁₋₆haloalkyl. In some embodiments of the methods for treating a disease or condition with a compound of Formula (Ib), R⁴ is —OR⁷ and R⁷ is —CF₃. In some embodiments of the methods for treating a disease or condition with a compound of Formula (Ib), R⁴ is —OR⁷ and R⁷ is C₆₋₁₀aryl or C₁₋₉heteroaryl, wherein C₆₋₁₀aryl or C₁₋₉heteroaryl are optionally substituted with 1 or 2 R¹⁴. In some embodiments of the methods for treating a disease or condition with a compound of Formula (Ib), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are optionally substituted with halogen or C₁₋₆haloalkyl. In some embodiments of the methods for treating a disease or condition with a compound of Formula (Ib), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are unsubstituted. In some embodiments of the methods for treating a disease or condition with a compound of Formula (Ib), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are substituted with halogen or C₁₋₆haloalkyl. In some embodiments of the methods for treating a disease or condition with a compound of Formula (Ib), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are substituted with —Cl or —CF₃. In some embodiments of the methods for treating a disease or condition with a compound of Formula (Ib), R⁴ is C₂₋₉ heterocycloalkyl optionally substituted with 1 or 2 R¹⁴. In some embodiments of the methods for treating a disease or condition with a compound of Formula (Ib), R⁴ is an unsubstituted C₂₋₉heterocycloalkyl. In some embodiments of the methods for treating a disease or condition with a compound of Formula (Ib), R⁴ is C₂₋₉heterocycloalkyl substituted with 1 or 2 R¹⁴. In some embodiments of the methods for treating a disease or condition with a compound of Formula (Ib), R⁴ is

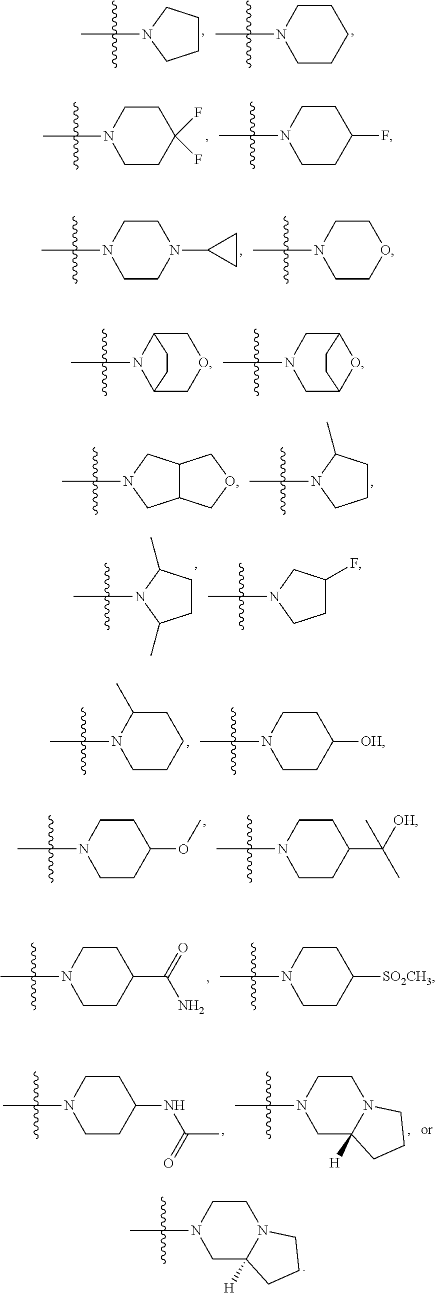

In some embodiments of the methods for treating a disease or condition with a compound of Formula (Ib), R⁴ is C₁₋₉heteroaryl optionally substituted with 1 or 2 R¹⁴. In some embodiments of the methods for treating a disease or condition with a compound of Formula (Ib), R⁴ is an unsubstituted C₁₋₉heteroaryl. In some embodiments of the methods for treating a disease or condition with a compound of Formula (Ib), R⁴ is C₂₋₉heteroaryl substituted with 1 or 2 R¹⁴. In some embodiments of the methods for treating a disease or condition with a compound of Formula (Ib), R⁴ is

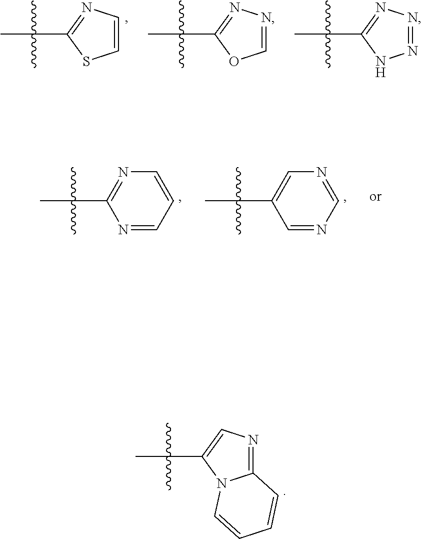 [SMILES: *c1nccs1 ; *c1nnco1 ; *c1nnn[nH]1 ; *c1ncccn1 ; *c1cncnc1 ; *c1cnc2ccccn12]

In some embodiments of the methods for treating a disease or condition with a compound of Formula (Ib), R⁵ is H, halogen, C₁₋₆alkyl, C₁₋₆haloalkyl, C₁₋₆haloalkoxy. In some embodiments of the methods for treating a disease or condition with a compound of Formula (Ib), R⁵ is H. In some embodiments of the methods for treating a disease or condition with a compound of Formula (Ib), R⁵ is halogen. In some embodiments of the methods for treating a disease or condition with a compound of Formula (Ib), R⁵ is —Cl. In some embodiments of the methods for treating a disease or condition with a compound of Formula (Ib), R⁵ is —F. In some embodiments of the methods for treating a disease or condition with a compound of Formula (Ib), R⁵ is C₁₋₆alkyl. In some embodiments of the methods for treating a disease or condition with a compound of Formula (Ib), R⁵ is —CH₃. In some embodiments of the methods for treating a disease or condition with a compound of Formula (Ib), R⁵ is C₁₋₆haloalkyl. In some embodiments of the methods for treating a disease or condition with a compound of Formula (Ib), R⁵ is —CF₃. In some embodiments of the methods for treating a disease or condition with a compound of Formula (Ib), R⁵ is C₁₋₆haloalkoxy. In some embodiments of the methods for treating a disease or condition with a compound of Formula (Ib), R⁵ is —OCF₃.

In some embodiments of the methods for treating a disease or condition with a compound of Formula (Ib), R⁶ is H. In some embodiments of the methods for treating a disease or condition with a compound of Formula (Ib), R⁶ is halogen. In some embodiments of the methods for treating a disease or condition with a compound of Formula (Ib), R⁶ is —Cl. In some embodiments of the methods for treating a disease or condition with a compound of Formula (Ib), R⁶ is —F. In some embodiments of the methods for treating a disease or condition with a compound of Formula (Ib), R⁶ is C₁₋₆alkyl. In some embodiments of the methods for treating a disease or condition with a compound of Formula (Ib), R⁶ is —CH₃.

In some embodiments is a method for treating a disease or a condition in a patient wherein the disease or condition is selected from atopic dermatitis, bladder dysfunction associated with multiple sclerosis, cardiovascular disease, contact dermatitis, cystic fibrosis, dermatomyositis, eczema, endometriosis, enteritis, fibromyalgia, Tourette syndrome, inflammatory bowel disease, interstitial cystitis, irritable bowel syndrome, ischemia, labor, abdominal pain, abdominal pain associated with irritable bowel syndrome, acute pain, back pain, cancer pain, chest pain, functional chest pain, joint pain, menstrual pain, metabolic disorders, musculoskeletal diseases, neuropathy, osteoarthritis, pancreatitis, pharyngitis, post mastectomy pain syndrome, post trigeminal neuralgia, post operative pain, renal ischemia, rheumatoid arthritis, skeletal muscle contusion, skin diseases, sunburn, systemic lupus erythematosus, toothache, vasoocclusive painful crises in sickle cell disease, and visceral pain, comprising administering to the patient in need thereof a therapeutically effective amount of a compound of Formula (Ic) having the structure:

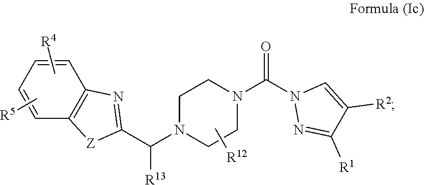

wherein:
R¹ is —C(O)OR¹⁵ or —C(O)NR¹⁰R¹¹;
R² is H, halogen, C₁₋₆alkyl, or C₁₋₆haloalkyl;
Z is —S—, —O—, or —N(R¹⁸)—;
R⁴ is H, halogen, —OR⁷, C₁₋₆alkyl, C₂₋₆alkenyl, C₂₋₆alkynyl, C₁₋₆haloalkyl, —C(O)NR⁸R⁹, C₂₋₉heterocycloalkyl, —C₁₋₆alkyl-C₂₋₉heterocycloalkyl, C₆₋₁₀aryl, or C₁₋₉heteroaryl, wherein C₂₋₉heterocycloalkyl, —C₁₋₆alkyl-C₂₋₉heterocycloalkyl, C₆₋₁₀aryl, or C₁₋₉heteroaryl are optionally substituted with 1 or 2 R¹⁴;
R⁵ is H, halogen, C₁₋₆alkyl, C₁₋₆haloalkyl, C₁₋₆haloalkoxy, or phenyl;
R⁷ is H, C₁₋₆alkyl, C₁₋₆haloalkyl, C₂₋₉heterocycloalkyl, C₆₋₁₀aryl, or C₁₋₉heteroaryl, wherein C₂₋₉heterocycloalkyl, C₆₋₁₀aryl, or C₁₋₉heteroaryl are optionally substituted with 1 or 2 R¹⁴;
each R⁸ and each R⁹ are independently selected from H and C₁₋₆alkyl; or R⁸ and R⁹ together with the nitrogen to which they are attached are combined to form a heterocycloalkyl ring;
R¹⁰ and R¹¹ are each independently H or C₁₋₆alkyl;
R¹² is H, halogen, or C₁₋₆alkyl;
R¹³ is H or C₁₋₆alkyl;
each R¹⁴ is independently selected from halogen, —OH, C₁₋₆alkyl, C₁₋₆haloalkyl, C₁₋₆alkoxy, —C₁₋₆alkyl-OH, C₃₋₈cycloalkyl, —C(O)NR⁸R⁹, —SO₂—C₁₋₆alkyl, and —N(R¹⁷)C(O)—C₁₋₆alkyl;
R¹⁵ is H or C₁₋₆alkyl;
R¹⁷ is H or C₁₋₆alkyl; and
R¹⁸ is H or C₁₋₆alkyl;
or a solvate, hydrate, tautomer, N-oxide, stereoisomer, or pharmaceutically acceptable salt thereof.

In some embodiments of the methods for treating a disease or condition with a compound of Formula (Ic), R is

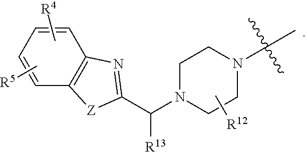

In some embodiments of the methods for treating a disease or condition with a compound of Formula (Ic), R³ is

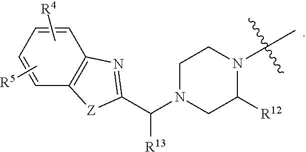

In some embodiments of the methods for treating a disease or condition with a compound of Formula (Ic), R³ is

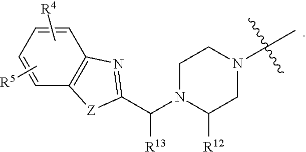

In some embodiments of the methods for treating a disease or condition with a compound of Formula (Ic), R¹ is —C(O)OR¹⁵. In some embodiments of the methods for treating a disease or condition with a compound of Formula (Ic), R¹ is —C(O)OR¹⁵ and R¹⁵ is H. In some embodiments of the methods for treating a disease or condition with a compound of Formula (Ic), R¹ is —C(O)OR¹⁵ and R¹⁵ is C₁₋₆alkyl. In some embodiments of the methods for treating a disease or condition with a compound of Formula (Ic), R¹ is —C(O)OR¹⁵ and R¹⁵ is —CH₃. In some embodiments of the methods for treating a disease or condition with a compound of Formula (Ic), R¹ is —C(O)NR¹⁰R¹¹. In some embodiments of the methods for treating a disease or condition with a compound of Formula (Ic), R¹ is —C(O)NR¹⁰R¹¹, R¹⁰ is H, and R¹¹ is H. In some embodiments of the methods for treating a disease or condition with a compound of Formula (Ic), R¹ is —C(O)NR¹⁰R¹¹, R¹⁰ is H, and R¹¹ is C₁₋₆alkyl. In some embodiments of the methods for treating a disease or condition with a compound of Formula (Ic), R¹ is —C(O)NR¹⁰R¹¹, R¹⁰ is H, and R¹¹ is —CH₃. In some embodiments of the methods for treating a disease or condition with a compound of Formula (Ic), R¹ is —C(O)NR¹⁰R¹¹, R¹⁰ is C₁₋₆alkyl, and R¹¹ is C₁₋₆alkyl. In some embodiments of the methods for treating a disease or condition with a compound of Formula (Ic), R¹ is —C(O)NR¹⁰R¹¹, R¹⁰ is —CH₃, and R¹¹ is —CH₃.

In some embodiments of the methods for treating a disease or condition with a compound of Formula (Ic), R² is H. In some embodiments of the methods for treating a disease or condition with a compound of Formula (Ic), R² is halogen. In some embodiments of the methods for treating a disease or condition with a compound of Formula (Ic), R² is —Cl. In some embodiments of the methods for treating a disease or condition with a compound of Formula (Ic), R² is C₁₋₆alkyl. In some embodiments of the methods for treating a disease or condition with a compound of Formula (Ic), R² is —CH₃. In some embodiments of the methods for treating a disease or condition with a compound of Formula (Ic), R² is C₁₋₆haloalkyl. In some embodiments of the methods for treating a disease or condition with a compound of Formula (Ic), R² is —CF₃.

In some embodiments of the methods for treating a disease or condition with a compound of Formula (Ic), R¹² is H. In some embodiments of the methods for treating a disease or condition with a compound of Formula (Ic), R¹² is halogen. In some embodiments of the methods for treating a disease or condition with a compound of Formula (Ic), R¹² is F. In some embodiments of the methods for treating a disease or condition with a compound of Formula (Ic), R¹² is C₁₋₆alkyl. In some embodiments of the methods for treating a disease or condition with a compound of Formula (Ic), R¹² is —CH₃. In some embodiments of the methods for treating a disease or condition with a compound of Formula (Ic), R¹³ is H. In some embodiments of the methods for treating a disease or condition with a compound of Formula (Ic), R¹³ is C₁₋₆alkyl. In some embodiments of the methods for treating a disease or condition with a compound of Formula (Ic), R¹³ is —CH₃. In some embodiments of the methods for treating a disease or condition with a compound of Formula (Ic), R¹² is H and R¹³ is H. In some embodiments of the methods for treating a disease or condition with a compound of Formula (Ic), R¹² is H and R¹³ is C₁₋₆alkyl. In some embodiments of the methods for treating a disease or condition with a compound of Formula (Ic), R¹² is H and R¹³ is —CH₃. In some embodiments of the methods for treating a disease or condition with a compound of Formula (Ic), R¹² is C₁₋₆alkyl and R¹³ is H. In some embodiments of the methods for treating a disease or condition with a compound of Formula (Ic), R¹² is —CH₃ and R¹³ is H. In some embodiments of the methods for treating a disease or condition with a compound of Formula (Ic), R¹² is C₁₋₆alkyl and R¹³ is C₁₋₆alkyl. In some embodiments of the methods for treating a disease or condition with a compound of Formula (Ic), R¹² is —CH₃ and R¹³ is —CH₃.

In some embodiments of the methods for treating a disease or condition with a compound of Formula (Ic), Z is —S—. In some embodiments of the methods for treating a disease or condition with a compound of Formula (Ic), Z is —O—. In some embodiments of the methods for treating a disease or condition with a compound of Formula (Ic), Z is —N(R¹⁸)—. In some embodiments of the methods for treating a disease or condition with a compound of Formula (Ic), Z is —N(H)—. In some embodiments of the methods for treating a disease or condition with a compound of Formula (Ic), Z is —N(CH₃)—.

In some embodiments of the methods for treating a disease or condition with a compound of Formula (Ic), R⁴ is halogen, —OR⁷, C₁₋₆alkyl, C₂₋₆alkynyl, C₁₋₆haloalkyl, —C(O)NR⁸R⁹, C₂₋₉heterocycloalkyl, C₆₋₁₀aryl, or C₁₋₉heteroaryl, wherein C₂₋₉ heterocycloalkyl, —C₁₋₆alkyl-C₂₋₉heterocycloalkyl, C₆₋₁₀aryl, or C₁₋₉heteroaryl are optionally substituted with 1 or 2 R¹⁴. In some embodiments of the methods for treating a disease or condition with a compound of Formula (Ic), R⁴ is halogen, —OR⁷, C₁₋₆haloalkyl, C₂₋₉heterocycloalkyl, C₆₋₁₀aryl, or C₁₋₉heteroaryl, wherein C₂₋₉ heterocycloalkyl, C₆₋₁₀aryl, or C₁₋₉heteroaryl are optionally substituted with 1 or 2 R¹⁴. In some embodiments of the methods for treating a disease or condition with a compound of Formula (Ic), R⁴ is halogen. In some embodiments of the methods for treating a disease or condition with a compound of Formula (Ic), R⁴ is —Cl. In some embodiments of the methods for treating a disease or condition with a compound of Formula (Ic), R⁴ is C₁₋₆haloalkyl. In some embodiments of the methods for treating a disease or condition with a compound of Formula (Ic), R⁴ is —CF₃. In some embodiments of the methods for treating a disease or condition with a compound of Formula (Ic), R⁴ is —OR⁷. In some embodiments of the methods for treating a disease or condition with a compound of Formula (Ic), R⁴ is —OR⁷ and R⁷ is C₁₋₆haloalkyl, C₆₋₁₀aryl, or C₁₋₉heteroaryl, wherein C₆₋₁₀aryl or C₁₋₉heteroaryl are optionally substituted with 1 or 2 R¹⁴. In some embodiments of the methods for treating a disease or condition with a compound of Formula (Ic), R⁴ is —OR⁷ and R⁷ is C₁₋₆haloalkyl. In some embodiments of the methods for treating a disease or condition with a compound of Formula (Ic), R⁴ is —OR⁷ and R⁷ is —CF₃. In some embodiments of the methods for treating a disease or condition with a compound of Formula (Ic), R⁴ is —OR⁷ and R⁷ is C₆₋₁₀aryl or C₁₋₉heteroaryl, wherein C₆₋₁₀aryl or C₁₋₉heteroaryl are optionally substituted with 1 or 2 R¹⁴. In some embodiments of the methods for treating a disease or condition with a compound of Formula (Ic), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are optionally substituted with halogen or C₁₋₆haloalkyl. In some embodiments of the methods for treating a disease or condition with a compound of Formula (Ic), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are unsubstituted. In some embodiments of the methods for treating a disease or condition with a compound of Formula (Ic), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are substituted with halogen or C₁₋₆haloalkyl. In some embodiments of the methods for treating a disease or condition with a compound of Formula (Ic), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are substituted with —Cl or —CF₃. In some embodiments of the methods for treating a disease or condition with a compound of Formula (Ic), R⁴ is C₂₋₉heterocycloalkyl optionally substituted with 1 or 2 R¹⁴. In some embodiments of the methods for treating a disease or condition with a compound of Formula (Ic), R⁴ is an unsubstituted C₂₋₉heterocycloalkyl. In some embodiments of the methods for treating a disease or condition with a compound of Formula (Ic), R⁴ is C₂₋₉heterocycloalkyl substituted with 1 or 2 R¹⁴. In some embodiments of the methods for treating a disease or condition with a compound of Formula (Ic), R⁴ is

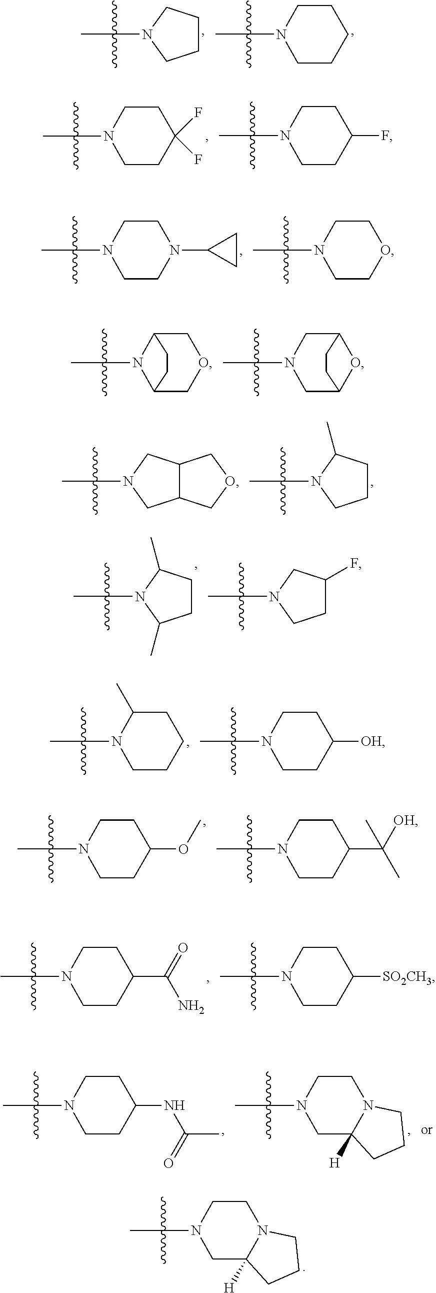

In some embodiments of the methods for treating a disease or condition with a compound of Formula (Ic), R⁴ is C₁₋₉ heteroaryl optionally substituted with 1 or 2 R¹⁴. In some embodiments of the methods for treating a disease or condition with a compound of Formula (Ic), R⁴ is an unsubstituted C₁₋₉heteroaryl. In some embodiments of the methods for treating a disease or condition with a compound of Formula (Ic), R⁴ is C₂₋₉heteroaryl substituted with 1 or 2 R¹⁴. In some embodiments of the methods for treating a disease or condition with a compound of Formula (Ic), R⁴ is

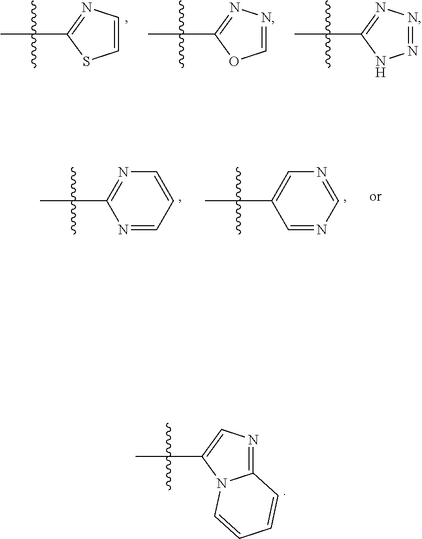 [SMILES: *c1nccs1 ; *c1nnco1 ; *c1nnn[nH]1 ; *c1ncccn1 ; *c1cncnc1 ; *c1cnc2ccccn12]

In some embodiments of the methods for treating a disease or condition with a compound of Formula (Ic), R⁵ is H, halogen, C₁₋₆alkyl, C₁₋₆haloalkyl, C₁₋₆haloalkoxy. In some embodiments of the methods for treating a disease or condition with a compound of Formula (Ic), R⁵ is H. In some embodiments of the methods for treating a disease or condition with a compound of Formula (Ic), R⁵ is halogen. In some embodiments of the methods for treating a disease or condition with a compound of Formula (Ic), R⁵ is —Cl. In some embodiments of the methods for treating a disease or condition with a compound of Formula (Ic), R⁵ is —F. In some embodiments of the methods for treating a disease or condition with a compound of Formula (Ic), R⁵ is C₁₋₆alkyl. In some embodiments of the methods for treating a disease or condition with a compound of Formula (Ic), R⁵ is —CH₃. In some embodiments of the methods for treating a disease or condition with a compound of Formula (Ic), R⁵ is C₁₋₆haloalkyl. In some embodiments of the methods for treating a disease or condition with a compound of Formula (Ic), R⁵ is —CF₃. In some embodiments of the methods for treating a disease or condition with a compound of Formula (Ic), R⁵ is C₁₋₆haloalkoxy. In some embodiments of the methods for treating a disease or condition with a compound of Formula (Ic), R⁵ is —OCF₃.

In some embodiments is a method for treating a disease or a condition in a patient wherein the disease or condition is selected from atopic dermatitis, bladder dysfunction associated with multiple sclerosis, cardiovascular disease, contact dermatitis, cystic fibrosis, dermatomyositis, eczema, endometriosis, enteritis, fibromyalgia, Tourette syndrome, inflammatory bowel disease, interstitial cystitis, irritable bowel syndrome, ischemia, labor, abdominal pain, abdominal pain associated with irritable bowel syndrome, acute pain, back pain, cancer pain, chest pain, functional chest pain, joint pain, menstrual pain, metabolic disorders, musculoskeletal diseases, neuropathy, osteoarthritis, pancreatitis, pharyngitis, post mastectomy pain syndrome, post trigeminal neuralgia, post operative pain, renal ischemia, rheumatoid arthritis, skeletal muscle contusion, skin diseases, sunburn, systemic lupus erythematosus, toothache, vasoocclusive painful crises in sickle cell disease, and visceral pain, comprising administering to the patient in need thereof a therapeutically effective amount of a compound of Formula (Id) having the structure:

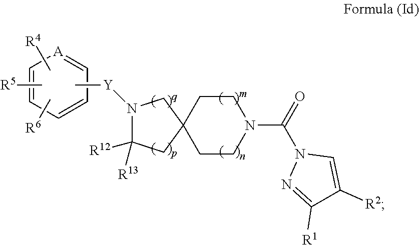

wherein: ‘ ’
R¹ is —C(O)OR¹⁵ or —C(O)NR¹⁰R¹¹;
R² is H, halogen, C₁₋₆alkyl, or C₁₋₆haloalkyl;
A is N or C(H);
Y is —CH₂— or —C(O)—;
R⁴ is H, halogen, —OR⁷, C₁₋₆alkyl, C₂₋₆alkenyl, C₂₋₆alkynyl, C₁₋₆haloalkyl, —C(O)NR⁸R⁹, C₂₋₉heterocycloalkyl, —C₁₋₆alkyl-C₂₋₉heterocycloalkyl, C₆₋₁₀aryl, or C₁₋₉heteroaryl, wherein C₂₋₉heterocycloalkyl, —C₁₋₆alkyl-C₂₋₉heterocycloalkyl, C₆₋₁₀aryl, or C₁₋₉heteroaryl are optionally substituted with 1 or 2 R¹⁴;
R⁵ is H, halogen, C₁₋₆alkyl, C₁₋₆haloalkyl, C₁₋₆haloalkoxy, or phenyl;
R⁶ is H, halogen or C₁₋₆alkyl;
R⁷ is H, C₁₋₆alkyl, C₁₋₆haloalkyl, C₂₋₉heterocycloalkyl, C₆₋₁₀aryl, or C₁₋₉heteroaryl, wherein C₂₋₉heterocycloalkyl, C₆₋₁₀aryl, or C₁₋₉heteroaryl are optionally substituted with 1 or 2 R¹⁴;
each R⁸ and each R⁹ are independently selected from H and C₁₋₆alkyl; or R⁸ and R⁹ together with the nitrogen to which they are attached are combined to form a heterocycloalkyl ring;
R¹⁰ and R¹¹ are each independently H or C₁₋₆alkyl;
R¹² is H, halogen, or C₁₋₆alkyl;
R¹³ is H or C₁₋₆alkyl;
each R¹⁴ is independently selected from halogen, —OH, C₁₋₆alkyl, C₁₋₆haloalkyl, C₁₋₆alkoxy, —C₁₋₆alkyl-OH, C₃₋₈cycloalkyl, —C(O)NR⁸R⁹, —SO₂—C₁₋₆alkyl, and —N(R¹⁷)C(O)—C₁₋₆alkyl;
R¹⁵ is H or C₁₋₆alkyl;
R¹⁷ is H or C₁₋₆alkyl;
n is 0 or 1;
m is 0 or 1;
p is 0, 1, or 2; and
q is 0, 1 or 2;
or a solvate, hydrate, tautomer, N-oxide, stereoisomer, or pharmaceutically acceptable salt thereof.

In some embodiments of the methods for treating a disease or condition with a compound of Formula (Id), R¹ is —C(O)OR¹⁵. In some embodiments of the methods for treating a disease or condition with a compound of Formula (Id), R¹ is —C(O)OR¹⁵ and R¹⁵ is H. In some embodiments of the methods for treating a disease or condition with a compound of Formula (Id), R¹ is —C(O)OR¹⁵ and R¹⁵ is C₁₋₆alkyl. In some embodiments of the methods for treating a disease or condition with a compound of Formula (Id), R¹ is —C(O)OR¹⁵ and R¹⁵ is —CH₃. In some embodiments of the methods for treating a disease or condition with a compound of Formula (Id), R¹ is —C(O)NR¹⁰R¹¹. In some embodiments of the methods for treating a disease or condition with a compound of Formula (Id), R¹ is —C(O)NR¹⁰R¹¹, R¹⁰ is H, and R¹¹ is H. In some embodiments of the methods for treating a disease or condition with a compound of Formula (Id), R¹ is —C(O)NR¹⁰R¹¹, R¹⁰ is H, and R¹¹ is C₁₋₆alkyl. In some embodiments of the methods for treating a disease or condition with a compound of Formula (Id), R¹ is —C(O)NR¹⁰R¹¹, R¹⁰ is H, and R¹¹ is —CH₃. In some embodiments of the methods for treating a disease or condition with a compound of Formula (Id), R¹ is —C(O)NR¹⁰R¹¹, R¹⁰ is C₁₋₆alkyl, and R¹¹ is C₁₋₆alkyl. In some embodiments of the methods for treating a disease or condition with a compound of Formula (Id), R¹ is —C(O)NR¹⁰R¹¹, R¹⁰ is —CH₃, and R¹¹ is —CH₃.

In some embodiments of the methods for treating a disease or condition with a compound of Formula (Id), R² is H. In some embodiments of the methods for treating a disease or condition with a compound of Formula (Id), R² is halogen. In some embodiments of the methods for treating a disease or condition with a compound of Formula (Id), R² is —Cl. In some embodiments of the methods for treating a disease or condition with a compound of Formula (Id), R² is C₁₋₆alkyl. In some embodiments of the methods for treating a disease or condition with a compound of Formula (Id), R² is —CH₃. In some embodiments of the methods for treating a disease or condition with a compound of Formula (Id), R² is C₁₋₆haloalkyl. In some embodiments of the methods for treating a disease or condition with a compound of Formula (Id), R² is —CF₃.

In some embodiments of the methods for treating a disease or condition with a compound of Formula (Id), R¹² is H. In some embodiments of the methods for treating a disease or condition with a compound of Formula (Id), R¹² is halogen. In some embodiments of the methods for treating a disease or condition with a compound of Formula (Id), R¹² is F. In some embodiments of the methods for treating a disease or condition with a compound of Formula (Id), R¹² is C₁₋₆alkyl. In some embodiments of the methods for treating a disease or condition with a compound of Formula (Id), R¹² is —CH₃. In some embodiments of the methods for treating a disease or condition with a compound of Formula (Id), R¹³ is H. In some embodiments of the methods for treating a disease or condition with a compound of Formula (Id), R¹³ is C₁₋₆alkyl. In some embodiments of the methods for treating a disease or condition with a compound of Formula (Id), R¹³ is —CH₃. In some embodiments of the methods for treating a disease or condition with a compound of Formula (Id), R¹² is H and R¹³ is H. In some embodiments of the methods for treating a disease or condition with a compound of Formula (Id), R¹² is H and R¹³ is C₁₋₆alkyl. In some embodiments of the methods for treating a disease or condition with a compound of Formula (Id), R¹² is H and R¹³ is —CH₃. In some embodiments of the methods for treating a disease or condition with a compound of Formula (Id), R¹² is C₁₋₆alkyl and R¹³ is H. In some embodiments of the methods for treating a disease or condition with a compound of Formula (Id), R¹² is —CH₃ and R¹³ is H. In some embodiments of the methods for treating a disease or condition with a compound of Formula (Id), R¹² is C₁₋₆alkyl and R¹³ is C₁₋₆alkyl. In some embodiments of the methods for treating a disease or condition with a compound of Formula (Id), R¹² is —CH₃ and R¹³ is —CH₃.

In some embodiments of the methods for treating a disease or condition with a compound of Formula (Id), A is C(H). In some embodiments of the methods for treating a disease or condition with a compound of Formula (Id), A is N.

In some embodiments of the methods for treating a disease or condition with a compound of Formula (Id), Y is —CH₂—. In some embodiments of the methods for treating a disease or condition with a compound of Formula (Id), Y is —C(O)—.

In some embodiments of the methods for treating a disease or condition with a compound of Formula (Id), m is 1, n is 1, q is 1, and p is 1. In some embodiments of the methods for treating a disease or condition with a compound of Formula (Id), m is 0, n is 1, q is 2, and p is 1. In some embodiments of the methods for treating a disease or condition with a compound of Formula (Id), m is 1, n is 1, q is 0, and p is 2. In some embodiments of the methods for treating a disease or condition with a compound of Formula (Id), m is 1, n is 1, q is 1, and p is 0. In some embodiments of the methods for treating a disease or condition with a compound of Formula (Id), m is 0, n is 0, q is 2, and p is 1.

In some embodiments of the methods for treating a disease or condition with a compound of Formula (Id), R⁴ is halogen, —OR⁷, C₁₋₆alkyl, C₂₋₆alkynyl, C₁₋₆haloalkyl, —C(O)NR⁸R⁹, C₂₋₉heterocycloalkyl, C₆₋₁₀aryl, or C₁₋₉heteroaryl, wherein C₂₋₉ heterocycloalkyl, —C₁₋₆alkyl-C₂₋₉heterocycloalkyl, C₆₋₁₀aryl, or C₁₋₉heteroaryl are optionally substituted with 1 or 2 R¹⁴. In some embodiments of the methods for treating a disease or condition with a compound of Formula (Id), R⁴ is halogen, —OR⁷, C₁₋₆haloalkyl, C₂₋₉heterocycloalkyl, C₆₋₁₀aryl, or C₁₋₉heteroaryl, wherein C₂₋₉ heterocycloalky, C₆₋₁₀aryl, or C₁₋₉heteroaryl are optionally substituted with 1 or 2 R¹⁴. In some embodiments of the methods for treating a disease or condition with a compound of Formula (Id), R⁴ is halogen. In some embodiments of the methods for treating a disease or condition with a compound of Formula (Id), R⁴ is —Cl. In some embodiments of the methods for treating a disease or condition with a compound of Formula (Id), R⁴ is C₁₋₆haloalkyl. In some embodiments of the methods for treating a disease or condition with a compound of Formula (Id), R⁴ is —CF₃. In some embodiments of the methods for treating a disease or condition with a compound of Formula (Id), R⁴ is —OR⁷. In some embodiments of the methods for treating a disease or condition with a compound of Formula (Id), R⁴ is —OR⁷ and R⁷ is C₁₋₆haloalkyl, C₆₋₁₀aryl, or C₁₋₉heteroaryl, wherein C₆₋₁₀aryl or C₁₋₉heteroaryl are optionally substituted with 1 or 2 R¹⁴. In some embodiments of the methods for treating a disease or condition with a compound of Formula (Id), R⁴ is —OR⁷ and R⁷ is C₁₋₆haloalkyl. In some embodiments of the methods for treating a disease or condition with a compound of Formula (Id), R⁴ is —OR⁷ and R⁷ is —CF₃. In some embodiments of the methods for treating a disease or condition with a compound of Formula (Id), R⁴ is —OR⁷ and R⁷ is C₆₋₁₀aryl or C₁₋₉heteroaryl, wherein C₆₋₁₀aryl or C₁₋₉heteroaryl are optionally substituted with 1 or 2 R¹⁴. In some embodiments of the methods for treating a disease or condition with a compound of Formula (Id), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are optionally substituted with halogen or C₁₋₆haloalkyl. In some embodiments of the methods for treating a disease or condition with a compound of Formula (Id), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are unsubstituted. In some embodiments of the methods for treating a disease or condition with a compound of Formula (Id), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are substituted with halogen or C₁₋₆haloalkyl. In some embodiments of the methods for treating a disease or condition with a compound of Formula (Id), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are substituted with —Cl or —CF₃. In some embodiments of the methods for treating a disease or condition with a compound of Formula (Id), R⁴ is C₂₋₉heterocycloalkyl optionally substituted with 1 or 2 R¹⁴. In some embodiments of the methods for treating a disease or condition with a compound of Formula (Id), R⁴ is an unsubstituted C₂₋₉ heterocycloalkyl. In some embodiments of the methods for treating a disease or condition with a compound of Formula (Id), R⁴ is C₂₋₉heterocycloalkyl substituted with 1 or 2 R¹⁴. In some embodiments of the methods for treating a disease or condition with a compound of Formula (Id), R⁴ is

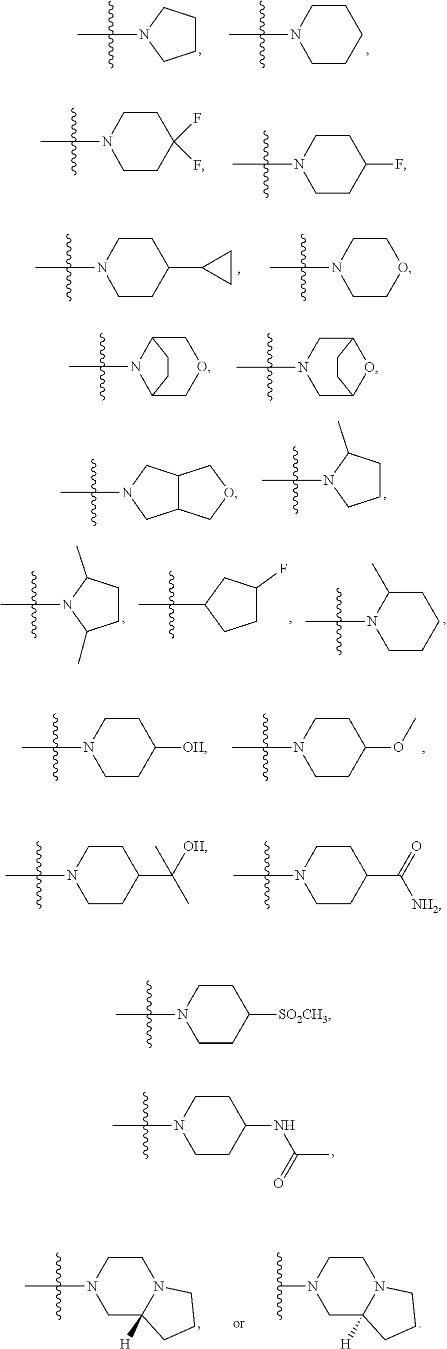

In some embodiments of the methods for treating a disease or condition with a compound of Formula (Id), R⁴ is C₁₋₉heteroaryl optionally substituted with 1 or 2 R¹⁴. In some embodiments of the methods for treating a disease or condition with a compound of Formula (Id), R⁴ is an unsubstituted C₁₋₉ heteroaryl. In some embodiments of the methods for treating a disease or condition with a compound of Formula (Id), R⁴ is C₂₋₉heteroaryl substituted with 1 or 2 R¹⁴. In some embodiments of the methods for treating a disease or condition with a compound of Formula (Id), R⁴ is

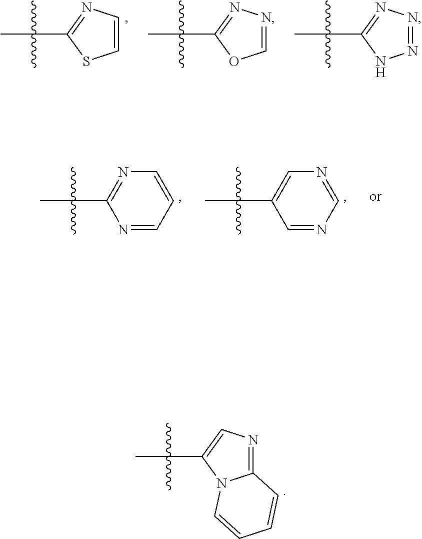 [SMILES: *c1nccs1 ; *c1nnco1 ; *c1nnn[nH]1 ; *c1ncccn1 ; *c1cncnc1 ; *c1cnc2ccccn12]

In some embodiments of the methods for treating a disease or condition with a compound of Formula (Id), R⁵ is H, halogen, C₁₋₆alkyl, C₁₋₆haloalkyl, C₁₋₆haloalkoxy. In some embodiments of the methods for treating a disease or condition with a compound of Formula (Id), R⁵ is H. In some embodiments of the methods for treating a disease or condition with a compound of Formula (Id), R⁵ is halogen. In some embodiments of the methods for treating a disease or condition with a compound of Formula (Id), R⁵ is —Cl. In some embodiments of the methods for treating a disease or condition with a compound of Formula (Id), R⁵ is —F. In some embodiments of the methods for treating a disease or condition with a compound of Formula (Id), R⁵ is C₁₋₆alkyl. In some embodiments of the methods for treating a disease or condition with a compound of Formula (Id), R⁵ is —CH₃. In some embodiments of the methods for treating a disease or condition with a compound of Formula (Id), R⁵ is C₁₋₆haloalkyl. In some embodiments of the methods for treating a disease or condition with a compound of Formula (Id), R⁵ is —CF₃. In some embodiments of the methods for treating a disease or condition with a compound of Formula (Id), R⁵ is C₁₋₆haloalkoxy. In some embodiments of the methods for treating a disease or condition with a compound of Formula (Id), R⁵ is —OCF₃.

In some embodiments of the methods for treating a disease or condition with a compound of Formula (Id), R⁶ is H. In some embodiments of the methods for treating a disease or condition with a compound of Formula (Id), R⁶ is halogen. In some embodiments of the methods for treating a disease or condition with a compound of Formula (Id), R⁶ is —Cl. In some embodiments of the methods for treating a disease or condition with a compound of Formula (Id), R⁶ is —F. In some embodiments of the methods for treating a disease or condition with a compound of Formula (Id), R⁶ is C₁₋₆alkyl. In some embodiments of the methods for treating a disease or condition with a compound of Formula (Id), R⁶ is —CH₃.

In some embodiments is a method for treating a disease or a condition in a patient wherein the disease or condition is selected from atopic dermatitis, bladder dysfunction associated with multiple sclerosis, cardiovascular disease, contact dermatitis, cystic fibrosis, dermatomyositis, eczema, endometriosis, enteritis, fibromyalgia, Tourette syndrome, inflammatory bowel disease, interstitial cystitis, irritable bowel syndrome, ischemia, labor, abdominal pain, abdominal pain associated with irritable bowel syndrome, acute pain, back pain, cancer pain, chest pain, functional chest pain, joint pain, menstrual pain, metabolic disorders, musculoskeletal diseases, neuropathy, osteoarthritis, pancreatitis, pharyngitis, post mastectomy pain syndrome, post trigeminal neuralgia, post operative pain, renal ischemia, rheumatoid arthritis, skeletal muscle contusion, skin diseases, sunburn, systemic lupus erythematosus, toothache, vasoocclusive painful crises in sickle cell disease, and visceral pain, comprising administering to the patient in need thereof a therapeutically effective amount of a compound of Formula (Ie) having the structure:

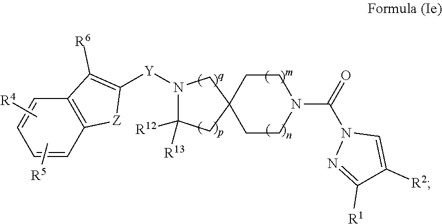

wherein:
R¹ is —C(O)OR¹⁵ or —C(O)NR¹⁰R¹¹;
R² is H, halogen, C₁₋₆alkyl, or C₁₋₆haloalkyl;
Y is —CH₂— or —C(O)—;
Z is —S—, —O—, or —N(R¹⁸)—;
R⁴ is H, halogen, —OR⁷, C₁₋₆alkyl, C₂₋₆alkenyl, C₂₋₆alkynyl, C₁₋₆haloalkyl, —C(O)NR⁸R⁹, C₂₋₉heterocycloalkyl, —C₁₋₆alkyl-C₂₋₉heterocycloalkyl, C₆₋₁₀aryl, or C₁₋₉heteroaryl, wherein C₂₋₉heterocycloalkyl, —C₁₋₆alkyl-C₂₋₉heterocycloalkyl, C₆₋₁₀aryl, or C₁₋₉heteroaryl are optionally substituted with 1 or 2 R¹⁴;
R⁵ is H, halogen, C₁₋₆alkyl, C₁₋₆haloalkyl, C₁₋₆haloalkoxy, or phenyl;
R⁶ is H, halogen or C₁₋₆alkyl;
R⁷ is H, C₁₋₆alkyl, C₁₋₆haloalkyl, C₂₋₉heterocycloalkyl, C₆₋₁₀aryl, or C₁₋₉heteroaryl, wherein C₂₋₉heterocycloalkyl, C₆₋₁₀aryl, or C₁₋₉heteroaryl are optionally substituted with 1 or 2 R¹⁴;
each R⁸ and each R⁹ are independently selected from H and C₁₋₆alkyl; or R⁸ and R⁹ together with the nitrogen to which they are attached are combined to form a heterocycloalkyl ring;
R¹⁰ and R¹¹ are each independently H or C₁₋₆alkyl;
R¹² is H, halogen, or C₁₋₆alkyl;
R¹³ is H or C₁₋₆alkyl;
each R¹⁴ is independently selected from halogen, —OH, C₁₋₆alkyl, C₁₋₆haloalkyl, C₁₋₆alkoxy, —C₁₋₆alkyl-OH, C₃₋₈cycloalkyl, —C(O)NR⁸R⁹, —SO₂—C₁₋₆alkyl, and —N(R¹⁷)C(O)—C₁₋₆alkyl;
R¹⁵ is H or C₁₋₆alkyl;
R¹⁷ is H or C₁₋₆alkyl;
R¹⁸ is H or C₁₋₆alkyl;
n is 0 or 1;
m is 0 or 1;
p is 0, 1, or 2; and
q is 0, 1 or 2;
or a solvate, hydrate, tautomer, N-oxide, stereoisomer, or pharmaceutically acceptable salt thereof.

In some embodiments of the methods for treating a disease or condition with a compound of Formula (Ie), R¹ is —C(O)OR¹⁵. In some embodiments of the methods for treating a disease or condition with a compound of Formula (Ie), R¹ is —C(O)OR¹⁵ and R¹⁵ is H. In some embodiments of the methods for treating a disease or condition with a compound of Formula (Ie), R¹ is —C(O)OR¹⁵ and R¹⁵ is C₁₋₆alkyl. In some embodiments of the methods for treating a disease or condition with a compound of Formula (Ie), R¹ is —C(O)OR¹⁵ and R¹⁵ is —CH₃. In some embodiments of the methods for treating a disease or condition with a compound of Formula (Ie), R¹ is —C(O)NR¹⁰R¹¹. In some embodiments of the methods for treating a disease or condition with a compound of Formula (Ie), R¹ is —C(O)NR¹⁰R¹¹, R¹⁰ is H, and R¹¹ is H. In some embodiments of the methods for treating a disease or condition with a compound of Formula (Ie), R¹ is —C(O)NR¹⁰R¹¹, R¹⁰ is H, and R¹¹ is C₁₋₆alkyl. In some embodiments of the methods for treating a disease or condition with a compound of Formula (Ie), R¹ is —C(O)NR¹⁰R¹¹, R¹⁰ is H, and R¹¹ is —CH₃. In some embodiments of the methods for treating a disease or condition with a compound of Formula (Ie), R¹ is —C(O)NR¹⁰R¹¹, R¹⁰ is C₁₋₆alkyl, and R¹¹ is C₁₋₆alkyl. In some embodiments of the methods for treating a disease or condition with a compound of Formula (Ie), R¹ is —C(O)NR¹⁰R¹¹, R¹⁰ is —CH₃, and R¹¹ is —CH₃.

In some embodiments of the methods for treating a disease or condition with a compound of Formula (Ie), R² is H. In some embodiments of the methods for treating a disease or condition with a compound of Formula (Ie), R² is halogen. In some embodiments of the methods for treating a disease or condition with a compound of Formula (Ie), R² is —Cl. In some embodiments of the methods for treating a disease or condition with a compound of Formula (Ie), R² is C₁₋₆alkyl. In some embodiments of the methods for treating a disease or condition with a compound of Formula (Ie), R² is —CH₃. In some embodiments of the methods for treating a disease or condition with a compound of Formula (Ie), R² is C₁₋₆haloalkyl. In some embodiments of the methods for treating a disease or condition with a compound of Formula (Ie), R² is —CF₃.

In some embodiments of the methods for treating a disease or condition with a compound of Formula (Ie), R¹² is H. In some embodiments of the methods for treating a disease or condition with a compound of Formula (Ie), R¹² is halogen. In some embodiments of the methods for treating a disease or condition with a compound of Formula (Ie), R¹² is F. In some embodiments of the methods for treating a disease or condition with a compound of Formula (Ie), R¹² is C₁₋₆alkyl. In some embodiments of the methods for treating a disease or condition with a compound of Formula (Ie), R¹² is —CH₃. In some embodiments of the methods for treating a disease or condition with a compound of Formula (Ie), R¹³ is H. In some embodiments of the methods for treating a disease or condition with a compound of Formula (Ie), R¹³ is C₁₋₆alkyl. In some embodiments of the methods for treating a disease or condition with a compound of Formula (Ie), R¹³ is —CH₃. In some embodiments of the methods for treating a disease or condition with a compound of Formula (Ie), R¹² is H and R¹³ is H. In some embodiments of the methods for treating a disease or condition with a compound of Formula (Ie), R¹² is H and R¹³ is C₁₋₆alkyl. In some embodiments of the methods for treating a disease or condition with a compound of Formula (Ie), R¹² is H and R¹³ is —CH₃. In some embodiments of the methods for treating a disease or condition with a compound of Formula (Ie), R¹² is C₁₋₆alkyl and R¹³ is H. In some embodiments of the methods for treating a disease or condition with a compound of Formula (Ie), R¹² is —CH₃ and R¹³ is H. In some embodiments of the methods for treating a disease or condition with a compound of Formula (Ie), R¹² is C₁₋₆alkyl and R¹³ is C₁₋₆alkyl. In some embodiments of the methods for treating a disease or condition with a compound of Formula (Ie), R¹² is —CH₃ and R¹³ is —CH₃.

In some embodiments of the methods for treating a disease or condition with a compound of Formula (Ie), Y is —CH₂—. In some embodiments of the methods for treating a disease or condition with a compound of Formula (Ie), Y is —C(O)—.

In some embodiments of the methods for treating a disease or condition with a compound of Formula (Ie), Z is —S—. In some embodiments of the methods for treating a disease or condition with a compound of Formula (Ie), Z is —O—. In some embodiments of the methods for treating a disease or condition with a compound of Formula (Ie), Z is —N(R¹⁸)—. In some embodiments of the methods for treating a disease or condition with a compound of Formula (Ie), Z is —N(H)—. In some embodiments of the methods for treating a disease or condition with a compound of Formula (Ie), Z is —N(CH₃)—.

In some embodiments of the methods for treating a disease or condition with a compound of Formula (Ie), m is 1, n is 1, q is 1, and p is 1. In some embodiments of the methods for treating a disease or condition with a compound of Formula (Ie), m is 0, n is 1, q is 2, and p is 1. In some embodiments of the methods for treating a disease or condition with a compound of Formula (Ie), m is 1, n is 1, q is 0, and p is 2. In some embodiments of the methods for treating a disease or condition with a compound of Formula (Ie), m is 1, n is 1, q is 1, and p is 0. In some embodiments of the methods for treating a disease or condition with a compound of Formula (Ie), m is 0, n is 0, q is 2, and p is 1.

In some embodiments of the methods for treating a disease or condition with a compound of Formula (Ie), R⁴ is halogen, —OR⁷, C₁₋₆alkyl, C₂₋₆alkynyl, C₁₋₆haloalkyl, —C(O)NR⁸R⁹, C₂₋₉heterocycloalkyl, C₆₋₁₀aryl, or C₁₋₉heteroaryl, wherein C₂₋₉ heterocycloalkyl, —C₁₋₆alkyl-C₂₋₉heterocycloalkyl, C₆₋₁₀aryl, or C₁₋₉heteroaryl are optionally substituted with 1 or 2 R¹⁴. In some embodiments of the methods for treating a disease or condition with a compound of Formula (Ie), R⁴ is halogen, —OR⁷, C₁₋₆haloalkyl, C₂₋₉heterocycloalkyl, C₆₋₁₀aryl, or C₁₋₉heteroaryl, wherein C₂₋₉ heterocycloalkyl, C₆₋₁₀aryl, or C₁₋₉heteroaryl are optionally substituted with 1 or 2 R¹⁴. In some embodiments of the methods for treating a disease or condition with a compound of Formula (Ie), R⁴ is halogen. In some embodiments of the methods for treating a disease or condition with a compound of Formula (Ie), R⁴ is —Cl. In some embodiments of the methods for treating a disease or condition with a compound of Formula (Ie), R⁴ is C₁₋₆haloalkyl. In some embodiments of the methods for treating a disease or condition with a compound of Formula (Ie), R⁴ is —CF₃. In some embodiments of the methods for treating a disease or condition with a compound of Formula (Ie), R⁴ is —OR⁷. In some embodiments of the methods for treating a disease or condition with a compound of Formula (Ie), R⁴ is —OR⁷ and R⁷ is C₁₋₆haloalkyl, C₆₋₁₀aryl, or C₁₋₉heteroaryl, wherein C₆₋₁₀aryl or C₁₋₉heteroaryl are optionally substituted with 1 or 2 R¹⁴. In some embodiments of the methods for treating a disease or condition with a compound of Formula (Ie), R⁴ is —OR⁷ and R⁷ is C₁₋₆haloalkyl. In some embodiments of the methods for treating a disease or condition with a compound of Formula (Ie), R⁴ is —OR⁷ and R⁷ is —CF₃. In some embodiments of the methods for treating a disease or condition with a compound of Formula (Ie), R⁴ is —OR⁷ and R⁷ is C₆₋₁₀aryl or C₁₋₉heteroaryl, wherein C₆₋₁₀aryl or C₁₋₉heteroaryl are optionally substituted with 1 or 2 R¹⁴. In some embodiments of the methods for treating a disease or condition with a compound of Formula (Ie), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are optionally substituted with halogen or C₁₋₆haloalkyl. In some embodiments of the methods for treating a disease or condition with a compound of Formula (Ie), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are unsubstituted. In some embodiments of the methods for treating a disease or condition with a compound of Formula (Ie), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are substituted with halogen or C₁₋₆haloalkyl. In some embodiments of the methods for treating a disease or condition with a compound of Formula (Ie), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are substituted with —Cl or —CF₃. In some embodiments of the methods for treating a disease or condition with a compound of Formula (Ie), R⁴ is C₂₋₉heterocycloalkyl optionally substituted with 1 or 2 R¹⁴. In some embodiments of the methods for treating a disease or condition with a compound of Formula (Ie), R⁴ is an unsubstituted C₂₋₉ heterocycloalkyl. In some embodiments of the methods for treating a disease or condition with a compound of Formula (Ie), R⁴ is C₂₋₉heterocycloalkyl substituted with 1 or 2 R¹⁴. In some embodiments of the methods for treating a disease or condition with a compound of Formula (Ie), R⁴ is

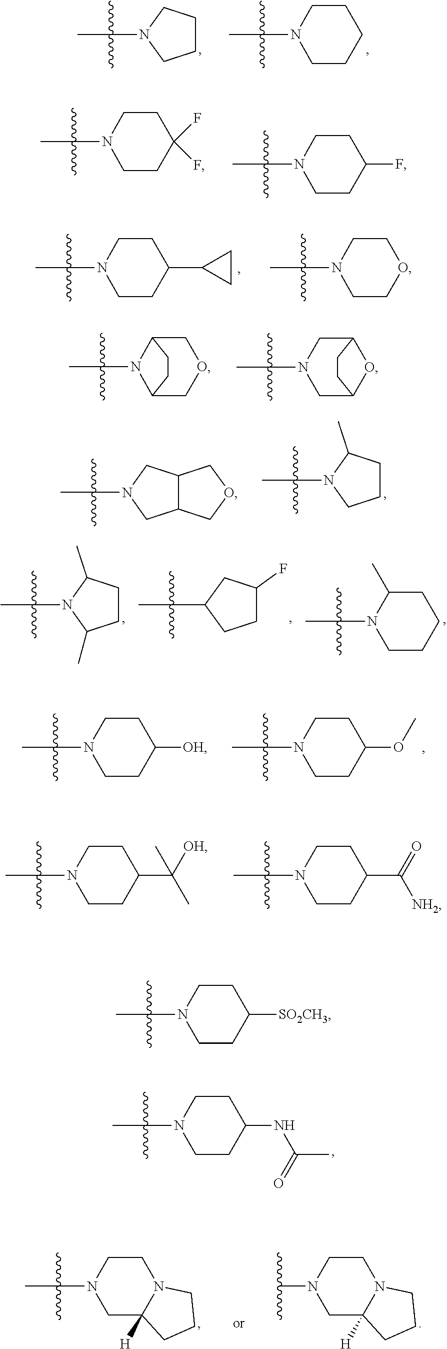

In some embodiments of the methods for treating a disease or condition with a compound of Formula (Ie), R⁴ is C₁₋₉heteroaryl optionally substituted with 1 or 2 R¹⁴. In some embodiments of the methods for treating a disease or condition with a compound of Formula (Ie), R⁴ is an unsubstituted C₁₋₉heteroaryl. In some embodiments of the methods for treating a disease or condition with a compound of Formula (Ie), R⁴ is C₂₋₉heteroaryl substituted with 1 or 2 R¹⁴. In some embodiments of the methods for treating a disease or condition with a compound of Formula (Ie), R⁴ is

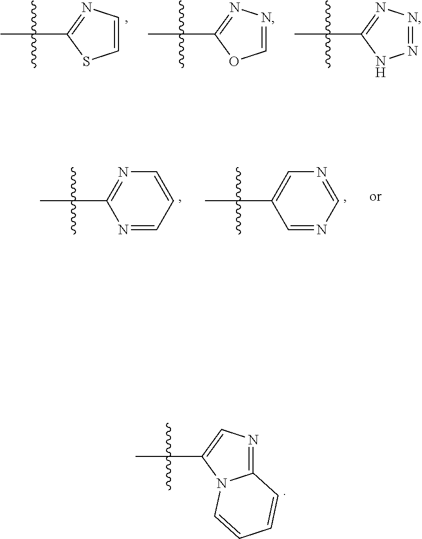 [SMILES: *c1nccs1 ; *c1nnco1 ; *c1nnn[nH]1 ; *c1ncccn1 ; *c1cncnc1 ; *c1cnc2ccccn12]

In some embodiments of the methods for treating a disease or condition with a compound of Formula (Ie), R⁵ is H, halogen, C₁₋₆alkyl, C₁₋₆haloalkyl, C₁₋₆haloalkoxy. In some embodiments of the methods for treating a disease or condition with a compound of Formula (Ie), R⁵ is H. In some embodiments of the methods for treating a disease or condition with a compound of Formula (Ie), R⁵ is halogen. In some embodiments of the methods for treating a disease or condition with a compound of Formula (Ie), R⁵ is —Cl. In some embodiments of the methods for treating a disease or condition with a compound of Formula (Ie), R⁵ is —F. In some embodiments of the methods for treating a disease or condition with a compound of Formula (Ie), R⁵ is C₁₋₆alkyl. In some embodiments of the methods for treating a disease or condition with a compound of Formula (Ie), R⁵ is —CH₃. In some embodiments of the methods for treating a disease or condition with a compound of Formula (Ie), R⁵ is C₁₋₆haloalkyl. In some embodiments of the methods for treating a disease or condition with a compound of Formula (Ie), R⁵ is —CF₃. In some embodiments of the methods for treating a disease or condition with a compound of Formula (Ie), R⁵ is C₁₋₆haloalkoxy. In some embodiments of the methods for treating a disease or condition with a compound of Formula (Ie), R⁵ is —OCF₃.

In some embodiments of the methods for treating a disease or condition with a compound of Formula (Ie), R⁶ is H. In some embodiments of the methods for treating a disease or condition with a compound of Formula (Ie), R⁶ is halogen. In some embodiments of the methods for treating a disease or condition with a compound of Formula (Ie), R⁶ is —Cl. In some embodiments of the methods for treating a disease or condition with a compound of Formula (Ie), R⁶ is —F. In some embodiments of the methods for treating a disease or condition with a compound of Formula (Ie), R⁶ is C₁₋₆alkyl. In some embodiments of the methods for treating a disease or condition with a compound of Formula (Ie), R⁶ is —CH₃.

In some embodiments is a method for treating a disease or a condition in a patient wherein the disease or condition is selected from atopic dermatitis, bladder dysfunction associated with multiple sclerosis, cardiovascular disease, contact dermatitis, cystic fibrosis, dermatomyositis, eczema, endometriosis, enteritis, fibromyalgia, Tourette syndrome, inflammatory bowel disease, interstitial cystitis, irritable bowel syndrome, ischemia, labor, abdominal pain, abdominal pain associated with irritable bowel syndrome, acute pain, back pain, cancer pain, chest pain, functional chest pain, joint pain, menstrual pain, metabolic disorders, musculoskeletal diseases, neuropathy, osteoarthritis, pancreatitis, pharyngitis, post mastectomy pain syndrome, post trigeminal neuralgia, post operative pain, renal ischemia, rheumatoid arthritis, skeletal muscle contusion, skin diseases, sunburn, systemic lupus erythematosus, toothache, vasoocclusive painful crises in sickle cell disease, and visceral pain, comprising administering to the patient in need thereof a therapeutically effective amount of a compound of Formula (If) having the structure:

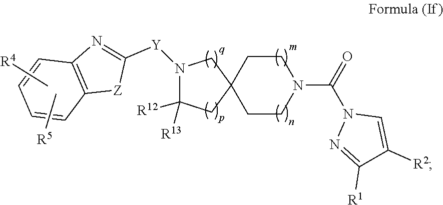

wherein:
R¹ is —C(O)OR¹⁵ or —C(O)NR¹⁰R¹¹;
R² is H, halogen, C₁₋₆alkyl, or C₁₋₆haloalkyl;
Y is —CH₂— or —C(O)—;
Z is —S—, —O—, or —N(R¹⁸)—;
R⁴ is H, halogen, —OR⁷, C₁₋₆alkyl, C₂₋₆alkenyl, C₂₋₆alkynyl, C₁₋₆haloalkyl, —C(O)NR⁸R⁹, C₂₋₉heterocycloalkyl, —C₁₋₆alkyl-C₂₋₉heterocycloalkyl, C₆₋₁₀aryl, or C₁₋₉heteroaryl, wherein C₂₋₉heterocycloalkyl, —C₁₋₆alkyl-C₂₋₉heterocycloalkyl, C₆₋₁₀aryl, or C₁₋₉heteroaryl are optionally substituted with 1 or 2 R¹⁴;
R⁵ is H, halogen, C₁₋₆alkyl, C₁₋₆haloalkyl, C₁₋₆haloalkoxy, or phenyl;
R⁷ is H, C₁₋₆alkyl, C₁₋₆haloalkyl, C₂₋₉heterocycloalkyl, C₆₋₁₀aryl, or C₁₋₉heteroaryl, wherein C₂₋₉heterocycloalkyl, C₆₋₁₀aryl, or C₁₋₉heteroaryl are optionally substituted with 1 or 2 R¹⁴;
each R⁸ and each R⁹ are independently selected from H and C₁₋₆alkyl; or R⁸ and R⁹ together with the nitrogen to which they are attached are combined to form a heterocycloalkyl ring;
R¹⁰ and R¹¹ are each independently H or C₁₋₆alkyl;
R¹² is H, halogen, or C₁₋₆alkyl;
R¹³ is H or C₁₋₆alkyl;
each R¹⁴ is independently selected from halogen, —OH, C₁₋₆alkyl, C₁₋₆haloalkyl, C₁₋₆alkoxy, —C₁₋₆alkyl-OH, C₃₋₈cycloalkyl, —C(O)NR⁸R⁹, —SO₂—C₁₋₆alkyl, and —N(R¹⁷)C(O)—C₁₋₆alkyl;
R¹⁵ is H or C₁₋₆alkyl;
R¹⁷ is H or C₁₋₆alkyl;
R¹⁸ is H or C₁₋₆alkyl;
n is 0 or 1;
m is 0 or 1;
p is 0, 1, or 2; and
q is 0, 1 or 2;
or a solvate, hydrate, tautomer, N-oxide, stereoisomer, or pharmaceutically acceptable salt thereof.

In some embodiments of the methods for treating a disease or condition with a compound of Formula (If), R¹ is —C(O)OR¹⁵. In some embodiments of the methods for treating a disease or condition with a compound of Formula (If), R¹ is —C(O)OR¹⁵ and R¹⁵ is H. In some embodiments of the methods for treating a disease or condition with a compound of Formula (If), R¹ is —C(O)OR¹⁵ and R¹⁵ is C₁₋₆alkyl. In some embodiments of the methods for treating a disease or condition with a compound of Formula (If), R¹ is —C(O)OR¹⁵ and R¹⁵ is —CH₃. In some embodiments of the methods for treating a disease or condition with a compound of Formula (If), R¹ is —C(O)NR¹⁰R¹¹. In some embodiments of the methods for treating a disease or condition with a compound of Formula (If), R¹ is —C(O)NR¹⁰R¹¹, R¹⁰ is H, and R¹¹ is H. In some embodiments of the methods for treating a disease or condition with a compound of Formula (If), R¹ is —C(O)NR¹⁰R¹¹, R¹⁰ is H, and R¹¹ is C₁₋₆alkyl. In some embodiments of the methods for treating a disease or condition with a compound of Formula (If), R¹ is —C(O)NR¹⁰R¹¹, R¹⁰ is H, and R¹¹ is —CH₃. In some embodiments of the methods for treating a disease or condition with a compound of Formula (If), R¹ is —C(O)NR¹⁰R¹¹, R¹⁰ is C₁₋₆alkyl, and R¹¹ is C₁₋₆alkyl. In some embodiments of the methods for treating a disease or condition with a compound of Formula (If), R¹ is —C(O)NR¹⁰R¹¹, R¹⁰ is —CH₃, and R¹¹ is —CH₃.

In some embodiments of the methods for treating a disease or condition with a compound of Formula (If), R² is H. In some embodiments of the methods for treating a disease or condition with a compound of Formula (If), R² is halogen. In some embodiments of the methods for treating a disease or condition with a compound of Formula (If), R² is —Cl. In some embodiments of the methods for treating a disease or condition with a compound of Formula (If), R² is C₁₋₆alkyl. In some embodiments of the methods for treating a disease or condition with a compound of Formula (If), R² is —CH₃. In some embodiments of the methods for treating a disease or condition with a compound of Formula (If), R² is C₁₋₆haloalkyl. In some embodiments of the methods for treating a disease or condition with a compound of Formula (If), R² is —CF₃.

In some embodiments of the methods for treating a disease or condition with a compound of Formula (If), R¹² is H. In some embodiments of the methods for treating a disease or condition with a compound of Formula (If), R¹² is halogen. In some embodiments of the methods for treating a disease or condition with a compound of Formula (If), R¹² is F. In some embodiments of the methods for treating a disease or condition with a compound of Formula (If), R¹² is C₁₋₆alkyl. In some embodiments of the methods for treating a disease or condition with a compound of Formula (If), R¹² is —CH₃. In some embodiments of the methods for treating a disease or condition with a compound of Formula (If), R¹³ is H. In some embodiments of the methods for treating a disease or condition with a compound of Formula (If), R¹³ is C₁₋₆alkyl. In some embodiments of the methods for treating a disease or condition with a compound of Formula (If), R¹³ is —CH₃. In some embodiments of the methods for treating a disease or condition with a compound of Formula (If), R¹² is H and R¹³ is H. In some embodiments of the methods for treating a disease or condition with a compound of Formula (If), R¹² is H and R¹³ is C₁₋₆alkyl. In some embodiments of the methods for treating a disease or condition with a compound of Formula (If), R¹² is H and R¹³ is —CH₃. In some embodiments of the methods for treating a disease or condition with a compound of Formula (If), R¹² is C₁₋₆alkyl and R¹³ is H. In some embodiments of the methods for treating a disease or condition with a compound of Formula (If), R¹² is —CH₃ and R¹³ is H. In some embodiments of the methods for treating a disease or condition with a compound of Formula (If), R¹² is C₁₋₆alkyl and R¹³ is C₁₋₆alkyl. In some embodiments of the methods for treating a disease or condition with a compound of Formula (If), R¹² is —CH₃ and R¹³ is-CH₃.

In some embodiments of the methods for treating a disease or condition with a compound of Formula (If), Y is —CH₂—. In some embodiments of the methods for treating a disease or condition with a compound of Formula (If), Y is —C(O)—.

In some embodiments of the methods for treating a disease or condition with a compound of Formula (If), Z is —S—. In some embodiments of the methods for treating a disease or condition with a compound of Formula (If), Z is —O—. In some embodiments of the methods for treating a disease or condition with a compound of Formula (If), Z is —N(R¹⁸)—. In some embodiments of the methods for treating a disease or condition with a compound of Formula (If), Z is —N(H)—. In some embodiments of the methods for treating a disease or condition with a compound of Formula (If), Z is —N(CH₃)—.

In some embodiments of the methods for treating a disease or condition with a compound of Formula (If), m is 1, n is 1, q is 1, and p is 1. In some embodiments of the methods for treating a disease or condition with a compound of Formula (If), m is 0, n is 1, q is 2, and p is 1. In some embodiments of the methods for treating a disease or condition with a compound of Formula (If), m is 1, n is 1, q is 0, and p is 2. In some embodiments of the methods for treating a disease or condition with a compound of Formula (If), m is 1, n is 1, q is 1, and p is 0. In some embodiments of the methods for treating a disease or condition with a compound of Formula (If), m is 0, n is 0, q is 2, and p is 1.

In some embodiments of the methods for treating a disease or condition with a compound of Formula (If), R⁴ is halogen, —OR⁷, C₁₋₆alkyl, C₂₋₆alkynyl, C₁₋₆haloalkyl, —C(O)NR⁸R⁹, C₂₋₉heterocycloalkyl, C₆₋₁₀aryl, or C₁₋₉heteroaryl, wherein C₂₋₉ heterocycloalkyl, —C₁₋₆alkyl-C₂₋₉heterocycloalkyl, C₆₋₁₀aryl, or C₁₋₉heteroaryl are optionally substituted with 1 or 2 R¹⁴. In some embodiments of the methods for treating a disease or condition with a compound of Formula (If), R⁴ is halogen, —OR⁷, C₁₋₆haloalkyl, C₂₋₉heterocycloalkyl, C₆₋₁₀aryl, or C₁₋₉heteroaryl, wherein C₂₋₉ heterocycloalkyl, C₆₋₁₀aryl, or C₁₋₉heteroaryl are optionally substituted with 1 or 2 R¹⁴. In some embodiments of the methods for treating a disease or condition with a compound of Formula (If), R⁴ is halogen. In some embodiments of the methods for treating a disease or condition with a compound of Formula (If), R⁴ is —Cl. In some embodiments of the methods for treating a disease or condition with a compound of Formula (If), R⁴ is C₁₋₆haloalkyl. In some embodiments of the methods for treating a disease or condition with a compound of Formula (If), R⁴ is —CF₃. In some embodiments of the methods for treating a disease or condition with a compound of Formula (If), R⁴ is —OR⁷. In some embodiments of the methods for treating a disease or condition with a compound of Formula (If), R⁴ is —OR⁷ and R⁷ is C₁₋₆haloalkyl, C₆₋₁₀aryl, or C₁₋₉heteroaryl, wherein C₆₋₁₀aryl or C₁₋₉heteroaryl are optionally substituted with 1 or 2 R¹⁴. In some embodiments of the methods for treating a disease or condition with a compound of Formula (If), R⁴ is —OR⁷ and R⁷ is C₁₋₆haloalkyl. In some embodiments of the methods for treating a disease or condition with a compound of Formula (If), R⁴ is —OR⁷ and R⁷ is —CF₃. In some embodiments of the methods for treating a disease or condition with a compound of Formula (If), R⁴ is —OR⁷ and R⁷ is C₆₋₁₀aryl or C₁₋₉heteroaryl, wherein C₆₋₁₀aryl or C₁₋₉heteroaryl are optionally substituted with 1 or 2 R¹⁴. In some embodiments of the methods for treating a disease or condition with a compound of Formula (If), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are optionally substituted with halogen or C₁₋₆haloalkyl. In some embodiments of the methods for treating a disease or condition with a compound of Formula (If), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are unsubstituted. In some embodiments of the methods for treating a disease or condition with a compound of Formula (If), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are substituted with halogen or C₁₋₆haloalkyl. In some embodiments of the methods for treating a disease or condition with a compound of Formula (If), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are substituted with —Cl or —CF₃. In some embodiments of the methods for treating a disease or condition with a compound of Formula (If), R⁴ is C₂₋₉heterocycloalkyl optionally substituted with 1 or 2 R¹⁴. In some embodiments of the methods for treating a disease or condition with a compound of Formula (If), R⁴ is an unsubstituted C₂₋₉ heterocycloalkyl. In some embodiments of the methods for treating a disease or condition with a compound of Formula (If), R⁴ is C₂₋₉heterocycloalkyl substituted with 1 or 2 R¹⁴. In some embodiments of the methods for treating a disease or condition with a compound of Formula (If), R⁴ is

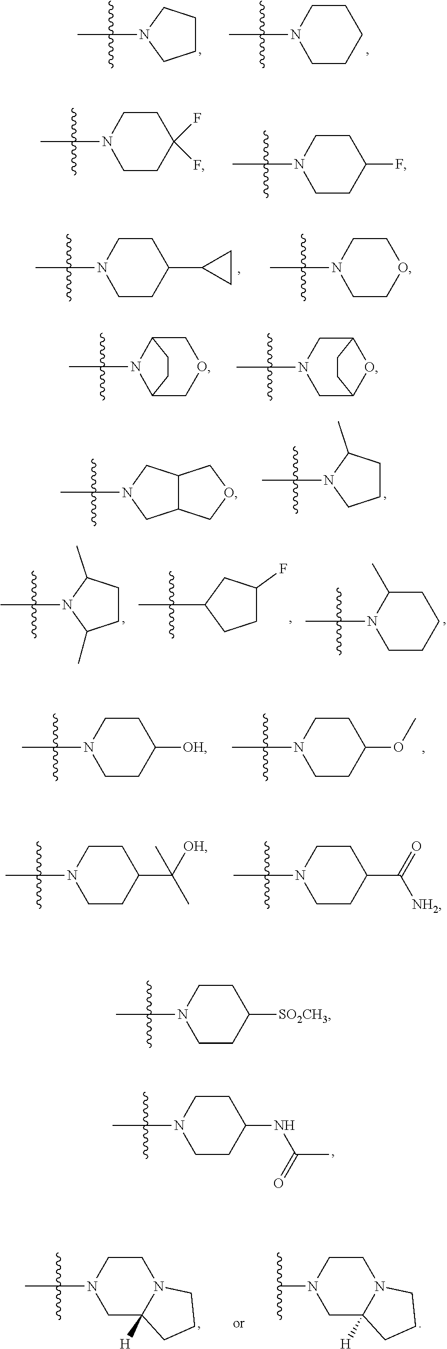

In some embodiments of the methods for treating a disease or condition with a compound of Formula (If), R⁴ is C₁₋₉heteroaryl optionally substituted with 1 or 2 R¹⁴. In some embodiments of the methods for treating a disease or condition with a compound of Formula (If), R⁴ is an unsubstituted C₁₋₉heteroaryl. In some embodiments of the methods for treating a disease or condition with a compound of Formula (If), R⁴ is C₂₋₉heteroaryl substituted with 1 or 2 R¹⁴. In some embodiments of the methods for treating a disease or condition with a compound of Formula (If), R⁴ is

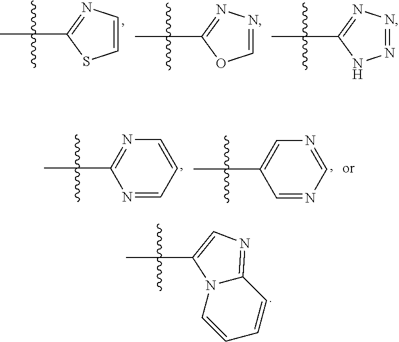 [SMILES: *c1nccs1 ; *c1nnco1 ; *c1nnn[nH]1 ; *c1ncccn1 ; *c1cncnc1 ; *c1cnc2ccccn12]

In some embodiments of the methods for treating a disease or condition with a compound of Formula (If), R⁵ is H, halogen, C₁₋₆alkyl, C₁₋₆haloalkyl, C₁₋₆haloalkoxy. In some embodiments of the methods for treating a disease or condition with a compound of Formula (If), R⁵ is H. In some embodiments of the methods for treating a disease or condition with a compound of Formula (If), R⁵ is halogen. In some embodiments of the methods for treating a disease or condition with a compound of Formula (If), R⁵ is —Cl. In some embodiments of the methods for treating a disease or condition with a compound of Formula (If), R⁵ is —F. In some embodiments of the methods for treating a disease or condition with a compound of Formula (If), R⁵ is C₁₋₆alkyl. In some embodiments of the methods for treating a disease or condition with a compound of Formula (If), R⁵ is —CH₃. In some embodiments of the methods for treating a disease or condition with a compound of Formula (If), R⁵ is C₁₋₆haloalkyl. In some embodiments of the methods for treating a disease or condition with a compound of Formula (If), R⁵ is —CF₃. In some embodiments of the methods for treating a disease or condition with a compound of Formula (If), R⁵ is C₁₋₆haloalkoxy. In some embodiments of the methods for treating a disease or condition with a compound of Formula (If), R⁵ is —OCF₃.

In some embodiments is a method for treating a disease or a condition in a patient wherein the disease or condition is selected from atopic dermatitis, bladder dysfunction associated with multiple sclerosis, cardiovascular disease, contact dermatitis, cystic fibrosis, dermatomyositis, eczema, endometriosis, enteritis, fibromyalgia, Tourette syndrome, inflammatory bowel disease, interstitial cystitis, irritable bowel syndrome, ischemia, labor, abdominal pain, abdominal pain associated with irritable bowel syndrome, acute pain, back pain, cancer pain, chest pain, functional chest pain, joint pain, menstrual pain, metabolic disorders, musculoskeletal diseases, neuropathy, osteoarthritis, pancreatitis, pharyngitis, post mastectomy pain syndrome, post trigeminal neuralgia, post operative pain, renal ischemia, rheumatoid arthritis, skeletal muscle contusion, skin diseases, sunburn, systemic lupus erythematosus, toothache, vasoocclusive painful crises in sickle cell disease, and visceral pain, comprising administering to the patient in need thereof a therapeutically effective amount of a compound of Formula (Ig) having the structure:

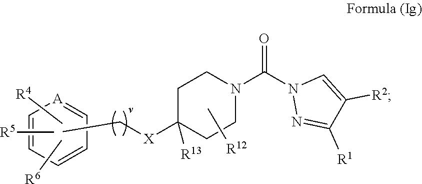

wherein:
R¹ is —C(O)OR¹⁵ or —C(O)NR¹⁰R¹¹;
R² is H, halogen, C₁₋₆alkyl, or C₁₋₆haloalkyl;
A is N or C(H);
X is —O—, —N(R¹⁶)—, or —CH₂N(R¹⁶)CH₂—;
R⁴ is H, halogen, —OR⁷, C₁₋₆alkyl, C₂₋₆alkenyl, C₂₋₆alkynyl, C₁₋₆haloalkyl, —C(O)NR⁸R⁹, C₂₋₉heterocycloalkyl, —C₁₋₆alkyl-C₂₋₉heterocycloalkyl, C₆₋₁₀aryl, or C₁₋₉heteroaryl, wherein C₂₋₉heterocycloalkyl, —C₁₋₆alkyl-C₂₋₉heterocycloalkyl, C₆₋₁₀aryl, or C₁₋₉heteroaryl are optionally substituted with 1 or 2 R¹⁴;
R⁵ is H, halogen, C₁₋₆alkyl, C₁₋₆haloalkyl, C₁₋₆haloalkoxy, or phenyl;
R⁶ is H, halogen or C₁₋₆alkyl;
R⁷ is H, C₁₋₆alkyl, C₁₋₆haloalkyl, C₂₋₉heterocycloalkyl, C₆₋₁₀aryl, or C₁₋₉heteroaryl, wherein C₂₋₉heterocycloalkyl, C₆₋₁₀aryl, or C₁₋₉heteroaryl are optionally substituted with 1 or 2 R¹⁴;
each R⁸ and each R⁹ are independently selected from H and C₁₋₆alkyl; or R⁸ and R⁹ together with the nitrogen to which they are attached are combined to form a heterocycloalkyl ring;
R¹⁰ and R¹¹ are each independently H or C₁₋₆alkyl;
R¹² is H, halogen, or C₁₋₆alkyl;
R¹³ is H or C₁₋₆alkyl;
each R¹⁴ is independently selected from halogen, —OH, C₁₋₆alkyl, C₁₋₆haloalkyl, C₁₋₆alkoxy, —C₁₋₆alkyl-OH, C₃₋₈cycloalkyl, —C(O)NR⁸R⁹, —SO₂—C₁₋₆alkyl, and —N(R¹⁷)C(O)—C₁₋₆alkyl;
R¹⁵ is H or C₁₋₆alkyl;
R¹⁶ is H, C₁₋₆alkyl, —C(O)—C₁₋₆alkyl, or —CH₂CO₂H;
R¹⁷ is H or C₁₋₆alkyl; and
v is 0 or 1;
or a solvate, hydrate, tautomer, N-oxide, stereoisomer, or pharmaceutically acceptable salt thereof.

In some embodiments of the methods for treating a disease or condition with a compound of Formula (Ig), R¹ is —C(O)OR¹⁵. In some embodiments of the methods for treating a disease or condition with a compound of Formula (Ig), R¹ is —C(O)OR¹⁵ and R¹⁵ is H. In some embodiments of the methods for treating a disease or condition with a compound of Formula (Ig), R¹ is —C(O)OR¹⁵ and R¹⁵ is C₁₋₆alkyl. In some embodiments of the methods for treating a disease or condition with a compound of Formula (Ig), R¹ is —C(O)OR¹⁵ and R¹⁵ is —CH₃. In some embodiments of the methods for treating a disease or condition with a compound of Formula (Ig), R¹ is —C(O)NR¹⁰R¹¹. In some embodiments of the methods for treating a disease or condition with a compound of Formula (Ig), R¹ is —C(O)NR¹⁰R¹¹, R¹⁰ is H, and R¹¹ is H. In some embodiments of the methods for treating a disease or condition with a compound of Formula (Ig), R¹ is —C(O)NR¹⁰R¹¹, R¹⁰ is H, and R¹¹ is C₁₋₆alkyl. In some embodiments of the methods for treating a disease or condition with a compound of Formula (Ig), R¹ is —C(O)NR¹⁰R¹¹, R¹⁰ is H, and R¹¹ is —CH₃. In some embodiments of the methods for treating a disease or condition with a compound of Formula (Ig), R¹ is —C(O)NR¹⁰R¹¹, R¹⁰ is C₁₋₆alkyl, and R¹¹ is C₁₋₆alkyl. In some embodiments of the methods for treating a disease or condition with a compound of Formula (Ig), R¹ is —C(O)NR¹⁰R¹¹, R¹⁰ is —CH₃, and R¹¹ is —CH₃.

In some embodiments of the methods for treating a disease or condition with a compound of Formula (Ig), R² is H. In some embodiments of the methods for treating a disease or condition with a compound of Formula (Ig), R² is halogen. In some embodiments of the methods for treating a disease or condition with a compound of Formula (Ig), R² is —Cl. In some embodiments of the methods for treating a disease or condition with a compound of Formula (Ig), R² is C₁₋₆alkyl. In some embodiments of the methods for treating a disease or condition with a compound of Formula (Ig), R² is —CH₃. In some embodiments of the methods for treating a disease or condition with a compound of Formula (Ig), R² is C₁₋₆haloalkyl. In some embodiments of the methods for treating a disease or condition with a compound of Formula (Ig), R² is —CF₃.

In some embodiments of the methods for treating a disease or condition with a compound of Formula (Ig), R¹² is H. In some embodiments of the methods for treating a disease or condition with a compound of Formula (Ig), R¹² is halogen. In some embodiments of the methods for treating a disease or condition with a compound of Formula (Ig), R¹² is F. In some embodiments of the methods for treating a disease or condition with a compound of Formula (Ig), R¹² is C₁₋₆alkyl. In some embodiments of the methods for treating a disease or condition with a compound of Formula (Ig), R¹² is —CH₃. In some embodiments of the methods for treating a disease or condition with a compound of Formula (Ig), R¹³ is H. In some embodiments of the methods for treating a disease or condition with a compound of Formula (Ig), R¹³ is C₁₋₆alkyl. In some embodiments of the methods for treating a disease or condition with a compound of Formula (Ig), R¹³ is —CH₃. In some embodiments of the methods for treating a disease or condition with a compound of Formula (Ig), R¹² is H and R¹³ is H. In some embodiments of the methods for treating a disease or condition with a compound of Formula (Ig), R¹² is H and R¹³ is C₁₋₆alkyl. In some embodiments of the methods for treating a disease or condition with a compound of Formula (Ig), R¹² is H and R¹³ is —CH₃. In some embodiments of the methods for treating a disease or condition with a compound of Formula (Ig), R¹² is C₁₋₆alkyl and R¹³ is H. In some embodiments of the methods for treating a disease or condition with a compound of Formula (Ig), R¹² is —CH₃ and R¹³ is H. In some embodiments of the methods for treating a disease or condition with a compound of Formula (Ig), R¹² is C₁₋₆alkyl and R¹³ is C₁₋₆alkyl. In some embodiments of the methods for treating a disease or condition with a compound of Formula (Ig), R¹² is —CH₃ and R¹³ is —CH₃.

In some embodiments of the methods for treating a disease or condition with a compound of Formula (Ig), A is N. In some embodiments of the methods for treating a disease or condition with a compound of Formula (Ig), A is C(H).

In some embodiments of the methods for treating a disease or condition with a compound of Formula (Ig), X is —O—. In some embodiments of the methods for treating a disease or condition with a compound of Formula (Ig), X is —N(R¹⁶)—. In some embodiments of the methods for treating a disease or condition with a compound of Formula (Ig), X is —N(H)—. In some embodiments of the methods for treating a disease or condition with a compound of Formula (Ig), X is —N(CH₃)—. In some embodiments of the methods for treating a disease or condition with a compound of Formula (Ig), X is —CH₂N(R¹⁶)CH₂—. In some embodiments of the methods for treating a disease or condition with a compound of Formula (Ig), X is —CH₂N(H)CH₂—. In some embodiments of the methods for treating a disease or condition with a compound of Formula (Ig), X is —CH₂N(CH₃)CH₂—.

In some embodiments of the methods for treating a disease or condition with a compound of Formula (Ig), v is 0. In some embodiments of the methods for treating a disease or condition with a compound of Formula (Ig), v is 1.

In some embodiments of the methods for treating a disease or condition with a compound of Formula (Ig), R⁴ is halogen, —OR⁷, C₁₋₆alkyl, C₂₋₆alkynyl, C₁₋₆haloalkyl, —C(O)NR⁸R⁹, C₂₋₉heterocycloalkyl, C₆₋₁₀aryl, or C₁₋₉heteroaryl, wherein C₂₋₉ heterocycloalkyl, —C₁₋₆alkyl-C₂₋₉heterocycloalkyl, C₆₋₁₀aryl, or C₁₋₉heteroaryl are optionally substituted with 1 or 2 R¹⁴. In some embodiments of the methods for treating a disease or condition with a compound of Formula (Ig), R⁴ is halogen, —OR⁷, C₁₋₆haloalkyl, C₂₋₉heterocycloalkyl, C₆₋₁₀aryl, or C₁₋₉heteroaryl, wherein C₂₋₉ heterocycloalkyl, C₆₋₁₀aryl, or C₁₋₉heteroaryl are optionally substituted with 1 or 2 R¹⁴. In some embodiments of the methods for treating a disease or condition with a compound of Formula (Ig), R⁴ is halogen. In some embodiments of the methods for treating a disease or condition with a compound of Formula (Ig), R⁴ is —Cl. In some embodiments of the methods for treating a disease or condition with a compound of Formula (Ig), R⁴ is C₁₋₆haloalkyl. In some embodiments of the methods for treating a disease or condition with a compound of Formula (Ig), R⁴ is —CF₃. In some embodiments of the methods for treating a disease or condition with a compound of Formula (Ig), R⁴ is —OR⁷. In some embodiments of the methods for treating a disease or condition with a compound of Formula (Ig), R⁴ is —OR⁷ and R⁷ is C₁₋₆haloalkyl, C₆₋₁₀aryl, or C₁₋₉heteroaryl, wherein C₆₋₁₀aryl or C₁₋₉heteroaryl are optionally substituted with 1 or 2 R¹⁴. In some embodiments of the methods for treating a disease or condition with a compound of Formula (Ig), R⁴ is —OR⁷ and R⁷ is C₁₋₆haloalkyl. In some embodiments of the methods for treating a disease or condition with a compound of Formula (Ig), R⁴ is —OR⁷ and R⁷ is —CF₃. In some embodiments of the methods for treating a disease or condition with a compound of Formula (Ig), R⁴ is —OR⁷ and R⁷ is C₆₋₁₀aryl or C₁₋₉heteroaryl, wherein C₆₋₁₀aryl or C₁₋₉heteroaryl are optionally substituted with 1 or 2 R¹⁴. In some embodiments of the methods for treating a disease or condition with a compound of Formula (Ig), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are optionally substituted with halogen or C₁₋₆haloalkyl. In some embodiments of the methods for treating a disease or condition with a compound of Formula (Ig), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are unsubstituted. In some embodiments of the methods for treating a disease or condition with a compound of Formula (Ig), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are substituted with halogen or C₁₋₆haloalkyl. In some embodiments of the methods for treating a disease or condition with a compound of Formula (Ig), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are substituted with —Cl or —CF₃. In some embodiments of the methods for treating a disease or condition with a compound of Formula (Ig), R⁴ is C₂₋₉heterocycloalkyl optionally substituted with 1 or 2 R¹⁴. In some embodiments of the methods for treating a disease or condition with a compound of Formula (Ig), R⁴ is an unsubstituted C₂₋₉ heterocycloalkyl. In some embodiments of the methods for treating a disease or condition with a compound of Formula (Ig), R⁴ is C₂₋₉heterocycloalkyl substituted with 1 or 2 R¹⁴. In some embodiments of the methods for treating a disease or condition with a compound of Formula (Ig), R⁴ is

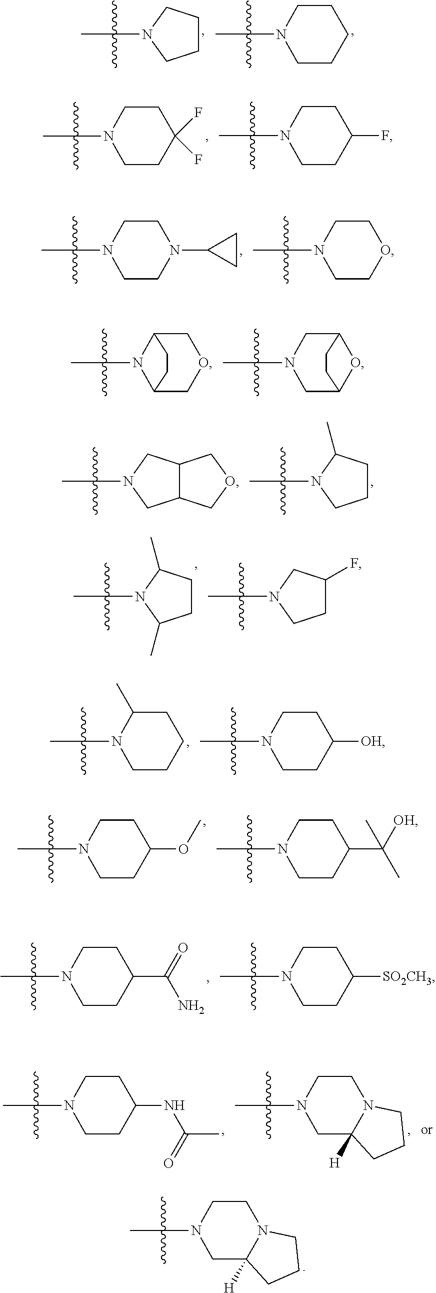

In some embodiments of the methods for treating a disease or condition with a compound of Formula (Ig), R⁴ is C₁₋₉heteroaryl optionally substituted with 1 or 2 R¹⁴. In some embodiments of the methods for treating a disease or condition with a compound of Formula (Ig), R⁴ is an unsubstituted C₁₋₉heteroaryl. In some embodiments of the methods for treating a disease or condition with a compound of Formula (Ig), R⁴ is C₂₋₉ heteroaryl substituted with 1 or 2 R¹⁴. In some embodiments of the methods for treating a disease or condition with a compound of Formula (Ig), R⁴ is

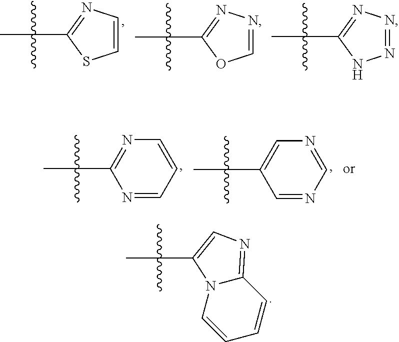 [SMILES: *c1nccs1 ; *c1nnco1 ; *c1nnn[nH]1 ; *c1ncccn1 ; *c1cncnc1 ; *c1cnc2ccccn12]

In some embodiments of the methods for treating a disease or condition with a compound of Formula (Ig), R⁵ is H, halogen, C₁₋₆alkyl, C₁₋₆haloalkyl, C₁₋₆haloalkoxy. In some embodiments of the methods for treating a disease or condition with a compound of Formula (Ig), R⁵ is H. In some embodiments of the methods for treating a disease or condition with a compound of Formula (Ig), R⁵ is halogen. In some embodiments of the methods for treating a disease or condition with a compound of Formula (Ig), R⁵ is —Cl. In some embodiments of the methods for treating a disease or condition with a compound of Formula (Ig), R⁵ is —F. In some embodiments of the methods for treating a disease or condition with a compound of Formula (Ig), R⁵ is C₁₋₆alkyl. In some embodiments of the methods for treating a disease or condition with a compound of Formula (Ig), R⁵ is —CH₃. In some embodiments of the methods for treating a disease or condition with a compound of Formula (Ig), R⁵ is C₁₋₆haloalkyl. In some embodiments of the methods for treating a disease or condition with a compound of Formula (Ig), R⁵ is —CF₃. In some embodiments of the methods for treating a disease or condition with a compound of Formula (Ig), R⁵ is C₁₋₆haloalkoxy. In some embodiments of the methods for treating a disease or condition with a compound of Formula (Ig), R⁵ is —OCF₃.

In some embodiments of the methods for treating a disease or condition with a compound of Formula (Ig), R⁶ is H. In some embodiments of the methods for treating a disease or condition with a compound of Formula (Ig), R⁶ is halogen. In some embodiments of the methods for treating a disease or condition with a compound of Formula (Ig), R⁶ is —Cl. In some embodiments of the methods for treating a disease or condition with a compound of Formula (Ig), R⁶ is —F. In some embodiments of the methods for treating a disease or condition with a compound of Formula (Ig), R⁶ is C₁₋₆alkyl. In some embodiments of the methods for treating a disease or condition with a compound of Formula (Ig), R⁶ is —CH₃.

In some embodiments is a method for treating a disease or a condition in a patient wherein the disease or condition is selected from atopic dermatitis, bladder dysfunction associated with multiple sclerosis, cardiovascular disease, contact dermatitis, cystic fibrosis, dermatomyositis, eczema, endometriosis, enteritis, fibromyalgia, Tourette syndrome, inflammatory bowel disease, interstitial cystitis, irritable bowel syndrome, ischemia, labor, abdominal pain, abdominal pain associated with irritable bowel syndrome, acute pain, back pain, cancer pain, chest pain, functional chest pain, joint pain, menstrual pain, metabolic disorders, musculoskeletal diseases, neuropathy, osteoarthritis, pancreatitis, pharyngitis, post mastectomy pain syndrome, post trigeminal neuralgia, post operative pain, renal ischemia, rheumatoid arthritis, skeletal muscle contusion, skin diseases, sunburn, systemic lupus erythematosus, toothache, vasoocclusive painful crises in sickle cell disease, and visceral pain, comprising administering to the patient in need thereof a therapeutically effective amount of a compound of Formula (Ih) having the structure:

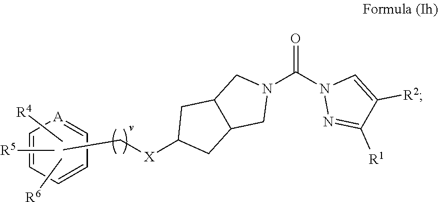

wherein:
R¹ is —C(O)OR¹⁵ or —C(O)NR¹⁰R¹¹;
R² is H, halogen, C₁₋₆alkyl, or C₁₋₆haloalkyl;
A is N or C(H);
X is —O—, —N(R¹⁶)—, or —CH₂N(R¹⁶)CH₂—;
R⁴ is H, halogen, —OR⁷, C₁₋₆alkyl, C₂₋₆alkenyl, C₂₋₆alkynyl, C₁₋₆haloalkyl, —C(O)NR⁸R⁹, C₂₋₉heterocycloalkyl, —C₁₋₆alkyl-C₂₋₉heterocycloalkyl, C₆₋₁₀aryl, or C₁₋₉heteroaryl, wherein C₂₋₉heterocycloalkyl, —C₁₋₆alkyl-C₂₋₉heterocycloalkyl, C₆₋₁₀aryl, or C₁₋₉heteroaryl are optionally substituted with 1 or 2 R¹⁴;
R⁵ is H, halogen, C₁₋₆alkyl, C₁₋₆haloalkyl, C₁₋₆haloalkoxy, or phenyl;
R⁶ is H, halogen or C₁₋₆alkyl;
R⁷ is H, C₁₋₆alkyl, C₁₋₆haloalkyl, C₂₋₉heterocycloalkyl, C₆₋₁₀aryl, or C₁₋₉heteroaryl, wherein C₂₋₉heterocycloalkyl, C₆₋₁₀aryl, or C₁₋₉heteroaryl are optionally substituted with 1 or 2 R¹⁴;
each R⁸ and each R⁹ are independently selected from H and C₁₋₆alkyl; or R⁸ and R⁹ together with the nitrogen to which they are attached are combined to form a heterocycloalkyl ring;
R¹⁰ and R¹¹ are each independently H or C₁₋₆alkyl;
each R¹⁴ is independently selected from halogen, —OH, C₁₋₆alkyl, C₁₋₆haloalkyl, C₁₋₆alkoxy, —C₁₋₆alkyl-OH, C₃₋₈cycloalkyl, —C(O)NR⁸R⁹, —SO₂—C₁₋₆alkyl, and —N(R¹⁷)C(O)—C₁₋₆alkyl;
R¹⁵ is H or C₁₋₆alkyl;
R¹⁶ is H, C₁₋₆alkyl, —C(O)—C₁₋₆alkyl, or —CH₂CO₂H;
R¹⁷ is H or C₁₋₆alkyl; and
v is 0 or 1;
or a solvate, hydrate, tautomer, N-oxide, stereoisomer, or pharmaceutically acceptable salt thereof.

In some embodiments of the methods for treating a disease or condition with a compound of Formula (Ih), R¹ is —C(O)OR¹⁵. In some embodiments of the methods for treating a disease or condition with a compound of Formula (Ih), R¹ is —C(O)OR¹⁵ and R¹⁵ is H. In some embodiments of the methods for treating a disease or condition with a compound of Formula (Ih), R¹ is —C(O)OR¹⁵ and R¹⁵ is C₁₋₆alkyl. In some embodiments of the methods for treating a disease or condition with a compound of Formula (Ih), R¹ is —C(O)OR¹⁵ and R¹⁵ is —CH₃. In some embodiments of the methods for treating a disease or condition with a compound of Formula (Ih), R¹ is —C(O)NR¹⁰R¹¹. In some embodiments of the methods for treating a disease or condition with a compound of Formula (Ih), R¹ is —C(O)NR¹⁰R¹¹, R¹⁰ is H, and R¹¹ is H. In some embodiments of the methods for treating a disease or condition with a compound of Formula (Ih), R¹ is —C(O)NR¹⁰R¹¹, R¹⁰ is H, and R¹¹ is C₁₋₆alkyl. In some embodiments of the methods for treating a disease or condition with a compound of Formula (Ih), R¹ is —C(O)NR¹⁰R¹¹, R¹⁰ is H, and R¹¹ is —CH₃. In some embodiments of the methods for treating a disease or condition with a compound of Formula (Ih), R¹ is —C(O)NR¹⁰R¹¹, R¹⁰ is C₁₋₆alkyl, and R¹¹ is C₁₋₆alkyl. In some embodiments of the methods for treating a disease or condition with a compound of Formula (Ih), R¹ is —C(O)NR¹⁰R¹¹, R¹⁰ is —CH₃, and R¹¹ is —CH₃.

In some embodiments of the methods for treating a disease or condition with a compound of Formula (Ih), R² is H. In some embodiments of the methods for treating a disease or condition with a compound of Formula (Ih), R² is halogen. In some embodiments of the methods for treating a disease or condition with a compound of Formula (Ih), R² is —Cl. In some embodiments of the methods for treating a disease or condition with a compound of Formula (Ih), R² is C₁₋₆alkyl. In some embodiments of the methods for treating a disease or condition with a compound of Formula (Ih), R² is —CH₃. In some embodiments of the methods for treating a disease or condition with a compound of Formula (Ih), R² is C₁₋₆haloalkyl. In some embodiments of the methods for treating a disease or condition with a compound of Formula (Ih), R² is —CF₃.

In some embodiments of the methods for treating a disease or condition with a compound of Formula (Ih), A is N. In some embodiments of the methods for treating a disease or condition with a compound of Formula (Ih), A is C(H).

In some embodiments of the methods for treating a disease or condition with a compound of Formula (Ih), X is —O—. In some embodiments of the methods for treating a disease or condition with a compound of Formula (Ih), X is —N(R¹⁶)—. In some embodiments of the methods for treating a disease or condition with a compound of Formula (Ih), X is —N(H)—. In some embodiments of the methods for treating a disease or condition with a compound of Formula (Ih), X is —N(CH₃)—. In some embodiments of the methods for treating a disease or condition with a compound of Formula (Ih), X is —CH₂N(R¹⁶)CH₂—. In some embodiments of the methods for treating a disease or condition with a compound of Formula (Ih), X is —CH₂N(H)CH₂—. In some embodiments of the methods for treating a disease or condition with a compound of Formula (Ih), X is —CH₂N(CH₃)CH₂—.

In some embodiments of the methods for treating a disease or condition with a compound of Formula (Ih), v is 0. In some embodiments of the methods for treating a disease or condition with a compound of Formula (Ih), v is 1.

In some embodiments of the methods for treating a disease or condition with a compound of Formula (Ih), R⁴ is halogen, —OR⁷, C₁₋₆alkyl, C₂₋₆alkynyl, C₁₋₆haloalkyl, —C(O)NR⁸R⁹, C₂₋₉heterocycloalkyl, C₆₋₁₀aryl, or C₁₋₉heteroaryl, wherein C₂₋₉ heterocycloalkyl, —C₁₋₆alkyl-C₂₋₉heterocycloalkyl, C₆₋₁₀aryl, or C₁₋₉heteroaryl are optionally substituted with 1 or 2 R¹⁴. In some embodiments of the methods for treating a disease or condition with a compound of Formula (Ih), R⁴ is halogen, —OR⁷, C₁₋₆haloalkyl, C₂₋₉heterocycloalkyl, C₆₋₁₀aryl, or C₁₋₉heteroaryl, wherein C₂₋₉ heterocycloalkyl, C₆₋₁₀aryl, or C₁₋₉heteroaryl are optionally substituted with 1 or 2 R¹⁴. In some embodiments of the methods for treating a disease or condition with a compound of Formula (Ih), R⁴ is halogen. In some embodiments of the methods for treating a disease or condition with a compound of Formula (Ih), R⁴ is —Cl. In some embodiments of the methods for treating a disease or condition with a compound of Formula (Ih), R⁴ is C₁₋₆haloalkyl. In some embodiments of the methods for treating a disease or condition with a compound of Formula (Ih), R⁴ is —CF₃. In some embodiments of the methods for treating a disease or condition with a compound of Formula (Ih), R⁴ is —OR⁷. In some embodiments of the methods for treating a disease or condition with a compound of Formula (Ih), R⁴ is —OR⁷ and R⁷ is C₁₋₆haloalkyl, C₆₋₁₀aryl, or C₁₋₉heteroaryl, wherein C₆₋₁₀aryl or C₁₋₉heteroaryl are optionally substituted with 1 or 2 R¹⁴. In some embodiments of the methods for treating a disease or condition with a compound of Formula (Ih), R⁴ is —OR⁷ and R⁷ is C₁₋₆haloalkyl. In some embodiments of the methods for treating a disease or condition with a compound of Formula (Ih), R⁴ is —OR⁷ and R⁷ is —CF₃. In some embodiments of the methods for treating a disease or condition with a compound of Formula (Ih), R⁴ is —OR⁷ and R⁷ is C₆₋₁₀aryl or C₁₋₉heteroaryl, wherein C₆₋₁₀aryl or C₁₋₉heteroaryl are optionally substituted with 1 or 2 R¹⁴. In some embodiments of the methods for treating a disease or condition with a compound of Formula (Ih), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are optionally substituted with halogen or C₁₋₆haloalkyl. In some embodiments of the methods for treating a disease or condition with a compound of Formula (Ih), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are unsubstituted. In some embodiments of the methods for treating a disease or condition with a compound of Formula (Ih), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are substituted with halogen or C₁₋₆haloalkyl. In some embodiments of the methods for treating a disease or condition with a compound of Formula (Ih), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are substituted with —Cl or —CF₃. In some embodiments of the methods for treating a disease or condition with a compound of Formula (Ih), R⁴ is C₂₋₉heterocycloalkyl optionally substituted with 1 or 2 R¹⁴. In some embodiments of the methods for treating a disease or condition with a compound of Formula (Ih), R⁴ is an unsubstituted C₂₋₉ heterocycloalkyl. In some embodiments of the methods for treating a disease or condition with a compound of Formula (Ih), R⁴ is C₂₋₉heterocycloalkyl substituted with 1 or 2 R¹⁴. In some embodiments of the methods for treating a disease or condition with a compound of Formula (Ih), R⁴ is

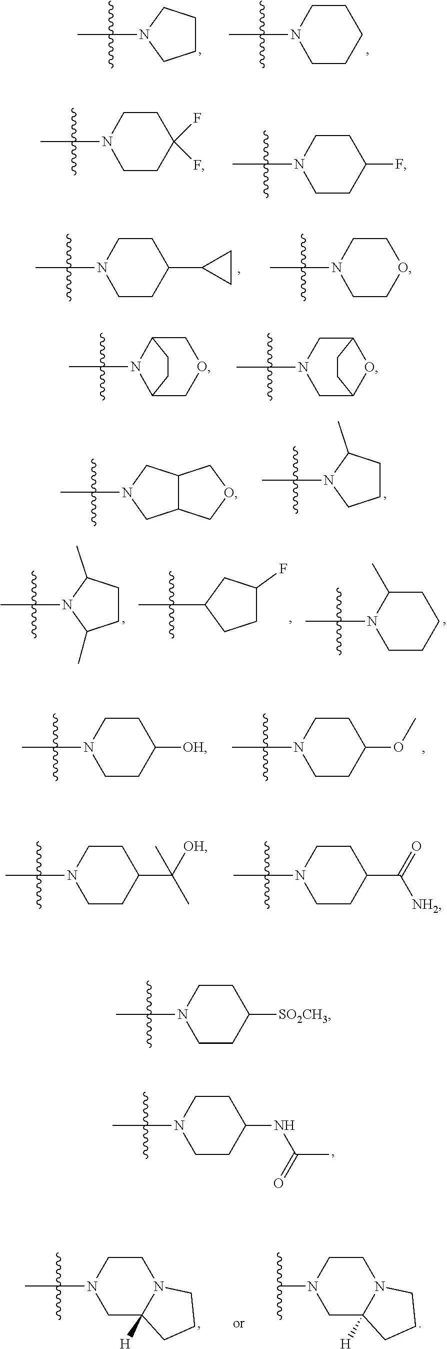

In some embodiments of the methods for treating a disease or condition with a compound of Formula (Ih), R⁴ is C₁₋₉ heteroaryl optionally substituted with 1 or 2 R¹⁴. In some embodiments of the methods for treating a disease or condition with a compound of Formula (Ih), R⁴ is an unsubstituted C₁₋₉ heteroaryl. In some embodiments of the methods for treating a disease or condition with a compound of Formula (Ih), R⁴ is C₂₋₉heteroaryl substituted with 1 or 2 R¹⁴. In some embodiments of the methods for treating a disease or condition with a compound of Formula (Ih), R⁴ is

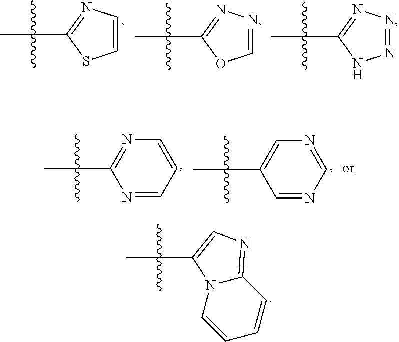 [SMILES: *c1nccs1 ; *c1nnco1 ; *c1nnn[nH]1 ; *c1ncccn1 ; *c1cncnc1 ; *c1cnc2ccccn12]

In some embodiments of the methods for treating a disease or condition with a compound of Formula (Ih), R⁵ is H, halogen, C₁₋₆alkyl, C₁₋₆haloalkyl, C₁₋₆haloalkoxy. In some embodiments of the methods for treating a disease or condition with a compound of Formula (Ih), R⁵ is H. In some embodiments of the methods for treating a disease or condition with a compound of Formula (Ih), R⁵ is halogen. In some embodiments of the methods for treating a disease or condition with a compound of Formula (Ih), R⁵ is —Cl. In some embodiments of the methods for treating a disease or condition with a compound of Formula (Ih), R⁵ is —F. In some embodiments of the methods for treating a disease or condition with a compound of Formula (Ih), R⁵ is C₁₋₆alkyl. In some embodiments of the methods for treating a disease or condition with a compound of Formula (Ih), R⁵ is —CH₃. In some embodiments of the methods for treating a disease or condition with a compound of Formula (Ih), R⁵ is C₁₋₆haloalkyl. In some embodiments of the methods for treating a disease or condition with a compound of Formula (Ih), R⁵ is —CF₃. In some embodiments of the methods for treating a disease or condition with a compound of Formula (Ih), R⁵ is C₁₋₆haloalkoxy. In some embodiments of the methods for treating a disease or condition with a compound of Formula (Ih), R⁵ is —OCF₃.

In some embodiments of the methods for treating a disease or condition with a compound of Formula (Ih), R⁶ is H. In some embodiments of the methods for treating a disease or condition with a compound of Formula (Ih), R⁶ is halogen. In some embodiments of the methods for treating a disease or condition with a compound of Formula (Ih), R⁶ is —Cl. In some embodiments of the methods for treating a disease or condition with a compound of Formula (Ih), R⁶ is —F. In some embodiments of the methods for treating a disease or condition with a compound of Formula (Ih), R⁶ is C₁₋₆alkyl. In some embodiments of the methods for treating a disease or condition with a compound of Formula (Ih), R⁶ is —CH₃.

In some embodiments is a method for treating a disease or a condition in a patient wherein the disease or condition is selected from atopic dermatitis, bladder dysfunction associated with multiple sclerosis, cardiovascular disease, contact dermatitis, cystic fibrosis, dermatomyositis, eczema, endometriosis, enteritis, fibromyalgia, Tourette syndrome, inflammatory bowel disease, interstitial cystitis, irritable bowel syndrome, ischemia, labor, abdominal pain, abdominal pain associated with irritable bowel syndrome, acute pain, back pain, cancer pain, chest pain, functional chest pain, joint pain, menstrual pain, metabolic disorders, musculoskeletal diseases, neuropathy, osteoarthritis, pancreatitis, pharyngitis, post mastectomy pain syndrome, post trigeminal neuralgia, post operative pain, renal ischemia, rheumatoid arthritis, skeletal muscle contusion, skin diseases, sunburn, systemic lupus erythematosus, toothache, vasoocclusive painful crises in sickle cell disease, and visceral pain, comprising administering to the patient in need thereof a therapeutically effective amount of a compound of Formula (Ii) having the structure:

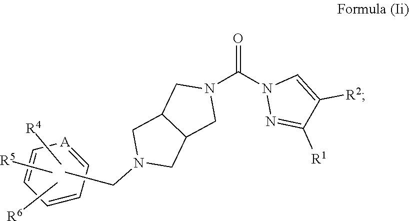

wherein:
R¹ is —C(O)OR¹⁵ or —C(O)NR¹⁰R¹¹;
R² is H, halogen, C₁₋₆alkyl, or C₁₋₆haloalkyl;
A is N or C(H);
R⁴ is H, halogen, —OR⁷, C₁₋₆alkyl, C₂₋₆alkenyl, C₂₋₆alkynyl, C₁₋₆haloalkyl, —C(O)NR⁸R⁹, C₂₋₉heterocycloalkyl, —C₁₋₆alkyl-C₂₋₉heterocycloalkyl, C₆₋₁₀aryl, or C₁₋₉heteroaryl, wherein C₂₋₉heterocycloalkyl, —C₁₋₆alkyl-C₂₋₉heterocycloalkyl, C₆₋₁₀aryl, or C₁₋₉heteroaryl are optionally substituted with 1 or 2 R¹⁴;
R⁵ is H, halogen, C₁₋₆alkyl, C₁₋₆haloalkyl, C₁₋₆haloalkoxy, or phenyl;
R⁶ is H, halogen or C₁₋₆alkyl;
R⁷ is H, C₁₋₆alkyl, C₁₋₆haloalkyl, C₂₋₉heterocycloalkyl, C₆₋₁₀aryl, or C₁₋₉heteroaryl, wherein C₂₋₉heterocycloalkyl, C₆₋₁₀aryl, or C₁₋₉heteroaryl are optionally substituted with 1 or 2 R¹⁴;
each R⁸ and each R⁹ are independently selected from H and C₁₋₆alkyl; or R⁸ and R⁹ together with the nitrogen to which they are attached are combined to form a heterocycloalkyl ring;
R¹⁰ and R¹¹ are each independently H or C₁₋₆alkyl;
each R¹⁴ is independently selected from halogen, —OH, C₁₋₆alkyl, C₁₋₆haloalkyl, C₁₋₆alkoxy, —C₁₋₆alkyl-OH, C₃₋₈cycloalkyl, —C(O)NR⁸R⁹, —SO₂—C₁₋₆alkyl, and —N(R¹⁷)C(O)—C₁₋₆alkyl;
R¹⁵ is H or C₁₋₆alkyl; and
R¹⁷ is H or C₁₋₆alkyl;
or a solvate, hydrate, tautomer, N-oxide, stereoisomer, or pharmaceutically acceptable salt thereof.

In some embodiments of the methods for treating a disease or condition with a compound of Formula (Ii), R¹ is —C(O)OR¹⁵. In some embodiments of the methods for treating a disease or condition with a compound of Formula (Ii), R¹ is —C(O)OR¹⁵ and R¹⁵ is H. In some embodiments of the methods for treating a disease or condition with a compound of Formula (Ii), R¹ is —C(O)OR¹⁵ and R¹⁵ is C₁₋₆alkyl. In some embodiments of the methods for treating a disease or condition with a compound of Formula (Ii), R¹ is —C(O)OR¹⁵ and R¹⁵ is —CH₃. In some embodiments of the methods for treating a disease or condition with a compound of Formula (Ii), R¹ is —C(O)NR¹⁰R¹¹. In some embodiments of the methods for treating a disease or condition with a compound of Formula (Ii), R¹ is —C(O)NR¹⁰R¹¹, R¹⁰ is H, and R¹¹ is H. In some embodiments of the methods for treating a disease or condition with a compound of Formula (Ii), R¹ is —C(O)NR¹⁰R¹¹, R¹⁰ is H, and R¹¹ is C₁₋₆alkyl. In some embodiments of the methods for treating a disease or condition with a compound of Formula (Ii), R¹ is —C(O)NR¹⁰R¹¹, R¹⁰ is H, and R¹¹ is —CH₃. In some embodiments of the methods for treating a disease or condition with a compound of Formula (Ii), R¹ is —C(O)NR¹⁰R¹¹, R¹⁰ is C₁₋₆alkyl, and R¹¹ is C₁₋₆alkyl. In some embodiments of the methods for treating a disease or condition with a compound of Formula (Ii), R¹ is —C(O)NR¹⁰R¹¹, R¹⁰ is —CH₃, and R¹¹ is —CH₃.

In some embodiments of the methods for treating a disease or condition with a compound of Formula (Ii), R² is H. In some embodiments of the methods for treating a disease or condition with a compound of Formula (Ii), R² is halogen. In some embodiments of the methods for treating a disease or condition with a compound of Formula (Ii), R² is —Cl. In some embodiments of the methods for treating a disease or condition with a compound of Formula (Ii), R² is C₁₋₆alkyl. In some embodiments of the methods for treating a disease or condition with a compound of Formula (Ii), R² is —CH₃. In some embodiments of the methods for treating a disease or condition with a compound of Formula (Ii), R² is C₁₋₆haloalkyl. In some embodiments of the methods for treating a disease or condition with a compound of Formula (Ii), R² is —CF₃.

In some embodiments of the methods for treating a disease or condition with a compound of Formula (Ii), A is N. In some embodiments of the methods for treating a disease or condition with a compound of Formula (Ii), A is C(H).

In some embodiments of the methods for treating a disease or condition with a compound of Formula (Ii), R⁴ is halogen, —OR⁷, C₁₋₆alkyl, C₂₋₆alkynyl, C₁₋₆haloalkyl, —C(O)NR⁸R⁹, C₂₋₉heterocycloalkyl, C₆₋₁₀aryl, or C₁₋₉heteroaryl, wherein C₂₋₉ heterocycloalkyl, —C₁₋₆alkyl-C₂₋₉heterocycloalkyl, C₆₋₁₀aryl, or C₁₋₉heteroaryl are optionally substituted with 1 or 2 R¹⁴. In some embodiments of the methods for treating a disease or condition with a compound of Formula (Ii), R⁴ is halogen, —OR⁷, C₁₋₆haloalkyl, C₂₋₉heterocycloalkyl, C₆₋₁₀aryl, or C₁₋₉heteroaryl, wherein C₂₋₉heterocycloalkyl, C₆₋₁₀aryl, or C₁₋₉heteroaryl are optionally substituted with 1 or 2 R¹⁴. In some embodiments of the methods for treating a disease or condition with a compound of Formula (Ii), R⁴ is halogen. In some embodiments of the methods for treating a disease or condition with a compound of Formula (Ii), R⁴ is —Cl. In some embodiments of the methods for treating a disease or condition with a compound of Formula (Ii), R⁴ is C₁₋₆haloalkyl. In some embodiments of the methods for treating a disease or condition with a compound of Formula (Ii), R⁴ is —CF₃. In some embodiments of the methods for treating a disease or condition with a compound of Formula (Ii), R⁴ is —OR⁷. In some embodiments of the methods for treating a disease or condition with a compound of Formula (Ii), R⁴ is —OR⁷ and R⁷ is C₁₋₆haloalkyl, C₆₋₁₀aryl, or C₁₋₉heteroaryl, wherein C₆₋₁₀aryl or C₁₋₉heteroaryl are optionally substituted with 1 or 2 R¹⁴. In some embodiments of the methods for treating a disease or condition with a compound of Formula (Ii), R⁴ is —OR⁷ and R⁷ is C₁₋₆haloalkyl. In some embodiments of the methods for treating a disease or condition with a compound of Formula (Ii), R⁴ is —OR⁷ and R⁷ is —CF₃. In some embodiments of the methods for treating a disease or condition with a compound of Formula (Ii), R⁴ is —OR⁷ and R⁷ is C₆₋₁₀aryl or C₁₋₉heteroaryl, wherein C₆₋₁₀aryl or C₁₋₉heteroaryl are optionally substituted with 1 or 2 R¹⁴. In some embodiments of the methods for treating a disease or condition with a compound of Formula (Ii), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are optionally substituted with halogen or C₁₋₆haloalkyl. In some embodiments of the methods for treating a disease or condition with a compound of Formula (Ii), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are unsubstituted. In some embodiments of the methods for treating a disease or condition with a compound of Formula (Ii), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are substituted with halogen or C₁₋₆haloalkyl. In some embodiments of the methods for treating a disease or condition with a compound of Formula (Ii), R⁴ is —OR⁷ and R⁷ is phenyl, pyridyl, pyrimidinyl, pyridizinyl, or pyrazinyl, wherein phenyl, pyridyl, pyrimidinyl, pyridizinyl, or pyrazinyl are substituted with —Cl or —CF₃. In some embodiments of the methods for treating a disease or condition with a compound of Formula (Ii), R⁴ is C₂₋₉heterocycloalkyl optionally substituted with 1 or 2 R¹⁴. In some embodiments of the methods for treating a disease or condition with a compound of Formula (Ii), R⁴ is an unsubstituted C₂₋₉ heterocycloalkyl. In some embodiments of the methods for treating a disease or condition with a compound of Formula (Ii), R⁴ is C₂₋₉heterocycloalkyl substituted with 1 or 2 R¹⁴. In some embodiments of the methods for treating a disease or condition with a compound of Formula (Ii), R⁴ is

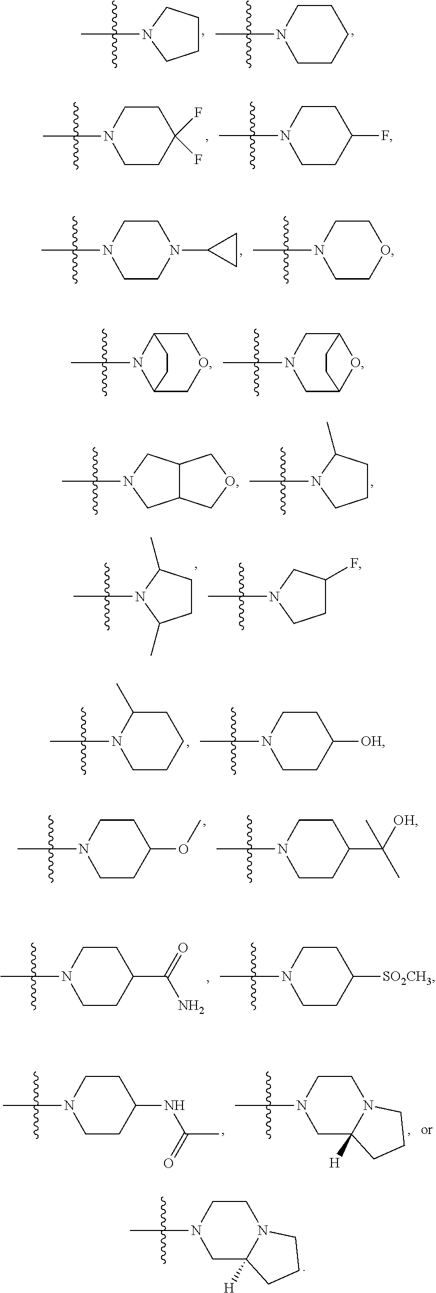

In some embodiments of the methods for treating a disease or condition with a compound of Formula (Ii), R⁴ is C₁₋₉heteroaryl optionally substituted with 1 or 2 R¹⁴. In some embodiments of the methods for treating a disease or condition with a compound of Formula (Ii), R⁴ is an unsubstituted C₁₋₉heteroaryl. In some embodiments of the methods for treating a disease or condition with a compound of Formula (Ii), R⁴ is C₂₋₉ heteroaryl substituted with 1 or 2 R¹⁴. In some embodiments of the methods for treating a disease or condition with a compound of Formula (Ii), R⁴ is

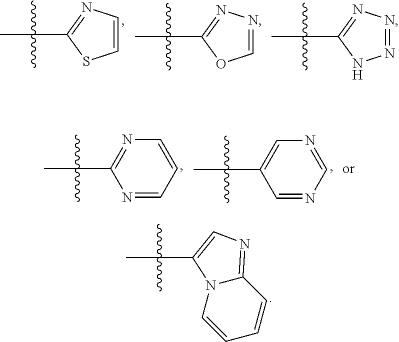 [SMILES: *c1nccs1 ; *c1nnco1 ; *c1nnn[nH]1 ; *c1ncccn1 ; *c1cncnc1 ; *c1cnc2ccccn12]

In some embodiments of the methods for treating a disease or condition with a compound of Formula (Ii), R⁵ is H, halogen, C₁₋₆alkyl, C₁₋₆haloalkyl, C₁₋₆haloalkoxy. In some embodiments of the methods for treating a disease or condition with a compound of Formula (Ii), R⁵ is H. In some embodiments of the methods for treating a disease or condition with a compound of Formula (Ii), R⁵ is halogen. In some embodiments of the methods for treating a disease or condition with a compound of Formula (Ii), R⁵ is —Cl. In some embodiments of the methods for treating a disease or condition with a compound of Formula (Ii), R⁵ is —F. In some embodiments of the methods for treating a disease or condition with a compound of Formula (Ii), R⁵ is C₁₋₆alkyl. In some embodiments of the methods for treating a disease or condition with a compound of Formula (Ii), R⁵ is —CH₃. In some embodiments of the methods for treating a disease or condition with a compound of Formula (Ii), R⁵ is C₁₋₆haloalkyl. In some embodiments of the methods for treating a disease or condition with a compound of Formula (Ii), R⁵ is —CF₃. In some embodiments of the methods for treating a disease or condition with a compound of Formula (Ii), R⁵ is C₁₋₆haloalkoxy. In some embodiments of the methods for treating a disease or condition with a compound of Formula (Ii), R⁵ is —OCF₃.

In some embodiments of the methods for treating a disease or condition with a compound of Formula (Ii), R⁶ is H. In some embodiments of the methods for treating a disease or condition with a compound of Formula (Ii), R⁶ is halogen. In some embodiments of the methods for treating a disease or condition with a compound of Formula (Ii), R⁶ is —Cl. In some embodiments of the methods for treating a disease or condition with a compound of Formula (Ii), R⁶ is —F. In some embodiments of the methods for treating a disease or condition with a compound of Formula (Ii), R⁶ is C₁₋₆alkyl. In some embodiments of the methods for treating a disease or condition with a compound of Formula (Ii), R⁶ is —CH₃.

Further embodiments provided herein include combinations of one or more of the particular embodiments set forth above.

In yet further embodiments, MAGL inhibitors described herein synergistically potentiate the activity of an opioid analgesic. In some embodiments, MAGL inhibitors described herein reduce the acute side-effects associated with an opioid analgesic. In some embodiments, disclosed herein is a method of synergistically potentiating the activity of an opioid analgesic in a patient being treated with an opioid analgesic, comprising administering to the patient a therapeutically effective amount of a compound of Formula (I), (I′), (Ia), (Ib), (Ic), (Id), (Ie), (If), (Ig), (Ih), or (Ii) described herein, or a pharmaceutically acceptable salt or solvate thereof. In some embodiments, disclosed herein is a method of reducing the acute side-effects associated with an opioid analgesic in a patient being treated with an opioid analgesic, comprising administering to the patient a therapeutically effective amount of a compound of Formula (I), (I′), (la), (Ib), (Ic), (Id), (Ie), (If), (Ig), (Ih), or (Ii) described herein, or a pharmaceutically acceptable salt or solvate thereof.

MAGL inhibitors are efficacious as monotherapy in multiple models of pain. MAGL inhibition has also been shown to produce opioid-sparing effects preclinical pain models. In the chronic constrictive injury (CCI) neuropathic pain model in mice, combined treatment with a MAGL inhibitor and the opioid morphine resulted in synergistic improvements in efficacy compared to treatment of either compound alone. The combination of MAGL inhibition and morphine did not produce opioid-like reductions in gastric motility, produce cannabimimetic effects in the drug discrimination assay or undergo tolerance following repeat dosing. In some embodiments, disclosed herein is a method of producing opioid-sparing effects in a patient being treated with an opioid analgesic, comprising administering to the patient a therapeutically effective amount of a compound of Formula (I), (I′), (Ia), (Ib), (Ic), (Id), (Ie), (If), (Ig), (Ih), or (Ii) described herein, or a pharmaceutically acceptable salt or solvate thereof.

In certain embodiments, a disclosed compound utilized by one or more of the foregoing methods is one of the generic, subgeneric, or specific compounds described herein, such as a compound of Formula (I), (I′), (Ia), (Ib), (Ic), (Id), (Ie), (If), (Ig), (Ih), or (Ii) described herein.

Disclosed compounds are administered to patients (animals and humans) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors, with the appropriate dosage ultimately being at the discretion of the attendant physician. For treating clinical conditions and diseases noted above, a contemplated compound disclosed herein is administered orally, subcutaneously, topically, parenterally, by inhalation spray or rectally in dosage unit formulations containing conventional non-toxic pharmaceutically acceptable carriers, adjuvants and vehicles. Parenteral administration include subcutaneous injections, intravenous or intramuscular injections or infusion techniques.

### Combination Therapies

Also contemplated herein are combination therapies, for example, co-administering a disclosed compound and an additional active agent, as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually weeks, months or years depending upon the combination selected). Combination therapy is intended to embrace administration of multiple therapeutic agents in a sequential manner, that is, wherein each therapeutic agent is administered at a different time, as well as administration of these therapeutic agents, or at least two of the therapeutic agents, in a substantially simultaneous manner.

Substantially simultaneous administration is accomplished, for example, by administering to the subject a single formulation or composition, (e.g., a tablet or capsule having a fixed ratio of each therapeutic agent or in multiple, single formulations (e.g., capsules) for each of the therapeutic agents. Sequential or substantially simultaneous administration of each therapeutic agent is effected by any appropriate route including, but not limited to, oral routes, intravenous routes, intramuscular routes, and direct absorption through mucous membrane tissues. The therapeutic agents are administered by the same route or by different routes. For example, a first therapeutic agent of the combination selected is administered by intravenous injection while the other therapeutic agents of the combination are administered orally. Alternatively, for example, all therapeutic agents are administered orally or all therapeutic agents are administered by intravenous injection.

Combination therapy also embraces the administration of the therapeutic agents as described above in further combination with other biologically active ingredients and non-drug therapies. Where the combination therapy further comprises a non-drug treatment, the non-drug treatment is conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

The components of the combination are administered to a patient simultaneously or sequentially. It will be appreciated that the components are present in the same pharmaceutically acceptable carrier and, therefore, are administered simultaneously. Alternatively, the active ingredients are present in separate pharmaceutical carriers, such as, conventional oral dosage forms, that are administered either simultaneously or sequentially.

For example, e.g., for contemplated treatment of pain, a disclosed compound is co-administered with another therapeutic for pain such as an opioid, a cannabinoid receptor (CB1 or CB2) modulator, a COX-2 inhibitor, acetaminophen, and/or a non-steroidal anti-inflammatory agent. Additional therapeutics e.g., for the treatment of pain that are co-administered, include morphine, codeine, hydromorphone, hydrocodone, oxymorphone, fentanyl, tramadol, and levorphanol.

Other contemplated therapeutics for co-administration include aspirin, naproxen, ibuprofen, salsalate, diflunisal, dexibuprofen, fenoprofen, ketoprofen, oxaprozin, loxoprofen, indomethacin, tolmetin, sulindac, etodolac, ketorolac, piroxicam, meloxicam, tenoxicam, droxicam, lornoxicam, celecoxib, parecoxib, rimonabant, and/or etoricoxib.

In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with a tricyclic antidepressant, such as imipramine, amitriptyline, or desipramine. In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with a serotonin-norepinephrine reuptake inhibitor, such as duloxetine, milnacipran, venlafaxine, or clomipramine. In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with an alpha-2-delta inhibitor, such as gabapentin or pregabalin. In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with an antiepileptic drug, such as topiramate, lamotrigine, levetiracetam, valproate, clonazepam, oxcarbazine, or carbamazepine.

In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with an opioid, such as morphine, codeine, oxycodone, oxymorphone, tramadol, tapentadol, methadone, or fentanyl.

In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with acetaminophen. In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with a nonsteroidal anti-inflammatory drug, such as ibuprofen, naproxen, celecoxib, or diclofenac. In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with a disease-modifying antirheumatic drug, such as tofacitinib, leflunomide, or methotrexate.

In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with exo-cannabinoids, such as oral delta-9-THC and nabiximols (Sativex).

In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with a muscle relaxant such as baclofen and tizanidine. In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with diazepam.

In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with a prokinetic agent, such as metoclopramide, domperidone, or itopride. In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with a 5-HT4 agonist, such as tegaserod or mosapride. In some embodiments, a compound of Formula (I), (I′), (la), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with buspirone.

In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with a neuroleptic, such as pimozide, olanzapine, risperidone, or quetiapine.

In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with a cholinesterase inhibitor, such as donepezil, rivastigmine, or galantamine. In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with a NMDA antagonist, such as memantine.

In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with dopamine replacement therapy, such as levodopa or carbidopa-levodopa. In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with a catechol-O-methyl transferase (COMT) inhibitor, such as tolcapone or entacapone. In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with a dopamine agonist, such as bromocriptine, pramipexole, or ropinirole. In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with a monamine oxidase (MAO) B inhibitor, such as selegiline or rasagiline. In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with an anticholinergic agent, such as benztropine, trihexyphenidyl, or procyclidine.

In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with a dopamine antagonist, such as haloperidol, pimozide, or fluphenazine. In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with a VMAT2 inhibitor which depletes dopamine, such as tetrabenazine. In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with an alpha adrenergic agonist, such as clonidine or guanfacine.

In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with a selective serotonin reuptake inhibitors (SSRI), such as fluoxetine, sertraline, paroxetine, citalopram or escitalopram.

In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with a stimulant, such as methylphenidate, dextroamphetamine, or lisdexamfetamine. In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with an antidepressant, such as bupropion or atomoxetine.

In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with a serotonin 1b/1d agonist. In some embodiments, a compound of Formula (I), (I′), (la), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with a triptan, such as sumatriptan or zolmitriptan.

In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with a glutamate inhibitor, such as riluzole.

In some embodiments, a compound of Formula (I), (I′), (Ia), (Ib), (Ic), (Id), (Ie), (If), (Ig), (Ih), or (Ii) described herein, or a solvate, hydrate, tautomer, N-oxide, stereoisomer, or a pharmaceutically acceptable salt thereof, is co-administered with an HI antihistamine, such as diphenhydramine, hydroxyzine, cetirizine, loratadine, or desloratadine.

In certain embodiments, a disclosed compound utilized by one or more of the foregoing methods is one of the generic, subgeneric, or specific compounds described herein, such as a compound of Formula (I), (I′), (Ia), (Ib), (Ic), (Id), (Ie), (If), (Ig), (Ih), or (Ii).

### Preparation of the Compounds

The compounds used in the methods described herein are made according to the examples and chemical synthesis disclosed in U.S. Ser. No. 15/986,747, which is herein incorporated by reference in its entirety, or by known organic synthesis techniques, starting from commercially available chemicals and/or from compounds described in the chemical literature. “Commercially available chemicals” are obtained from standard commercial sources including Acros Organics (Geel, Belgium), Aldrich Chemical (Milwaukee, Wis., including Sigma Chemical and Fluka), Apin Chemicals Ltd. (Milton Park, UK), Ark Pharm, Inc. (Libertyville, Ill.), Avocado Research (Lancashire, U.K.), BDH Inc. (Toronto, Canada), Bionet (Cornwall, U.K.), Chemservice Inc. (West Chester, Pa.), Combi-blocks (San Diego, Calif.), Crescent Chemical Co. (Hauppauge, N.Y.), eMolecules (San Diego, Calif.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Matrix Scientific, (Columbia, S.C.), Maybridge Chemical Co. Ltd. (Cornwall, U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hanover, Germany), Ryan Scientific, Inc. (Mount Pleasant, S.C.), Spectrum Chemicals (Gardena, Calif.), Sundia Meditech, (Shanghai, China), TCI America (Portland, Oreg.), Trans World Chemicals, Inc. (Rockville, Md.), and WuXi (Shanghai, China).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modem Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are also identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### MAGL Data for Compounds Disclosed Herein

Compounds disclosed herein are MAGL inhibitors as disclosed in the biological evaluation section in U.S. Ser. No. 15/986,747, which is herein incorporated by reference in its entirety.

### Further Forms of Compounds Disclosed Herein

### Isomers

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that are incorporated into compounds of the invention include isotopes of hydrogen, carbon, nitrogen, oxygen, phosphorous, sulfur, fluorine and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, esters, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, solvate, hydrate or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds of the invention, or by separately reacting a purified compound in its free form with a suitable acid or base, and isolating the salt thus formed.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran or methanol. In addition, the compounds provided herein exist in unsolvated as well as solvated forms. In general, the solvated forms are considered equivalent to the unsolvated forms for the purposes of the compounds and methods provided herein.

### Pharmaceutical Compositions

In certain embodiments, the compounds described herein are administered as a pure chemical. In other embodiments, the compounds described herein are combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington; The Science and Practice of Pharmacy (Gennaro, 21^{st} Ed. Mack Pub. Co., Easton, Pa. (2005)).

Accordingly, provided herein is a pharmaceutical composition comprising at least one compound described herein, or a stereoisomer, pharmaceutically acceptable salt, hydrate, solvate, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject) of the composition.

One embodiment provides a pharmaceutical composition comprising a pharmaceutically acceptable carrier and a compound of Formula (I), or a pharmaceutically acceptable salt thereof.

One embodiment provides a pharmaceutical composition comprising a pharmaceutically acceptable carrier and a compound of Formula (I′), or a pharmaceutically acceptable salt thereof.

One embodiment provides a pharmaceutical composition comprising a pharmaceutically acceptable carrier and a compound of Formula (Ia), or a pharmaceutically acceptable salt thereof.

One embodiment provides a pharmaceutical composition comprising a pharmaceutically acceptable carrier and a compound of Formula (Ib), or a pharmaceutically acceptable salt thereof.

One embodiment provides a pharmaceutical composition comprising a pharmaceutically acceptable carrier and a compound of Formula (Ic), or a pharmaceutically acceptable salt thereof.

One embodiment provides a pharmaceutical composition comprising a pharmaceutically acceptable carrier and a compound of Formula (Id), or a pharmaceutically acceptable salt thereof.

One embodiment provides a pharmaceutical composition comprising a pharmaceutically acceptable carrier and a compound of Formula (Ie), or a pharmaceutically acceptable salt thereof.

One embodiment provides a pharmaceutical composition comprising a pharmaceutically acceptable carrier and a compound of Formula (If), or a pharmaceutically acceptable salt thereof.

One embodiment provides a pharmaceutical composition comprising a pharmaceutically acceptable carrier and a compound of Formula (Ig), or a pharmaceutically acceptable salt thereof.

One embodiment provides a pharmaceutical composition comprising a pharmaceutically acceptable carrier and a compound of Formula (Ih), or a pharmaceutically acceptable salt thereof.

One embodiment provides a pharmaceutical composition comprising a pharmaceutically acceptable carrier and a compound of Formula (Ii), or a pharmaceutically acceptable salt thereof.

Another embodiment provides a pharmaceutical composition consisting essentially of a pharmaceutically acceptable carrier and a compound of Formula (I), or a pharmaceutically acceptable salt thereof. Another embodiment provides a pharmaceutical composition consisting essentially of a pharmaceutically acceptable carrier and a compound of Formula (I′), or a pharmaceutically acceptable salt thereof. Another embodiment provides a pharmaceutical composition consisting essentially of a pharmaceutically acceptable carrier and a compound of Formula (Ia), or a pharmaceutically acceptable salt thereof. Another embodiment provides a pharmaceutical composition consisting essentially of a pharmaceutically acceptable carrier and a compound of Formula (Ib), or a pharmaceutically acceptable salt thereof. Another embodiment provides a pharmaceutical composition consisting essentially of a pharmaceutically acceptable carrier and a compound of Formula (Ic), or a pharmaceutically acceptable salt thereof. Another embodiment provides a pharmaceutical composition consisting essentially of a pharmaceutically acceptable carrier and a compound of Formula (Id), or a pharmaceutically acceptable salt thereof. Another embodiment provides a pharmaceutical composition consisting essentially of a pharmaceutically acceptable carrier and a compound of Formula (Ie), or a pharmaceutically acceptable salt thereof. Another embodiment provides a pharmaceutical composition consisting essentially of a pharmaceutically acceptable carrier and a compound of Formula (If), or a pharmaceutically acceptable salt thereof. Another embodiment provides a pharmaceutical composition consisting essentially of a pharmaceutically acceptable carrier and a compound of Formula (Ig), or a pharmaceutically acceptable salt thereof. Another embodiment provides a pharmaceutical composition consisting essentially of a pharmaceutically acceptable carrier and a compound of Formula (Ih), or a pharmaceutically acceptable salt thereof. Another embodiment provides a pharmaceutical composition consisting essentially of a pharmaceutically acceptable carrier and a compound of Formula (Ii), or a pharmaceutically acceptable salt thereof.

In certain embodiments, the compound as described herein is substantially pure, in that it contains less than about 5%, or less than about 1%, or less than about 0.1%, of other organic small molecules, such as contaminating intermediates or by-products that are created, for example, in one or more of the steps of a synthesis method.

These formulations include those suitable for oral, rectal, topical, buccal, parenteral (e.g., subcutaneous, intramuscular, intradermal, or intravenous), vaginal, or aerosol administration.

Exemplary pharmaceutical compositions are used in the form of a pharmaceutical preparation, for example, in solid, semisolid or liquid form, which includes one or more of a disclosed compound, as an active ingredient, in a mixture with an organic or inorganic carrier or excipient suitable for external, enteral or parenteral applications. In some embodiments, the active ingredient is compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

In some embodiments for preparing solid compositions such as tablets, the principal active ingredient is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a disclosed compound or a non-toxic pharmaceutically acceptable salt thereof. When referring to these preformulation compositions as homogeneous, it is meant that the active ingredient is dispersed evenly throughout the composition so that the composition is readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, cellulose, microcrystalline cellulose, silicified microcrystalline cellulose, lactose, sucrose, glucose, mannitol, and/or silicic acid; (2) binders, such as, for example, carboxymethylcellulose, hypromellose, alginates, gelatin, polyvinyl pyrrolidone, sucrose and/or acacia; (3) humectants, such as glycerol; (4) disintegrating agents, such as crospovidone, croscarmellose sodium, sodium starch glycolate, agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, docusate sodium, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, in some embodiments, the compositions comprise buffering agents. In some embodiments, solid compositions of a similar type are also employed as fillers in soft and hard-filled gelatin capsules using such excipients as lactose or milk sugars, as well as high molecular weight polyethylene glycols and the like.

In some embodiments, a tablet is made by compression or molding, optionally with one or more accessory ingredients. In some embodiments, compressed tablets are prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. In some embodiments, molded tablets are made by molding in a suitable machine a mixture of the subject composition moistened with an inert liquid diluent. In some embodiments, tablets, and other solid dosage forms, such as dragees, capsules, pills and granules, are scored or prepared with coatings and shells, such as enteric coatings and other coatings.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, in some embodiments, the liquid dosage forms contain inert diluents, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

In some embodiments, suspensions, in addition to the subject composition, contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In some embodiments, formulations for rectal or vaginal administration are presented as a suppository, which are prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches and inhalants. In some embodiments, the active component is mixed under sterile conditions with a pharmaceutically acceptable carrier, and with any preservatives, buffers, or propellants as required.

In some embodiments, the ointments, pastes, creams and gels contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

In some embodiments, powders and sprays contain, in addition to a subject composition, excipients such as lactose, talc, silicic acid, aluminum hydroxide, calcium silicates and polyamide powder, or mixtures of these substances. In some embodiments, sprays additionally contain customary propellants, such as chlorofluorohydrocarbons and volatile unsubstituted hydrocarbons, such as butane and propane.

In some embodiments, the compounds described herein are formulated as eye drops for ophthalmic administration.

Compositions and compounds disclosed herein alternatively are administered by aerosol. This is accomplished by preparing an aqueous aerosol, liposomal preparation or solid particles containing the compound. In some embodiments, a non-aqueous (e.g., fluorocarbon propellant) suspension is used. In some embodiments, sonic nebulizers are used because they minimize exposing the agent to shear, which results in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions suitable for parenteral administration comprise a subject composition in combination with one or more pharmaceutically-acceptable sterile isotonic aqueous or non-aqueous solutions, dispersions, suspensions or emulsions, or sterile powders which are reconstituted into sterile injectable solutions or dispersions just prior to use, which, in some embodiments, contain antioxidants, buffers, bacteriostats, solutes which render the formulation isotonic with the blood of the intended recipient or suspending or thickening agents.

Examples of suitable aqueous and non-aqueous carriers which are employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity is maintained, for example, by the use of coating materials, such as lecithin, by the maintenance of the required particle size in the case of dispersions, and by the use of surfactants

Also contemplated are enteral pharmaceutical formulations including a disclosed compound and an enteric material; and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro.

The dose of the composition comprising at least one compound described herein differs, depending upon the patient's (e.g., human) condition, that is, stage of the disease, general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. In some embodiments, the optimal dose depends upon the body mass, weight, or blood volume of the patient.

Oral doses typically range from about 1.0 mg to about 1000 mg, one to four times, or more, per day.

We claim:

1. A method for treating a disease or a condition in a patient wherein the disease or condition is selected from atopic dermatitis, bladder dysfunction associated with multiple sclerosis, cardiovascular disease, contact dermatitis, cystic fibrosis, dermatomyositis, eczema, endometriosis, enteritis, fibromyalgia, Tourette syndrome, inflammatory bowel disease, interstitial cystitis, irritable bowel syndrome, ischemia, labor, abdominal pain, abdominal pain associated with irritable bowel syndrome, acute pain, back pain, cancer pain, chest pain, functional chest pain, joint pain, menstrual pain, metabolic disorders, musculoskeletal diseases, neuropathy, osteoarthritis, pancreatitis, pharyngitis, post mastectomy pain syndrome, post trigeminal neuralgia, post operative pain, renal ischemia, rheumatoid arthritis, skeletal muscle contusion, skin diseases, sunburn, systemic lupus erythematosus, toothache, vasoocclusive painful crises in sickle cell disease, and visceral pain, comprising administering to the patient in need thereof a therapeutically effective amount of a compound of Formula (I) having the structure: 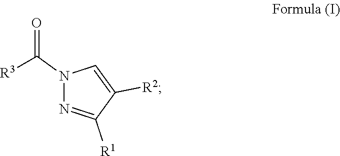
wherein:
R¹ is —C(O)OR¹⁵ or —C(O)NR¹⁰R¹¹;
R² is H, halogen, C₁₋₆alkyl, or C₁₋₆haloalkyl;
R³ is 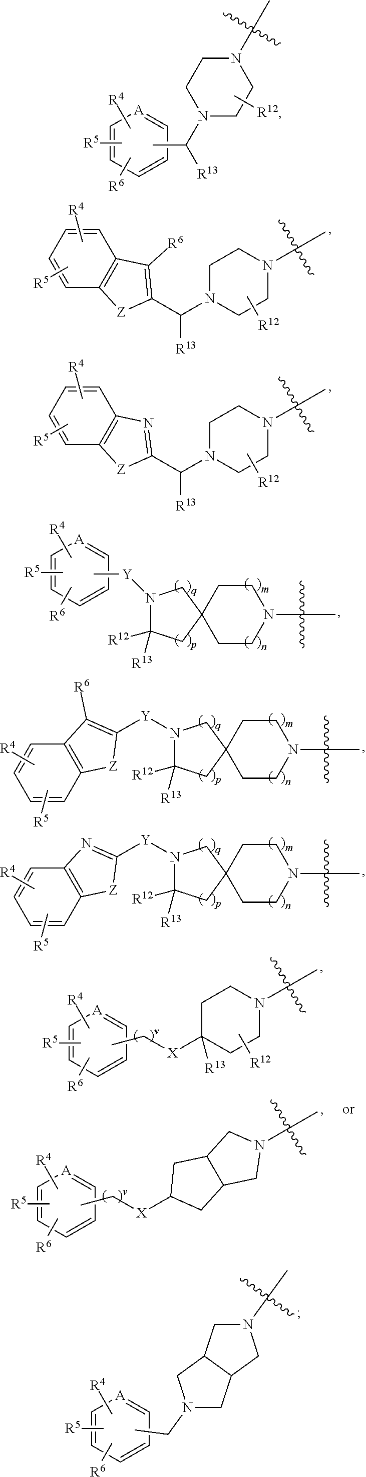
A is N or C(H);
X is —O—, —N(R¹⁶)—, or —CH₂N(R¹⁶)CH₂—;
Y is —CH₂— or —C(O)—;
Z is —S—, —O—, or —N(R¹⁸)—;
R⁴ is H, halogen, —OR⁷, C₁₋₆alkyl, C₂₋₆alkenyl, C₂₋₆alkynyl, C₁₋₆haloalkyl, —C(O)NR⁸R⁹, C₃₋₈cycloalkyl, C₂₋₉heterocycloalkyl, —C₁₋₆alkyl-C₂₋₉heterocycloalkyl, C₆₋₁₀aryl, or C₁₋₉heteroaryl, wherein C₃₋₈cycloalkyl, C₂₋₉heterocycloalkyl, —C₁₋₆alkyl-C₂₋₉ heterocycloalkyl, C₆₋₁₀aryl, or C₁₋₉heteroaryl are optionally substituted with 1 or 2 R¹⁴;
R⁵ is H, halogen, C₁₋₆alkyl, C₁₋₆haloalkyl, C₁₋₆haloalkoxy, or phenyl;
R⁶ is H, halogen or C₁₋₆alkyl;
R⁷ is H, C₁₋₆alkyl, C₁₋₆haloalkyl, —C₁₋₆alkyl-OH, C₂₋₉heterocycloalkyl, C₆₋₁₀aryl, or C₁₋₉heteroaryl, wherein C₂₋₉heterocycloalkyl, C₆₋₁₀aryl, or C₁₋₉heteroaryl are optionally substituted with 1 or 2 R¹⁴;
each R⁸ and each R⁹ are independently selected from H and C₁₋₆alkyl; or R⁸ and R⁹ together with the nitrogen to which they are attached are combined to form a heterocycloalkyl ring;
R¹⁰ and R¹¹ are each independently H or C₁₋₆alkyl;
R¹² is H, halogen, or C₁₋₆alkyl;
R¹³ is H or C₁₋₆alkyl;
each R¹⁴ is independently selected from halogen, —OH, C₁₋₆alkyl, C₁₋₆haloalkyl, C₁₋₆alkoxy, —C₁₋₆alkyl-OH, C₃₋₈cycloalkyl, —C(O)OH, —C(O)NR⁸R⁹, —SO₂—C₁₋₆alkyl, and —N(R¹⁷)C(O)—C₁₋₆alkyl;
R¹⁵ is H or C₁₋₆alkyl;
R¹⁶ is H, C₁₋₆alkyl, —C(O)—C₁₋₆alkyl, —C₁₋₆alkyl-OH, or —CH₂CO₂H;
R¹⁷ is H or C₁₋₆alkyl;
R¹⁸ is H or C₁₋₆alkyl;
v is 0 or 1;
n is 0 or 1;
m is 0 or 1;
p is 0, 1, or 2; and
q is 0, 1 or 2;
or a solvate, hydrate, tautomer, N-oxide, stereoisomer, or pharmaceutically acceptable salt thereof.

2. The method of claim 1, or a solvate, hydrate, tautomer, N-oxide, stereoisomer, or pharmaceutically acceptable salt thereof, wherein R is

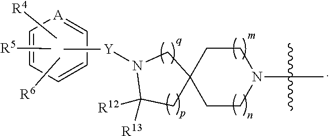

3. The method of claim 1 or 2, or a solvate, hydrate, tautomer, N-oxide, stereoisomer, or pharmaceutically acceptable salt thereof, wherein m is 1, n is 1, q is 0, and p is 2.

4. The method of any one of claims 1-3, or a solvate, hydrate, tautomer, N-oxide, stereoisomer, or pharmaceutically acceptable salt thereof, wherein Y is —CH₂— and A is C(H).

5. The method of any one of claims 1-4, or a solvate, hydrate, tautomer, N-oxide, stereoisomer, or pharmaceutically acceptable salt thereof, wherein R¹² is H and R¹³ is H.

6. The method of any one of claims 1-5, or a solvate, hydrate, tautomer, N-oxide, stereoisomer, or pharmaceutically acceptable salt thereof, wherein R⁴ is halogen, —OR⁷, C₁₋₆haloalkyl, C₂₋₉heterocycloalkyl, C₆₋₁₀aryl, or C₁₋₉heteroaryl, wherein C₂₋₉ heterocycloalkyl, C₆₋₁₀aryl, or C₁₋₉heteroaryl are optionally substituted with 1 or 2 R¹⁴.

7. The method of any one of claims 1-6, or a solvate, hydrate, tautomer, N-oxide, stereoisomer, or pharmaceutically acceptable salt thereof, wherein R⁴ is halogen or R⁴ is C₂₋₉heterocycloalkyl optionally substituted with 1 or 2 R¹⁴.

8. The method of any one of claims 1-7, or a solvate, hydrate, tautomer, N-oxide, stereoisomer, or pharmaceutically acceptable salt thereof, wherein R² is H, R⁶ is H, and R⁵ is H, halogen, C₁₋₆alkyl, C₁₋₆haloalkyl, or C₁₋₆haloalkoxy.

9. The method of any one of claims 1-8, or a solvate, hydrate, tautomer, N-oxide, stereoisomer, or pharmaceutically acceptable salt thereof, wherein R¹ is —C(O)OR¹⁵ and R¹⁵ is H.

10. The method of claim 1, or a solvate, hydrate, tautomer, N-oxide, stereoisomer, or pharmaceutically acceptable salt thereof, wherein the compound is 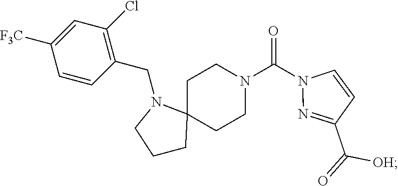 [SMILES: O=C(O)c1ccn(C(=O)N2CCC3(CCCN3Cc3ccc(C(F)(F)F)cc3Cl)CC2)n1]
or a solvate, hydrate, tautomer, N-oxide, stereoisomer, or pharmaceutically acceptable salt thereof.

11. The method of claim 1, or a solvate, hydrate, tautomer, N-oxide, stereoisomer, or pharmaceutically acceptable salt thereof, wherein the compound is 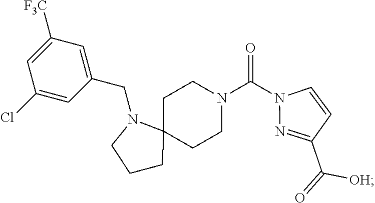 [SMILES: O=C(O)c1ccn(C(=O)N2CCC3(CCCN3Cc3cc(Cl)cc(C(F)(F)F)c3)CC2)n1]
or a solvate, hydrate, tautomer, N-oxide, stereoisomer, or pharmaceutically acceptable salt thereof.

12. The method of claim 1, or a solvate, hydrate, tautomer, N-oxide, stereoisomer, or pharmaceutically acceptable salt thereof, wherein the compound is 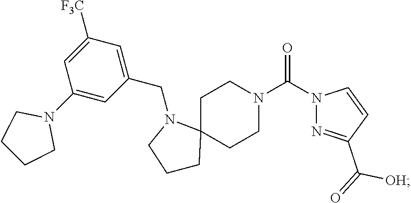 [SMILES: O=C(O)c1ccn(C(=O)N2CCC3(CCCN3Cc3cc(N4CCCC4)cc(C(F)(F)F)c3)CC2)n1]
or a solvate, hydrate, tautomer, N-oxide, stereoisomer, or pharmaceutically acceptable salt thereof.

13. The method of claim 1, or a solvate, hydrate, tautomer, N-oxide, stereoisomer, or pharmaceutically acceptable salt thereof, wherein the compound is 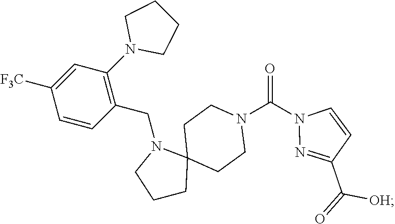 [SMILES: O=C(O)c1ccn(C(=O)N2CCC3(CCCN3Cc3ccc(C(F)(F)F)cc3N3CCCC3)CC2)n1]
or a solvate, hydrate, tautomer, N-oxide, stereoisomer, or pharmaceutically acceptable salt thereof.

14. The method of claim 1, or a solvate, hydrate, tautomer, N-oxide, stereoisomer, or pharmaceutically acceptable salt thereof, wherein the compound is 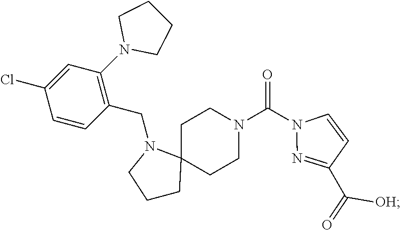 [SMILES: O=C(O)c1ccn(C(=O)N2CCC3(CCCN3Cc3ccc(Cl)cc3N3CCCC3)CC2)n1]
or a solvate, hydrate, tautomer, N-oxide, stereoisomer, or pharmaceutically acceptable salt thereof.

15. The method of claim 1, wherein the compound is selected from: 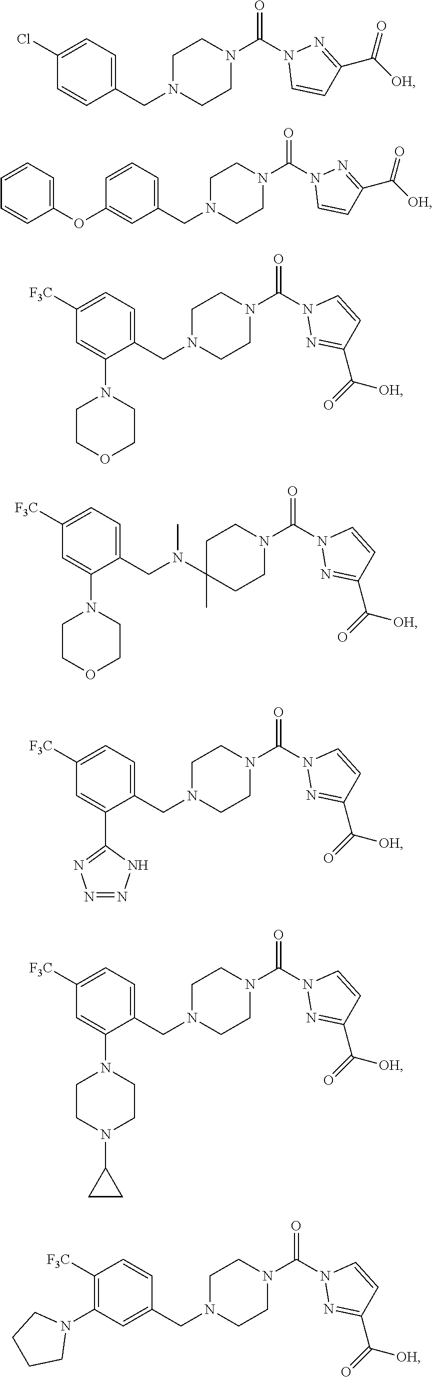 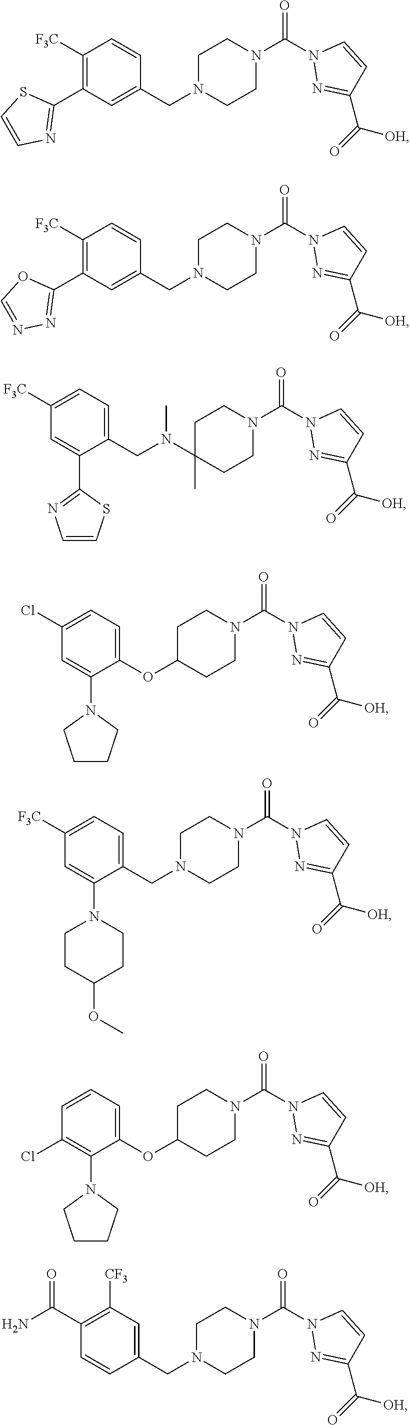 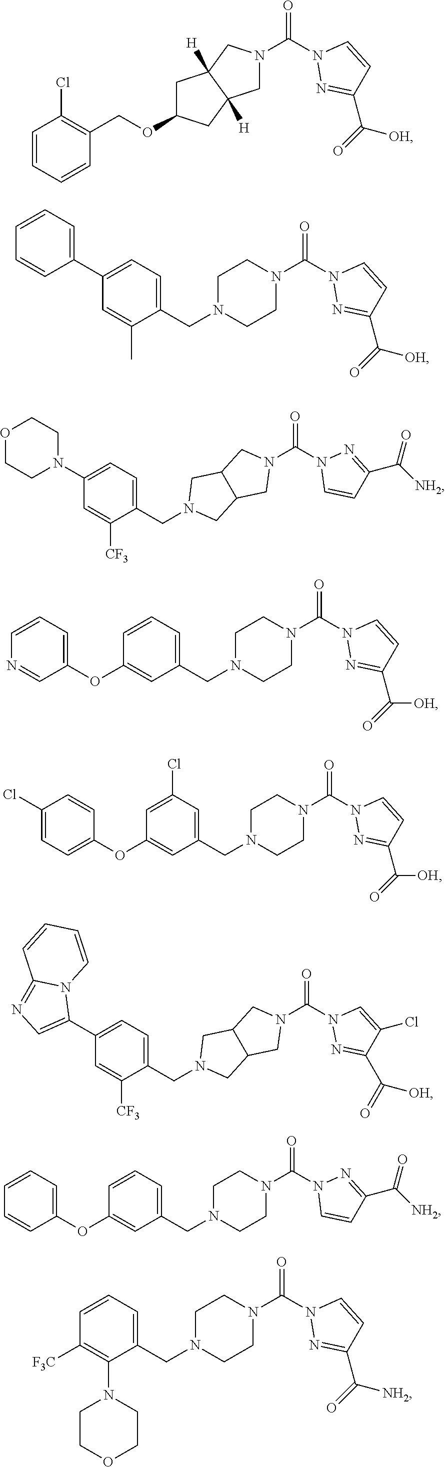 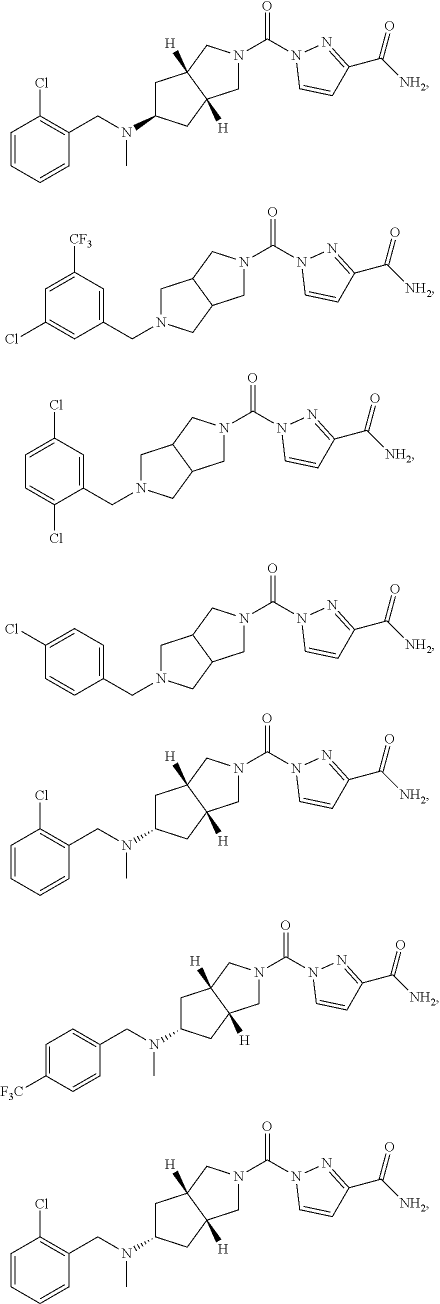 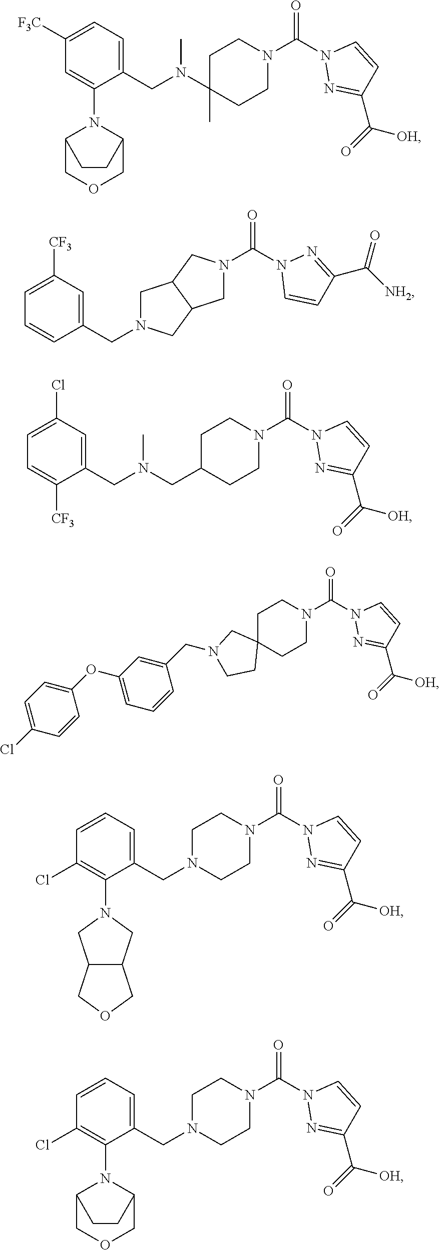 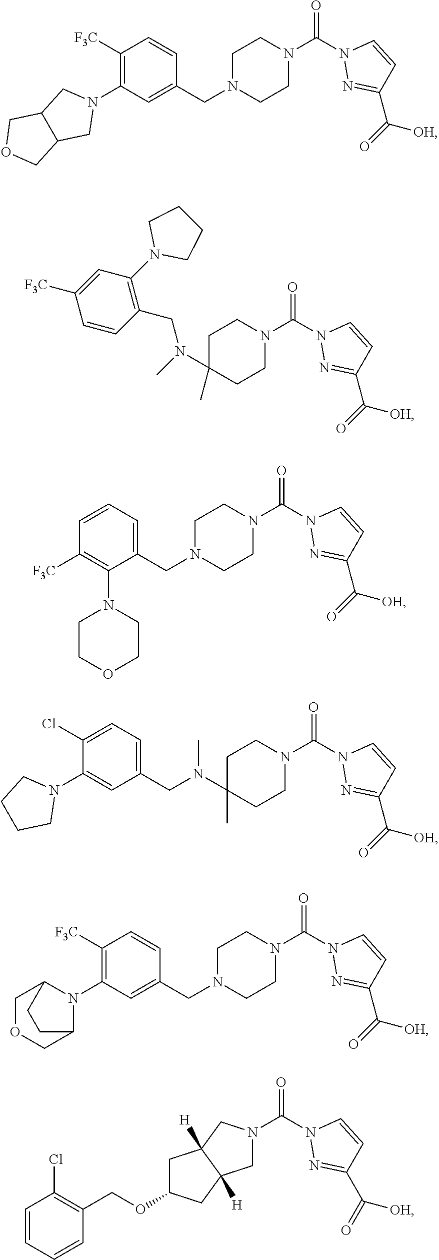 [SMILES: O=C(O)c1ccn(C(=O)N2CCN(Cc3ccc(C(F)(F)F)c(N4CC5COCC5C4)c3)CC2)n1 ; CN(Cc1ccc(C(F)(F)F)cc1N1CCCC1)C1(C)CCN(C(=O)n2ccc(C(=O)O)n2)CC1 ; O=C(O)c1ccn(C(=O)N2CCN(Cc3cccc(C(F)(F)F)c3N3CCOCC3)CC2)n1 ; CN(Cc1ccc(Cl)c(N2CCCC2)c1)C1(C)CCN(C(=O)n2ccc(C(=O)O)n2)CC1 ; O=C(O)c1ccn(C(=O)N2CCN(Cc3ccc(C(F)(F)F)c(N4C5CCC4COC5)c3)CC2)n1 ; O=C(O)c1ccn(C(=O)N2C[C@H]3C[C@@H](OCc4ccccc4Cl)C[C@H]3C2)n1] 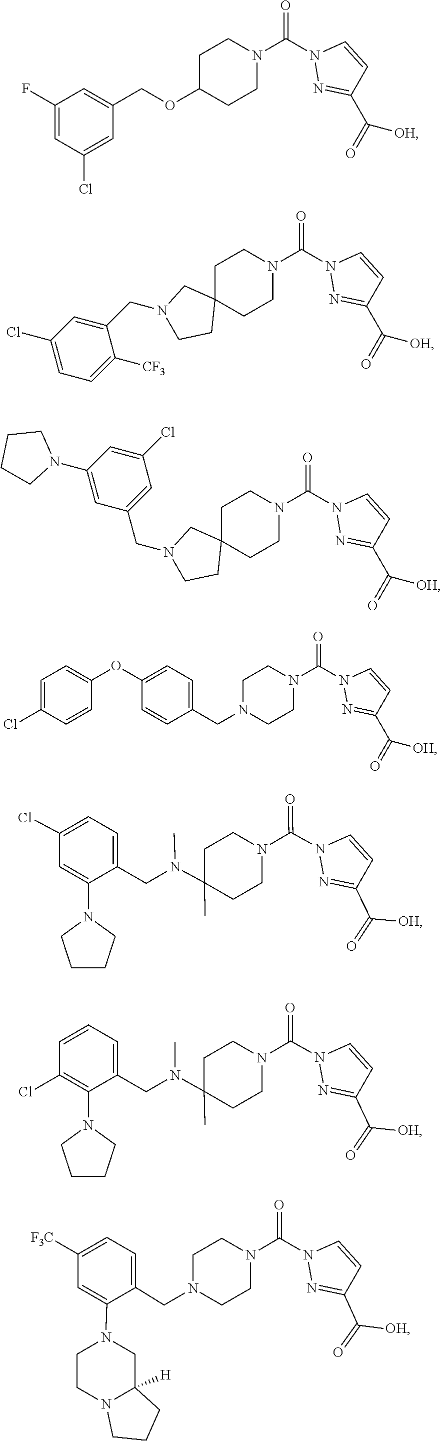 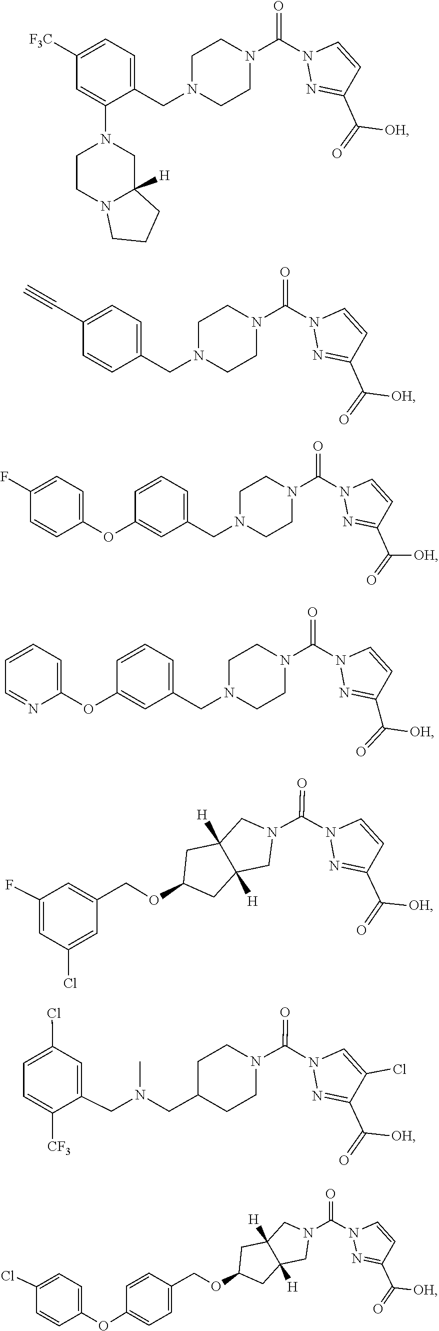 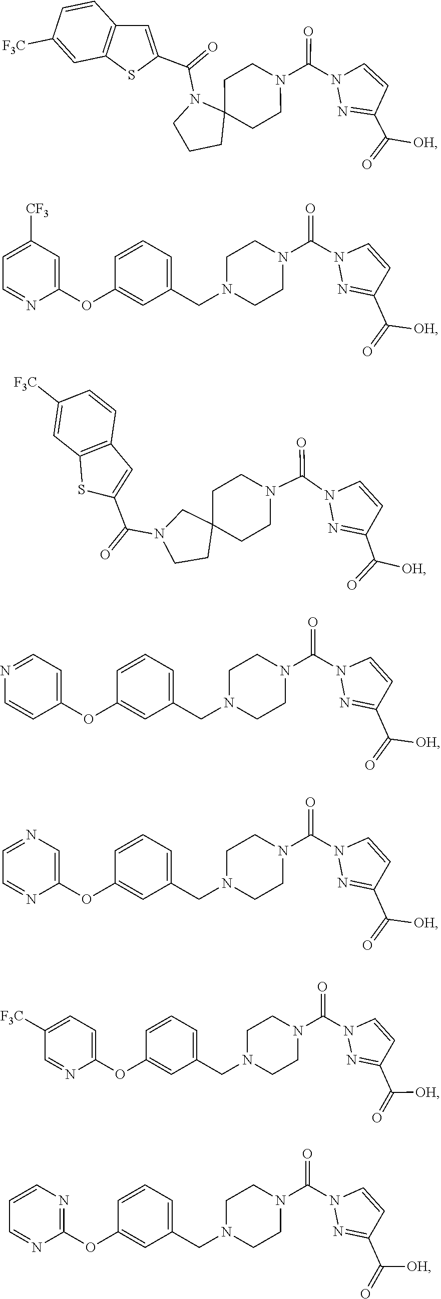 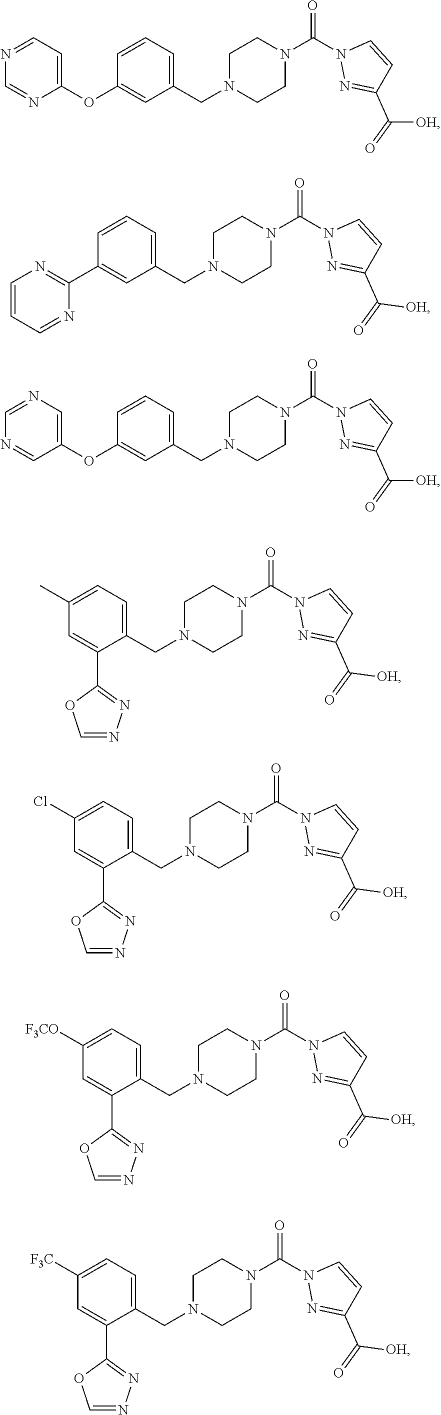 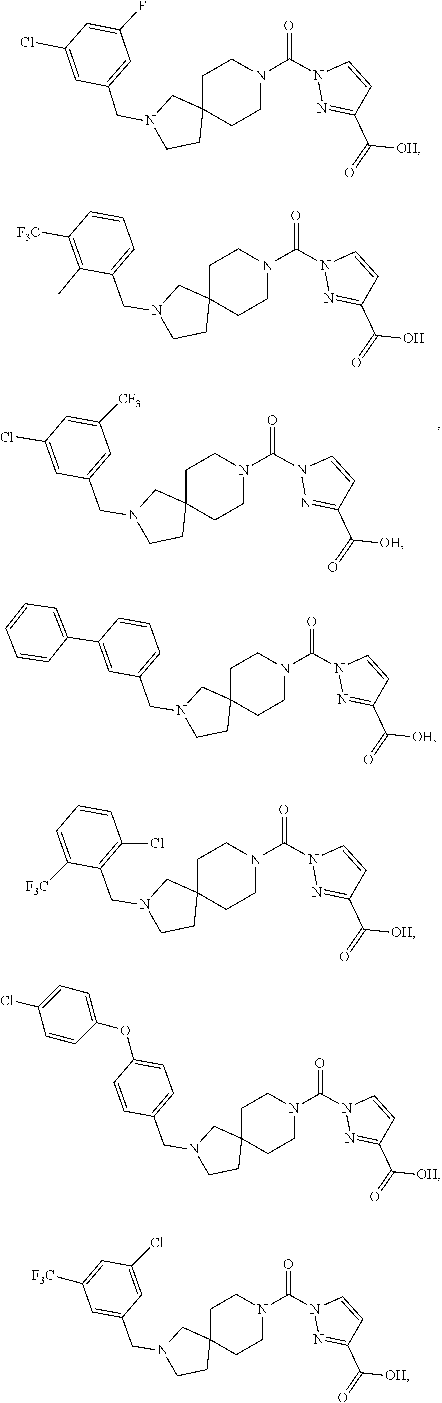 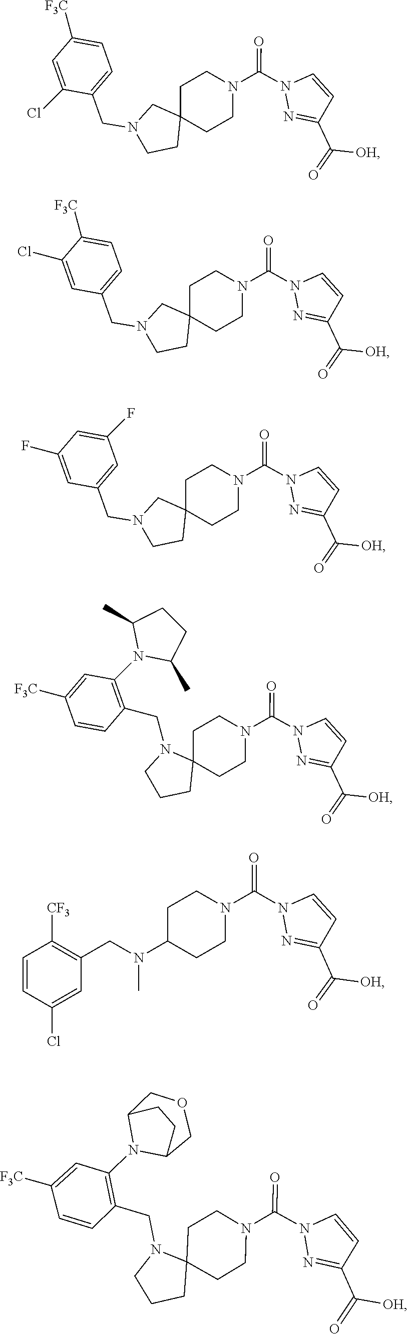 [SMILES: O=C(O)c1ccn(C(=O)N2CCC3(CCN(Cc4ccc(C(F)(F)F)cc4Cl)C3)CC2)n1 ; O=C(O)c1ccn(C(=O)N2CCC3(CCN(Cc4ccc(C(F)(F)F)c(Cl)c4)C3)CC2)n1 ; O=C(O)c1ccn(C(=O)N2CCC3(CCN(Cc4cc(F)cc(F)c4)C3)CC2)n1 ; C[C@@H]1CC[C@H](C)N1c1cc(C(F)(F)F)ccc1CN1CCCC12CCN(C(=O)n1ccc(C(=O)O)n1)CC2 ; CN(Cc1cc(Cl)ccc1C(F)(F)F)C1CCN(C(=O)n2ccc(C(=O)O)n2)CC1 ; O=C(O)c1ccn(C(=O)N2CCC3(CCCN3Cc3ccc(C(F)(F)F)cc3N3C4CCC3COC4)CC2)n1] 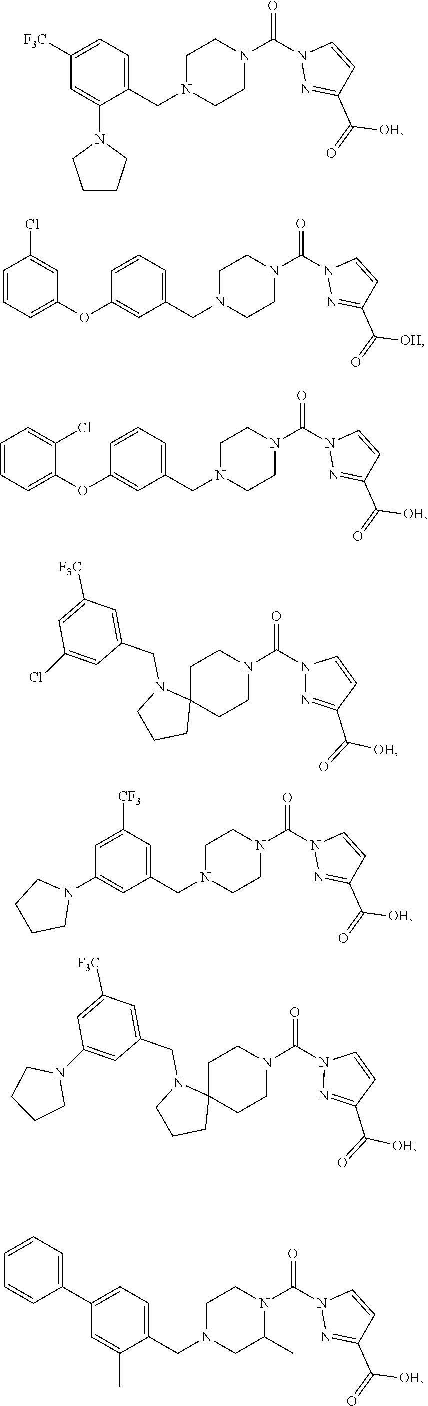 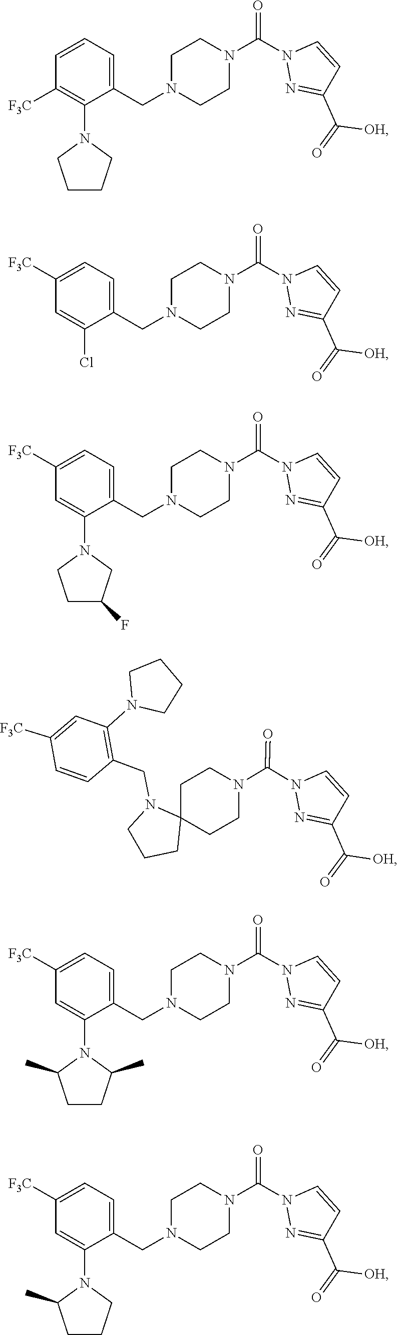 [SMILES: O=C(O)c1ccn(C(=O)N2CCN(Cc3cccc(C(F)(F)F)c3N3CCCC3)CC2)n1 ; O=C(O)c1ccn(C(=O)N2CCN(Cc3ccc(C(F)(F)F)cc3Cl)CC2)n1 ; O=C(O)c1ccn(C(=O)N2CCN(Cc3ccc(C(F)(F)F)cc3N3CC[C@H](F)C3)CC2)n1 ; O=C(O)c1ccn(C(=O)N2CCC3(CCCN3Cc3ccc(C(F)(F)F)cc3N3CCCC3)CC2)n1 ; C[C@@H]1CC[C@H](C)N1c1cc(C(F)(F)F)ccc1CN1CCN(C(=O)n2ccc(C(=O)O)n2)CC1 ; C[C@@H]1CCCN1c1cc(C(F)(F)F)ccc1CN1CCN(C(=O)n2ccc(C(=O)O)n2)CC1] 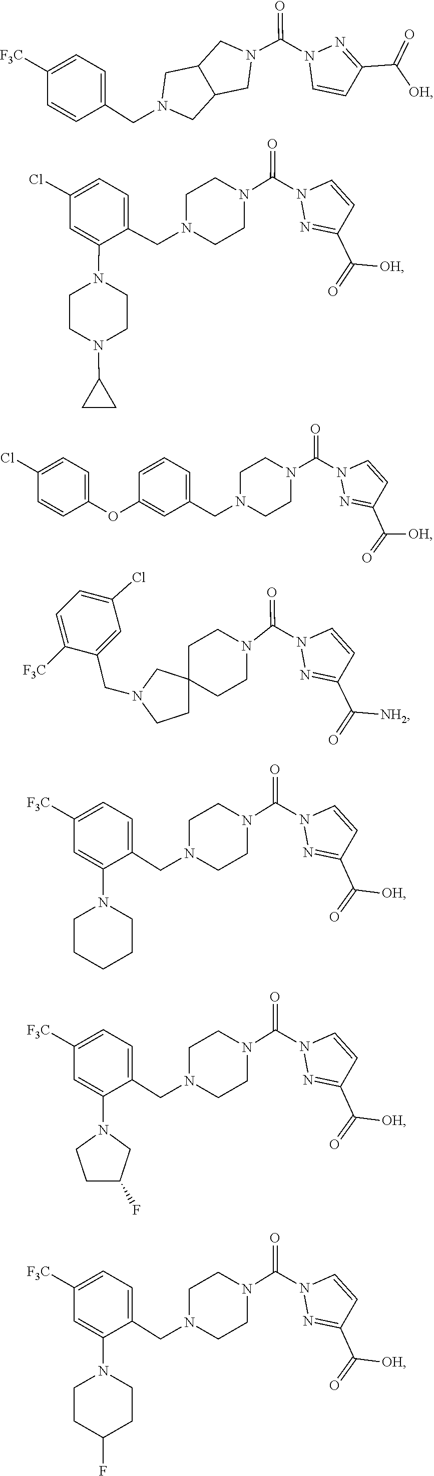 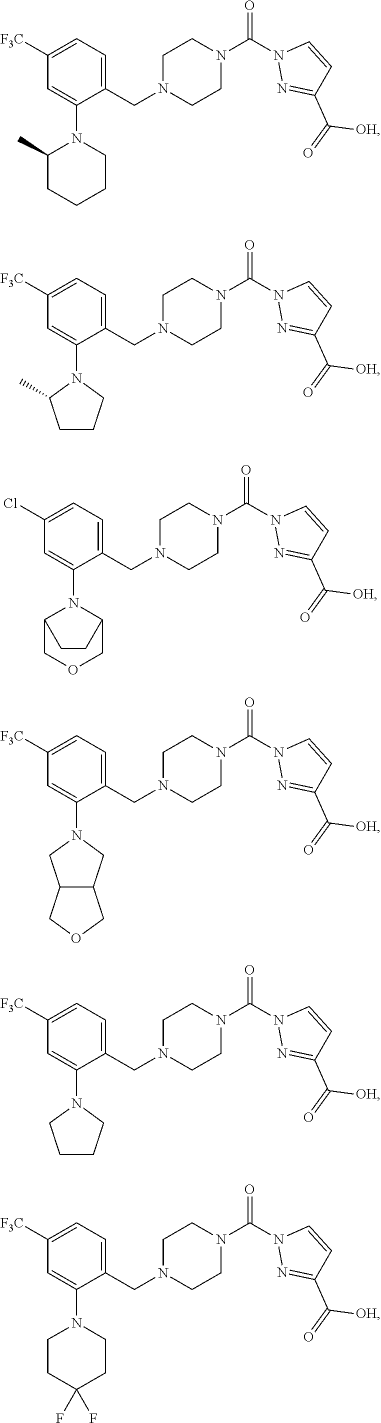 [SMILES: C[C@@H]1CCCCN1c1cc(C(F)(F)F)ccc1CN1CCN(C(=O)n2ccc(C(=O)O)n2)CC1 ; C[C@H]1CCCN1c1cc(C(F)(F)F)ccc1CN1CCN(C(=O)n2ccc(C(=O)O)n2)CC1 ; O=C(O)c1ccn(C(=O)N2CCN(Cc3ccc(Cl)cc3N3C4CCC3COC4)CC2)n1 ; O=C(O)c1ccn(C(=O)N2CCN(Cc3ccc(C(F)(F)F)cc3N3CC4COCC4C3)CC2)n1 ; O=C(O)c1ccn(C(=O)N2CCN(Cc3ccc(C(F)(F)F)cc3N3CCCC3)CC2)n1 ; O=C(O)c1ccn(C(=O)N2CCN(Cc3ccc(C(F)(F)F)cc3N3CCC(F)(F)CC3)CC2)n1] 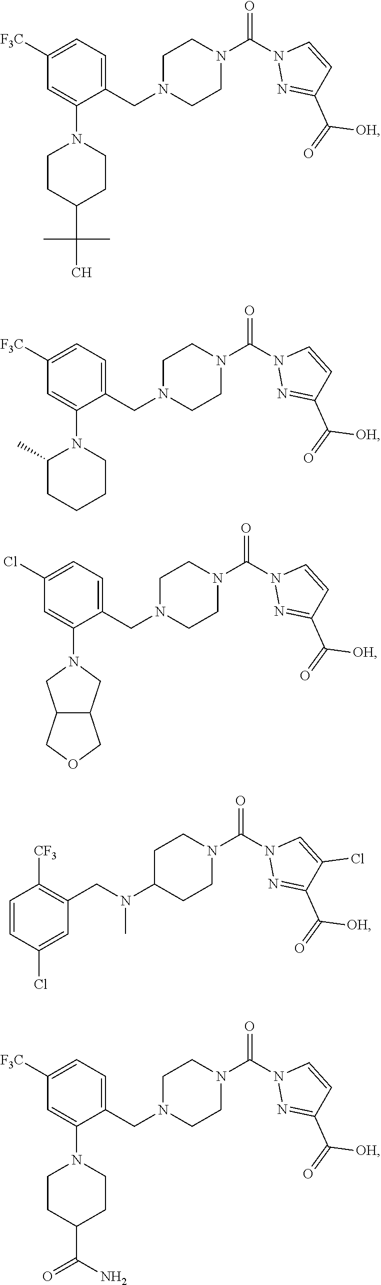 [SMILES: CC(C)(C)C1CCN(c2cc(C(F)(F)F)ccc2CN2CCN(C(=O)n3ccc(C(=O)O)n3)CC2)CC1 ; C[C@H]1CCCCN1c1cc(C(F)(F)F)ccc1CN1CCN(C(=O)n2ccc(C(=O)O)n2)CC1 ; O=C(O)c1ccn(C(=O)N2CCN(Cc3ccc(Cl)cc3N3CC4COCC4C3)CC2)n1 ; CN(Cc1cc(Cl)ccc1C(F)(F)F)C1CCN(C(=O)n2cc(Cl)c(C(=O)O)n2)CC1 ; NC(=O)C1CCN(c2cc(C(F)(F)F)ccc2CN2CCN(C(=O)n3ccc(C(=O)O)n3)CC2)CC1] 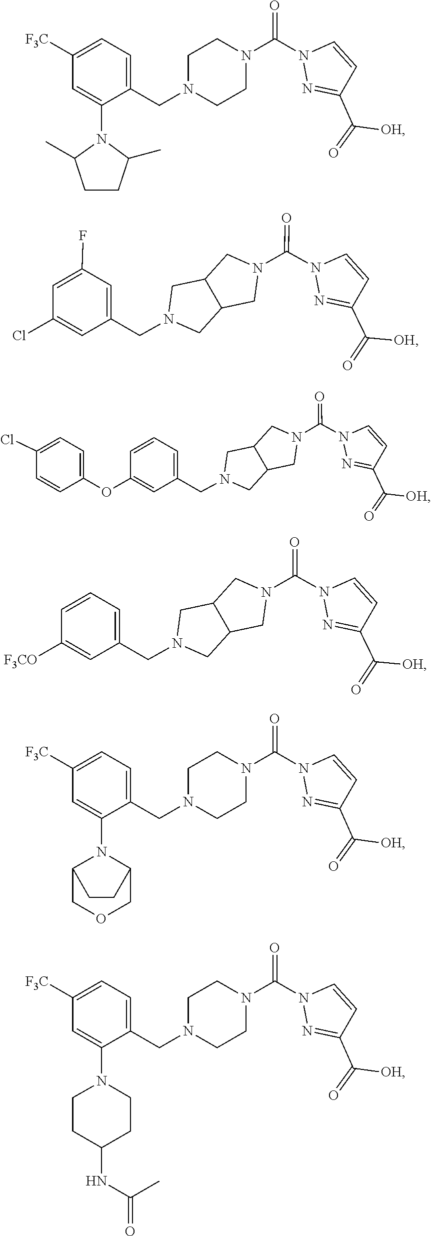 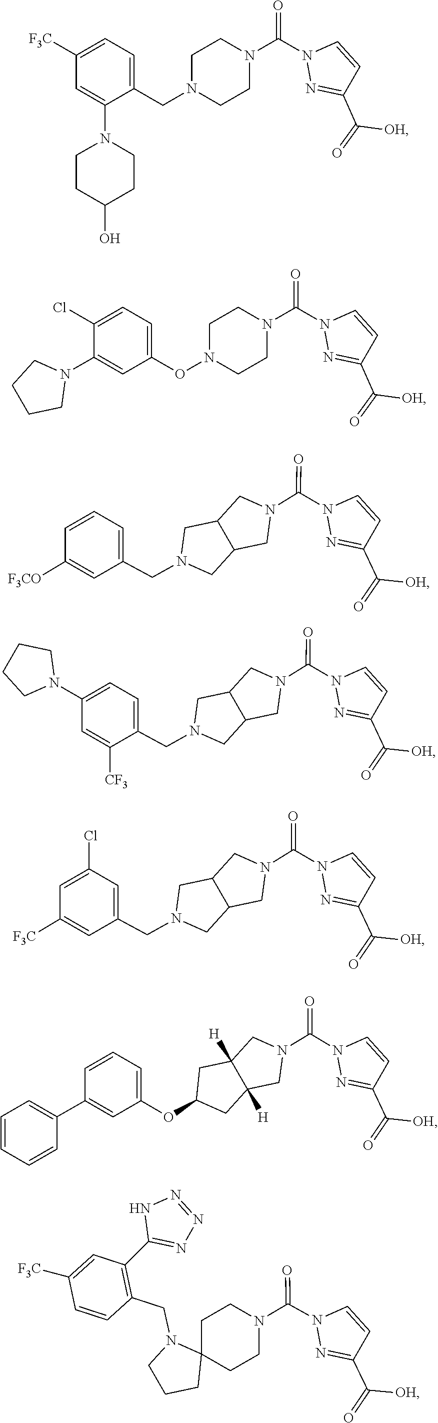 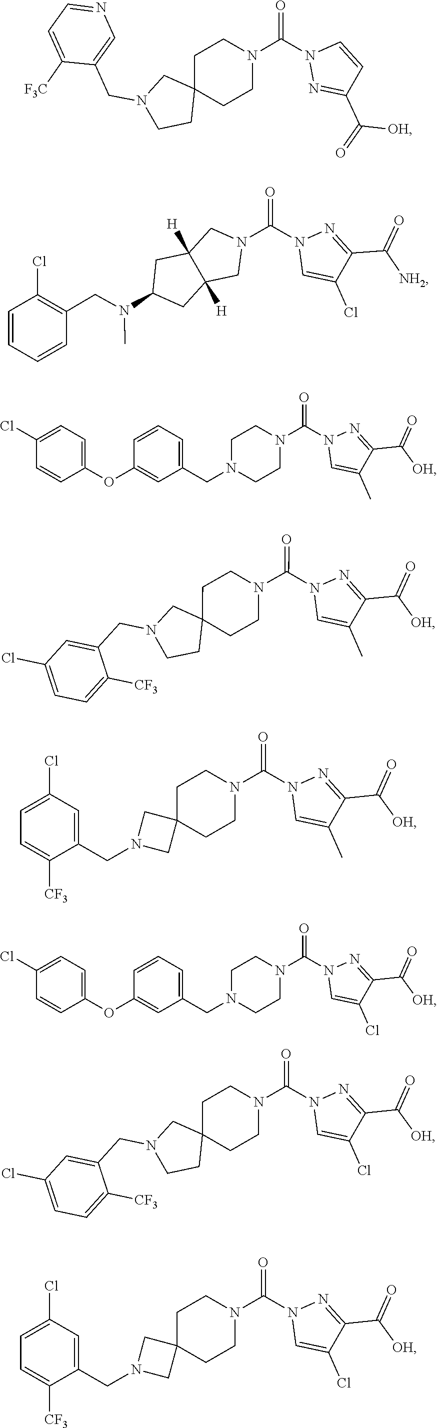 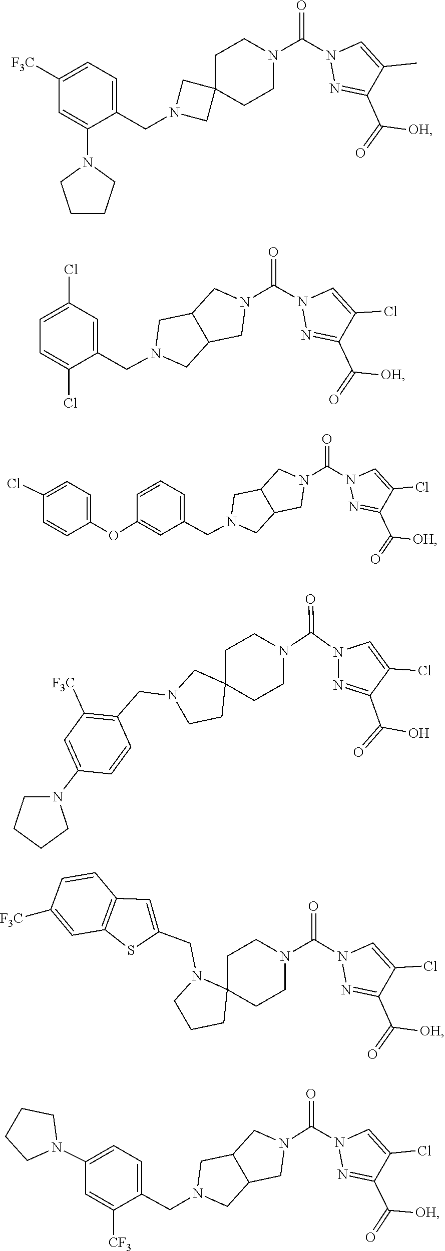 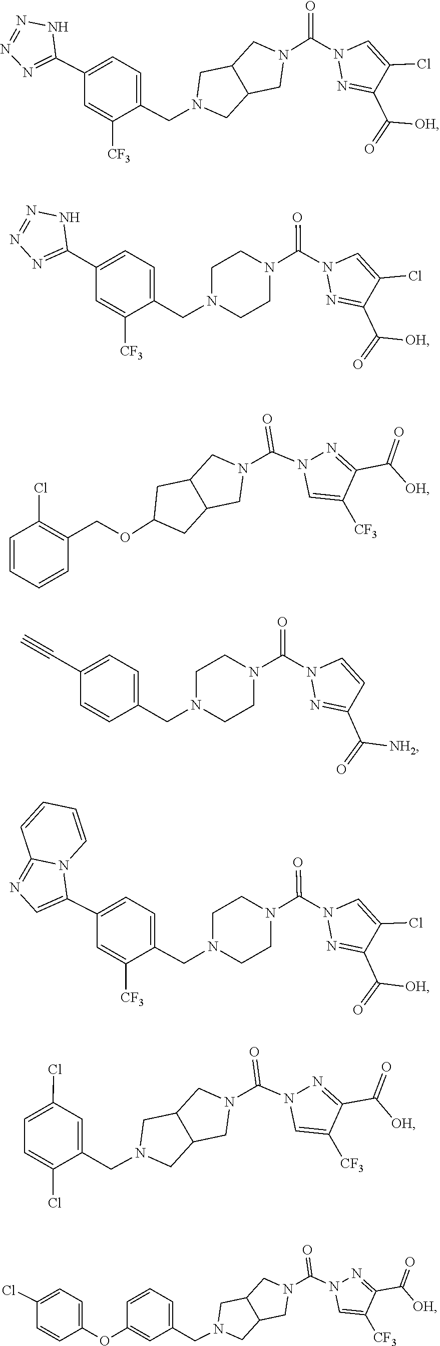 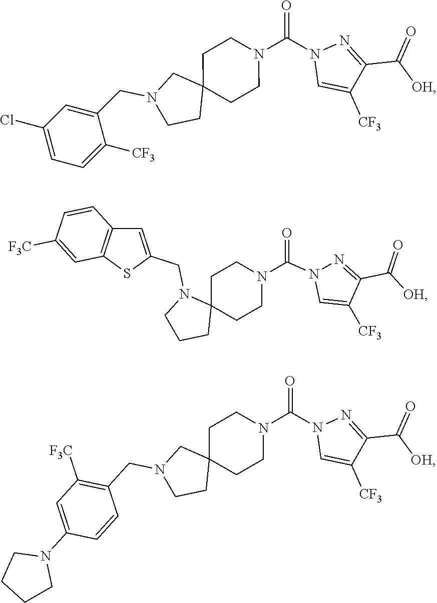 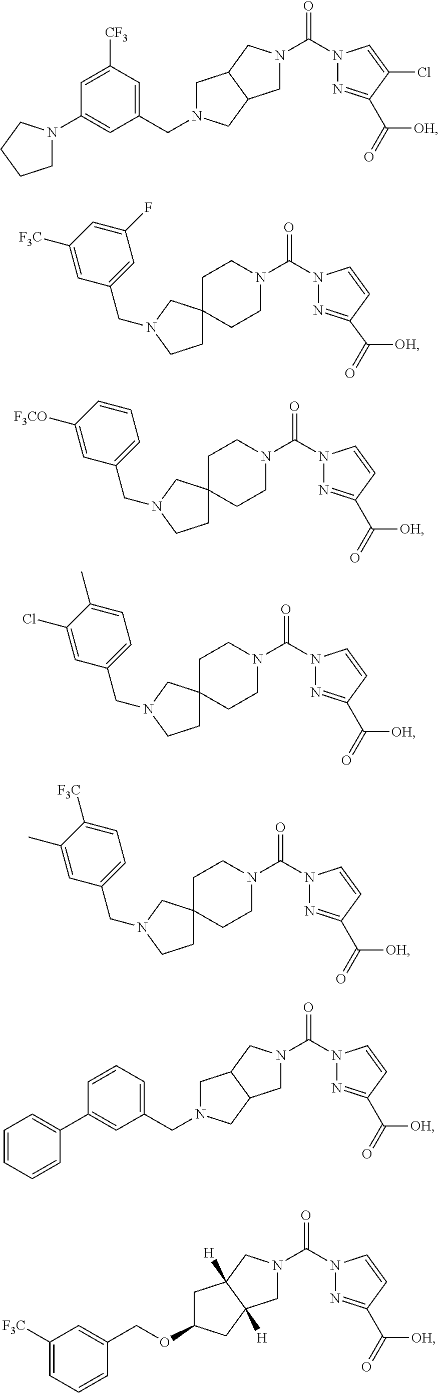 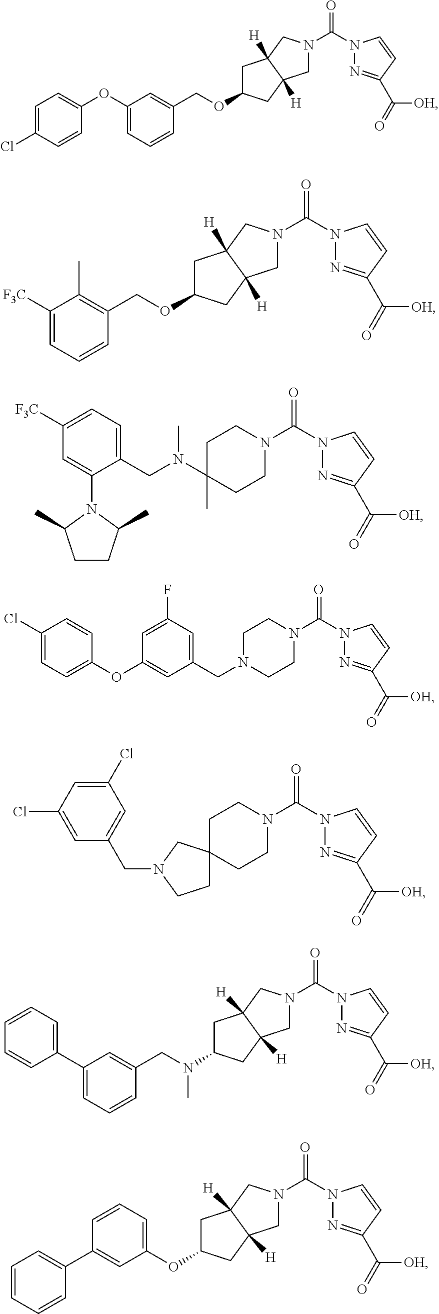 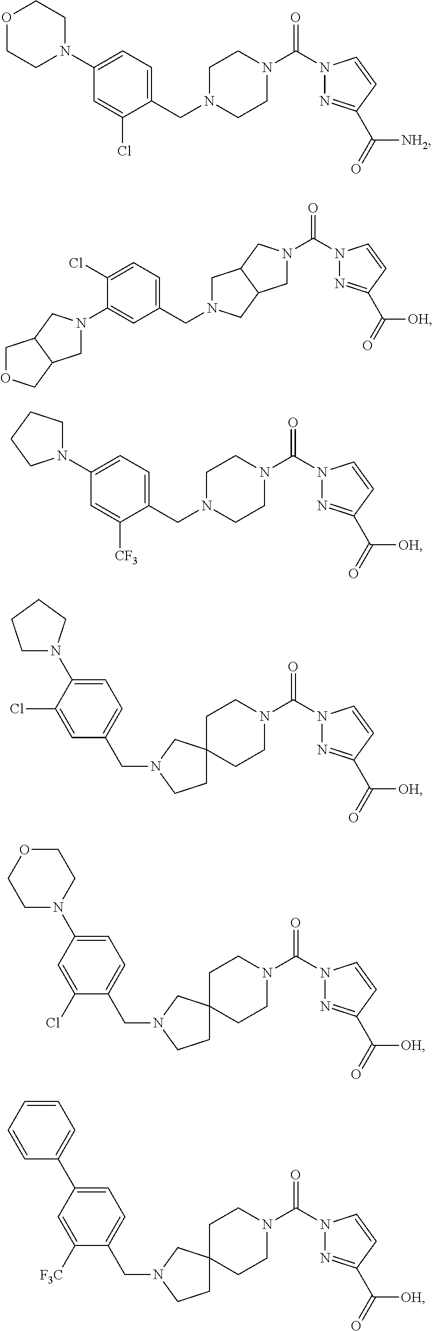 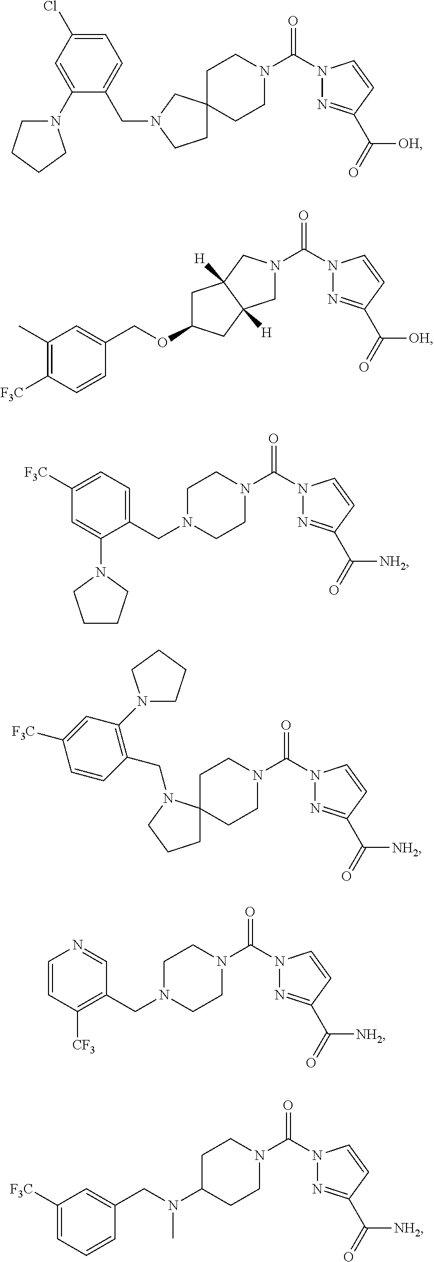 [SMILES: O=C(O)c1ccn(C(=O)N2CCC3(CCN(Cc4ccc(Cl)cc4N4CCCC4)C3)CC2)n1 ; Cc1cc(CO[C@H]2C[C@@H]3CN(C(=O)n4ccc(C(=O)O)n4)C[C@@H]3C2)ccc1C(F)(F)F ; NC(=O)c1ccn(C(=O)N2CCN(Cc3ccc(C(F)(F)F)cc3N3CCCC3)CC2)n1 ; NC(=O)c1ccn(C(=O)N2CCC3(CCCN3Cc3ccc(C(F)(F)F)cc3N3CCCC3)CC2)n1 ; NC(=O)c1ccn(C(=O)N2CCN(Cc3cnccc3C(F)(F)F)CC2)n1 ; CN(Cc1cccc(C(F)(F)F)c1)C1CCN(C(=O)n2ccc(C(N)=O)n2)CC1] 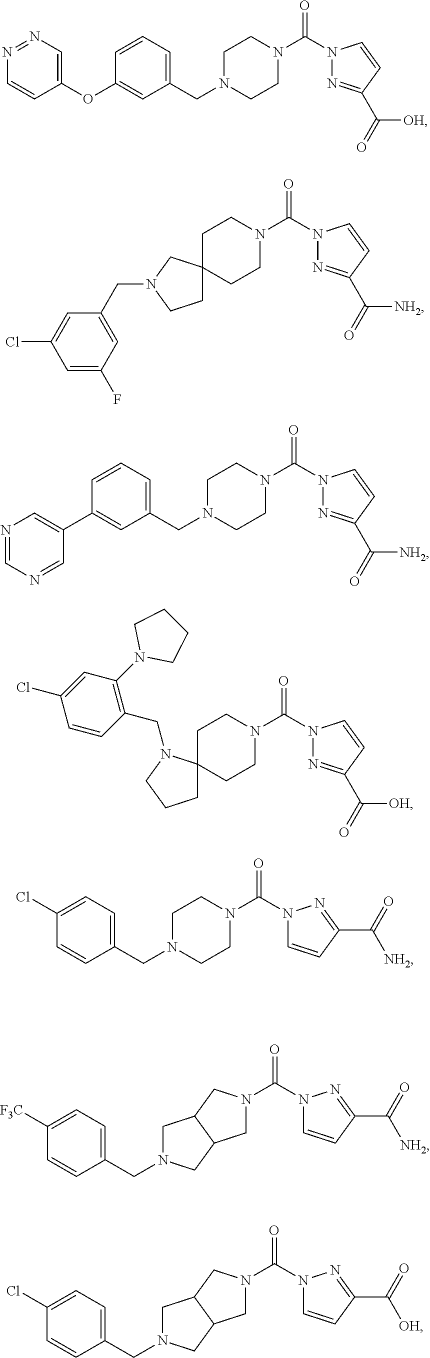 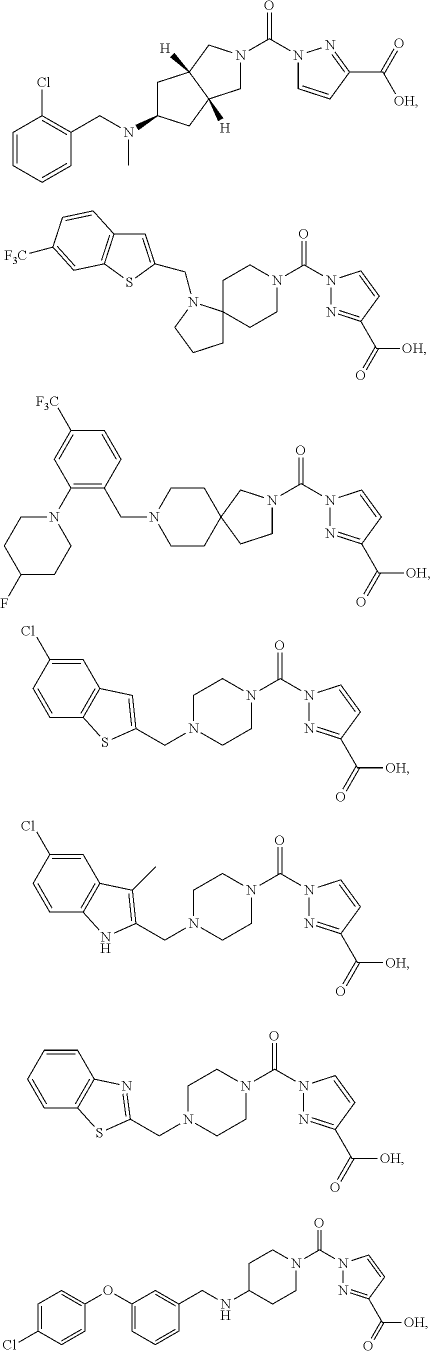 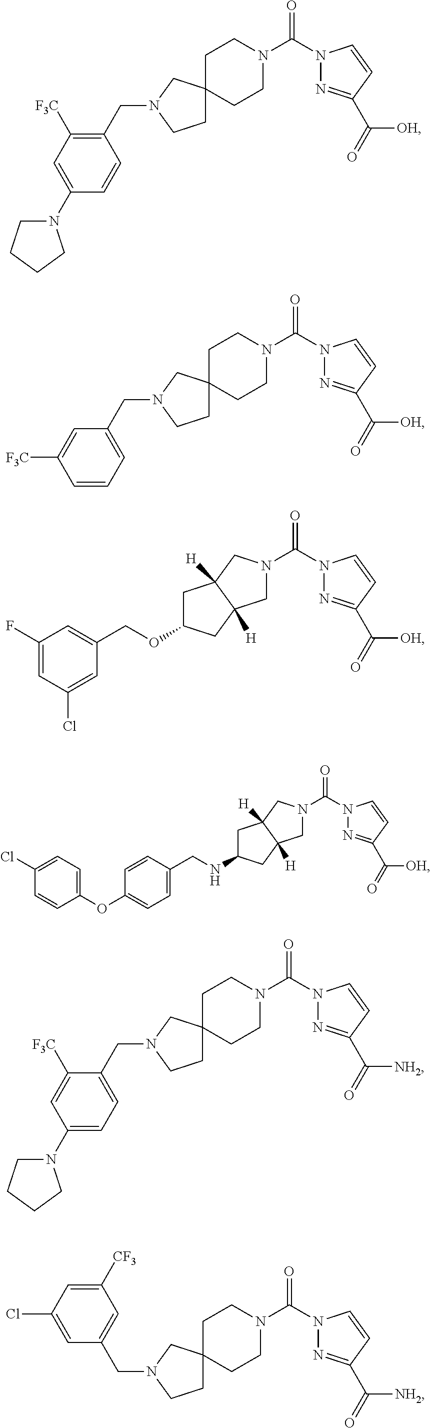 [SMILES: O=C(O)c1ccn(C(=O)N2CCC3(CCN(Cc4ccc(N5CCCC5)cc4C(F)(F)F)C3)CC2)n1 ; O=C(O)c1ccn(C(=O)N2CCC3(CCN(Cc4cccc(C(F)(F)F)c4)C3)CC2)n1 ; O=C(O)c1ccn(C(=O)N2C[C@H]3C[C@@H](OCc4cc(F)cc(Cl)c4)C[C@H]3C2)n1 ; O=C(O)c1ccn(C(=O)N2C[C@H]3C[C@H](NCc4ccc(Oc5ccc(Cl)cc5)cc4)C[C@H]3C2)n1 ; NC(=O)c1ccn(C(=O)N2CCC3(CCN(Cc4ccc(N5CCCC5)cc4C(F)(F)F)C3)CC2)n1 ; NC(=O)c1ccn(C(=O)N2CCC3(CCN(Cc4cc(Cl)cc(C(F)(F)F)c4)C3)CC2)n1] 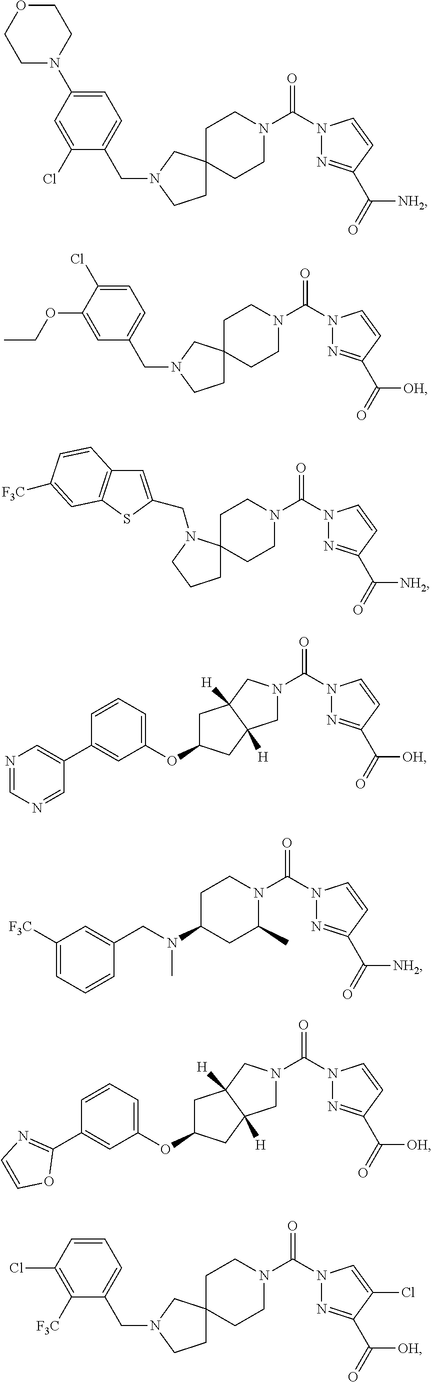 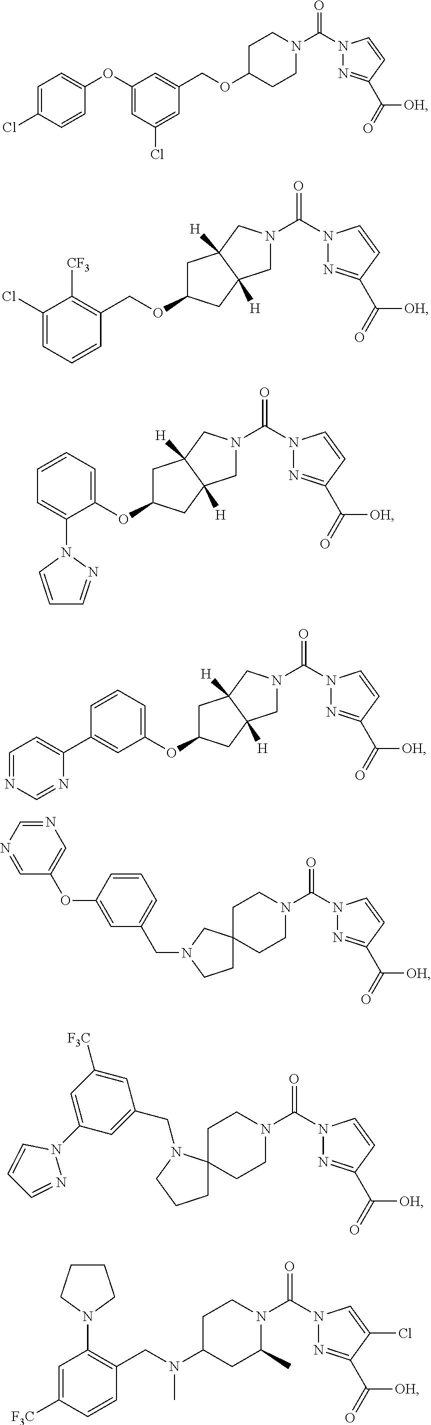 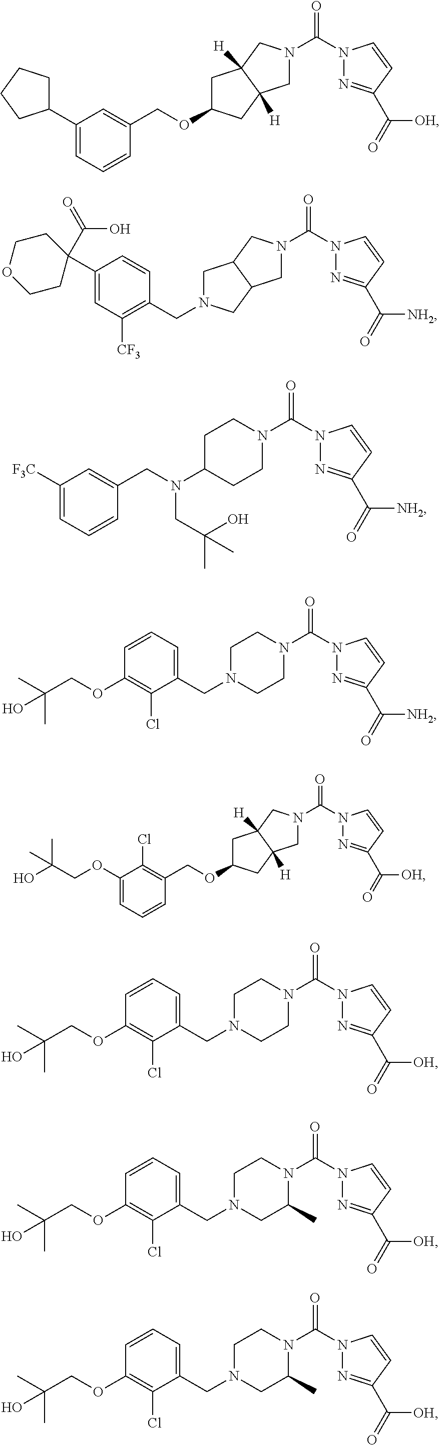 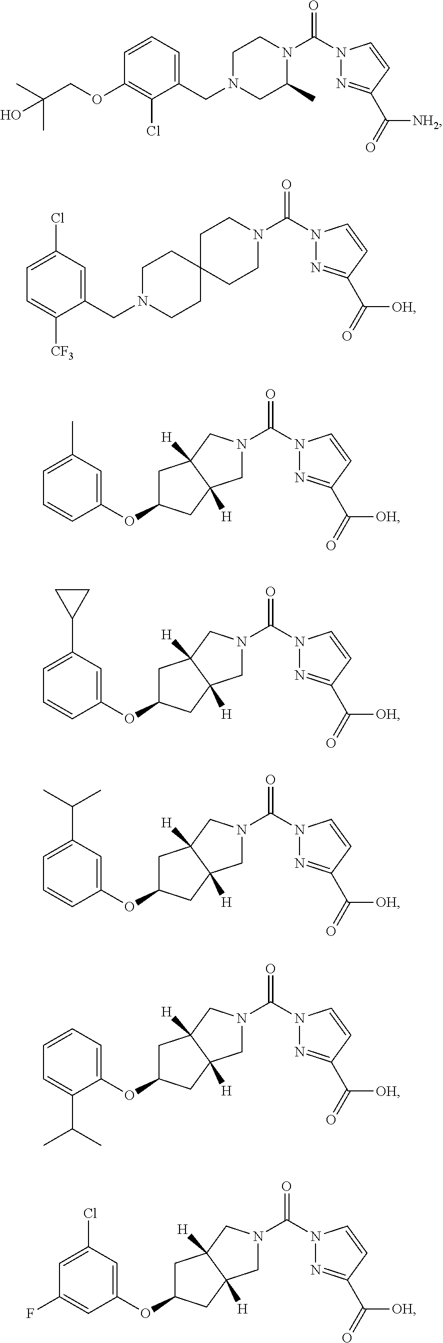 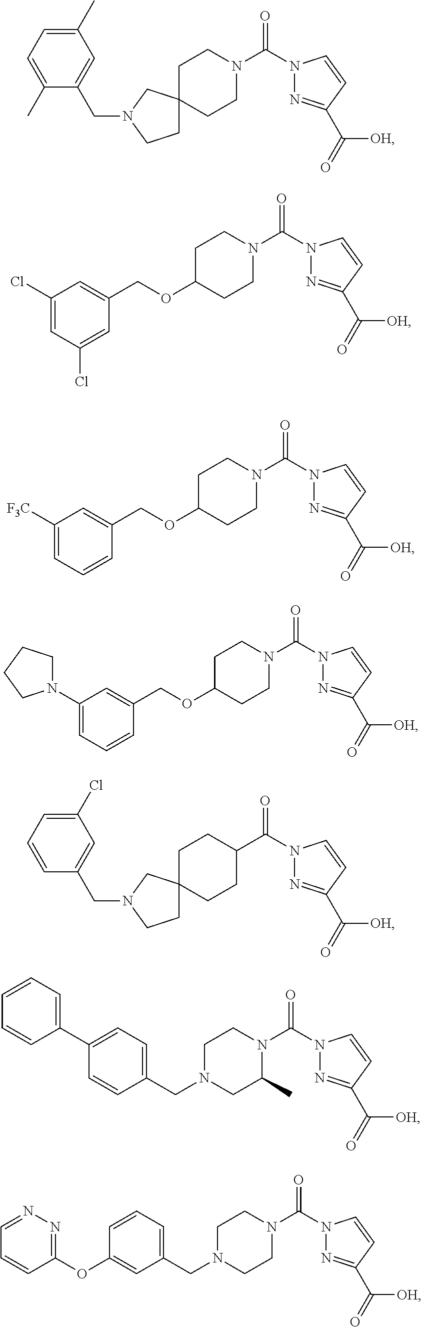 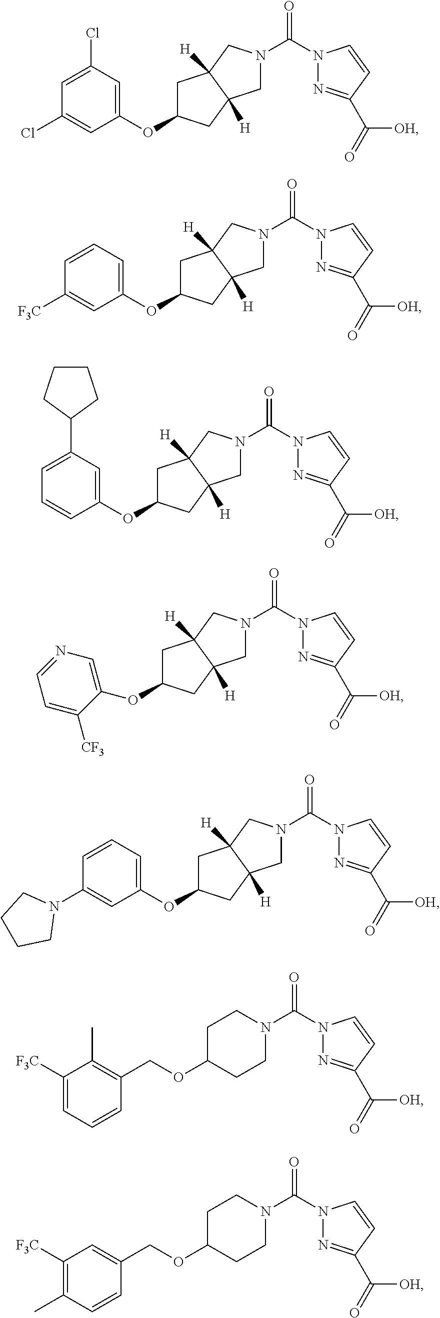 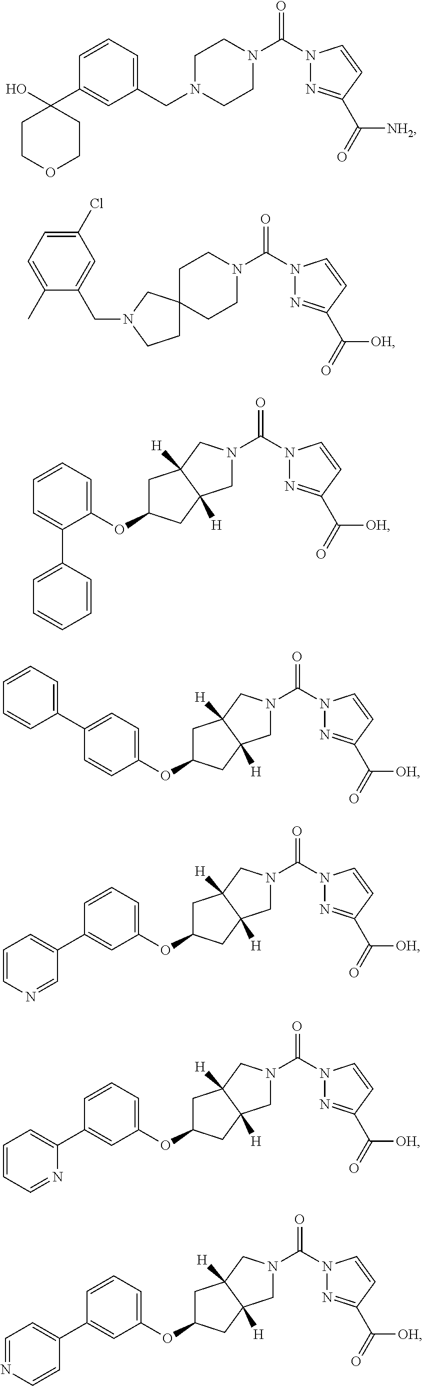 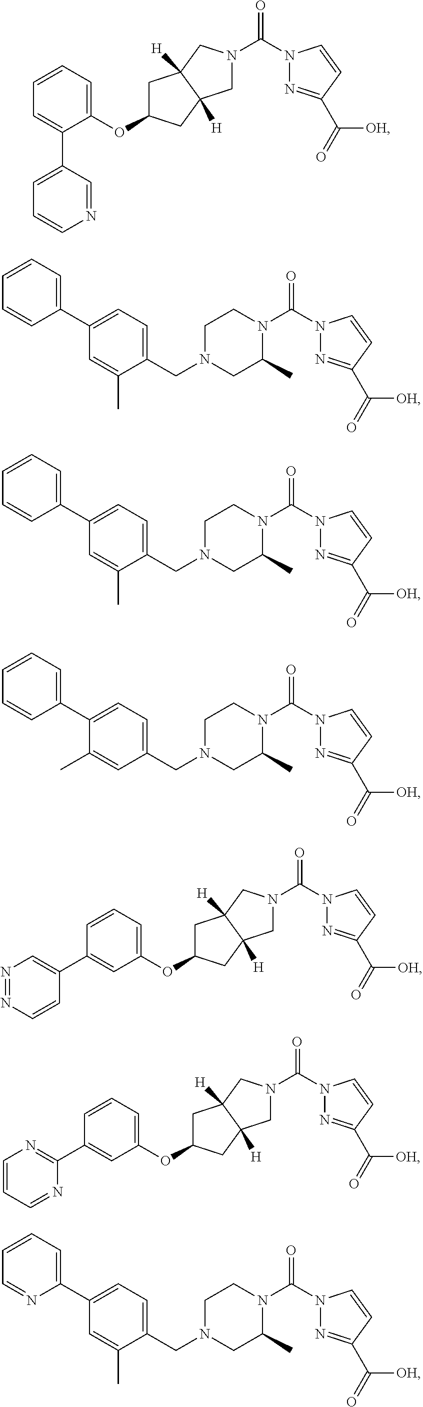 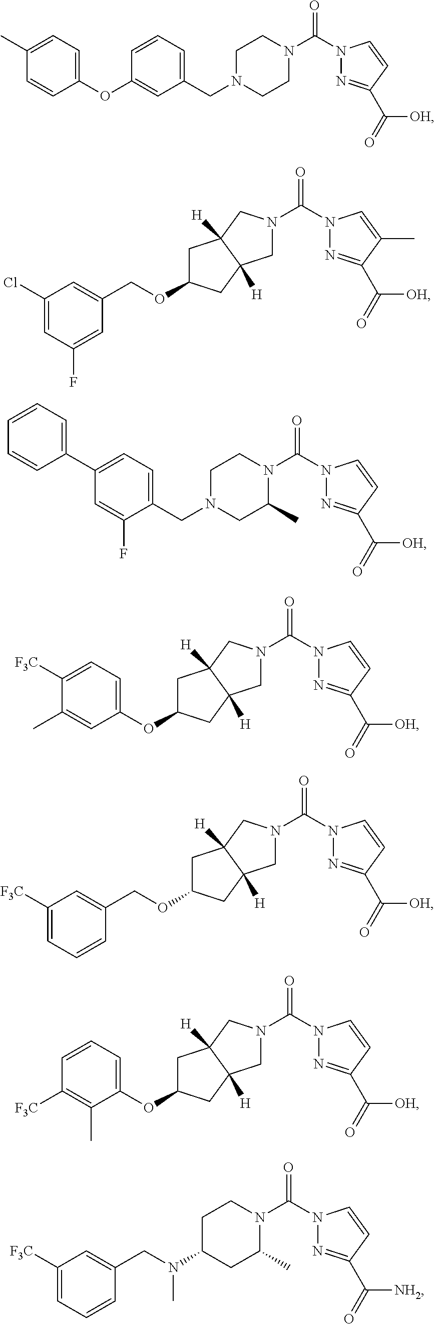 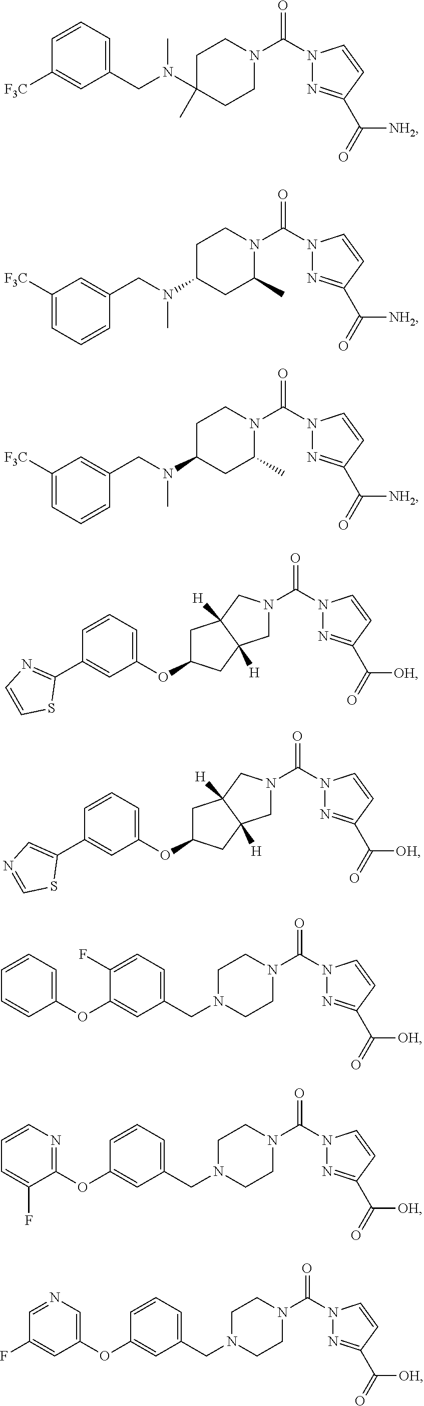 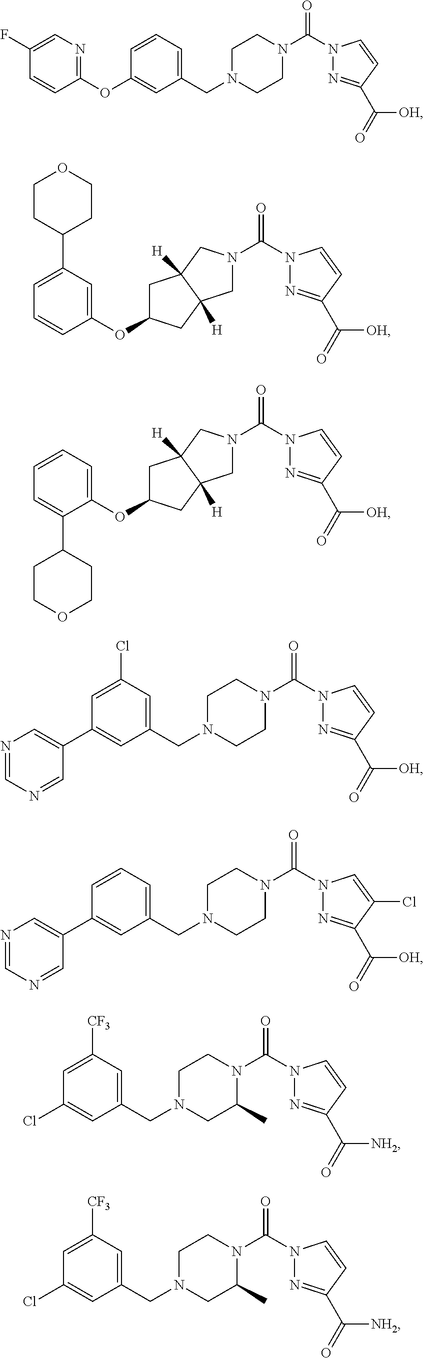 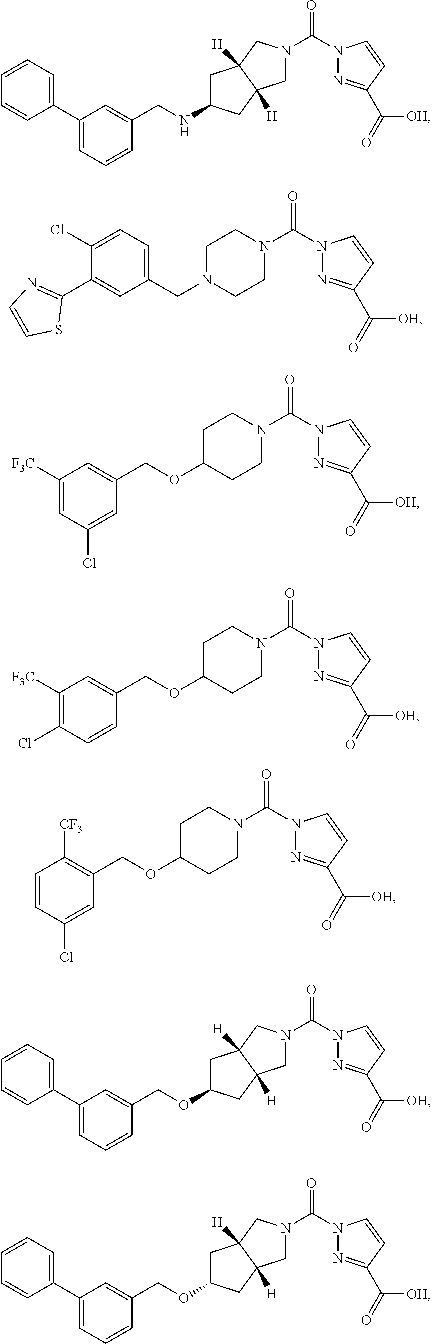 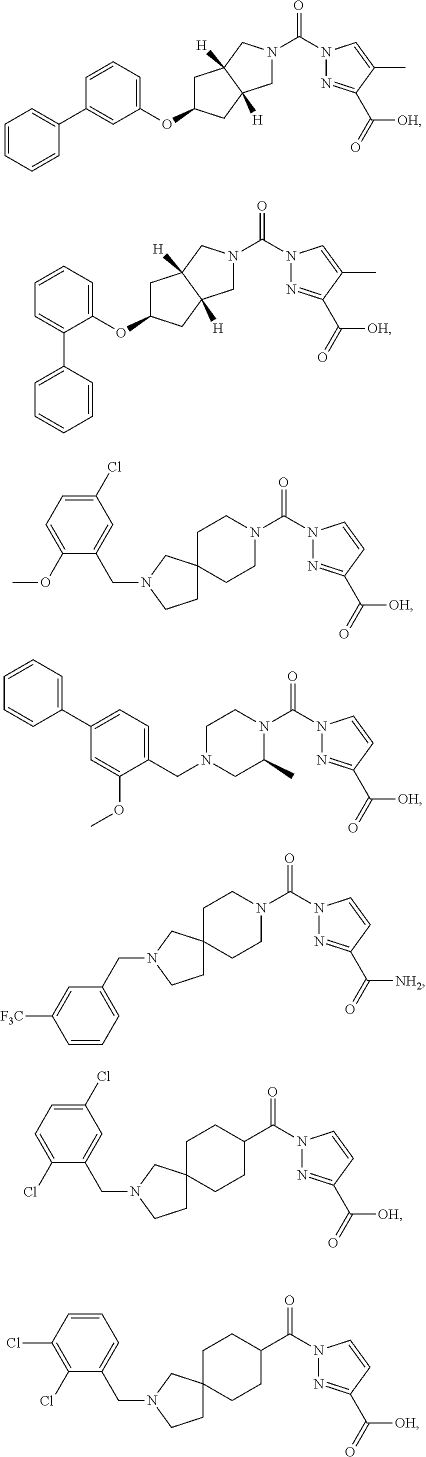 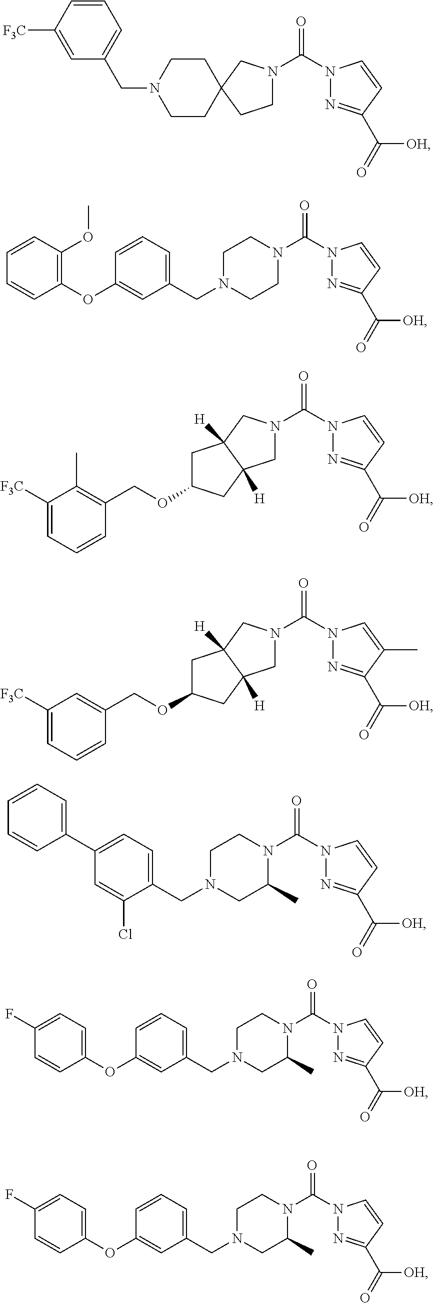 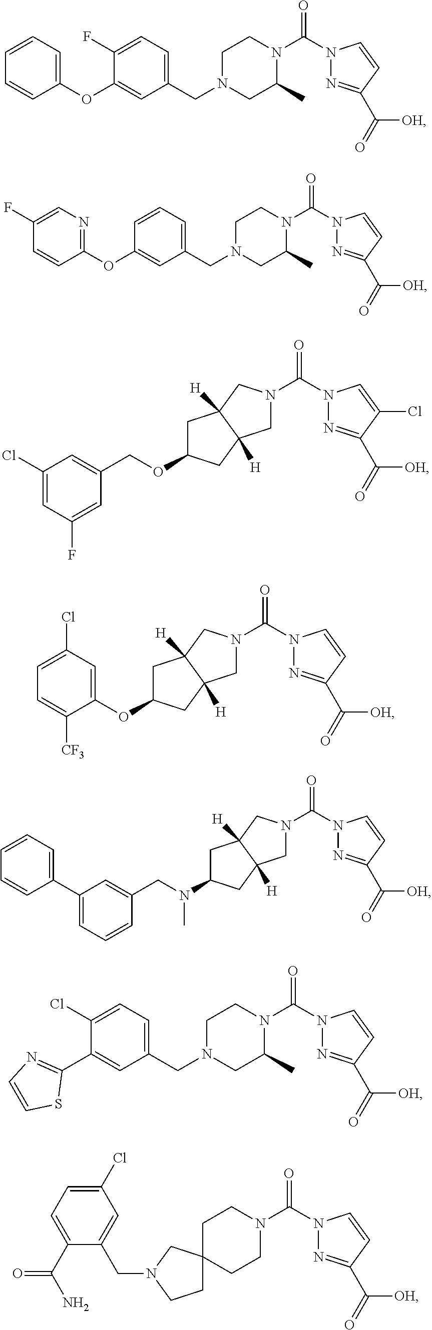 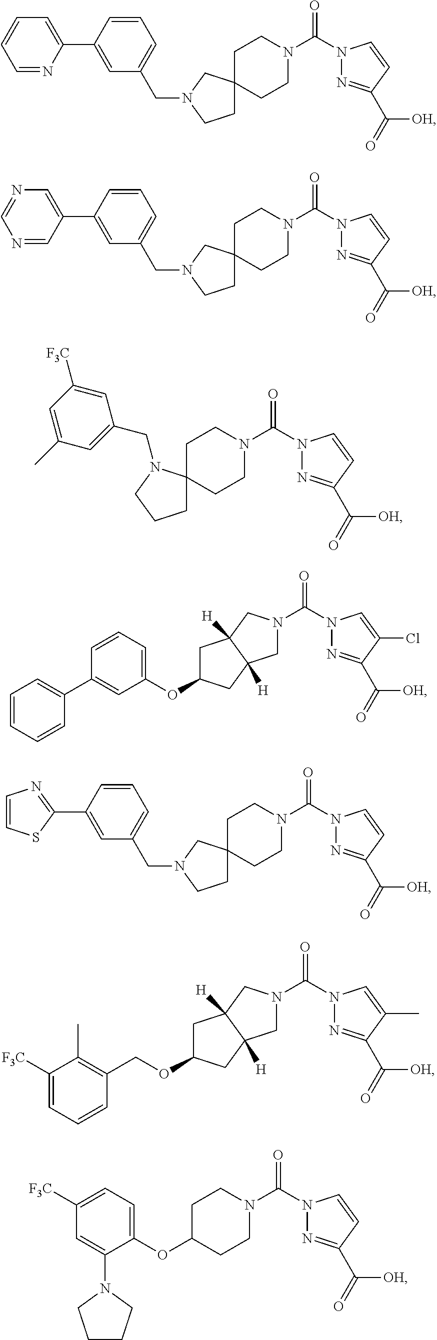 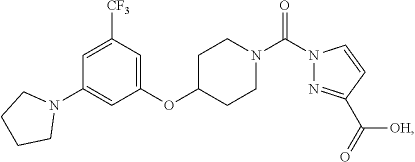 [SMILES: O=C(O)c1ccn(C(=O)N2CCC(Oc3cc(N4CCCC4)cc(C(F)(F)F)c3)CC2)n1] 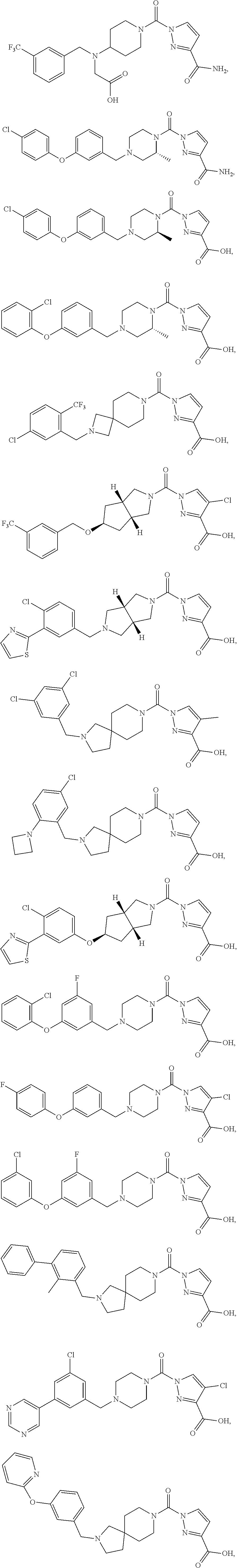 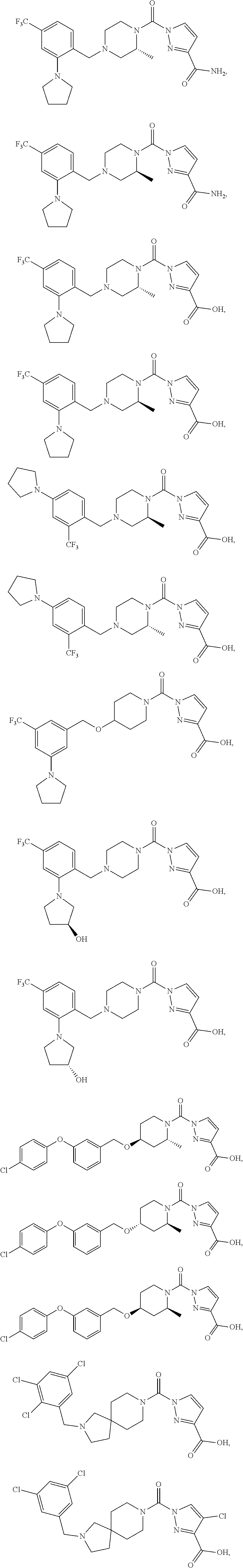 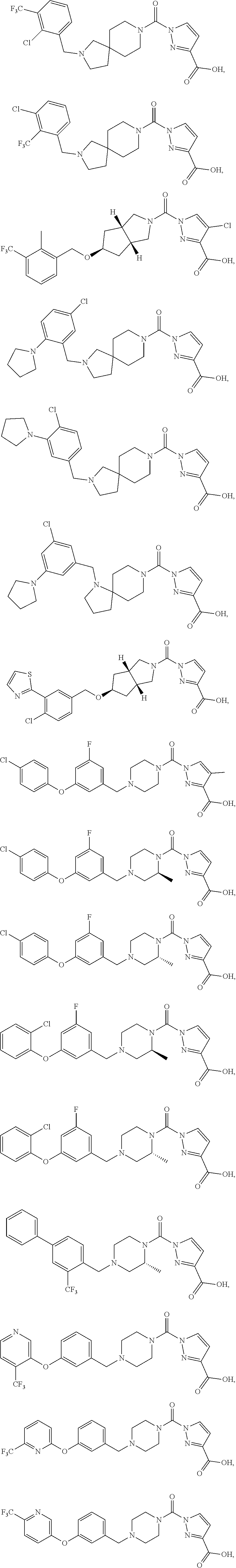 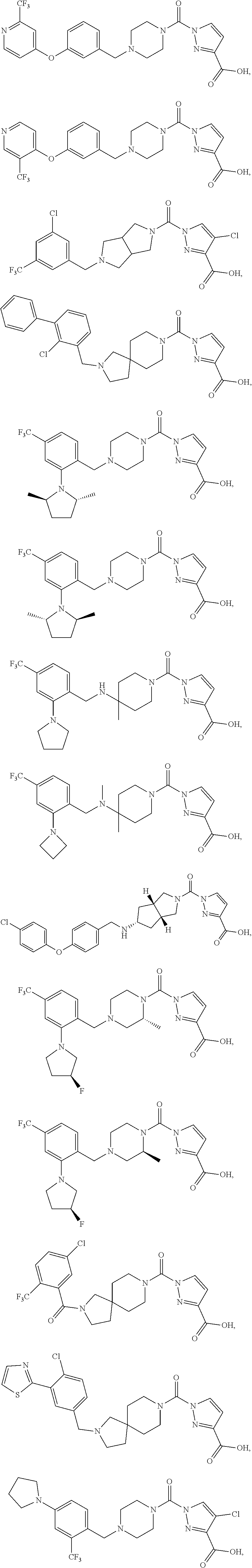 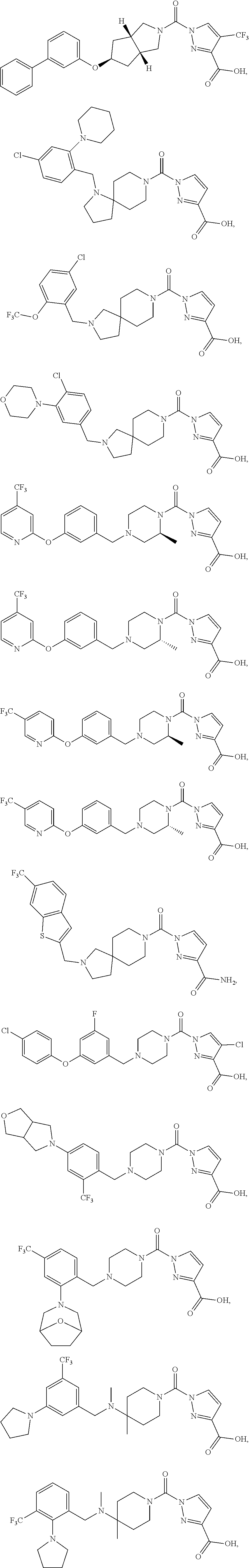 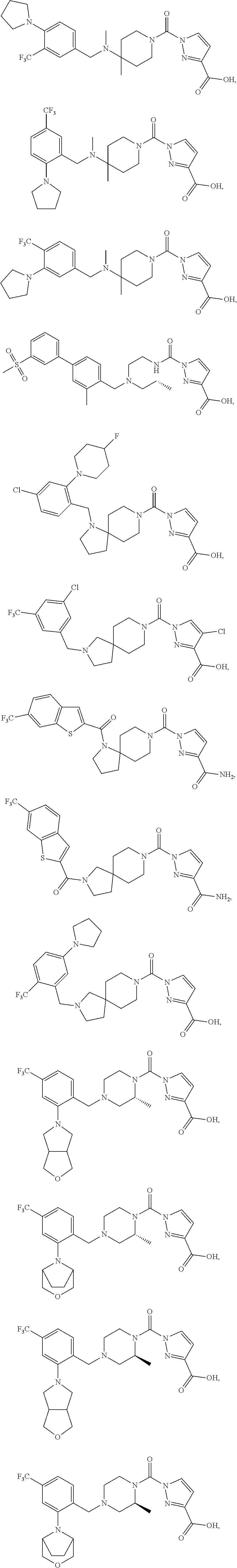 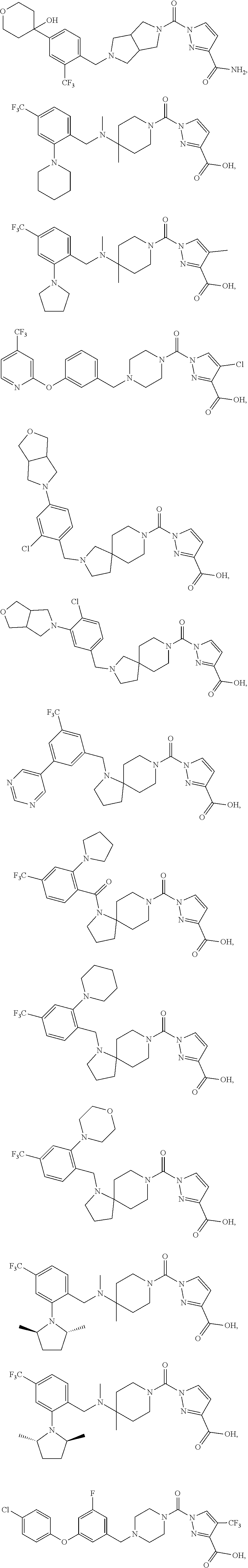 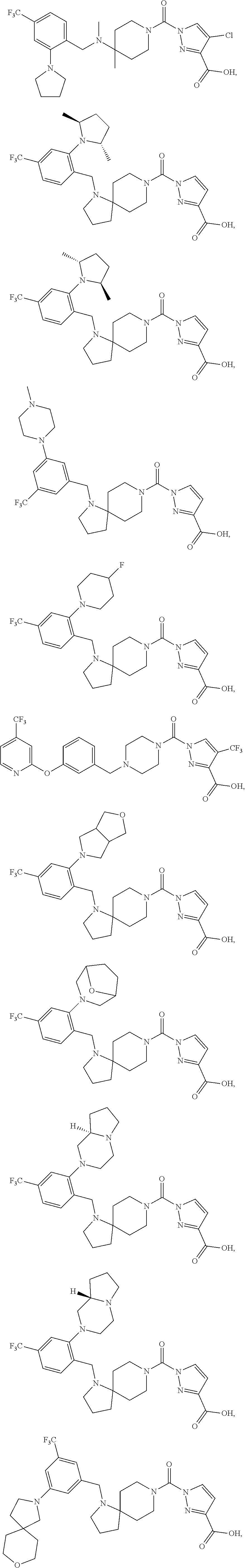 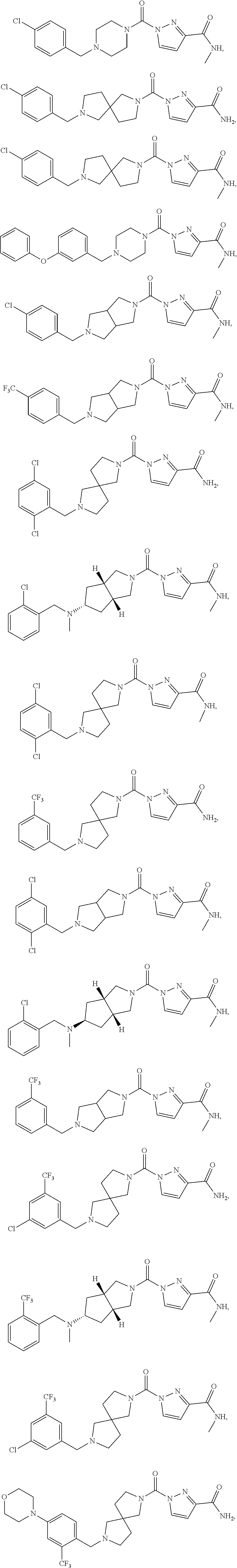 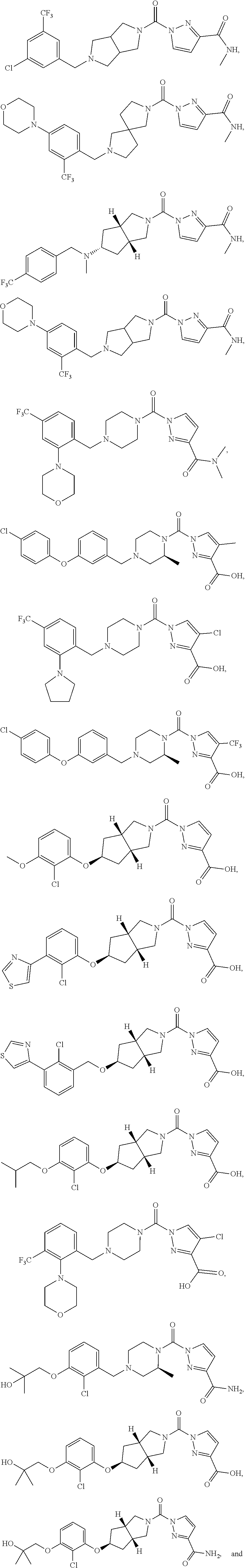 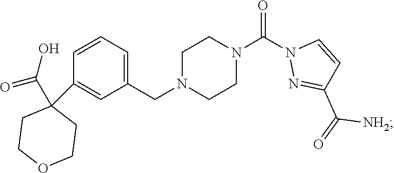 [SMILES: NC(=O)c1ccn(C(=O)N2CCN(Cc3cccc(C4(C(=O)O)CCOCC4)c3)CC2)n1]
or a solvate, hydrate, tautomer, N-oxide, stereoisomer, or pharmaceutically acceptable salt thereof.
